SEQUENCE LISTING (207 sequences)

SEQ ID NO: 1 - protein, 19 aa, synthetic construct
MEFGLSWLFLVAILKGVQC

SEQ ID NO: 2 - protein, 11 aa, synthetic construct
RASQDISNYLN

SEQ ID NO: 3 - protein, 7 aa, synthetic construct
YTSILHS

SEQ ID NO: 4 - protein, 9 aa, synthetic construct
QQGNTLPWT

SEQ ID NO: 5 - protein, 108 aa, synthetic construct
DIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKR

SEQ ID NO: 6 - protein, 10 aa, synthetic construct
GFAFSIYDMS

SEQ ID NO: 7 - protein, 17 aa, synthetic construct
YISSGGGTTYYPDTVKG

SEQ ID NO: 8 - protein, 14 aa, synthetic construct
HSGYGSSYGVLFAY

SEQ ID NO: 9 - protein, 122 aa, synthetic construct
EVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCAHSGYGSSYGVLFAYWGQGTLVTVSS

SEQ ID NO: 10 - protein, 15 aa, synthetic construct
GGGGSGGGGSGGGGS

SEQ ID NO: 11 - protein, 23 aa, synthetic construct
DIQMTQSPSSLSASVGDRVTITC

SEQ ID NO: 12 - protein, 15 aa, synthetic construct
WLQQKPGKAPKLLIY

SEQ ID NO: 13 - protein, 32 aa, synthetic construct
GVPSRFSGSGSGTEFTLTISSLQPEDFATYYC

SEQ ID NO: 14 - protein, 11 aa, synthetic construct
FGQGTKLEIKR

SEQ ID NO: 15 - protein, 25 aa, synthetic construct [sequence omitted]

SEQ ID NO: 16 - protein, 14 aa, synthetic construct
WVRQVPGKGLEWVS

SEQ ID NO: 17 - protein, 31 aa, synthetic construct
RFTISRDNSRNTLDLQMNSLRVEDTAVYYCA

SEQ ID NO: 18 - protein, 11 aa, synthetic construct
WGQGTLVTVSS

SEQ ID NO: 19 - protein, 19 aa, synthetic construct
MERHWIFLFLLSVTAGVHS

SEQ ID NO: 20 - protein, 12 aa, synthetic construct
QSVLYSANHKNY

SEQ ID NO: 21 - skipped

SEQ ID NO: 22 - protein, 8 aa, synthetic construct
HQYLSSWT

SEQ ID NO: 23 - protein, 112 aa, synthetic construct
DIQLTQSPSSLAVSAGENVTMSCKSSQSVLYSANHKNYLAWYQQKPGQSPKLLIYWASTRESGVPDRFTGSGSGTDFTLTISRVQVEDLAIYYCHQYLSSWTFGGGTKLEIK

SEQ ID NO: 24 - protein, 8 aa, synthetic construct
GYTFTSYW

SEQ ID NO: 25 - protein, 8 aa, synthetic construct
INPRNDYT

SEQ ID NO: 26 - protein, 9 aa, synthetic construct
ARRDITTFY

SEQ ID NO: 27 - protein, 116 aa, synthetic construct
QVQLQQSGAELVKPGASVKMSCKASGYTFTSYWLHWIKQRPGQGLEWIGYINPRNDYTEYNQKFKDKATLTADKSSSTAYMQLSSLTSEDSAVYYCARRDITTFYWGQGTTLTVSS

SEQ ID NO: 28 - protein, 15 aa, synthetic construct
GGGGSGGGGSGGGGS

SEQ ID NO: 29 - protein, 26 aa, synthetic construct
DIQLTQSPSSLAVSAGENVTMSCKSS

SEQ ID NO: 30 - protein, 17 aa, synthetic construct
LAWYQQKPGQSPKLLIY

SEQ ID NO: 31 - protein, 36 aa, synthetic construct
TRESGVPDRFTGSGSGTDFTLTISRVQVEDLAIYYC

SEQ ID NO: 32 - protein, 10 aa, synthetic construct
FGGGTKLEIK

SEQ ID NO: 33 - protein, 25 aa, synthetic construct
QVQLQQSGAELVKPGASVKMSCKAS

SEQ ID NO: 34 - protein, 17 aa, synthetic construct
LHWIKQRPGQGLEWIGY

SEQ ID NO: 35 - protein, 38 aa, synthetic construct
EYNQKFKDKATLTADKSSSTAYMQLSSLTSEDSAVYYC

SEQ ID NO: 36 - protein, 11 aa, synthetic construct
WGQGTTLTVSS

SEQ ID NO: 37 - protein, 22 aa, synthetic construct
MLLLVTSLLLCELPHPAFLLIP

SEQ ID NO: 38 - protein, 6 aa, synthetic construct
QDISKY

SEQ ID NO: 39 - skipped

SEQ ID NO: 40 - protein, 9 aa, synthetic construct
QQGNTLPYT

SEQ ID NO: 41 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 42 - protein, 8 aa, synthetic construct
GVSLPDYG

SEQ ID NO: 43 - protein, 7 aa, synthetic construct
IWGSETT

SEQ ID NO: 44 - protein, 14 aa, synthetic construct
AKHYYYGGSYAMDY

SEQ ID NO: 45 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 46 - protein, 18 aa, synthetic construct
GSTSGSGKPGSGEGSTKG

SEQ ID NO: 47 - protein, 26 aa, synthetic construct
DIQMTQTTSSLSASLGDRVTISCRAS

SEQ ID NO: 48 - protein, 17 aa, synthetic construct
LNWYQQKPDGTVKLLIY

SEQ ID NO: 49 - protein, 36 aa, synthetic construct
RLHSGVPSRFSGSGSGTDYSLTISNLEQEDIATYFC

SEQ ID NO: 50 - protein, 10 aa, synthetic construct
FGGGTKLEIT

SEQ ID NO: 51 - protein, 25 aa, synthetic construct
EVKLQESGPGLVAPSQSLSVTCTVS

SEQ ID NO: 52 - protein, 17 aa, synthetic construct
VSWIRQPPRKGLEWLGV

SEQ ID NO: 53 - protein, 38 aa, synthetic construct
YYNSALKSRLTIIKDNSKSQVFLKMNSLQTDDTAIYYC

SEQ ID NO: 54 - protein, 11 aa, synthetic construct
WGQGTSVTVSS

SEQ ID NO: 55 - protein, 19 aa, synthetic construct
MGWSCIILFLVATATGVHS

SEQ ID NO: 56 - protein, 7 aa, synthetic construct
SSVSYMH

SEQ ID NO: 57 - protein, 11 aa, synthetic construct
LLIYSTSNLAS

SEQ ID NO: 58 - protein, 7 aa, synthetic construct
HQWSSYP

SEQ ID NO: 59 - protein, 105 aa, synthetic construct
DIQMTQSPSSLSASVGDRVTITCSTSSSVSYMHWYQQKPGKAPRLLIYSTSNLASGVPSRFSGSGSGTDFTFTISSLQPEDIATYYCHQWSSYPTFGQGTKVEIK

SEQ ID NO: 60 - protein, 10 aa, synthetic construct
FTISSGYSWH

SEQ ID NO: 61 - protein, 13 aa, synthetic construct
WIGYIQYSGITNY

SEQ ID NO: 62 - protein, 13 aa, synthetic construct
AREDYDYHWYFDV

SEQ ID NO: 63 - protein, 120 aa, synthetic construct
QVQLQESGPGLVRPSQTLSLTCTVSGFTISSGYSWHWVRQPPGRGLEWIGYIQYSGITNYNPSLKSRVTMLVDTSKNQFSLRLSSVTAADTAVYYCAREDYDYHWYFDVWGQGSTVTVSS

SEQ ID NO: 64 - protein, 18 aa, synthetic construct
GSTSGSGKPGSGEGSTKG

SEQ ID NO: 65 - protein, 26 aa, synthetic construct
DIQMTQSPSSLSASVGDRVTITCSTS

SEQ ID NO: 66 - protein, 11 aa, synthetic construct
WYQQKPGKAPR

SEQ ID NO: 67 - protein, 32 aa, synthetic construct
GVPSRFSGSGSGTDFTFTISSLQPEDIATYYC

SEQ ID NO: 68 - protein, 11 aa, synthetic construct
TFGQGTKVEIK

SEQ ID NO: 69 - protein, 26 aa, synthetic construct
QVQLQESGPGLVRPSQTLSLTCTVSG

SEQ ID NO: 70 - protein, 11 aa, synthetic construct
WVRQPPGRGLE

SEQ ID NO: 71 - protein, 36 aa, synthetic construct
NPSLKSRVTMLVDTSKNQFSLRLSSVTAADTAVYYC

SEQ ID NO: 72 - protein, 11 aa, synthetic construct
WGQGSTVTVSS

SEQ ID NO: 73 - protein, 55 aa, synthetic construct
FVPVFLPAKPTTTPAPRPPTPAPTIASQPLSLRPEACRPAAGGAVHTRGLDFACD

SEQ ID NO: 74 - protein, 28 aa, synthetic construct
IYIWAPLAGTCGVLLLSLVITLYCNHRN

SEQ ID NO: 75 - protein, 15 aa, synthetic construct
EPKSPDKTHTCPPCP

SEQ ID NO: 76 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 77 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 39 aa, synthetic construct
IEVMYPPPYLDNEKSNGTIIHVKGKHLCPSPLFPGPSKP

SEQ ID NO: 79 - protein, 27 aa, synthetic construct
FWVLVVVGGVLACYSLLVTVAFIIFWV

SEQ ID NO: 80 - protein, 47 aa, synthetic construct
RFSVVKRGRKKLLYIFKQPFMRPVQTTQEEDGCSCRFPEEEEGGCEL

SEQ ID NO: 81 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 22 aa, synthetic construct
SGQVLLESNIKVLPTWSTPVQP

SEQ ID NO: 83 - protein, 22 aa, synthetic construct
MALIVLGGVAGLLLFIGLGIFF

SEQ ID NO: 84 - protein, 40 aa, synthetic construct
CVRCRHRRRQAERMSQIKRLLSEKKTCQCPHRFQKTCSPI

SEQ ID NO: 85 - protein, 45 aa, synthetic construct
TTTPAPRPPTPAPTIASQPLSLRPEACRPAAGGAVHTRGLDFACD

SEQ ID NO: 86 - protein, 21 aa, synthetic construct
IYIWAPLAGTCGVLLLSLVIT

SEQ ID NO: 87 - protein, 32 aa, synthetic construct
LYCNHRNRRRVCKCPRPVVKSGDKPSLSARYV

SEQ ID NO: 88 - protein, 22 aa, synthetic construct
SGQVLLESNIKVLPTWSTPVQP

SEQ ID NO: 89 - skipped

SEQ ID NO: 90 - protein, 4 aa, synthetic construct
CQCP

SEQ ID NO: 91 - protein, 4 aa, synthetic construct
CKCP

SEQ ID NO: 92 - protein, 50 aa, synthetic construct
KNRKAKAKPVTRGAGAGGRQRGQNKERPPPVPNPDYEPIRKGQRDLYSGL

SEQ ID NO: 93 - protein, 8 aa, synthetic construct
RKXQRXXY

SEQ ID NO: 94 - protein, 8 aa, synthetic construct
RKGQRDLY

SEQ ID NO: 95 - protein, 41 aa, synthetic construct
RSKRSRLLHSDYMNMTPRRPGPTRKHYQPYAPPRDFAAYRS

SEQ ID NO: 96 - protein, 6 aa, synthetic construct
RSKRSR

SEQ ID NO: 97 - protein, 8 aa, synthetic construct
RRPGPTRK

SEQ ID NO: 98 - protein, 5 aa, synthetic construct
PYAPP

SEQ ID NO: 99 - protein, 72 aa, synthetic construct
NSRRRCGQKKKLVINSGNGAVEDRKPSGLNGEASKSQEMVHLVNKESSETPDQFMTADETRNLQNVDMKIGV

SEQ ID NO: 100 - protein, 31 aa, synthetic construct
NSRRRCGQKKKLVINSGNGAVEDRKPSGLNG

SEQ ID NO: 101 - protein, 63 aa, synthetic construct
KKGKLPCRRSGKQEITLPPSRKSELVVEVKSDKLPEEMGLLQGSSGDKRAPGDQGEKYIDLRH

SEQ ID NO: 102 - protein, 25 aa, synthetic construct
KKGKLPCRRSGKQEITLPPSRKSEL

SEQ ID NO: 103 - protein, 509 aa, synthetic construct
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 104 - protein, 510 aa, synthetic construct
MEFGLSWLFLVAILKGVQCEVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVSSGGGGSGGGGSGGGGSDIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKRAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 686 aa, synthetic construct
MEFGLSWLFLVAILKGVQCEVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVSSGGGGSGGGGSGGGGSDIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKRAAAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 356 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVAAAASGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 107 - protein, 349 aa, synthetic construct
MERHWIFLFLLSVTAGVHSQVQLQQSGAELVKPGASVKMSCKASGYTFTSYWLHWIKQRPGQGLEWIGYINPRNDYTEYNQKFKDKATLTADKSSSTAYMQLSSLTSEDSAVYYCARRDITTFYWGQGTTLTVSSGGGGSGGGGSGGGGSDIQLTQSPSSLAVSAGENVTMSCKSSQSVLYSANHKNYLAWYQQKPGQSPKLLIYWASTRESGVPDRFTGSGSGTDFTLTISRVQVEDLAIYYCHQYLSSWTFGGGTKLEIKAAASGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 108 - protein, 350 aa, synthetic construct
MGWSCIILFLVATATGVHSDIQMTQSPSSLSASVGDRVTITCSTSSSVSYMHWYQQKPGKAPRLLIYSTSNLASGVPSRFSGSGSGTDFTFTISSLQPEDIATYYCHQWSSYPTFGQGTKVEIKGSTSGSGKPGSGEGSTKGQVQLQESGPGLVRPSQTLSLTCTVSGFTISSGYSWHWVRQPPGRGLEWIGYIQYSGITNYNPSLKSRVTMLVDTSKNQFSLRLSSVTAADTAVYYCAREDYDYHWYFDVWGQGSTVTVSSGAAASGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 109 - protein, 1059 aa, synthetic construct
MEFGLSWLFLVAILKGVQCEVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVSSGGGGSGGGGSGGGGSDIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKRAAAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREFGGGEGRGSLLTCGDVEENPGPRMERHWIFLFLLSVTAGVHSQVQLQQSGAELVKPGASVKMSCKASGYTFTSYWLHWIKQRPGQGLEWIGYINPRNDYTEYNQKFKDKATLTADKSSSTAYMQLSSLTSEDSAVYYCARRDITTFYWGQGTTLTVSSGGGGSGGGGSGGGGSDIQLTQSPSSLAVSAGENVTMSCKSSQSVLYSANHKNYLAWYQQKPGQSPKLLIYWASTRESGVPDRFTGSGSGTDFTLTISRVQVEDLAIYYCHQYLSSWTFGGGTKLEIKAAASGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 110 - DNA, 1533 bp, synthetic construct
  source 1..1533: mol_type = other DNA
atggagtttggcctgagctggctgttcctggtggccatcctgaagggcgtgcagtgtgaggtgcagctggtggagtctgggggaggcttggtccagcctggggggtccctgagactctcctgtgcagcctctggattcgctttcagtatctatgacatgtcttgggtccgccaggttccagggaaggggctggagtgggtctcatacattagtagtggtggtggtaccacctactatccagacactgtgaagggcagattcaccatctccagagacaattccaggaacactctggatcttcaaatgaacagtctgagagtcgaggacacggctgtctattattgtgcgagacatagtggctacggtagtagctacggggttttgtttgcttactggggccaaggaaccctggtcaccgtctcctcaggtggaggcggttcaggcggaggtggctctggcggtggcggatcggacatccagatgactcagtctccatcctccctgtctgcatctgtaggagacagagtcaccatcacctgcagggcaagtcaggacattagcaattatttaaactggcttcaacagaaaccagggaaagcccctaagctcctgatttactacacatcaatattacactcaggagtcccatcaaggttcagcggcagtggatctgggacagagttcactctcacaatcagcagcctgcagcctgaagattttgcaacttattactgtcaacagggtaatacgcttccgtggacgtttggccaggggaccaaactggaaatcaaacgtgcggccgcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 111 - DNA, 2061 bp, synthetic construct
  source 1..2061: mol_type = other DNA
atggagtttggcctgagctggctgttcctggtggccatcctgaagggcgtgcagtgtgaggtgcagctggtggagtctgggggaggcttggtccagcctggggggtccctgagactctcctgtgcagcctctggattcgctttcagtatctatgacatgtcttgggtccgccaggttccagggaaggggctggagtgggtctcatacattagtagtggtggtggtaccacctactatccagacactgtgaagggcagattcaccatctccagagacaattccaggaacactctggatcttcaaatgaacagtctgagagtcgaggacacggctgtctattattgtgcgagacatagtggctacggtagtagctacggggttttgtttgcttactggggccaaggaaccctggtcaccgtctcctcaggtggaggcggttcaggcggaggtggctctggcggtggcggatcggacatccagatgactcagtctccatcctccctgtctgcatctgtaggagacagagtcaccatcacctgcagggcaagtcaggacattagcaattatttaaactggcttcaacagaaaccagggaaagcccctaagctcctgatttactacacatcaatattacactcaggagtcccatcaaggttcagcggcagtggatctgggacagagttcactctcacaatcagcagcctgcagcctgaagattttgcaacttattactgtcaacagggtaatacgcttccgtggacgtttggccaggggaccaaactggaaatcaaacgtgcggccgctgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaagccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaattttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 112 - DNA, 1071 bp, synthetic construct
  source 1..1071: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagtagcagcggccgcatccggccaagtgcttctggagagcaacatcaaggtactgccaacatggagcactcctgtacagccaatggccctcatcgttctcggaggtgttgcgggtcttttgctctttatcgggcttgggattttcttctgcgtgagatgtcgccatagaaggcgacaggcagaacgcatgagtcagatcaagcgcttgctgtccgagaagaaaacctgtcagtgtccccataggttccagaaaacttgcagccctatataa

SEQ ID NO: 113 - DNA, 1050 bp, synthetic construct
  source 1..1050: mol_type = other DNA
atggaaaggcactggatctttctcttcctgttgtcagtaactgcaggtgtgcactcgcaggtccagctgcagcagtcaggggctgaactggtgaaacctggggcctcagtgaagatgtcctgcaaggcttctggctacacctttactagctactggctgcactggataaaacagaggcctggacagggtctggaatggattggatacattaatcctaggaatgattatactgagtacaatcagaaattcaaggacaaggccacattgactgcagacaaatcctccagcacagcctatatgcaactgagcagcctgacatctgaggactctgcagtctattactgcgcgcgaagggatattactacgttctactggggccaaggcaccactctcacagtctcctcgggtggaggcggttcaggcggaggtggctctggcggtggcggatcagatatccagctgacccagtctccatcatctctggctgtgtctgcaggagaaaacgtcactatgagctgtaagtccagtcaaagtgttttatacagtgcaaatcacaagaactacttggcctggtaccagcagaaaccagggcagtctcctaaactgctgatctactgggcatccactagggaatctggtgtccctgatcgcttcacaggcagcggatctgggacagattttactcttaccatcagcagagtacaagttgaagacctggcaatttattattgtcaccaatacctctcctcgtggacgttcggtggagggaccaagctggagatcaaagcggccgcatccggccaagtgcttctggagagcaacatcaaggtactgccaacatggagcactcctgtacagccaatggccctcatcgttctcggaggtgttgcgggtcttttgctctttatcgggcttgggattttcttctgcgtgagatgtcgccatagaaggcgacaggcagaacgcatgagtcagatcaagcgcttgctgtccgagaagaaaacctgtcagtgtccccataggttccagaaaacttgcagccctatataa

SEQ ID NO: 114 - DNA, 1053 bp, synthetic construct
  source 1..1053: mol_type = other DNA
atgggatggagctgtatcatcctcttcctggtagcaacagctacaggcgtgcacagtgacatccagatgacccagagcccaagcagcctgagcgccagcgtgggtgacagagtgaccatcacctgtagtaccagctcaagtgtaagttacatgcactggtaccagcagaagccaggtaaggctccaaggctgctgatctacagcacatccaacctggcttctggtgtgccaagcagattcagcggtagcggtagcggtaccgacttcaccttcaccatcagcagcctccagccagaggacatcgccacctactactgccatcagtggagtagttatcccacgttcggccaagggaccaaggtggaaatcaaaggatctacttccggttcaggaaagcccgggagtggtgaaggtagcactaaaggccaggtccagctgcaggagagcggtccaggtcttgtgagacctagccagaccctgagcctgacctgcaccgtgtctggcttcaccatcagcagtggttatagctggcactgggtgagacagccacctggacgaggtcttgagtggattggatacatacagtacagtggtatcactaactacaacccctctctcaaaagtagagtgacaatgctggtagacaccagcaagaaccagttcagcctgagactcagcagcgtgacagccgccgacaccgcggtctattattgtgcaagagaagactatgattaccactggtacttcgatgtctggggtcaaggcagcacggtcaccgtctcctcaggtgcggccgcttccggccaagtgcttctggagagcaacatcaaggtactgccaacatggagcactcctgtacagccaatggccctcatcgttctcggaggtgttgcgggtcttttgctctttatcgggcttgggattttcttctgcgtgagatgtcgccatagaaggcgacaggcagaacgcatgagtcagatcaagcgcttgctgtccgagaagaaaacctgtcagtgtccccataggttccagaaaacttgcagccctatataa

SEQ ID NO: 115 - DNA, 3180 bp, synthetic construct
  source 1..3180: mol_type = other DNA
atggaaaggcactggatctttctcttcctgttgtcagtaactgcaggtgtgcactcggaggtgcagctggtggagtctgggggaggcttggtccagcctggggggtccctgagactctcctgtgcagcctctggattcgctttcagtatctatgacatgtcttgggtccgccaggttccagggaaggggctggagtgggtctcatacattagtagtggtggtggtaccacctactatccagacactgtgaagggcagattcaccatctccagagacaattccaggaacactctggatcttcaaatgaacagtctgagagtcgaggacacggctgtctattattgtgcgagacatagtggctacggtagtagctacggggttttgtttgcttactggggccaaggaaccctggtcaccgtctcctcaggtggaggcggttcaggcggaggtggctctggcggtggcggatcggacatccagatgactcagtctccatcctccctgtctgcatctgtaggagacagagtcaccatcacctgcagggcaagtcaggacattagcaattatttaaactggcttcaacagaaaccagggaaagcccctaagctcctgatttactacacatcaatattacactcaggagtcccatcaaggttcagcggcagtggatctgggacagagttcactctcacaatcagcagcctgcagcctgaagattttgcaacttattactgtcaacagggtaatacgcttccgtggacgtttggccaggggaccaaactggaaatcaaacgtgcggccgctgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaagccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaattttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgaattcggcggcggagagggcagaggcagtctgctgacatgcggtgacgtggaagagaatcccggccctaggatggaaaggcactggatctttctcttcctgttgtcagtaactgcaggtgtgcactcgcaggtccagctgcagcagtcaggggctgaactggtgaaacctggggcctcagtgaagatgtcctgcaaggcttctggctacacctttactagctactggctgcactggataaaacagaggcctggacagggtctggaatggattggatacattaatcctaggaatgattatactgagtacaatcagaaattcaaggacaaggccacattgactgcagacaaatcctccagcacagcctatatgcaactgagcagcctgacatctgaggactctgcagtctattactgcgcgcgaagggatattactacgttctactggggccaaggcaccactctcacagtctcctcgggtggaggcggttcaggcggaggtggctctggcggtggcggatcagatatccagctgacccagtctccatcatctctggctgtgtctgcaggagaaaacgtcactatgagctgtaagtccagtcaaagtgttttatacagtgcaaatcacaagaactacttggcctggtaccagcagaaaccagggcagtctcctaaactgctgatctactgggcatccactagggaatctggtgtccctgatcgcttcacaggcagcggatctgggacagattttactcttaccatcagcagagtacaagttgaagacctggcaatttattattgtcaccaatacctctcctcgtggacgttcggtggagggaccaagctggagatcaaagcggccgcatccggccaagtgcttctggagagcaacatcaaggtactgccaacatggagcactcctgtacagccaatggccctcatcgttctcggaggtgttgcgggtcttttgctctttatcgggcttgggattttcttctgcgtgagatgtcgccatagaaggcgacaggcagaacgcatgagtcagatcaagcgcttgctgtccgagaagaaaacctgtcagtgtccccataggttccagaaaacttgcagccctatataa

SEQ ID NO: 116 - protein, 22 aa, synthetic construct
  source 1..22: note = CSFR2a leader sequence
MLLLVTSLLLCELPHPAFLLIP

SEQ ID NO: 117 - protein, 6 aa, synthetic construct
  source 1..6: note = human m971 scFv VL CDR1
QTIWSY

SEQ ID NO: 118 - skipped

SEQ ID NO: 119 - protein, 10 aa, synthetic construct
  source 1..10: note = human m971 scFv VL CDR3
QQSYSIPQTF

SEQ ID NO: 120 - protein, 107 aa, synthetic construct
  source 1..107: note = human m971 scFv VL
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 121 - protein, 10 aa, synthetic construct
  source 1..10: note = human m971 scFv VH CDR1
GDSVSSNSAA

SEQ ID NO: 122 - protein, 9 aa, synthetic construct
  source 1..9: note = human m971 scFv VH CDR2
TYYRSKWYN

SEQ ID NO: 123 - protein, 14 aa, synthetic construct
  source 1..14: note = human m971 scFv CDR3
AREVTGDLEDAFDI

SEQ ID NO: 124 - protein, 124 aa, synthetic construct
  source 1..124: note = human m971 scFv VH
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 125 - protein, 15 aa, synthetic construct
  source 1..15: note = Linker L1
GGGGSGGGGSGGGGS

SEQ ID NO: 126 - protein, 26 aa, synthetic construct [sequence omitted]

SEQ ID NO: 127 - protein, 17 aa, synthetic construct
  source 1..17: note = human m971 scFv LFR2
LNWYQQRPGKAPNLLIY

SEQ ID NO: 128 - protein, 36 aa, synthetic construct
  source 1..36: note = human m971 scFv LFR3
SLQSGVPSRFSGRGSGTDFTLTISSLQAEDFATYYC

SEQ ID NO: 129 - protein, 9 aa, synthetic construct
  source 1..9: note = human m971 scFv LFR4
GQGTKLEIK

SEQ ID NO: 130 - protein, 25 aa, synthetic construct
  source 1..25: note = human m971 scFv HFR1
QVQLQQSGPGLVKPSQTLSLTCAIS

SEQ ID NO: 131 - protein, 17 aa, synthetic construct
  source 1..17: note = human m971 scFv HFR2
WNWIRQSPSRGLEWLGR

SEQ ID NO: 132 - protein, 38 aa, synthetic construct
  source 1..38: note = human m971 scFv HFR3
DYAVSVKSRITINPDTSKNQFSLQLNSVTPEDTAVYYC

SEQ ID NO: 133 - protein, 11 aa, synthetic construct
  source 1..11: note = human m971 scFv HFR4
WGQGTMVTVSS

SEQ ID NO: 134 - protein, 474 aa, synthetic construct
  source 1..474: note = anti-CD22 CAR with m971 scFv and CD28 backbone [22-BBz]
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 116 aa, synthetic construct
  source 1..116: note = CD2 intracellular domain
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 136 - protein, 21 aa, synthetic construct
  source 1..21: note = CD44 transmembrane domain
WLIILASLLALALILAVCIAV

SEQ ID NO: 137 - protein, 25 aa, synthetic construct
  source 1..25: note = CD146 transmembrane domain
GVVIVAVIVCILVLAVLGAVLYFLY

SEQ ID NO: 138 - protein, 429 aa, synthetic construct
  source 1..429: note = 19-2 CoCAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 139 - protein, 363 aa, synthetic construct
  source 1..363: note = 19-3e CoCAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITKNRKAKAKPVTRGAGAGGRQRGQNKERPPPVPNPDYEPIRKGQRDLYSGL

SEQ ID NO: 140 - protein, 357 aa, synthetic construct
  source 1..357: note = 19-28 CoCAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 141 - protein, 385 aa, synthetic construct
  source 1..385: note = 19-44 CoCAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITNSRRRCGQKKKLVINSGNGAVEDRKPSGLNGEASKSQEMVHLVNKESSETPDQFMTADETRNLQNVDMKIGV

SEQ ID NO: 142 - protein, 376 aa, synthetic construct
  source 1..376: note = 19-146 CoCAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITKKGKLPCRRSGKQEITLPPSRKSELVVEVKSDKLPEEMGLLQGSSGDKRAPGDQGEKYIDLRH

SEQ ID NO: 143 - protein, 385 aa, synthetic construct
  source 1..385: note = 19-44v2 CoCAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 144 - protein, 380 aa, synthetic construct
  source 1..380: note = 19-146v2 CoCAR
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 145 - protein, 970 aa, synthetic construct
  source 1..970: note = 22-BBz/19-2
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 146 - protein, 904 aa, synthetic construct
  source 1..904: note = 22-BBz/19-3e
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITKNRKAKAKPVTRGAGAGGRQRGQNKERPPPVPNPDYEPIRKGQRDLYSGL

SEQ ID NO: 147 - protein, 898 aa, synthetic construct
  source 1..898: note = 22-BBz/19-28
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 148 - protein, 926 aa, synthetic construct
  source 1..926: note = 22-BBz/19-44
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITNSRRRCGQKKKLVINSGNGAVEDRKPSGLNGEASKSQEMVHLVNKESSETPDQFMTADETRNLQNVDMKIGV

SEQ ID NO: 149 - protein, 917 aa, synthetic construct
  source 1..917: note = 22-BBz/19-146
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITKKGKLPCRRSGKQEITLPPSRKSELVVEVKSDKLPEEMGLLQGSSGDKRAPGDQGEKYIDLRH

SEQ ID NO: 150 - protein, 926 aa, synthetic construct
  source 1..926: note = 22-BBz/19-44v2
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 151 - protein, 921 aa, synthetic construct
  source 1..921: note = 22-BBz/19-146v2
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 152 - DNA, 1491 bp, synthetic construct
  source 1..1491: mol_type = other DNA; note = anti-CD22 CAR with m971 scFv and CD28 backbone [22-BBz]
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggtggaggcggttcaggcggaggtggctctggcggtggcggatcggacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgttaa

SEQ ID NO: 153 - DNA, 1356 bp, synthetic construct
  source 1..1356: mol_type = other DNA; note = 19-2 CoCAR
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccaaacgtaaaaagcagagaagccgccgtaacgatgaagagctcgaaacccgcgctcatcgtgtggcgacggaggaacgcggtcggaagcctcaccagatcccagccagcacgccacagaatcctgctacatctcagcaccctccgccccctcccggccatagatcccaggcccccagccacaggccgcccccgcccgggcatagggtccaacatcaacctcagaagcgcccacccgctcccagcggaacccaggttcaccaacagaagggcccacctctgccacggccccgggttcagccaaagccaccccacggagccgctgaaaacagcctgagtcccagttcaaactaa

SEQ ID NO: 154 - DNA, 1158 bp, synthetic construct
  source 1..1158: mol_type = other DNA; note = 19-3e CoCAR
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgtag

SEQ ID NO: 155 - DNA, 1140 bp, synthetic construct
  source 1..1140: mol_type = other DNA; note = 19-28 CoCAR
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactcccagacgccctggccccacccgcaagcattaccagccctatgcccccccacgcgacttcgcagcctatagatcttag

SEQ ID NO: 156 - DNA, 1224 bp, synthetic construct
  source 1..1224: mol_type = other DNA; note = 19-44 CoCAR
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccaacagccgtcgcaggtgcggtcaaaagaaaaagctggtgatcaactctggcaacggagccgtcgaggacaggaagccttccggtctgaacggcgaggctagtaagtcccaggagatggtccacctcgtgaacaaggagagctccgaaacacccgaccagttcatgactgcggacgagacccgcaaccttcagaacgttgatatgaagatcggtgtgtaa

SEQ ID NO: 157 - DNA, 1197 bp, synthetic construct
  source 1..1197: mol_type = other DNA; note = 19-146 CoCAR
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccaagaaaggaaagctgccgtgtcgccgtagcggcaagcaggagatcactttgcctccctctcgcaaatcagagctggtggtcgaagtcaaaagcgacaagctccctgaagagatgggcttgctgcagggtagtagcggtgataagcgcgcccctggtgaccagggagagaagtacatcgacctgagacactaa

SEQ ID NO: 158 - DNA, 1224 bp, synthetic construct
  source 1..1224: mol_type = other DNA; note = 19-44v2 CoCAR
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattggcttattatcctggcgagtctcctggccctggcgttgatcctggcagtgtgtatcgccgtgaacagccgtcgcaggtgcggtcaaaagaaaaagctggtgatcaactctggcaacggagccgtcgaggacaggaagccttccggtctgaacggcgaggctagtaagtcccaggagatggtccacctcgtgaacaaggagagctccgaaacacccgaccagttcatgactgcggacgagacccgcaaccttcagaacgttgatatgaagatcggtgtgtaa

SEQ ID NO: 159 - DNA, 1209 bp, synthetic construct
  source 1..1209: mol_type = other DNA; note = 19-146v2 CoCAR
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatggcgttgtgatcgtggccgtgattgtgtgcattctggttttggctgtactgggcgctgtgctttactttttgtacaagaaaggaaagctgccgtgtcgccgtagcggcaagcaggagatcactttgcctccctctcgcaaatcagagctggtggtcgaagtcaaaagcgacaagctccctgaagagatgggcttgctgcagggtagtagcggtgataagcgcgcccctggtgaccagggagagaagtacatcgacctgagacactaa

SEQ ID NO: 160 - DNA, 2913 bp, synthetic construct
  source 1..2913: mol_type = other DNA; note = 22-BBz/19-2
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggtggaggcggttcaggcggaggtggctctggcggtggcggatcggacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccaaacgtaaaaagcagagaagccgccgtaacgatgaagagctcgaaacccgcgctcatcgtgtggcgacggaggaacgcggtcggaagcctcaccagatcccagccagcacgccacagaatcctgctacatctcagcaccctccgccccctcccggccatagatcccaggcccccagccacaggccgcccccgcccgggcatagggtccaacatcaacctcagaagcgcccacccgctcccagcggaacccaggttcaccaacagaagggcccacctctgccacggccccgggttcagccaaagccaccccacggagccgctgaaaacagcctgagtcccagttcaaactaa

SEQ ID NO: 161 - DNA, 2715 bp, synthetic construct
  source 1..2715: mol_type = other DNA; note = 22-BBz/19-3e
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggtggaggcggttcaggcggaggtggctctggcggtggcggatcggacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgtag

SEQ ID NO: 162 - DNA, 2697 bp, synthetic construct
  source 1..2697: mol_type = other DNA; note = 22-BBz/19-28
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggtggaggcggttcaggcggaggtggctctggcggtggcggatcggacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactcccagacgccctggccccacccgcaagcattaccagccctatgcccccccacgcgacttcgcagcctatagatcttag

SEQ ID NO: 163 - DNA, 2781 bp, synthetic construct
  source 1..2781: mol_type = other DNA; note = 22-BBz/19-44
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggtggaggcggttcaggcggaggtggctctggcggtggcggatcggacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccaacagccgtcgcaggtgcggtcaaaagaaaaagctggtgatcaactctggcaacggagccgtcgaggacaggaagccttccggtctgaacggcgaggctagtaagtcccaggagatggtccacctcgtgaacaaggagagctccgaaacacccgaccagttcatgactgcggacgagacccgcaaccttcagaacgttgatatgaagatcggtgtgtaa

SEQ ID NO: 164 - DNA, 2754 bp, synthetic construct
  source 1..2754: mol_type = other DNA; note = 22-BBz/19-146
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggtggaggcggttcaggcggaggtggctctggcggtggcggatcggacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccaagaaaggaaagctgccgtgtcgccgtagcggcaagcaggagatcactttgcctccctctcgcaaatcagagctggtggtcgaagtcaaaagcgacaagctccctgaagagatgggcttgctgcagggtagtagcggtgataagcgcgcccctggtgaccagggagagaagtacatcgacctgagacactaa

SEQ ID NO: 165 - DNA, 2781 bp, synthetic construct
  source 1..2781: mol_type = other DNA; note = 22-BBz/19-44v2
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggtggaggcggttcaggcggaggtggctctggcggtggcggatcggacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattggcttattatcctggcgagtctcctggccctggcgttgatcctggcagtgtgtatcgccgtgaacagccgtcgcaggtgcggtcaaaagaaaaagctggtgatcaactctggcaacggagccgtcgaggacaggaagccttccggtctgaacggcgaggctagtaagtcccaggagatggtccacctcgtgaacaaggagagctccgaaacacccgaccagttcatgactgcggacgagacccgcaaccttcagaacgttgatatgaagatcggtgtgtaa

SEQ ID NO: 166 - DNA, 2766 bp, synthetic construct
  source 1..2766: mol_type = other DNA; note = 22-BBz/19-146v2
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggtggaggcggttcaggcggaggtggctctggcggtggcggatcggacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatggcgttgtgatcgtggccgtgattgtgtgcattctggttttggctgtactgggcgctgtgctttactttttgtacaagaaaggaaagctgccgtgtcgccgtagcggcaagcaggagatcactttgcctccctctcgcaaatcagagctggtggtcgaagtcaaaagcgacaagctccctgaagagatgggcttgctgcagggtagtagcggtgataagcgcgcccctggtgaccagggagagaagtacatcgacctgagacactaa

SEQ ID NO: 167 - protein, 19 aa, synthetic construct
  source 1..19: note = anti-HSV IGHV3-23 leader sequence
MEFGLSWLFLVAILKGVQC

SEQ ID NO: 168 - protein, 11 aa, synthetic construct
  source 1..11: note = anti-HSV VL CDR1
QSIVHSNGNTY

SEQ ID NO: 169 - skipped

SEQ ID NO: 170 - protein, 9 aa, synthetic construct
  source 1..9: note = anti-HSV CDR3
FQGSHVPWS

SEQ ID NO: 171 - protein, 112 aa, synthetic construct
  source 1..112: note = anti-HSV VL
DIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWSFGQGTKLEIK

SEQ ID NO: 172 - protein, 10 aa, synthetic construct
  source 1..10: note = anti-HSV VH CDR1
GFSLSTSGMS

SEQ ID NO: 173 - protein, 7 aa, synthetic construct
  source 1..7: note = anti-HSV VH CDR2
IWWNNDK

SEQ ID NO: 174 - protein, 14 aa, synthetic construct
  source 1..14: note = anti-HSV VH CDR3
ARIYYGYRPYAMDY

SEQ ID NO: 175 - protein, 122 aa, synthetic construct
  source 1..122: note = anti-HSV - VH
QVTLKESGPALVKPTQTLTLTCTFSGFSLSTSGMSVGWIRQPPGKALEWLAHIWWNNDKYYKPALKSRLTISKDTSKNQVVLTMTNMDPVDTATYYCARIYYGYRPYAMDYWGQGTLVTVSS

SEQ ID NO: 176 - protein, 15 aa, synthetic construct
  source 1..15: note = linker L1
GGGGSGGGGSGGGGS

SEQ ID NO: 177 - protein, 26 aa, synthetic construct
  source 1..26: note = anti-HSV LFR1
DIVMTQTPLSLPVTPGEPASISCRSS

SEQ ID NO: 178 - protein, 17 aa, synthetic construct
  source 1..17: note = anti-HSV LFR2
LEWYLQKPGQSPQLLIY

SEQ ID NO: 179 - protein, 36 aa, synthetic construct
  source 1..36: note = anti-HSV LFR3
NRFSGVPDRFSGSGSGTDFTLKISRVEAEDVGVYYC

SEQ ID NO: 180 - protein, 10 aa, synthetic construct
  source 1..10: note = anti-HSV LFR4
FGQGTKLEIK

SEQ ID NO: 181 - protein, 25 aa, synthetic construct
  source 1..25: note = anti-HSV HFR1
QVTLKESGPALVKPTQTLTLTCTFS

SEQ ID NO: 182 - protein, 17 aa, synthetic construct
  source 1..17: note = anti-HSV HFR2
VGWIRQPPGKALEWLAH

SEQ ID NO: 183 - protein, 38 aa, synthetic construct
  source 1..38: note = anti-HSV HFR3
YYKPALKSRLTISKDTSKNQVVLTMTNMDPVDTATYYC

SEQ ID NO: 184 - protein, 11 aa, synthetic construct
  source 1..11: note = anti-HSV HFR4
WGQGTLVTVSS

SEQ ID NO: 185 - protein, 669 aa, synthetic construct
  source 1..669: note = anti-CD22 CAR with m971 scFv and IgG1-Fc spacer [m971-Fc-BBz]
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAEPKSPDKTHTCPPCPAPPVAGPSVFLFPPKPKDTLMIARTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 186 - protein, 474 aa, synthetic construct
  source 1..474: note = anti-HSV gB CAR with CD28 hinge/transmembrane domains [aHSV-BBz]
QVTLKESGPALVKPTQTLTLTCTFSGFSLSTSGMSVGWIRQPPGKALEWLAHIWWNNDKYYKPALKSRLTISKDTSKNQVVLTMTNMDPVDTATYYCARIYYGYRPYAMDYWGQGTLVTVSSGGGGSGGGGSGGGGSDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWSFGQGTKLEIKIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 187 - protein, 470 aa, synthetic construct
  source 1..470: note = anti-HER CAR with CD28 hinge/transmembrane domains [aHER2-BBz]
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 188 - protein, 357 aa, synthetic construct
  source 1..357: note = SGIII-28 CoCAR
EVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVSSGGGGSGGGGSGGGGSDIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 189 - protein, 385 aa, synthetic construct
  source 1..385: note = SGIII-44v2 CoCAR
EVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVSSGGGGSGGGGSGGGGSDIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKGSTTTPAPRPPTPAPTIASQPLSLRPEACRPAAGGAVHTRGLDFACDWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 190 - protein, 380 aa, synthetic construct
  source 1..380: note = SGIII-146v2 CoCAR
EVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVSSGGGGSGGGGSGGGGSDIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKGSTTTPAPRPPTPAPTIASQPLSLRPEACRPAAGGAVHTRGLDFACDGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 191 - protein, 898 aa, synthetic construct
  source 1..898: note = 22-BBz(xxx)/19-28
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLFNELNLGRREEFDVLDKRRGRDPEMGGKPRRKNPQEGLFNELQKDKMAEAFSEIGMKGERRRGKGHDGLFQGLSTATKDTFDALHMQALPPRTGGGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 192 - protein, 898 aa, synthetic construct
  source 1..898: note = aHSV-BBz(xxx)/19-28
MEFGLSWLFLVAILKGVQCQVTLKESGPALVKPTQTLTLTCTFSGFSLSTSGMSVGWIRQPPGKALEWLAHIWWNNDKYYKPALKSRLTISKDTSKNQVVLTMTNMDPVDTATYYCARIYYGYRPYAMDYWGQGTLVTVSSGGGGSGGGGSGGGGSDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWSFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLFNELNLGRREEFDVLDKRRGRDPEMGGKPRRKNPQEGLFNELQKDKMAEAFSEIGMKGERRRGKGHDGLFQGLSTATKDTFDALHMQALPPRTGGGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 193 - protein, 926 aa, synthetic construct
  source 1..926: note = HSV-BBz/19-44v2
MEFGLSWLFLVAILKGVQCQVTLKESGPALVKPTQTLTLTCTFSGFSLSTSGMSVGWIRQPPGKALEWLAHIWWNNDKYYKPALKSRLTISKDTSKNQVVLTMTNMDPVDTATYYCARIYYGYRPYAMDYWGQGTLVTVSSGGGGSGGGGSGGGGSDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWSFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 194 - protein, 921 aa, synthetic construct
  source 1..921: note = HSV-BBz/19-146v2
MEFGLSWLFLVAILKGVQCQVTLKESGPALVKPTQTLTLTCTFSGFSLSTSGMSVGWIRQPPGKALEWLAHIWWNNDKYYKPALKSRLTISKDTSKNQVVLTMTNMDPVDTATYYCARIYYGYRPYAMDYWGQGTLVTVSSGGGGSGGGGSGGGGSDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWSFGQGTKLEIKAAAIEVMYPPPYLDNEKSNGTIIHVKGKHLCPSPLFPGPSKPFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 195 - protein, 1090 aa, synthetic construct
  source 1..1090: note = m971-Fc-BBz/SGIII-28
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAEPKSPDKTHTCPPCPAPPVAGPSVFLFPPKPKDTLMIARTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMEFGLSWLFLVAILKGVQCEVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVSSGGGGSGGGGSGGGGSDIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 196 - protein, 1118 aa, synthetic construct
  source 1..1118: note = m971-Fc-BBz/SGIII-44v2
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAEPKSPDKTHTCPPCPAPPVAGPSVFLFPPKPKDTLMIARTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMEFGLSWLFLVAILKGVQCEVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVSSGGGGSGGGGSGGGGSDIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKGSTTTPAPRPPTPAPTIASQPLSLRPEACRPAAGGAVHTRGLDFACDWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 197 - protein, 1113 aa, synthetic construct
  source 1..1113: note = m971-Fc-BBz/SGIII-146v2
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAEPKSPDKTHTCPPCPAPPVAGPSVFLFPPKPKDTLMIARTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTGGGEGRGSLLTCGDVEENPGPRMEFGLSWLFLVAILKGVQCEVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVSSGGGGSGGGGSGGGGSDIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKGSTTTPAPRPPTPAPTIASQPLSLRPEACRPAAGGAVHTRGLDFACDGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 198 - DNA, 1491 bp, synthetic construct
  source 1..1491: mol_type = other DNA; note = anti-HSV gB CAR [aHSV-BBz]
atggagtttggcctgagctggctgttcctggtggccatcctgaagggcgtgcagtgtcaggttactctgaaagaatctggtccggccctggttaaaccgactcagactctgaccctgacgtgtactttctccggtttcagcctgtctacctccggtatgtctgttggttggattcgccaaccaccgggtaaagcactggaatggctggcgcacatctggtggaacaacgacaaatactataaaccggcactgaaatcccgtctgacgatctccaaagacacttctaagaaccaggttgttctgaccatgaccaacatggacccggtcgacaccgcaacctattactgcgcgcgtatctattacggttaccgtccgtatgcaatggattattggggtcagggtacgctggtcaccgtctcgagtggtggaggtggctcagggggcggaggcagcggaggcgggggcagcgacatcgttatgacccaaactccgctgtctctgccggttactccgggtgaaccggcgtctatctcctgtcgttcctcccagtccatcgttcactccaacggcaatacttacctggagtggtacctgcaaaaaccgggccaatctccacagctgctgatctacaaagttagcaaccgtttttctggcgtaccggatcgtttttctggttccggctctggtaccgacttcaccctgaagatttctcgtgtagaagctgaagatgtaggtgtgtactattgcttccagggctctcatgtaccgtggtctttcggccagggcaccaaactggagattaaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgttag

SEQ ID NO: 199 - DNA, 1470 bp, synthetic construct
  source 1..1470: mol_type = other DNA; note = anti-HER2 CAR [aHER2-BBz]
atggagtttggcctgagctggctgttcctggtggccatcctgaagggcgtgcagtgtgatatccagatgacccagagccctagctctctgagcgcctccgtgggagaccgcgtcacgatcacatgtcgcgcttcccaggacgtgaacaccgcggtagcctggtaccagcaaaagcccggcaaggcgcccaagctcctgatttactccgcctctttcctgtacagcggcgtgccaagccgtttcagcgggagccgtagcggcacggacttcaccctgactattagctccctccagccggaggatttcgcaacgtattactgccaacagcactacaccactccccctacttttggacagggcaccaaggtcgaaatcaagggctccaccagcgggggaggctctggcggagggagcggcggtgggggaagctctgaagtacagctcgtcgaatccgggggagggctggtccagcccggcggttctctgaggctgagttgcgcggcctccggatttaacatcaaagacacttacatccactgggtgaggcaggctccagggaagggtctggagtgggtggcgcgcatctatccgaccaacggctacacccgctatgctgactccgttaaggggcggtttaccatctccgctgacacctccaagaacactgcttacctgcaaatgaactccctgcgcgcggaggataccgctgtatattactgctcccgctggggtggggacggcttctatgccatggactattggggacagggcaccttggttacagtctcatcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgttag

SEQ ID NO: 200 - DNA, 2076 bp, synthetic construct
  source 1..2076: mol_type = other DNA; note = anti-CD22 CAR with m971 scFv and IgG1-Fc spacer [m971-Fc-BBz]
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggttccacaagcggcagtggcaagccgggaagcggcgaggggtcaactaagggcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgctgagcccaaatctcctgacaaaactcacacatgcccaccgtgcccagcacctccagtcgcgggaccgtcagtcttcctcttccccccaaaacccaaggacaccctcatgatcgcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaagccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaattttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgttag

SEQ ID NO: 201 - DNA, 2697 bp, synthetic construct
  source 1..2697: mol_type = other DNA; note = 22-BBz(xxx)/19-28
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggtggaggcggttcaggcggaggtggctctggcggtggcggatcggacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctttaacgagctcaatctaggacgaagagaggagttcgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgtacgggcggcggagagggcagaggcagtctgctgacatgcggtgacgtggaagagaatcccggccctaggatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactcccagacgccctggccccacccgcaagcattaccagccctatgcccccccacgcgacttcgcagcctatagatcttag

SEQ ID NO: 202 - DNA, 2697 bp, synthetic construct
  source 1..2697: mol_type = other DNA; note = aHSV-BBz(xxx)/19-28
atggagtttggcctgagctggctgttcctggtggccatcctgaagggcgtgcagtgtcaggttactctgaaagaatctggtccggccctggttaaaccgactcagactctgaccctgacgtgtactttctccggtttcagcctgtctacctccggtatgtctgttggttggattcgccaaccaccgggtaaagcactggaatggctggcgcacatctggtggaacaacgacaaatactataaaccggcactgaaatcccgtctgacgatctccaaagacacttctaagaaccaggttgttctgaccatgaccaacatggacccggtcgacaccgcaacctattactgcgcgcgtatctattacggttaccgtccgtatgcaatggattattggggtcagggtacgctggtcaccgtctcgagtggtggaggtggctcagggggcggaggcagcggaggcgggggcagcgacatcgttatgacccaaactccgctgtctctgccggttactccgggtgaaccggcgtctatctcctgtcgttcctcccagtccatcgttcactccaacggcaatacttacctggagtggtacctgcaaaaaccgggccaatctccacagctgctgatctacaaagttagcaaccgtttttctggcgtaccggatcgtttttctggttccggctctggtaccgacttcaccctgaagatttctcgtgtagaagctgaagatgtaggtgtgtactattgcttccagggctctcatgtaccgtggtctttcggccagggcaccaaactggagattaaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctttaacgagctcaatctaggacgaagagaggagttcgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgtacgggcggcggagagggcagaggcagtctgctgacatgcggtgacgtggaagagaatcccggccctaggatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactcccagacgccctggccccacccgcaagcattaccagccctatgcccccccacgcgacttcgcagcctatagatcttag

SEQ ID NO: 203 - DNA, 2781 bp, synthetic construct
  source 1..2781: mol_type = other DNA; note = aHSV-BBz/19-44v2
atggagtttggcctgagctggctgttcctggtggccatcctgaagggcgtgcagtgtcaggttactctgaaagaatctggtccggccctggttaaaccgactcagactctgaccctgacgtgtactttctccggtttcagcctgtctacctccggtatgtctgttggttggattcgccaaccaccgggtaaagcactggaatggctggcgcacatctggtggaacaacgacaaatactataaaccggcactgaaatcccgtctgacgatctccaaagacacttctaagaaccaggttgttctgaccatgaccaacatggacccggtcgacaccgcaacctattactgcgcgcgtatctattacggttaccgtccgtatgcaatggattattggggtcagggtacgctggtcaccgtctcgagtggtggaggtggctcagggggcggaggcagcggaggcgggggcagcgacatcgttatgacccaaactccgctgtctctgccggttactccgggtgaaccggcgtctatctcctgtcgttcctcccagtccatcgttcactccaacggcaatacttacctggagtggtacctgcaaaaaccgggccaatctccacagctgctgatctacaaagttagcaaccgtttttctggcgtaccggatcgtttttctggttccggctctggtaccgacttcaccctgaagatttctcgtgtagaagctgaagatgtaggtgtgtactattgcttccagggctctcatgtaccgtggtctttcggccagggcaccaaactggagattaaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattggcttattatcctggcgagtctcctggccctggcgttgatcctggcagtgtgtatcgccgtgaacagccgtcgcaggtgcggtcaaaagaaaaagctggtgatcaactctggcaacggagccgtcgaggacaggaagccttccggtctgaacggcgaggctagtaagtcccaggagatggtccacctcgtgaacaaggagagctccgaaacacccgaccagttcatgactgcggacgagacccgcaaccttcagaacgttgatatgaagatcggtgtgtaa

SEQ ID NO: 204 - DNA, 2766 bp, synthetic construct
  source 1..2766: mol_type = other DNA; note = aHSV-BBz/19-146v2
atggagtttggcctgagctggctgttcctggtggccatcctgaagggcgtgcagtgtcaggttactctgaaagaatctggtccggccctggttaaaccgactcagactctgaccctgacgtgtactttctccggtttcagcctgtctacctccggtatgtctgttggttggattcgccaaccaccgggtaaagcactggaatggctggcgcacatctggtggaacaacgacaaatactataaaccggcactgaaatcccgtctgacgatctccaaagacacttctaagaaccaggttgttctgaccatgaccaacatggacccggtcgacaccgcaacctattactgcgcgcgtatctattacggttaccgtccgtatgcaatggattattggggtcagggtacgctggtcaccgtctcgagtggtggaggtggctcagggggcggaggcagcggaggcgggggcagcgacatcgttatgacccaaactccgctgtctctgccggttactccgggtgaaccggcgtctatctcctgtcgttcctcccagtccatcgttcactccaacggcaatacttacctggagtggtacctgcaaaaaccgggccaatctccacagctgctgatctacaaagttagcaaccgtttttctggcgtaccggatcgtttttctggttccggctctggtaccgacttcaccctgaagatttctcgtgtagaagctgaagatgtaggtgtgtactattgcttccagggctctcatgtaccgtggtctttcggccagggcaccaaactggagattaaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggttccggcaagcccggatctggcgagggttccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatggcgttgtgatcgtggccgtgattgtgtgcattctggttttggctgtactgggcgctgtgctttactttttgtacaagaaaggaaagctgccgtgtcgccgtagcggcaagcaggagatcactttgcctccctctcgcaaatcagagctggtggtcgaagtcaaaagcgacaagctccctgaagagatgggcttgctgcagggtagtagcggtgataagcgcgcccctggtgaccagggagagaagtacatcgacctgagacactaa

SEQ ID NO: 205 - DNA, 3273 bp, synthetic construct
  source 1..3273: mol_type = other DNA; note = m971-Fc-BBz/SGIII-28
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggttccacaagcggcagtggcaagccgggaagcggcgaggggtcaactaagggcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgctgagcccaaatctcctgacaaaactcacacatgcccaccgtgcccagcacctccagtcgcgggaccgtcagtcttcctcttccccccaaaacccaaggacaccctcatgatcgcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaagccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaattttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatggagtttggcctgagctggctgttcctggtggccatcctgaagggcgtgcagtgtgaggtgcagctggtggagtctgggggaggcttggtccagcctggggggtccctgagactctcctgtgcagcctctggattcgctttcagtatctatgacatgtcttgggtccgccaggttccagggaaggggctggagtgggtctcatacattagtagtggtggtggtaccacctactatccagacactgtgaagggcagattcaccatctccagagacaattccaggaacactctggatcttcaaatgaacagtctgagagtcgaggacacggctgtctattattgtgcgagacatagtggctacggtagtagctacggggttttgtttgcttactggggccaaggaaccctggtcaccgtctcctcaggtggaggcggttcaggcggaggtggctctggcggtggcggatcggacatccagatgactcagtctccatcctccctgtctgcatctgtaggagacagagtcaccatcacctgcagggcaagtcaggacattagcaattatttaaactggcttcaacagaaaccagggaaagcccctaagctcctgatttactacacatcaatattacactcaggagtcccatcaaggttcagcggcagtggatctgggacagagttcactctcacaatcagcagcctgcagcctgaagattttgcaacttattactgtcaacagggtaatacgcttccgtggacgtttggccaggggaccaaactggaaatcaaaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactcccagacgccctggccccacccgcaagcattaccagccctatgcccccccacgcgacttcgcagcctatagatcttag

SEQ ID NO: 206 - DNA, 3357 bp, synthetic construct
  source 1..3357: mol_type = other DNA; note = m971-Fc-BBz/SGIII-44v2
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggttccacaagcggcagtggcaagccgggaagcggcgaggggtcaactaagggcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgctgagcccaaatctcctgacaaaactcacacatgcccaccgtgcccagcacctccagtcgcgggaccgtcagtcttcctcttccccccaaaacccaaggacaccctcatgatcgcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaagccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaattttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatggagtttggcctgagctggctgttcctggtggccatcctgaagggcgtgcagtgtgaggtgcagctggtggagtctgggggaggcttggtccagcctggggggtccctgagactctcctgtgcagcctctggattcgctttcagtatctatgacatgtcttgggtccgccaggttccagggaaggggctggagtgggtctcatacattagtagtggtggtggtaccacctactatccagacactgtgaagggcagattcaccatctccagagacaattccaggaacactctggatcttcaaatgaacagtctgagagtcgaggacacggctgtctattattgtgcgagacatagtggctacggtagtagctacggggttttgtttgcttactggggccaaggaaccctggtcaccgtctcctcaggtggaggcggttcaggcggaggtggctctggcggtggcggatcggacatccagatgactcagtctccatcctccctgtctgcatctgtaggagacagagtcaccatcacctgcagggcaagtcaggacattagcaattatttaaactggcttcaacagaaaccagggaaagcccctaagctcctgatttactacacatcaatattacactcaggagtcccatcaaggttcagcggcagtggatctgggacagagttcactctcacaatcagcagcctgcagcctgaagattttgcaacttattactgtcaacagggtaatacgcttccgtggacgtttggccaggggaccaaactggaaatcaaaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattggcttattatcctggcgagtctcctggccctggcgttgatcctggcagtgtgtatcgccgtgaacagccgtcgcaggtgcggtcaaaagaaaaagctggtgatcaactctggcaacggagccgtcgaggacaggaagccttccggtctgaacggcgaggctagtaagtcccaggagatggtccacctcgtgaacaaggagagctccgaaacacccgaccagttcatgactgcggacgagacccgcaaccttcagaacgttgatatgaagatcggtgtgtaa

SEQ ID NO: 207 - DNA, 3342 bp, synthetic construct
  source 1..3342: mol_type = other DNA; note = m971-Fc-BBz/SGIII-146v2
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggttccacaagcggcagtggcaagccgggaagcggcgaggggtcaactaagggcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgctgagcccaaatctcctgacaaaactcacacatgcccaccgtgcccagcacctccagtcgcgggaccgtcagtcttcctcttccccccaaaacccaaggacaccctcatgatcgcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaagccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaattttgggtgctggtggtggttggtggagtcctggcttgctatagcttgctagtaacagtggcctttattattttctgggtgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgtacgggcggcggagagggcagaggcagtctgctgacatgcggtgacgtggaagagaatcccggccctaggatggagtttggcctgagctggctgttcctggtggccatcctgaagggcgtgcagtgtgaggtgcagctggtggagtctgggggaggcttggtccagcctggggggtccctgagactctcctgtgcagcctctggattcgctttcagtatctatgacatgtcttgggtccgccaggttccagggaaggggctggagtgggtctcatacattagtagtggtggtggtaccacctactatccagacactgtgaagggcagattcaccatctccagagacaattccaggaacactctggatcttcaaatgaacagtctgagagtcgaggacacggctgtctattattgtgcgagacatagtggctacggtagtagctacggggttttgtttgcttactggggccaaggaaccctggtcaccgtctcctcaggtggaggcggttcaggcggaggtggctctggcggtggcggatcggacatccagatgactcagtctccatcctccctgtctgcatctgtaggagacagagtcaccatcacctgcagggcaagtcaggacattagcaattatttaaactggcttcaacagaaaccagggaaagcccctaagctcctgatttactacacatcaatattacactcaggagtcccatcaaggttcagcggcagtggatctgggacagagttcactctcacaatcagcagcctgcagcctgaagattttgcaacttattactgtcaacagggtaatacgcttccgtggacgtttggccaggggaccaaactggaaatcaaaggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatggcgttgtgatcgtggccgtgattgtgtgcattctggttttggctgtactgggcgctgtgctttactttttgtacaagaaaggaaagctgccgtgtcgccgtagcggcaagcaggagatcactttgcctccctctcgcaaatcagagctggtggtcgaagtcaaaagcgacaagctccctgaagagatgggcttgctgcagggtagtagcggtgataagcgcgcccctggtgaccagggagagaagtacatcgacctgagacactaa

(19) United States
(12) Patent Application Publication
(10) Pub. No.: US 2026/0284111 A1
(43) Pub. Date: Sep. 24, 2026

(54) CORECEPTOR CHIMERIC ANTIGEN RECEPTOR

(71) Applicant: VANUDIS GMBH, Stuttgart (DE)

(72) Inventor: Rudolf ÜBELHART, Bad Schönborn (DE)

(21) Appl. No.: 19/477,898

(22) PCT Filed: Apr. 29, 2024
(86) PCT No.: PCT/EP2024/061710

§ 371 Date: Oct. 23, 2025

(30) Foreign Application Priority Data
Apr. 27, 2023 (EP) 23170480.0

(51) Int. Cl. A61K 35/17; A61K 40/11; A61K 40/31; A61K 40/42; A61P 35/00; C07K 14/705; C07K 16/28; C07K 16/32

(52) U.S. Cl. CPC A61K 35/17; A61K 40/11; A61K 40/31; A61K 40/4204; A61K 40/4205; A61P 35/00; C07K 14/7051; C07K 14/70514; C07K 14/70517; C07K 14/70521; C07K 14/70585; C07K 16/2863; C07K 16/32; A61K 2239/13; A61K 2239/17; A61K 2239/21; A61K 2239/22; A61K 2239/28; A61K 2239/48; C07K 2317/622

(57) ABSTRACT

The present invention relates to a co-receptor chimeric antigen receptor (CoCAR) capable of binding LCK, combinations of a co-receptor chimeric antigen receptor and a CAR, nucleic acid constructs encoding such CoCAR, recombinant cells engineered to express such CoCAR and pharmaceutical compositions comprising the same. The present invention further provides the use thereof in the treatment of diseases preferably characterized by expression of a tumor-associated antigen.

### TECHNICAL FIELD OF THE INVENTION

The present invention relates to a coreceptor CAR (CoCAR), a combination of a CoCAR and a CAR, nucleic acids encoding such antigen receptor, recombinant cells engineered to express such CoCAR and pharmaceutical compositions comprising the same. Further disclosed is the use thereof in the treatment of diseases preferably characterized by expression of at least one tumor-associated antigen.

### BACKGROUND OF THE INVENTION

Chimeric antigen receptor (CAR)-T cells represent an innovative immunotherapeutic approach and such therapeutics have shown tremendous potential for the treatment of cancer. CAR-T cells are generated from patient-derived autologous T cells which are genetically modified to express a CAR. After genetic modification and expansion ex vivo, CAR-T cells are reinfused into the patients where they bind to a specific structure exposed on the surface of tumor cells. The antigen specificity of CAR-T cells is provided by the CAR, which is an artificially constructed fusion protein containing an extracellular antigen-binding domain (e.g. a single chain variable fragment, scFv) derived from an antibody linked to T cell-derived transmembrane and intracellular signaling domains. A classical CAR consists of an antigen-specific single chain antibody (scFv) fragment, fused to a transmembrane and signaling domain such as CD3ζ. Upon introduction into T cells it is expressed as a membrane-bound protein and induces immune responses upon binding to its cognate antigen (Eshhar et al., (1993) PNAS, (90) 720-724).

CARs exploit the antigen-binding properties of the parental monoclonal antibody and enable the T cells to respond to native antigens expressed on the surface of tumor cells in a non-MHC restricted manner. Therefore, the mechanism of CAR-T cell activation is fundamentally different from that of conventional T cells, the latter of which exclusively respond to processed peptide antigens presented by MHC molecules. The CAR-dependent activation of the CAR-T cell elicits an immune response against the antigen-expressing cells resulting in T cell-mediated destruction of the tumor.

Current CAR-T cell therapies show limited efficacy when the specific target antigen recognized by the CAR is expressed at low densities. Initially, tumor cells often express high amounts of tumor-associated antigens, but under the pressure of the therapeutic intervention, tumor cells can downregulate the amount of the target antigen expressed on their surface, with the consequence that CARs become unable to activate T cells to kill the tumor cells. In other scenarios, CARs may inhibit T cell functions even when antigen density is normal or high, for example when CARs induce T cell exhaustion, thereby attenuating or terminating the T cell therapeutic effects eventually. Therefore, there remains a need for modulating T cell activation to overcome these limitations to enhance the reach of these therapeutics.

Early CAR designs comprised an antibody single-chain variable fragment (scFv) as antigen-recognition domain, which is fused through a hinge and transmembrane domain to the cytoplasmic tail of the TCR signalling component CD3ζ. T cells expressing such first-generation CARs can induce cytotoxicity toward antigen-positive tumor cells, however, they are not able to efficiently control tumor growth in vivo due to their poor persistence capacities (Eshhar et al., (1993) PNAS, (90) 720-724). Current CAR designs incorporate one or more costimulatory domains in addition to an intracellular CD3ζ T cell activation domain, which classify second-(one costimulatory domain) and third-generation (two costimulatory domains) CAR architectures. This design principle aims at combining signal 1 (from the TCR) and signal 2 (from a costimulatory-receptor) required to unleash full T cell activation, and indeed this significantly improved in vivo persistence and clinical efficacy. In most cases, the costimulatory domains derive either from the CD28 receptor family (CD28, ICOS) or the tumor necrosis factor receptor (TNFR) family (4-1BB, CD27, OX40), whereas CD28/CD3ζ (28z) and 4-1BB/CD3ζ (BBz) second-generation CARs are the most frequently used combinations in clinically tested cell products. Notably, all the six currently FDA-approved CAR-T cell products use second-generation CAR designs with four of them contain BBz, and two 28z, signalling domains.

Growing practical experience with BBz- and 28z-CARs indicate fundamental functional differences between these two CAR designs, which affect the in vivo persistence of the CAR-T cells on the one hand, and the antigen-sensitivity on the other hand. BBz-CAR-T cells typically show enhanced in vivo expansion and persistence capacities in mice and humans compared to 28z CAR-T cells, the latter of which rarely persist for more than one or two months in patients. In contrast, 28z-CARs outperform BBz-CARs in tumors where antigen density is low, and the likelihood for developing antigen-low relapses is lower with 28z-CAR-T cells compared to their BBz counterparts. Therefore, there is a need for reengineering BBz-CARs to enhance antitumor immune responses against tumor cells with low antigen density, while maintaining their unique capacity for persistence.

28z-CARs typically induce faster and larger-magnitude activation of downstream signalling events, which result from an association of the Src-family kinase LCK with the CD28 endodomain of the CAR (Salter et al., (2018) Sci Signal. 11(544)). LCK association promotes increased basal CAR-CD35 phosphorylation and more intense antigen-dependent T cell activation, and this probably accounts for the high sensitivity observed for CD28-based anti-CD19 CAR-T cells against target cells with decreased antigen density (Hamieh et al., (2019) Nature, (568) 112-116). 28z-CARs induce glycolytic metabolism and an effector T cell-like phenotype, which translate into high effector functions, but at the expense of rapid exhaustion after repeated stimulation and induction of activation-induced cell death, and low persistence in vivo. In fact, 28z-CAR-T cells do not persist in patients more than 60 days after infusion and disappearance of CAR-T cells is a major source of relapse (Cappell & Kochenderfer, (2021) Nat Rev Clin Oncol. (11) 715-727; Neelapu et al., (2017) N Engl J Med, (377) 2531-2544). In contrast, BBz-CARs induce weaker downstream signalling events after stimulation, which is because the 4-1BB endodomain of the CAR facilitates the recruitment of the THEMIS-SHP1 phosphatase complex to the CAR synapse, where SHP1 attenuates LCK-mediated phosphorylation of the CAR-CD3ζ signalling domains (Sun et al., (2020) Cancer Cell, (2) 216-225). Nevertheless, 28z- and BBz-CARs induce equal antitumor responses against target cells with high antigen expression, yet BBz-CAR-T cells show better expansion and persistence capacities in vivo. It has been demonstrated that the inclusion of the 4-1BB endodomain into the CAR synapse induces fatty acid oxidation metabolism and increases mitochondrial biogenesis, thereby significantly enhancing the respiratory capacity and differentiation toward an T cell memory-like phenotype (Kawalekar et al, (2016) Immunity, (2) 380-390). Since the endodomain of 4-1BB contains binding sites for TRAF1, TRAF2 and TRAF3, which activate canonical and non-canonical NFκB-pathways and expression of anti-apoptotic proteins such as BCL-2 and BCL-XL, BBz CAR-T cells are more resistant to exhaustion and activation-induced cell death (Li et al., (2018) JCI Insight, (18) 121322). Therefore, BBz-CAR-T cells exhibit sustained antitumor activity against established tumors in vivo and can persist for months or even years in responding patients (Melenhorst et al., (2022) Nature, (602) 503-509). A parallel comparison of BBz- and 28z-anti-CD19 CAR-T cells in patients with B cell lymphoma revealed similar antitumor efficacies, yet the 28z-CAR product induced severe cytokine release syndrome (CRS) and neurotoxicities, which led to termination of further evaluation of 28z-CAR-T cells (Ying et al., (2019) Mol Ther Oncolytics, (15) 60-68). Moreover, compared with their 28z counterparts, BBz-anti-CD19 CAR-T cells resulted in higher antitumor efficacy and less severe adverse events in patients with B cell acute lymphoblastic leukemia (B-ALL) (Zhao et al, (2020) Mol Ther Oncolytics, (18) 272-281), indicating superior functions of BBz-CARs in the treatment of hematological malignancies. However, relapses are common with BBz-CAR-T cells, most of them because of a mechanism called antigen escape, which refers to relapsing tumor cells that display reduced numbers of the target antigen or which have completely lost antigen expression. For instance, clinical trials with anti-BCMA BBz-CAR-T cells in patients with multiple myeloma have demonstrated outgrowth of tumor cells expressing low levels of BCMA (Brudno et al., (2018) J Clin Oncol, (22) 2267-2280; Cohen et al., (2019) J Clin Invest, (6) 2210-2221). Furthermore, anti-CD22 BBz-CAR-T cells that utilize the m971 antigen-recognition domain showed highly efficient antitumor responses in preclinical models (Haso et al, (2013) Blood, (7) 1165-1174) and induced more than 70% complete response rates in B-ALL patients, but the majority of responding patients relapsed because of the outgrowth of tumor cells with reduced CD22 antigen densities (Fry et al., (2017) Nat Med, (1) 20-28; Shah et al., (2020) J Clin Oncol. (17) 1938-1959). Preclinical modelling using engineered variants of the B-ALL tumor cell line Nalm6 revealed that a reduction in CD22 site densities to approximately 1,800 molecules per cell was sufficient to blunt curative antitumor responses by anti-CD22 BBz-CAR-T cells (Fry et al., (2017) Nat Med, (1) 20-28). A similar insufficient reactivity of BBz-CAR-T cells against target cells with reduced antigen densities has also been shown for other specificities, including anti-CD19 and anti-GPC2 CARs that failed to eradicate tumor cells with 2,053 CD19 and 6,000 GPC2 molecules per cell, respectively (Majzner et al., (2020) Cancer Discov. (5) 702-723; Heitzeneder et al, (2022) Cancer Cell, (1) 53-69). These results indicate that antigen downmodulation to a few thousand molecules per cell is a general cause of resistance to BBz-CAR-T cells and indicates only insufficient sensitivity of BBz-CARs for antigen-low target cells. Therefore, reengineering BBz-CARs to enhance their sensitivity, while preserving their capacity for expansion and persistence, could provide a solution to this problem and may prevent relapses with antigen-low tumors.

Since CARs incorporate well characterized T cell signalling components, it has been assumed that CARs signal analogously to conventional TCRs. In fact, both CARs and TCRs require CD3ζ ITAM phosphorylation to initiate signalling, which is mediated by Src-family kinases, the most important of which is LCK. LCK can be found either free in the cytosol, anchored to the plasma membrane though N-terminal palmitoylation and myristoylation, or associated with the intracellular tails of CD4 or CD8 coreceptors through a “zinc-clasp” structure (Kim et al, (2003) Science, (5640) 1725-1728), and each of these LCK forms are able to mediate phosphorylation of the CD3 ITAMs of the TCR. Engagement of TCR with peptide-MHC complexes also facilitates the recruitment of CD4 or CD8 coreceptors that bind to the same MHC molecule as the TCR, and which creates a positive feedback loop to recruit more LCK that phosphorylate more CD3 ITAMs. Phosphorylated ITAMs then serve as docking sites for the SH2 domains of ZAP-70, a kinase whose activation is enhanced by phosphorylation, primarily by CD4 or CD8 coreceptor bound LCK. ZAP-70 then phosphorylates the adaptor proteins LAT and SLP-76, which form signalling scaffolds to which downstream signalling proteins such as Phospholipase C (PLC)-γ and Grb2 are recruited, this complex is referred as to the signalosome. Activated PLC-y catalyzes the formation of second-messengers inositol-1,4,5-triphosphate (IP3) and diacylglycerol (DAG) from the membrane lipid phosphatidylinositol-4,5-biphosphate, which leads to intracellular calcium mobilization and the activation of several signalling pathways that result in altered gene expression, proliferation, and differentiation.

CAR ligation also induces phosphorylation of CD3ζ ITAMs, which is critical for initiation of CAR activity, but CARs cannot engage CD4 or CD8 coreceptors. ZAP-70, SLP-76 and PLC-γ also become phosphorylated upon CAR ligation, and this phosphorylation, as with CD3ζ, is more robust in 28z- than in BBz-CAR-T cells, owed to the presence of a LCK recruitment site in the CD28 endodomain. However, the precise mechanisms of how LCK, ZAP-70, LAT, SLP-76 and other signalling proteins are recruited to the CAR synapse remains unclear, especially when considering that BBz-CARs do not contain any LCK binding sites.

To design BBz-CARs with improved antigen sensitivity, it was tested if the addition of the intracellular tail of the CD38-subunit of the TCR to the CAR sequence improves CAR-T cell function, as this CD3 subunit contains internal proline-rich sequence (PRS) and receptor kinase (RK) motifs that facilitate the recruitment of the kinases Nck and LCK, respectively, which cooperatively promote initial CD3 ITAM phosphorylation in the context of a TCR (Borroto et al., (2014) J Immunol, (5) 2042-2053; Hartl et al., (2021) Cells, (4) 834). Indeed, the addition of the CD38 domain into BBz-CARs had beneficial effects on T cell activation and antitumor immune responses in vitro and in vivo, and improved the antigen sensitivity (Hartl et al., (2020) Nat Immunol, (8) 902-913; Salter et al., (2021) Sci Signal. (697) 2606). Other studies showed that CAR function could be improved by modifying the receptor backbone to include cytokine signalling domains (Kagoya et al., (2018) Nat Med. (3) 352-359), a second CD3ζ endodomain, or altered hinge and transmembrane domains (Majzner et al., (2020) Cancer Discov. (5) 702-723)

In another study it was tested if the global overexpression of the kinase LCK in BBz-CAR-T cells could compensate for the enhanced activity of the phosphatase SHP1 at the CAR synapse, thereby enhancing basal CAR-CD3ζ phosphorylation and the function of T cells expressing a BBz-CAR. Indeed, overexpression of LCK led to increased basal CAR-CD3ζ phosphorylation and intracellular calcium mobilization after antigenic stimulation, and these LCK-BBz-CAR-T cells showed improved expansion capacities and tumor control in vivo as compared to conventional BBz-CAR-T cell lacking LCK overexpression (Sun et al., (2020) Cancer Cell, (2) 216-225; WO2021087183). However, in that study it was not shown whether overexpression of LCK improved the sensitivity of BBz-CAR-T cells against antigen-low tumor cells, and if increased LCK-mediated basal CAR-CD3ζ phosphorylation eventually caused tonic CAR signalling resulting in antigen-independent T cell exhaustion and T cell differentiation, which is a common problem seen with 28z-CAR constructs (Long et al., (2015) Nat Med, (6) 581-590).

Yet another study described the expression of a chimeric molecule called T cell antigen coupler (TAC), a chimeric receptor comprising an antigen-recognition domain, a TCR-recruitment domain (an anti-CD3ε scFv), and a coreceptor domain (hinge, transmembrane and cytosolic regions from CD4 or CD8α) (Helsen et al., (2018) Nat Commun, (1) 3049; WO2015117229). Rather than modifying current CAR designs, the TAC receptor was designed to engage both the coreceptor and endogenously expressed TCRs in response to binding to a single antigen, aiming to recapitulate the architecture of a TCR-coreceptor complex to activate natural signalling pathways and T cell responses.

### SUMMARY OF INVENTION

The present disclosure relates generally to the development of immunotherapeutics, including recombinant cells, containing polypeptides or polynucleotides encoding such polypeptides, such as a chimeric antigen receptor (CAR) containing an antigen-recognition domain fused to hinge, transmembrane and T cell activating domains, and a chimeric coreceptor containing an antigen-recognition domain fused to an extracellular domain, a hinge domain, a transmembrane domain and a cytosolic domain (a) capable of binding LCK and/or (b) comprising LCK (CoCAR), as well as pharmaceutical compositions containing the same for use in enhancing immune therapies in e.g. treating various conditions, such as diseases (e.g. cancer). As described in greater detail below, different CAR and CoCAR constructs have been prepared and found to have dramatic effects to enhance activation and increase the potency of immune cells (e.g. T cells) co-expressing a 4-1BB-based second-generation CAR and a CoCAR in response to tumor cells, even when the antigen is expressed at a very low density.

According to a first aspect, the present invention provides a coreceptor-CAR (CoCAR), comprising (i) a first extracellular antigen-recognition domain, (ii) a first hinge domain, (iii) a first transmembrane domain, and (iv) at least one cytosolic domain (a) capable of binding LCK, and/or (b) comprising LCK, or a variant or fragment thereof.

In particular, (i) the first extracellular antigen-recognition domain, (ii) the first hinge domain, (iii) the first transmembrane domain, and (iv) the at least one cytosolic domain are arranged from N to C terminal.

In particular, the coreceptor-CAR does not comprise a CD3zeta domain.

According to an embodiment, the first antigen-recognition domain is a scFv.

According to an embodiment, the first hinge domain is derived from a CD4, CD8, CD28 or IgG-Fc hinge domain.

According to another embodiment the first transmembrane domain is derived from a CD4, CD8, CD28, or CD3zeta transmembrane domains.

According to yet another embodiment the at least one cytosolic domain (a) capable of binding LCK is (1) an intracellular signaling domain derived from a CD4, CD8α, CD3ε, CD28, CD44, or CD146 intracellular signaling domain, or a variant or fragment thereof, and/or (2) comprises one or more motifs capable of binding LCK.

In particular, the intracellular signaling domain (1) is derived from a CD4, CD8α, CD3ε, CD44, or CD146 intracellular signaling domain, or a variant or fragment thereof.

In particular, the intracellular signaling domain (1) is derived from a CD4, CD8α, CD28, CD44, or CD146 intracellular signaling domain, or a variant or fragment thereof.

In particular, the intracellular domain (1) is derived from a CD4, CD8α, CD44, or CD146 intracellular signaling domain, or a variant or fragment thereof.

In particular, the intracellular domain (1) is derived from a CD4 or CD8α intracellular signaling domain, or a variant or fragment thereof,

In particular, the motif is derived from a CD4, CD8α, CD28, CD3ε, CD44, or CD146 intracellular signaling domain.

In particular, the motif capable of binding LCK is derived from a CD4, CD8α, CD28, CD3ε, CD44, or CD146 intracellular signaling domain.

In particular, the motif capable of binding LCK is derived from a CD4, CD8α, CD3ε, CD44, or CD146 intracellular signaling domain, or a variant or fragment thereof.

In particular, the motif capable of binding LCK is derived from a CD4, CD8α, CD28, CD44, or CD146 intracellular signaling domain, or a variant or fragment thereof.

In particular, the motif capable of binding LCK is derived from a CD4, CD8α, CD44, or CD146 intracellular signaling domain, or a variant or fragment thereof.

In particular, the motif capable of binding LCK is derived from a CD28, CD3ε, CD44, or CD146 intracellular signaling domain. In particular, the motif capable of binding LCK is derived from a CD28, CD44, or CD146 intracellular signaling domain.

In particular, the motif capable of binding LCK is derived from a CD4 or CD8α intracellular signaling domain, or a variant or fragment thereof,

According to yet another embodiment, the CoCAR is capable of specifically binding to an antigen on a target cell.

According to a second aspect, the present invention provides a combination comprising (a) the CoCAR of the present invention, and (b) a chimeric antigen receptor (CAR).

According to an embodiment, the CAR (b) comprises (i) a second extracellular antigen-recognition domain, (ii) a second hinge region, (iii) a second transmembrane domain, (iv) a 4-1BB domain and/or a CD28 domain, and (v) a CD3zeta domain.

In particular, in the CAR, (i) the second extracellular antigen-recognition domain, (ii) the second hinge region, (iii) the second transmembrane domain, (iv) the 4-1BB domain and/or a CD28 domain, and (v) the CD3zeta domain are arranged from N to C terminal.

According to a third aspect, the present invention provides a nucleic acid, nucleic acids, a nucleic acid construct and nucleic acid constructs encoding the CoCAR of the present invention or the combination of the present invention.

According to a fourth aspect, the present invention provides a vector or a combination of vectors comprising the nucleic acid, nucleic acids, the nucleic acid construct and/or nucleic acid constructs of the present invention.

According to a fifth aspect, the present invention provides a recombinant cell, comprising the nucleic acid construct of the present invention

According to an embodiment, the cell of the present invention is an immune effector cell.

According to an embodiment, the cell of the present invention expresses the CoCAR of the present invention on the cell surface.

According to an embodiment, the cell of the present invention expresses the combination of the present invention on the cell surface.

According to yet another embodiment, in the cell of the present invention, (i) the CAR and (ii) the CoCAR recognize different epitopes located in the same antigen expressed on the surface of a target cell.

According to yet another embodiment, in the cell of the present invention, (i) the CAR and (ii) the CoCAR recognize different antigens expressed on the surface of a target cell.

According to a sixth aspect, the present invention provides a pharmaceutical composition comprising (i) the CoCAR of the present invention, (ii) the combination of the present invention, (iii) the nucleic acid construct of the present invention, and/or (iv) the recombinant cell of the present invention, and a pharmaceutically acceptable carrier.

According to a seventh aspect, the present invention provides the CoCAR of the present invention, the combination of the present invention, the nucleic acid construct according of the present invention, the recombinant cell of the present invention, or the pharmaceutical composition of the present invention, for use in medicine.

According to an eighth aspect, the present invention provides the CoCAR of the present invention, the combination of the present invention, the nucleic acid construct according of the present invention, the recombinant cell of the present invention, or the pharmaceutical composition of the present invention, for use in treating cancer.

According to an embodiment, the CoCAR, the combination, the nucleic acid construct, the recombinant cell, or the pharmaceutical composition for use of the present invention, in treating a malignancy with cell-surface expression is preferably selected from the group consisting of B7-H3 (CD276), BCMA, CD3, CD5, CD19, CD20, CD22, CD23, CD30, CD33, CD38, CD44, CD44v6, CD52, CD70, CD79A, CD79B, CD123, CD138, CD171, CEA, Claudin-6, Claudin-18.2, CLL1, CXCR5, EGFR, EGFRvIII, EPH-receptor A2, IGLV3-21, GP-2, GP-40, HER2, ErbB3, ErbB4, FBP, AchR, Fr-a, GD2, GD3, HMW-MAA, IL13Ra2, Kappa-LC, IGLV3-21-R110, Lewis Y, Mesothelin, MUC1, MUC16, NKG2D Ligands, NCAM, NY-ESO1, Oncofetal antigen h5T4, PSCA, PSMA, ROR1, TAG-72, VEGFR, GOLPH2, and SLAMF7.

According to a ninth aspect, the present invention provides the CoCAR, the combination, the nucleic acid construct, the recombinant cell, and/or the pharmaceutical composition of the invention, for use in treating a B cell malignancy.

According to a tenth aspect, the present invention provides a method of treating cancer in a subject in need thereof, comprising the step of administering to the subject an effective amount of (i) the CoCAR of the present invention, (ii) the combination of the present invention, (iii) the nucleic acid construct of the present invention, and/or (iv) the recombinant cell of the present invention.

According to an embodiment, the cancer is a malignancy positive for at least one tumor-associated antigen preferably selected from the group consisting of B7-H3 (CD276), BCMA, CD3, CD5, CD19, CD20, CD22, CD23, CD30, CD33, CD38, CD44, CD44v6, CD52, CD70, CD79A, CD79B, CD123, CD138, CD171, CEA, Claudin-6, Claudin-18.2, CLL1, CXCR5, EGFR, EGFRvIII, EPH-receptor A2, IGLV3-21, GP-2, GP-40, HER2, ErbB3, ErbB4, FBP, AchR, Fr-a, GD2, GD3, HMW-MAA, IL13Ra2, Kappa-LC, IGLV3-21-R110, Lewis Y, Mesothelin, MUC1, MUC16, NKG2D Ligands, NCAM, NY-ESO1, Oncofetal antigen h5T4, PSCA, PSMA, ROR1, TAG-72, VEGFR, GOLPH2, and SLAMF7.

According to another embodiment, the cancer is a B cell malignancy.

Other features and advantages of the instant invention will be apparent from the following detailed description, the figures and the claims.

### FIGURES

FIG. 1 shows the properties of untransduced (UTD) T cells, or T cells expressing an anti-CD22 BBz-CAR with or without overexpression of LCK. (A) Co-expressing LCK with a BBz-CAR may increases basal CAR-CD3ζ phosphorylation, thereby enhancing the activity of CAR-T cells. (B) The phenotype of T cells was analyzed by flow cytometry on day 14 after initial T cell activation, and T cell subsets are defined according to expression of CAR, CD4, CD8, CCR7 (CD197) and CD45R0. CCR7+/CD45R0− cells represent naïve-like T cells, CCR7+/CD45R0+ cells represent central-memory T cells, CCR7−/CD45R0+ cells represent effector-memory T cells, and CCR7−/CD45R0− cells represent effector T cells. These data show that overexpression of LCK together with a BBz-CAR leads to T cell differentiation towards effector-memory and effector T cells. (C) shows the ex vivo expansion of T cells expressing the indicated constructs. While untransduced and anti-CD22 BBz-CAR-transduced T cells efficiently proliferate in the first 14 days after T cell activation by CD3/CD28 agonistic agents on day 0, CAR-T cells overexpressing LCK show only poor expansion capacities.

FIG. 2 illustrates the concept of the present invention of co-expressing a BBz-CAR and a CoCAR. The CoCAR comprises an antigen-recognition domain fused to a coreceptor, in this example to the hinge, transmembrane and cytosolic domains of CD4. The antigen-recognition domains of the BBz-CAR and the coreceptor-CAR recognize different epitopes present either in the same antigen, or in two different antigens expressed by the same target cell. (A) In unstimulated T cells, the CAR and the CoCAR molecules are spatially separated from each other, for which reason CoCAR bound LCK is not available for basal CAR-CD3z phosphorylation. (B) After binding to cognate antigens, BBz-CAR and coreceptor-CAR relocalize to get in proximity, and the coreceptor CAR recruits coupled LCK to the BBz-CAR synapse, where it supports CAR-CD3z phosphorylation. ZAP-70 binds to phosphorylated ITAMs of CAR-CD3z domains and recruited ZAP-70 molecules then become efficiently phosphorylated by CoCAR-associated LCK. Activated ZAP-70 in turn phosphorylates its substrates including LAT and SLP-76, which form of the signalosome and activate various downstream signalling pathways.

FIG. 3 shows the properties of T cells transduced to express an anti-CD19 CoCAR or an anti-CD22 BBz-CAR, or co-transduced with both lentiviral vectors to co-express the BBz-CAR with the CoCAR. (A) Schematic illustration of the CAR and CoCAR molecules. (B) Flow cytometry analysis shows cell-surface expression of the anti-CD22 BBz-CAR and the anti-CD19 CoCAR molecules. (C) Ex vivo expansion of T cells in the first 14 days after activation by anti-CD3/CD28 agonists (TransAct, Miltenyi). (D) Quantification of CD22 receptor densities on the cell-surface of Nalm6 and Raji tumor cell lines using Quantibrite PE Quantification Beads (Becton Dickinson). (E) shows the percentage of CD69/CD25 double-positive T cells expressing the indicated constructs 24 hours after co-culture with the indicated target cell lines as determined by flow cytometry analysis. (F) shows the amount of IFN-gamma produced by T cells expressing the indicated constructs 24 hours after co-culture with the indicated target cell lines as determined by flow cytometry analysis using LEGENDplex Kits (BioLegend).

FIG. 4 shows the antigen-dependent activity of T cells transduced to express an anti-CD22 CoCAR, an anti-CD22 BBz-CAR, or co-expressing the BBz-CAR with the CoCAR from a bicistronic vector. (A) Schematic illustration of the CAR and CoCAR molecules. (B) Flow cytometry analysis shows cell-surface expression of the anti-CD22 BBz-CAR and CoCAR molecules. (C) T cells were incubated in 96-well cell culture plates coated with recombinant human CD22 protein at indicated concentrations. After 24 hours, the concentration of effector cytokines in the cell culture supernatant was determined by flow cytometry using the LEGENDPlex multiplex assay (BioLegend). (D) shows the amount of IFNγ, TNFα and IL-2 produced by T cells expressing the indicated constructs 24 hours after co-culture with CD22-positive Nalm6 cells (or unstimulated T cells), as determined by flow cytometry analysis using the LEGENDplex multiplex assay (BioLegend). (E) shows CD22 site densities on the surface of wild-type Nalm6 cells, CD22 knockout Nalm6 cells, and Nalm6 cell clone #3 engineered to express ultra-low CD22 antigen-levels. CD22 site densities were quantified using Quantibrite PE Quantification Beads (Becton Dickinson). (F) Untransduced T cells (UTD) or T cells T cells expressing the indicated constructs were co-cultured with GFP-positive Nalm6 cells clone #3 (expressing ~400 CD22 molecules per cell) in a T cell to target cell ratio of 1:10, and the growth of GFP-positive target cells was monitored over two days in an Incucyte instrument (Sartorius).

FIG. 5 shows the antigen-dependent and -independent activation of T cells transduced to express an anti-CD22 CoCAR, an anti-CD22 BBz-CAR, or co-expressing the BBz-CAR with the CoCAR from a bicistronic vector. (A) Expression of activation markers CD69 and CD25 is shown for unstimulated T cells and T cells stimulated with the CD22-negative myeloid cell line K562 or with the CD22-positive B-ALL cell line Nalm6 for 24 hours. (B) shows expression of inhibitory receptors LAG-3, TIM-3 and PD-1 on unstimulated T cells or T cells stimulated with the CD22-negative myeloid cell line K562 or with the CD22-positive B-ALL cell line Nalm6 for 24 hours. (C) Flow cytometry analysis of T cell subsets on day 14 after initial T cell activation. T cell subsets are defined according to expression CD4, CD8, CD62L and CD45RA. CD62L+/CD45RA+ cells represent stem cell memory-like T cells, CD62L+/CD45RA− cells represent central-memory T cells, CD62L−/CD45RA− cells represent effector-memory T cells, and CD62L−/CD45RA+ cells represent effector T cells.

FIG. 6 shows the CoCAR system with an anti-EGFR-CAR and an anti-CEA-CoCAR. (A) displays the schematic CAR/CoCAR design on the immune cell. (B) shows the flow cytometric measurement of receptor expression in transduced T cells. CAR expression was detected using a fluorescently labelled anti-IgG antibody and CoCAR expression was detected by biotinylated ProteinL, followed by streptavidin-PE (C) displays an increased expression of the exhaustion marker LAG-3 and TIM-3 three weeks after transduction in T cells only expressing the anti-EGFR-CAR compared to T cells co-expressing the anti-EGFR-CAR with the anti-CEA-CoCAR.

FIG. 7 shows the advantageous functional properties of anti-EGFR-CAR T cells when co-expressing an anti-CEA CoCAR. (A) shows the low expression of EGFR by MCF-7 compared to other cell lines (A-431, SKOV-3) assessed by flow cytometry, which is further quantified in (B) using Quantibrite PE Quantification Beads (Becton Dickinson). (C) shows the increased secretion of IL-2 by anti-EGFR-CAR T cells co-expressing the anti-CEA-CoCAR compared to the anti-EGFR-CAR alone, as measured after 1:1 co-culture with MCF-7 cells using a LEGENDplex assay. (D) monitors the cell growth of MCF-7 in co-culture with T cells expressing the different CAR constructs at a ratio of 1:1 using an Incucyte system (Sartorius). The graph clearly shows that T cells expressing an anti-EGFR-CAR have an enhanced cytolytic activity long-term when co-expressing an anti-CEA-CoCAR. This is further confirmed in (E) showing the induction of apoptosis of MCF-7 cells as measured using an AnnexinV binding dye.

FIG. 8 schematically illustrates the difference between an exemplary CAR and a chimeric co receptor (CoCAR). The CoCAR lacks a CD3ζ T cell activation domain, so it only acts in trans to amplify the CAR. Note that both CAR and CoCAR polypeptides can form covalently linked homodimers in T cells, which is due to the presence of cysteine residues in the hinge domains of CD28 and CD8α that form a disulfide bond.

FIG. 9 shows expression of exemplary CAR and CoCAR polypeptides on the surface of transduced primary human T cells, as determined by flow cytometry. The anti-CD19 CoCAR with an intracellular CD28 domain was efficiently expressed on the T cell surface, whereas the CoCARs with an intracellular CD2, CD3ε, CD44 or CD146 domain were only weakly visible on the T cell surface.

FIG. 10 shows expression of CD69 and CD25 activation markers on unstimulated CAR-T cells, or CAR-T cells co-cultured with wild-type or CD22UL cl.3 NALM6 cells at an effector-to-target ratio of 1:1 for 24 hours. The CoCAR with an intracellular CD28 domain was the only polypeptide tested that significantly enhanced CAR-T cell activation in response to NALM6-CD22UL tumor cells expressing 400 CD22 antigens per cell.

FIG. 11 shows the concentration of (A) IFNγ, (B) IL-2 and (C) TNFα in supernatants of the indicated CAR-T cell lines co-cultured with the indicated tumor cell lines for 24 hours. The concentration of indicated cytokines were measured by flow cytometry using a LegendPlex assay kit (BioLegend). The CoCAR with an intracellular CD28 domain was the only polypeptide tested that significantly enhanced effector cytokine secretion by CAR-T cells in response to NALM6-CD22UL tumor cells expressing 400 CD22 antigens per cell in a strictly antigen-dependent manner.

FIG. 12 shows the cytotoxicity of the indicated CAR-T cell lines with or without coexpression of a CoCAR co-cultured with (A) NALM6 cells, (B) NALM6-CD22UL cl.3 cells, or (C) K562 cells at different effector-to-target ratios for 18 hours. CAR-T cells coexpressing the CoCAR with intracellular CD28 domain outperformed all tested CoCAR-T cells and conventional CAR-T cells against NALM6-CD22UL tumor cells expressing 400 CD22 antigens per cell.

FIG. 13 shows schematically the coexpression of exemplary CAR and CoCAR polypeptides. In the CoCAR polypeptides with intracellular CD44 and CD146 domain, the CD8α transmembrane domain was replaced by CD44 or CD146 transmembrane domains, respectively. These version 2 CoCARs are termed 19-44v2 and 19-146v2.

FIG. 14 shows expression of exemplary CAR and CoCAR polypeptides on the surface of transduced primary human T cells, as determined by flow cytometry. The modification of the transmembrane domains restored cell-surface expression of CoCARs with intracellular CD44 and CD146 domains in transduced human T cells.

FIG. 15 shows the differentiation status of CD4+ or CD8+ CAR-T cells expressing the indicated constructs, as determined by flow cytometric analysis of CD62L and CD45RA expression. CD62L+CD45RA+ T cells represent stem cell-like memory T cells (Tscm), CD62L+CD45RA-T cells represent central memory T cells (Tcm), CD62L-CD45RA-T cells represent effector memory T cells (Tem), and CD62L-CD45RA+ T cells represent effector T cells (Teff). CAR-T cells coexpressing a 19-28, 19-44v2 or 19-146v2 CoCAR showed a less differentiated phenotype compared to conventional CAR-T cells or untransduced T cells.

FIG. 16 shows expression of (A) CD22 and (B) CD19 molecules on the cell-surface of different NALM6 cell clones. The calculated number of CD22 or CD19 molecules per cell is depicted on the right. Quantification of CD22 or CD19 molecules per cell was performed by flow cytometry using PE-labeled anti-CD22 and CD19 antibodies and Quantibrite™ PE quantification beads (BD Biosciences).

FIG. 17 shows expression of CD69 and CD25 activation markers on unstimulated CAR-T cells, or CAR-T cells co-cultured with wild-type or CD22UL cl.3 NALM6 cells at an effector-to-target ratio of 1:1 for 24 hours. All tested CoCARs enhanced CAR-T cell activation in response to NALM6 cells, whereas the CD28- and CD44-based CoCARs were capable to enhance anti-CD22 CAR-T cell activation in response to tumor cells expressing only 40 CD22 antigens per cell.

FIG. 18 shows the cytotoxicity of the indicated CAR-T cell lines co-cultured with different NALM6 cell clones expressing varying amounts of CD22 molecules per cell at different effector-to-target ratios for 18 hours. CAR-T cells coexpressing a 19-28, 19-44v2 or 19-146v2 CoCAR outperformed conventional CAR-T cells against NALM6-CD22UL tumor cells expressing ultra-low CD22 antigen densities.

FIG. 19 shows the concentration of (A) IFNγ, (B) IL-2 and (C) TNFα in supernatants of the indicated CAR-T cell lines co-cultured with the indicated tumor cell lines for 24 hours. The concentration of indicated cytokines were measured by Bio-Plex using a Bio-Plex assay kit (Bio-Rad). CAR-T cells coexpressing a 19-28, 19-44v2 or 19-146v2 CoCAR outperformed conventional CAR-T cells in effector cytokine release against CD22 ultra-low NALM6 tumor cells, whereas the CAR-T cells coexpressing the 19-28 CoCAR secreted the highest amounts of cytokines in the CD22 ultra-low tumor setting.

FIG. 20 shows expression of exemplary CoCAR and CAR polypeptides carrying wild-type or mutated CAR-CD3ζ ITAMs (indicated with xxx) on the surface of transduced primary human T cells, as determined by flow cytometry. Mutation of the CAR-CD3ζ ITAMs had no influence on the cell-surface expression of the CAR or CoCAR polypeptides.

FIG. 21 shows expression of CD69 and CD25 activation markers on unstimulated CAR-T cells, or CAR-T cells co-cultured with wild-type NALM6 cells at an effector-to-target ratio of 1:1 for 24 hours. Engaged 19-28 CoCAR was unable to enhance CAR-T cell activation in the absence of CAR signaling.

FIG. 22 shows the cytotoxicity of the indicated CAR-T cell lines containing wild-type or mutated ITAMs co-cultured with NALM6 cells at an effector-to-target ratio of 1:1 for 48 hours. Shown is the growth of GFP-positive NALM6 cells, as measured by live-cell imaging in an IncuCyte instrument (Sartorius). Engaged 19-28 CoCAR was unable to promote CAR-T cell cytotoxicity in the absence of CAR signaling.

FIG. 23 shows the concentration of effector cytokines IFNγ, IL-2 and TNFα in supernatants of the indicated CAR-T cell lines co-cultured with NALM6 cells at an effector-to-target ration of 1:1 for 24 hours. The concentration of indicated cytokines were measured by flow cytometry using a LegendPlex assay kit (BioLegend). Engaged 19-28 CoCAR was unable to promote cytokine release by CAR-T cells in the absence of CAR signaling.

FIG. 24 shows expression of exemplary CoCAR and CAR polypeptides with an anti-HSV gB specificity on the surface of transduced primary human T cells, as determined by flow cytometry.

FIG. 25 shows expression of CD69 and CD25 activation markers on unstimulated CAR-T cells, or CAR-T cells co-cultured with wild-type NALM6 cells at an effector-to-target ratio of 1:1 for 24 hours. Engaged 19-44v2 and 19-146v2 CoCARs were unable to promote CAR-T cell activation in the absence of CAR engagement.

FIG. 26 shows the cytotoxicity of the indicated CAR-T cell lines co-cultured with NALM6 cells at an effector-to-target ratio of 1:1 for 48 hours. Shown is the growth of GFP-positive NALM6 cells, as measured by live-cell imaging in an IncuCyte instrument (Sartorius). Engaged 19-44v2 and 19-146v2 CoCARs were unable to promote CAR-T cell cytotoxicity in the absence of CAR engagement.

FIG. 27 shows the concentration of effector cytokines (A) IFNγ, (B) IL-2 and (C) TNFα in supernatants of the indicated CAR-T cell lines co-cultured with NALM6 cells at an effector-to-target ration of 1:1 for 24 hours. The concentration of indicated cytokines were measured by flow cytometry using a LegendPlex assay kit (BioLegend). Engaged 19-44v2 and 19-146v2 CoCARs were unable to promote cytokine release by CAR-T cells in the absence of CAR engagement.

FIG. 28 shows coexpression of exemplary CAR and CoCAR polypeptides targeting different epitopes on the same antigen CD22 on the surface of transduced primary human T cells, as determined by flow cytometry.

FIG. 29 shows the concentration of effector cytokines (A) IFNγ, (B) IL-2 and (C) TNFα in supernatants of the indicated T cell lines co-expressing anti-CD22 CAR and CoCAR constructs, co-cultured with CD22 ultra-low NALM6 cell lines at an effector-to-target ration of 1:1 for 24 hours. The concentration of indicated cytokines was measured by flow cytometry using a LegendPlex assay kit (BioLegend). Co-expression of a CoCAR with CD28 or CD146 signaling domain enhanced the release of effector cytokines by CAR-T cells stimulated with antigen-low tumor cells.

FIG. 30 shows the cytotoxicity of CAR/CoCAR-T cells targeting different epitopes on CD22 against parental and CD22 ultra-low NALM6 cells. Anti-CD22 CAR/CoCAR-T cells having a CD28- or CD146-CoCAR showed enhanced killing of CD22 ultra-low NALM6 cells compared to conventional anti-CD22 CAR-T cells and to CAR/CoCAR-T cells having a CD44-CoCAR.

FIG. 31 shows the densities of HER2 and EGFR molecules on the cell-surface of SKOV-3 and MCF-7 cells. Quantification of HER2 and EGFR molecules per cell was performed by flow cytometry using PE-labeled anti-HER2 and anti-EGFR antibodies and Quantibrite™ PE quantification beads (BD Biosciences). SKOV-3 cells express high amounts of HER2 and EGFR molecules, while MCF-7 cells express significantly lower amounts of these tumor-associated antigens.

FIG. 32 shows cell-surface expression of exemplary CAR and CoCAR polypeptides targeting the tumor-associated antigens HER2 and EGFR of solid tumor cells on transduced primary human T cells, as determined by flow cytometry.

FIG. 33 shows the concentration of IL-2 in supernatants of the indicated CAR-T cell lines co-cultured with SKOV-3 or MCF-7 cells at an effector-to-target ration of 1:1 for 24 hours. The concentration of indicated cytokines were measured by ELISA (ACROBiosystems). Co-expression of a CoCAR enhanced the release of IL-2 by CAR-T cells stimulated with SKOV-3 or MCF-7 solid tumor cells expressing high or low densities of the target antigens, respectively.

FIG. 34 shows the cytotoxicity of the indicated CAR-T cell lines co-cultured with SKOV-3 (A) or MCF-7 (B) solid tumor cells at an effector-to-target ratio of 1:1 for 72 hours. Shown is the growth of GFP-positive tumor cells, as measured by live-cell imaging in an IncuCyte instrument (Sartorius). Co-expression of 19-28, 19-44v2 or 19-146v2 CoCARs enhanced the cytotoxicity of CAR-T cells against SKOV-3 or MCF-7 solid tumor cells expressing high or low densities of the target antigens, respectively.

### SEQUENCES

Sequence anti-CD22 antigen-recognition domain (humanized RFB4 scFv)
(IGHV3-23 leader sequence):
SEQ ID NO: 1
MEFGLSWLFLVAILKGVQC
(VL CDR1):
SEQ ID NO: 2
RASQDISNYLN
(VL CDR2):
SEQ ID NO: 3
YTSILHS
(VL CDR3):
SEQ ID NO: 4
QQGNTLPWT
(VL):
SEQ ID NO: 5
DIQMTQSPSSLSASVGDRVTITCRASQDISNYLNWLQQKPGK
APKLLIYYTSILHSGVPSRFSGSGSGTEFTLTISSLQPEDFATYYCQQGNTLPWTFGQGTKLEIKR
(CDRs 1 to 3 in bold, framework regions 1, 2, 3 and 4 underlined;
numbering from N- to C-terminus (left to right))
(VH CDR1):
SEQ ID NO: 6
GFAFSIYDMS
(VH CDR2):
SEQ ID NO: 7
YISSGGGTTYYPDTVKG
(VH CDR3):
SEQ ID NO: 8
HSGYGSSYGVLFAY
(VH):
SEQ ID NO: 9
EVQLVESGGGLVQPGGSLRLSCAASGFAFSIYDMSWVRQV
PGKGLEWVSYISSGGGTTYYPDTVKGRFTISRDNSRNTLDLQMNSLRVEDTAVYYCAHSGYGS
SYGVLFAYWGQGTLVTVSS
(CDRs 1 to 3 in bold, framework regions 1, 2, 3 and 4 underlined;
numbering from N- to C-terminus (left to right))
(linker L1):
SEQ ID NO: 10
GGGGGGGGSGGGGS
(LFR1):
SEQ ID NO: 11
DIQMTQSPSSLSASVGDRVTITC
(LFR2):
SEQ ID NO: 12
WLQQKPGKAPKLLIY
(LFR3):
SEQ ID NO: 13
GVPSRFSGSGSGTEFTLTISSLQPEDFATYYC
(LFR4):
SEQ ID NO: 14
FGQGTKLEIKR
(HFR1):
SEQ ID NO: 15
EVQLVESGGGLVQPGGSLRLSCAAS
(HFR2):
SEQ ID NO: 16
WVRQV PGKGLEWVS
(HFR3):
SEQ ID NO: 17
RFTISRDNSRNTLDLQMNSLRVEDTAVYYCA
(HFR4):
SEQ ID NO: 18
WGQGTLVTVSS
Sequence anti-CD22 antigen-recognition domain (mouse LL2 scFv)
(mouse IgH leader sequence):
SEQ ID NO: 19
MERHWIFLFLLSVTAGVHS
(VL CDR1):
SEQ ID NO: 20
QSVLYSANHKNY
(VL CDR2):
SEQ ID NO: 21
WAS
(VL CDR3):
SEQ ID NO: 22
HQYLSSWT
(VL):
SEQ ID NO: 23
DIQLTQSPSSLAVSAGENVTMSCKSSQSVLYSANHKNYLAWYQQKPGQSPKLLIYWASTRESGV
PDRFTGSGSGTDFTLTISRVQVEDLAIYYCHQYLSSWTFGGGTKLEIK
(CDRs 1 to 3 in bold, framework regions 1, 2, 3 and 4 underlined;
numbering from N- to C-terminus (left to right))
(VH CDR1):
SEQ ID NO: 24
GYTFTSYW
(VH CDR2):
SEQ ID NO: 25
INPRNDYT
(VH CDR3):
SEQ ID NO: 26
ARRDITTFY
(VH):
SEQ ID NO: 27
QVQLQQSGAELVKPGASVKMSCKASGYTFTSYWLHWIKQRPGQGLEWIGYINPRNDYTEYNQ
KFKDKATLTADKSSSTAYMQLSSLTSEDSAVYYCARRDITTFYWGQGTTLTVSS
(CDRs 1 to 3 in bold, framework regions 1, 2, 3 and 4 underlined;
numbering from N- to C-terminus (left to right))
(linker L1):
SEQ ID NO: 28
GGGGGGGGSGGGGS
(LFR1):
SEQ ID NO: 29
DIQLTQSPSSLAVSAGENVTMSCKSS
(LFR2):
SEQ ID NO: 30
LAWYQQKPGQSPKLLIY
(LFR3):
SEQ ID NO: 31
TRESGVPDRFTGSGSGTDFTLTISRVQVEDLAIYYC
(LFR4):
SEQ ID NO: 32
FGGGTKLEIK
(HFR1):
SEQ ID NO: 33
QVQLQQSGAELVKPGASVKMSCKAS
(HFR2):
SEQ ID NO: 34
LHWIKQRPGQGLEWIGY
(HFR3):
SEQ ID NO: 35
EYNQKFKDKATLTADKSSSTAYMQLSSLTSEDSAVYYC
(HFR4):
SEQ ID NO: 36
WGQGTTLTVSS
Sequence anti-CD19 antigen-recognition domain (mouse FMC63 scFv)
(CSF2Ra leader sequence):
SEQ ID NO: 37
MLLLVTSLLLCELPHPAFLLIP
(VL CDR1):
SEQ ID NO: 38
QDISKY
(VL CDR2):
SEQ ID NO: 39
HTS
(VL CDR3):
SEQ ID NO: 40
QQGNTLPYT
(VL):
SEQ ID NO: 41
DIQMTQTTSSLSASLGDRVTISCRASQDISKYLNWYQQKPDGTVKLLIYHTSRLHSGVPSRFSGS
GSGTDYSLTISNLEQEDIATYFCQQGNTLPYTFGGGTKLEIT
(CDRs 1 to 3 in bold, framework regions 1, 2, 3 and 4 underlined;
numbering from N- to C-terminus (left to right))
(VH CDR1):
SEQ ID NO: 42
GVSLPDYG
(VH CDR2):
SEQ ID NO: 43
IWGSETT
(VH CDR3):
SEQ ID NO: 44
AKHYYYGGSYAMDY
(VH):
SEQ ID NO: 45
EVKLQESGPGLVAPSQSLSVTCTVSGVSLPDYGVSWIRQPPRKGLEWLGVIWGSETTYYNSALK
SRLTIIKDNSKSQVFLKMNSLOTDDTAIYYCAKHYYYGGSYAMDYWGQGTSVTVSS
(CDRs 1 to 3 in bold, framework regions 1, 2, 3 and 4 underlined;
numbering from N- to C-terminus (left to right))
(Whitlow linker L2):
SEQ ID NO: 46
GSTSGSGKPGSGEGSTKG
(LFR1):
SEQ ID NO: 47
DIQMTQTTSSLSASLGDRVTISCRAS
(LFR2):
SEQ ID NO: 48
LNWYQQKPDGTVKLLIY
(LFR3):
SEQ ID NO: 49
RLHSGVPSRFSGSGSGTDYSLTISNLEQEDIATYFC
(LFR4):
SEQ ID NO: 50
FGGGTKLEIT
(HFR1):
SEQ ID NO: 51
EVKLQESGPGLVAPSQSLSVTCTVS
(HFR2):
SEQ ID NO: 52
VSWIRQPPRKGLEWLGV
(HFR3):
SEQ ID NO: 53
YYNSALKSRLTIIKDNSKSQVFLKMNSLQTDDTAIYYC
(HFR4):
SEQ ID NO: 54
WGQGTSVTVSS
Sequence anti-CEA antigen-recognition domain (human scFv)
(mouse IGHV1-61 leader sequence):
SEQ ID NO: 55
MGWSCIILFLVATATGVHS
(VL CDR1):
SEQ ID NO: 56
SSVSYMH
(VL CDR2):
SEQ ID NO: 57
LLIYSTSNLAS
(VL CDR3):
SEQ ID NO: 58
HQWSSYP
(VL):
SEQ ID NO: 59
DIQMTQSPSSLSASVGDRVTITCSTSSSVSYMHWYQQKPGKAPRLLIYSTSNLASGVPSRFSGSG
SGTDFTFTISSLQPEDIATYYCHQWSSYPTFGQGTKVEIK
(CDRs 1 to 3 in bold, framework regions 1, 2, 3 and 4 underlined;
numbering from N- to C-terminus (left to right))
(VH CDR1):
SEQ ID NO: 60
FTISSGYSWH
(VH CDR2):
SEQ ID NO: 61
WIGYIQYSGITNY
(VH CDR3):
SEQ ID NO: 62
AREDYDYHWYFDV
(VH):
SEQ ID NO: 63
QVQLQESGPGLVRPSQTLSLTCTVSGFTISSGYSWHWVRQPPGRGLEWIGYIQYSGITNYNPSL
KSRVTMLVDTSKNQFSLRLSSVTAADTAVYYCAREDYDYHWYFDVWGQGSTVTVSS
(CDRs 1 to 3 in bold, framework regions 1, 2, 3 and 4 underlined;
numbering from N- to C-terminus (left to right))
(Whitlow linker L2):
SEQ ID NO: 64
GSTSGSGKPGSGEGSTKG
(LFR1):
SEQ ID NO: 65
DIQMTQSPSSLSASVGDRVTITCSTS
(LFR2):
SEQ ID NO: 66
WYQQKPGKAPR
(LFR3):
SEQ ID NO: 67
GVPSRFSGSGSGTDFTFTISSLQPEDIATYYC
(LFR4):
SEQ ID NO: 68
TFGQGTKVEIK
(HFR1):
SEQ ID NO: 69
QVQLQESGPGLVRPSQTLSLTCTVSG
(HFR2):
SEQ ID NO: 70
WVRQPPGRGLE
(HFR3):
SEQ ID NO: 71
NPSLKSRVTMLVDTSKNQFSLRLSSVTAADTAVYYC
(HFR4):
SEQ ID NO: 72
WGQGSTVTVSS
Sequence CAR backbone
(CD8a hinge):
SEQ ID NO: 73
FVPVFLPAKPTTTPAPRPPTPAPTIASQPLSLRPEACRPAAGGAVHTRGLDFACD
(CD8a transmembrane):
SEQ ID NO: 74
IYIWAPLAGTCGVLLLSLVITLYCNHRN
(IgG1 hinge):
SEQ ID NO: 75
EPKSPDKTHTCPPCP
(IgG1 CH2 domain):
SEQ ID NO: 76
APELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPA
PIEKTISKAK
(IgG1 CH3 domain):
SEQ ID NO: 77
GQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQ
KSLSLSPGK
(CD28 hinge):
SEQ ID NO: 78
IEVMYPPPYLDNEKSNGTIIHVKGKHLCPSPLFPGPSKP
(CD28 transmembrane domain):
SEQ ID NO: 79
FWVLVVVGGVLACYSLLVTVAFIIFWV
(4-1BB domain):
SEQ ID NO: 80
RFSVVKRGRKKLLYIFKQPFMRPVQTTQEEDGCS
CRFPEEEEGGCEL
(CD3ζ domain):
SEQ ID NO: 81
RVKFSRSADAPAYQQGQNQLYNELNLGRREEYD
VLDKRRGRDPEMGGKPRRKNPQEGLYNELQKDKMAEAYSEIGMKGERRRGKGHDGLYQGLST
ATKDTYDALHMQALPPR
Sequence CoCAR backbone
(CD4 hinge):
SEQ ID NO: 82
SGQVLLESNIKVLPTWSTPVQP
(CD4 transmembrane):
SEQ ID NO: 83
MALIVLGGVAGLLLFIGLGIFF
(CD4 intracellular):
SEQ ID NO: 84
CVRCRHRRRQAERMSQIKRLLSEKKTCQCPHRFQKTCSPI
(CD8a hinge):
SEQ ID NO: 85
TTTPAPRPPTPAPTIASQPLSLRPEACRPAAGGAVHTRGLDFACD
(CD8a transmembrane):
SEQ ID NO: 86
IYIWAPLAGTCGVLLLSLVIT
(CD8a intracellular):
SEQ ID NO: 87
LYCNHRNRRRVCKCPRPVVKSGDKPSLSARYV
(T2A):
SEQ ID NO: 88
SGQVLLESNIKVLPTWSTPVQP
Sequence of intracellular domains capable of binding LCK, motifs
capable of binding LCK, or intracellular domains comprising LCK
(CD4 intracellular):
SEQ ID NO: 84
CVRCRHRRRQAERMSQIKRLLSEKKTCQCPHRFQKTCSPI
(CD8α intracellular):
SEQ ID NO: 87
LYCNHRNRRRVCKCPRPVVKSGDKPSLSARYV
(CD4 and CD8α LCK binding motif):
SEQ ID NO: 89
CxCP
(x = any amino acid; Kim, 2003, Science)
(CD4 LCK binding motif):
SEQ ID NO: 90
CQCP
(CD8a LCK binding motif):
SEQ ID NO: 91
CKCP
(CD3ε intracellular):
SEQ ID NO: 92
KNRKAKAKPVTRGAGAGGRQRGQNKERPPPVPNPDYEPIRKGQRDLYSGL
(CD3ε LCK binding motif):
SEQ ID NO: 93
RKxQRxxY
(x = any amino acid, Hartl, 2020, Nat Immunol)
(CD3ε LCK binding motif):
SEQ ID NO: 94
RKGQRDLY
(CD28 intracellular):
SEQ ID NO: 95
RSKRSRLLHSDYMNMTPRRPGPTRKHYQPYAPPRDFAAYRS
(CD28 LCK binding motif):
SEQ ID NO: 96
RSKRSR
(Dobbins, 2018, Sci Signal; Holdorf, 1999, J Exp Med)
(CD28 LCK binding motif):
SEQ ID NO: 97
RRPGPTRK
(Dobbins, 2018, Sci Signal; Holdorf, 1999, J Exp Med)
(CD28 LCK binding motif):
SEQ ID NO: 98
PYAPP
(Dobbins, 2018, Sci Signal; Holdorf, 1999, J Exp Med)
(CD44 intracellular):
SEQ ID NO: 99
NSRRRCGQKKKLVINSGNGAVEDRKPSGLNGEASKSQEMVHLVNKESSETPDQFMTADETRN
LQNVDMKIGV
(CD44 LCK binding motif):
SEQ ID NO: 100
NSRRRCGQKKKLVINSGNGAVEDRKPSGLNG
(Lefebvre, 2010, Mol Immunol)
(CD146 intracellular):
SEQ ID NO: 101
KKGKLPCRRSGKQEITLPPSRKSELVVEVKSDKLPEEMGLLQGSSGDKRAPGDQGEKYIDLRH
(CD146 LCK binding motif):
SEQ ID NO: 102
KKGKLPCRRSGKQEITLPPSRKSEL
(Duan, J Clin Invest, 2021)
(LCK):
SEQ ID NO: 103
MGCGCSSHPEDDWMENIDVCENCHYPIVPLDGKGTLLIRNGSEVRDPLVTYEGSNPPASPLQDN
LVIALHSYEPSHDGDLGFEKGEQLRILEQSGEWWKAQSLTTGQEGFIPFNFVAKANSLEPEPWFF
KNLSRKDAERQLLAPGNTHGSFLIRESESTAGSFSLSVRDFDQNQGEVVKHYKIRNLDNGGFYIS
PRITFPGLHELVRHYTNASDGLCTRLSRPCQTQKPQKPWWEDEWEVPRETLKLVERLGAGQFGE
VWMGYYNGHTKVAVKSLKQGSMSPDAFLAEANLMKQLQHQRLVRLYAVVTQEPIYIITEYME
NGSLVDFLKTPSGIKLTINKLLDMAAQIAEGMAFIEERNYIHRDLRAANILVSDTLSCKIADFGLA
RLIEDNEYTAREGAKFPIKWTAPEAINYGTFTIKSDVWSFGILLTEIVTHGRIPYPGMTNPEVIQNL
ERGYRMVRPDNCPEELYQLMRLCWKERPEDRPTFDYLRSVLEDFFTATEGQYQPQP
Sequence CAR constructs
(anti-CD22 CAR with humanized RFB4 scFv and CD8a backbone):
SEQ ID NO: 104
MEFGLSWLFLVAILKGVQCEVQLVESGGGLVQPGGSLRLSCAASGFAFSIYDMSWVRQVPGKGL
EWVSYISSGGGTTYYPDTVKGRFTISRDNSRNTLDLQMNSLRVEDTAVYYCARHSGYGSSYGVL
FAYWGQGTLVTVSSGGGGSGGGGSGGGGSDIQMTQSPSSLSASVGDRVTITCRASQDISNYLNW
LQQKPGKAPKLLIYYTSILHSGVPSRFSGSGSGTEFTLTISSLQPEDFATYYCQQGNTLPWTFGQG
TKLEIKRAAAFVPVFLPAKPTTTPAPRPPTPAPTIASQPLSLRPEACRPAAGGAVHTRGLDFACDIY
IWAPLAGTCGVLLLSLVITLYCNHRNRFSVVKRGRKKLLYIFKQPFMRPVQTTQEEDGCSCRFPE
EEEGGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
(anti-CD22 CAR with humanized RFB4 scFv and IgG-Fc backbone):
SEQ ID NO: 105
MEFGLSWLFLVAILKGVQCEVQLVESGGGLVQPGGSLRLSCAASGFAFSIYDMSWVRQVPGKGL
EWVSYISSGGGTTYYPDTVKGRFTISRDNSRNTLDLQMNSLRVEDTAVYYCARHSGYGSSYGVL
FAYWGQGTLVTVSSGGGGSGGGGSGGGGSDIQMTQSPSSLSASVGDRVTITCRASQDISNYLNW
LQQKPGKAPKLLIYYTSILHSGVPSRFSGSGSGTEFTLTISSLQPEDFATYYCQQGNTLPWTFGQG
TKLEIKRAAAEPKSPDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGKFWVLVVVGGVLACY
SLLVTVAFIIFWVRFSVVKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
Sequence CoCAR constructs
(anti-CD19 CD4-CoCAR):
SEQ ID NO: 106
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITGSTS
GSGKPGSGEGSTKGEVKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYYGGSYAMDYWGQGT
SVTVSSVAAAASGQVLLESNIKVLPTWSTPVQPMALIVLGGVAGLLLFIGLGIFFCVRCRHRRRQ
AERMSQIKRLLSEKKTCQCPHRFQKTCSPI
(anti-CD22 CD4-CoCAR):
SEQ ID NO: 107
MERHWIFLFLLSVTAGVHSQVQLQQSGAELVKPGASVKMSCKASGYTFTSYWLHWIKQRPGQG
LEWIGYINPRNDYTEYNQKFKDKATLTADKSSSTAYMQLSSLTSEDSAVYYCARRDITTFYWGQ
GTTLTVSSGGGGSGGGGSGGGGSDIQLTQSPSSLAVSAGENVTMSCKSSQSVLYSANHKNYLAW
YQQKPGQSPKLLIYWASTRESGVPDRFTGSGSGTDFTLTISRVQVEDLAIYYCHQYLSSWTFGGG
TKLEIKAAASGQVLLESNIKVLPTWSTPVQPMALIVLGGVAGLLLFIGLGIFFCVRCRHRRRQAER
MSQIKRLLSEKKTCQCPHRFQKTCSPI
(anti-CEA CD4-CoCAR):
SEQ ID NO: 108
MGWSCIILFLVATATGVHSDIQMTQSPSSLSASVGDRVTITCSTSSSVSYMHWYQQKPGKAPRLL
IYSTSNLASGVPSRFSGSGSGTDFTFTISSLQPEDIATYYCHQWSSYPTFGQGTKVEIKGSTSGSGK
PGSGEGSTKGQVQLQESGPGLVRPSQTLSLTCTVSGFTISSGYSWHWVRQPPGRGLEWIGYIQYS
GITNYNPSLKSRVTMLVDTSKNQFSLRLSSVTAADTAVYYCAREDYDYHWYFDVWGQGSTVTV
SSGAAASGQVLLESNIKVLPTWSTPVQPMALIVLGGVAGLLLFIGLGIFFCVRCRHRRRQAERMS
QIKRLLSEKKTCQCPHRFQKTCSPI
Sequence bicistronic CAR-CoCAR constructs
(anti-CD22-CAR-T2A-anti-CD22-CD4-CoCAR):
SEQ ID NO: 109
MEFGLSWLFLVAILKGVQCEVQLVESGGGLVQPGGSLRLSCAASGFAFSIYDMSWVRQVPGKGL
EWVSYISSGGGTTYYPDTVKGRFTISRDNSRNTLDLQMNSLRVEDTAVYYCARHSGYGSSYGVL
FAYWGQGTLVTVSSGGGGSGGGGSGGGGSDIQMTQSPSSLSASVGDRVTITCRASQDISNYLNW
LQQKPGKAPKLLIYYTSILHSGVPSRFSGSGSGTEFTLTISSLQPEDFATYYCQQGNTLPWTFGQG
TKLEIKRAAAEPKSPDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGKFWVLVVVGGVLACY
SLLVTVAFIIFWVRFSVVKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPREFGGGEGRGSLLTCGDVE
ENPGPRMERHWIFLFLLSVTAGVHSQVQLQQSGAELVKPGASVKMSCKASGYTFTSYWLHWIK
QRPGQGLEWIGYINPRNDYTEYNQKFKDKATLTADKSSSTAYMQLSSLTSEDSAVYYCARRDIT
TFYWGQGTTLTVSSGGGGSGGGGSGGGGSDIQLTQSPSSLAVSAGENVTMSCKSSQSVLYSANH
KNYLAWYQQKPGQSPKLLIYWASTRESGVPDRFTGSGSGTDFTLTISRVQVEDLAIYYCHQYLSS
WTFGGGTKLEIKAAASGQVLLESNIKVLPTWSTPVQPMALIVLGGVAGLLLFIGLGIFFCVRCRH
RRRQAERMSQIKRLLSEKKTCQCPHRFQKTCSPI
Nucleic acid sequences
(anti-CD22 CAR with humanized RFB4 scFv and CD8a backbone)
SEQ ID NO: 110
ATGGAGTTTGGCCTGAGCTGGCTGTTCCTGGTGGCCATCCTGAAGGGCGTGCAGTGTGAGGT
GCAGCTGGTGGAGTCTGGGGGAGGCTTGGTCCAGCCTGGGGGGTCCCTGAGACTCTCCTGTG
CAGCCTCTGGATTCGCTTTCAGTATCTATGACATGTCTTGGGTCCGCCAGGTTCCAGGGAAG
GGGCTGGAGTGGGTCTCATACATTAGTAGTGGTGGTGGTACCACCTACTATCCAGACACTGT
GAAGGGCAGATTCACCATCTCCAGAGACAATTCCAGGAACACTCTGGATCTTCAAATGAAC
AGTCTGAGAGTCGAGGACACGGCTGTCTATTATTGTGCGAGACATAGTGGCTACGGTAGTAG
CTACGGGGTTTTGTTTGCTTACTGGGGCCAAGGAACCCTGGTCACCGTCTCCTCAGGTGGAG
GCGGTTCAGGCGGAGGTGGCTCTGGCGGTGGCGGATCGGACATCCAGATGACTCAGTCTCC
ATCCTCCCTGTCTGCATCTGTAGGAGACAGAGTCACCATCACCTGCAGGGCAAGTCAGGACA
TTAGCAATTATTTAAACTGGCTTCAACAGAAACCAGGGAAAGCCCCTAAGCTCCTGATTTAC
TACACATCAATATTACACTCAGGAGTCCCATCAAGGTTCAGCGGCAGTGGATCTGGGACAGA
GTTCACTCTCACAATCAGCAGCCTGCAGCCTGAAGATTTTGCAACTTATTACTGTCAACAGG
GTAATACGCTTCCGTGGACGTTTGGCCAGGGGACCAAACTGGAAATCAAACGTGCGGCCGC
ATTCGTGCCGGTCTTCCTGCCAGCGAAGCCCACCACGACGCCAGCGCCGCGACCACCAACAC
CGGCGCCCACCATCGCGTCGCAGCCCCTGTCCCTGCGCCCAGAGGCGTGCCGGCCAGCGGCG
GGGGGCGCAGTGCACACGAGGGGGCTGGACTTCGCCTGTGATATCTACATCTGGGCGCCCTT
GGCCGGGACTTGTGGGGTCCTTCTCCTGTCACTGGTTATCACCCTTTACTGCAACCACAGGA
ACCGTTTCTCTGTTGTTAAACGGGGCAGAAAGAAGCTCCTGTATATATTCAAACAACCATTT
ATGAGACCAGTACAAACTACTCAAGAGGAAGATGGCTGTAGCTGCCGATTTCCAGAAGAAG
AAGAAGGAGGATGTGAACTGAGAGTGAAGTTCAGCAGGAGCGCAGACGCCCCCGCGTACCA
GCAGGGCCAGAACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTACAGTGAGATTG
GGATGAAAGGCGAGCGCCGGAGGGGCAAGGGGCACGATGGCCTTTACCAGGGTCTCAGTAC
AGCCACCAAGGACACCTACGACGCCCTTCACATGCAGGCCCTGCCCCCTCGCTAA
(anti-CD22 CAR with humanized RFB4 scFv and IgG-Fc backbone)
SEQ ID NO: 111
ATGGAGTTTGGCCTGAGCTGGCTGTTCCTGGTGGCCATCCTGAAGGGCGTGCAGTGTGAGGT
GCAGCTGGTGGAGTCTGGGGGAGGCTTGGTCCAGCCTGGGGGGTCCCTGAGACTCTCCTGTG
CAGCCTCTGGATTCGCTTTCAGTATCTATGACATGTCTTGGGTCCGCCAGGTTCCAGGGAAG
GGGCTGGAGTGGGTCTCATACATTAGTAGTGGTGGTGGTACCACCTACTATCCAGACACTGT
GAAGGGCAGATTCACCATCTCCAGAGACAATTCCAGGAACACTCTGGATCTTCAAATGAAC
AGTCTGAGAGTCGAGGACACGGCTGTCTATTATTGTGCGAGACATAGTGGCTACGGTAGTAG
CTACGGGGTTTTGTTTGCTTACTGGGGCCAAGGAACCCTGGTCACCGTCTCCTCAGGTGGAG
GCGGTTCAGGCGGAGGTGGCTCTGGCGGTGGCGGATCGGACATCCAGATGACTCAGTCTCC
ATCCTCCCTGTCTGCATCTGTAGGAGACAGAGTCACCATCACCTGCAGGGCAAGTCAGGACA
TTAGCAATTATTTAAACTGGCTTCAACAGAAACCAGGGAAAGCCCCTAAGCTCCTGATTTAC
TACACATCAATATTACACTCAGGAGTCCCATCAAGGTTCAGCGGCAGTGGATCTGGGACAGA
GTTCACTCTCACAATCAGCAGCCTGCAGCCTGAAGATTTTGCAACTTATTACTGTCAACAGG
GTAATACGCTTCCGTGGACGTTTGGCCAGGGGACCAAACTGGAAATCAAACGTGCGGCCGC
TGAGCCCAAATCTCCTGACAAAACTCACACATGCCCACCGTGCCCAGCACCTGAACTCCTGG
GGGGACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCAGTACA
ACAGCACGTACCGTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCAAGCCGGGATGAGCT
GACCAAGAACCAGGTCAGCCTGACCTGCCTGGTCAAAGGCTTCTATCCCAGCGACATCGCCG
TGGAGTGGGAGAGCAATGGGCAGCCGGAGAACAACTACAAGACCACGCCTCCCGTGTTGGA
CTCCGACGGCTCCTTCTTCCTCTACAGCAAGCTCACCGTGGACAAGAGCAGGTGGCAGCAGG
GGAACGTCTTCTCATGCTCCGTGATGCATGAGGCTCTGCACAACCACTACACGCAGAAGAGC
CTCTCCCTGTCTCCCGGGAAATTTTGGGTGCTGGTGGTGGTTGGTGGAGTCCTGGCTTGCTAT
AGCTTGCTAGTAACAGTGGCCTTTATTATTTTCTGGGTGCGTTTCTCTGTTGTTAAACGGGGC
AGAAAGAAGCTCCTGTATATATTCAAACAACCATTTATGAGACCAGTACAAACTACTCAAGA
GGAAGATGGCTGTAGCTGCCGATTTCCAGAAGAAGAAGAAGGAGGATGTGAACTGAGAGTG
AAGTTCAGCAGGAGCGCAGACGCCCCCGCGTACCAGCAGGGCCAGAACCAGCTCTATAACG
AGCTCAATCTAGGACGAAGAGAGGAGTACGATGTTTTGGACAAGAGACGTGGCCGGGACCC
TGAGATGGGGGGAAAGCCGAGAAGGAAGAACCCTCAGGAAGGCCTGTACAATGAACTGCA
GAAAGATAAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTACGACGCC
CTTCACATGCAGGCCCTGCCCCCTCGCTAA
(anti-CD19 CD4-CoCAR):
SEQ ID NO: 112
ATGCTTCTCCTGGTGACAAGCCTTCTGCTCTGTGAGTTACCACACCCAGCATTCCTCCTGATC
CCAGACATCCAGATGACACAGACTACATCCTCCCTGTCTGCCTCTCTGGGAGACAGAGTCAC
CATCAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATCAAG
GTTCAGTGGCAGTGGGTCTGGAACAGATTATTCTCTCACCATTAGCAACCTGGAGCAAGAAG
ATATTGCCACTTACTTTTGCCAACAGGGTAATACGCTTCCGTACACGTTCGGAGGGGGGACT
AAGTTGGAAATAACAGGCTCCACCTCTGGATCCGGCAAGCCCGGATCTGGCGAGGGATCCA
CCAAGGGCGAGGTGAAACTGCAGGAGTCAGGACCTGGCCTGGTGGCGCCCTCACAGAGCCT
GTCCGTCACATGCACTGTCTCAGGGGTCTCATTACCCGACTATGGTGTAAGCTGGATTCGCC
AGCCTCCACGAAAGGGTCTGGAGTGGCTGGGAGTAATATGGGGTAGTGAAACCACATACTA
TAATTCAGCTCTCAAATCCAGACTGACCATCATCAAGGACAACTCCAAGAGCCAAGTTTTCT
TAAAAATGAACAGTCTGCAAACTGATGACACAGCCATTTACTACTGTGCCAAACATTATTAC
TACGGTGGTAGCTATGCTATGGACTACTGGGGTCAAGGAACCTCAGTCACCGTCTCCTCAGT
AGCAGCGGCCGCATCCGGCCAAGTGCTTCTGGAGAGCAACATCAAGGTACTGCCAACATGG
AGCACTCCTGTACAGCCAATGGCCCTCATCGTTCTCGGAGGTGTTGCGGGTCTTTTGCTCTTT
ATCGGGCTTGGGATTTTCTTCTGCGTGAGATGTCGCCATAGAAGGCGACAGGCAGAACGCAT
GAGTCAGATCAAGCGCTTGCTGTCCGAGAAGAAAACCTGTCAGTGTCCCCATAGGTTCCAGA
AAACTTGCAGCCCTATATAA
(anti-CD22 CD4-CoCAR):
SEQ ID NO: 113
ATGGAAAGGCACTGGATCTTTCTCTTCCTGTTGTCAGTAACTGCAGGTGTGCACTCGCAGGT
CCAGCTGCAGCAGTCAGGGGCTGAACTGGTGAAACCTGGGGCCTCAGTGAAGATGTCCTGC
AAGGCTTCTGGCTACACCTTTACTAGCTACTGGCTGCACTGGATAAAACAGAGGCCTGGACA
GGGTCTGGAATGGATTGGATACATTAATCCTAGGAATGATTATACTGAGTACAATCAGAAAT
TCAAGGACAAGGCCACATTGACTGCAGACAAATCCTCCAGCACAGCCTATATGCAACTGAG
CAGCCTGACATCTGAGGACTCTGCAGTCTATTACTGCGCGCGAAGGGATATTACTACGTTCT
ACTGGGGCCAAGGCACCACTCTCACAGTCTCCTCGGGTGGAGGCGGTTCAGGCGGAGGTGG
CTCTGGCGGTGGCGGATCAGATATCCAGCTGACCCAGTCTCCATCATCTCTGGCTGTGTCTGC
AGGAGAAAACGTCACTATGAGCTGTAAGTCCAGTCAAAGTGTTTTATACAGTGCAAATCACA
AGAACTACTTGGCCTGGTACCAGCAGAAACCAGGGCAGTCTCCTAAACTGCTGATCTACTGG
GCATCCACTAGGGAATCTGGTGTCCCTGATCGCTTCACAGGCAGCGGATCTGGGACAGATTT
TACTCTTACCATCAGCAGAGTACAAGTTGAAGACCTGGCAATTTATTATTGTCACCAATACC
TCTCCTCGTGGACGTTCGGTGGAGGGACCAAGCTGGAGATCAAAGCGGCCGCATCCGGCCA
AGTGCTTCTGGAGAGCAACATCAAGGTACTGCCAACATGGAGCACTCCTGTACAGCCAATG
GCCCTCATCGTTCTCGGAGGTGTTGCGGGTCTTTTGCTCTTTATCGGGCTTGGGATTTTCTTCT
GCGTGAGATGTCGCCATAGAAGGCGACAGGCAGAACGCATGAGTCAGATCAAGCGCTTGCT
GTCCGAGAAGAAAACCTGTCAGTGTCCCCATAGGTTCCAGAAAACTTGCAGCCCTATATAA
(anti-CEA CD4-CoCAR):
SEQ ID NO: 114
ATGGGATGGAGCTGTATCATCCTCTTCCTGGTAGCAACAGCTACAGGCGTGCACAGTGACAT
CCAGATGACCCAGAGCCCAAGCAGCCTGAGCGCCAGCGTGGGTGACAGAGTGACCATCACC
TGTAGTACCAGCTCAAGTGTAAGTTACATGCACTGGTACCAGCAGAAGCCAGGTAAGGCTCC
AAGGCTGCTGATCTACAGCACATCCAACCTGGCTTCTGGTGTGCCAAGCAGATTCAGCGGTA
GCGGTAGCGGTACCGACTTCACCTTCACCATCAGCAGCCTCCAGCCAGAGGACATCGCCACC
TACTACTGCCATCAGTGGAGTAGTTATCCCACGTTCGGCCAAGGGACCAAGGTGGAAATCAA
AGGATCTACTTCCGGTTCAGGAAAGCCCGGGAGTGGTGAAGGTAGCACTAAAGGCCAGGTC
CAGCTGCAGGAGAGCGGTCCAGGTCTTGTGAGACCTAGCCAGACCCTGAGCCTGACCTGCA
CCGTGTCTGGCTTCACCATCAGCAGTGGTTATAGCTGGCACTGGGTGAGACAGCCACCTGGA
CGAGGTCTTGAGTGGATTGGATACATACAGTACAGTGGTATCACTAACTACAACCCCTCTCT
CAAAAGTAGAGTGACAATGCTGGTAGACACCAGCAAGAACCAGTTCAGCCTGAGACTCAGC
AGCGTGACAGCCGCCGACACCGCGGTCTATTATTGTGCAAGAGAAGACTATGATTACCACTG
GTACTTCGATGTCTGGGGTCAAGGCAGCACGGTCACCGTCTCCTCAGGTGCGGCCGCTTCCG
GCCAAGTGCTTCTGGAGAGCAACATCAAGGTACTGCCAACATGGAGCACTCCTGTACAGCC
AATGGCCCTCATCGTTCTCGGAGGTGTTGCGGGTCTTTTGCTCTTTATCGGGCTTGGGATTTT
CTTCTGCGTGAGATGTCGCCATAGAAGGCGACAGGCAGAACGCATGAGTCAGATCAAGCGC
TTGCTGTCCGAGAAGAAAACCTGTCAGTGTCCCCATAGGTTCCAGAAAACTTGCAGCCCTAT
ATAA
(anti-CD22-CAR-T2A-anti-CD22-CD4-CoCAR):
SEQ ID NO: 115
ATGGAAAGGCACTGGATCTTTCTCTTCCTGTTGTCAGTAACTGCAGGTGTGCACTCGGAGGT
GCAGCTGGTGGAGTCTGGGGGAGGCTTGGTCCAGCCTGGGGGGTCCCTGAGACTCTCCTGTG
CAGCCTCTGGATTCGCTTTCAGTATCTATGACATGTCTTGGGTCCGCCAGGTTCCAGGGAAG
GGGCTGGAGTGGGTCTCATACATTAGTAGTGGTGGTGGTACCACCTACTATCCAGACACTGT
GAAGGGCAGATTCACCATCTCCAGAGACAATTCCAGGAACACTCTGGATCTTCAAATGAAC
AGTCTGAGAGTCGAGGACACGGCTGTCTATTATTGTGCGAGACATAGTGGCTACGGTAGTAG
CTACGGGGTTTTGTTTGCTTACTGGGGCCAAGGAACCCTGGTCACCGTCTCCTCAGGTGGAG
GCGGTTCAGGCGGAGGTGGCTCTGGCGGTGGCGGATCGGACATCCAGATGACTCAGTCTCC
ATCCTCCCTGTCTGCATCTGTAGGAGACAGAGTCACCATCACCTGCAGGGCAAGTCAGGACA
TTAGCAATTATTTAAACTGGCTTCAACAGAAACCAGGGAAAGCCCCTAAGCTCCTGATTTAC
TACACATCAATATTACACTCAGGAGTCCCATCAAGGTTCAGCGGCAGTGGATCTGGGACAGA
GTTCACTCTCACAATCAGCAGCCTGCAGCCTGAAGATTTTGCAACTTATTACTGTCAACAGG
GTAATACGCTTCCGTGGACGTTTGGCCAGGGGACCAAACTGGAAATCAAACGTGCGGCCGC
TGAGCCCAAATCTCCTGACAAAACTCACACATGCCCACCGTGCCCAGCACCTGAACTCCTGG
GGGGACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCAGTACA
ACAGCACGTACCGTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCAAGCCGGGATGAGCT
GACCAAGAACCAGGTCAGCCTGACCTGCCTGGTCAAAGGCTTCTATCCCAGCGACATCGCCG
TGGAGTGGGAGAGCAATGGGCAGCCGGAGAACAACTACAAGACCACGCCTCCCGTGTTGGA
CTCCGACGGCTCCTTCTTCCTCTACAGCAAGCTCACCGTGGACAAGAGCAGGTGGCAGCAGG
GGAACGTCTTCTCATGCTCCGTGATGCATGAGGCTCTGCACAACCACTACACGCAGAAGAGC
CTCTCCCTGTCTCCCGGGAAATTTTGGGTGCTGGTGGTGGTTGGTGGAGTCCTGGCTTGCTAT
AGCTTGCTAGTAACAGTGGCCTTTATTATTTTCTGGGTGCGTTTCTCTGTTGTTAAACGGGGC
AGAAAGAAGCTCCTGTATATATTCAAACAACCATTTATGAGACCAGTACAAACTACTCAAGA
GGAAGATGGCTGTAGCTGCCGATTTCCAGAAGAAGAAGAAGGAGGATGTGAACTGAGAGTG
AAGTTCAGCAGGAGCGCAGACGCCCCCGCGTACCAGCAGGGCCAGAACCAGCTCTATAACG
AGCTCAATCTAGGACGAAGAGAGGAGTACGATGTTTTGGACAAGAGACGTGGCCGGGACCC
TGAGATGGGGGGAAAGCCGAGAAGGAAGAACCCTCAGGAAGGCCTGTACAATGAACTGCA
GAAAGATAAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTACGACGCC
CTTCACATGCAGGCCCTGCCCCCTCGCGAATTCGGCGGCGGAGAGGGCAGAGGCAGTCTGCT
GACATGCGGTGACGTGGAAGAGAATCCCGGCCCTAGGATGGAAAGGCACTGGATCTTTCTC
TTCCTGTTGTCAGTAACTGCAGGTGTGCACTCGCAGGTCCAGCTGCAGCAGTCAGGGGCTGA
ACTGGTGAAACCTGGGGCCTCAGTGAAGATGTCCTGCAAGGCTTCTGGCTACACCTTTACTA
GCTACTGGCTGCACTGGATAAAACAGAGGCCTGGACAGGGTCTGGAATGGATTGGATACAT
TAATCCTAGGAATGATTATACTGAGTACAATCAGAAATTCAAGGACAAGGCCACATTGACTG
CAGACAAATCCTCCAGCACAGCCTATATGCAACTGAGCAGCCTGACATCTGAGGACTCTGCA
GTCTATTACTGCGCGCGAAGGGATATTACTACGTTCTACTGGGGCCAAGGCACCACTCTCAC
AGTCTCCTCGGGTGGAGGCGGTTCAGGCGGAGGTGGCTCTGGCGGTGGCGGATCAGATATC
CAGCTGACCCAGTCTCCATCATCTCTGGCTGTGTCTGCAGGAGAAAACGTCACTATGAGCTG
TAAGTCCAGTCAAAGTGTTTTATACAGTGCAAATCACAAGAACTACTTGGCCTGGTACCAGC
AGAAACCAGGGCAGTCTCCTAAACTGCTGATCTACTGGGCATCCACTAGGGAATCTGGTGTC
CCTGATCGCTTCACAGGCAGCGGATCTGGGACAGATTTTACTCTTACCATCAGCAGAGTACA
AGTTGAAGACCTGGCAATTTATTATTGTCACCAATACCTCTCCTCGTGGACGTTCGGTGGAG
GGACCAAGCTGGAGATCAAAGCGGCCGCATCCGGCCAAGTGCTTCTGGAGAGCAACATCAA
GGTACTGCCAACATGGAGCACTCCTGTACAGCCAATGGCCCTCATCGTTCTCGGAGGTGTTG
CGGGTCTTTTGCTCTTTATCGGGCTTGGGATTTTCTTCTGCGTGAGATGTCGCCATAGAAGGC
GACAGGCAGAACGCATGAGTCAGATCAAGCGCTTGCTGTCCGAGAAGAAAACCTGTCAGTG
TCCCCATAGGTTCCAGAAAACTTGCAGCCCTATATAA

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., (1995) Helvetica Chimica Acta, CH-4010 Basel, Switzerland.

The practice of the present invention will employ, unless otherwise indicated, conventional methods of biochemistry, cell biology, immunology, and recombinant DNA techniques which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

The term “about” means approximately or nearly, and in the context of a numerical value or range set forth herein in one embodiment means±20%, ±10%, ±5%, or ±3% of the numerical value or range recited or claimed.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, is hereby incorporated by reference in its entirety. Nothing herein is to be construed as an admission that the invention is not entitled to antedate such disclosure by virtue of prior invention.

The CAR-CoCAR concept according to the present invention is fundamentally different from the previously published concepts because it describes the co-expression of a conventional CAR containing an intracellular CD3ζ T cell activation domain and a coreceptor-based chimeric antigen receptor (CoCAR or “chimeric coreceptor”) containing at least one intracellular domain capable of binding LCK in a given immune effector cell (e.g. T cells or NK cells), aiming to regulate the recruitment of the kinase LCK to the CAR synapse by antigen binding. The regulated recruitment of CoCAR-associated LCK to the CAR synapse in response to antigen binding may provide a technical solution to the general problems with i) the antigen-sensitivity of BBz-CARs, which often require thousands of antigen molecules per cell to activate T cell responses, and ii) with CARs inducing T cell exhaustion as a result of tonic signalling due to high basal CAR-CD3ζ phosphorylation. The exemplary results presented herein demonstrate that co-expressing a CAR recognizing a first antigen and a CoCAR recognizing a second antigen expressed on the same target cell (e.g. a tumor cell) were capable of enhancing cell (e.g. CAR-T cells) activation, resulting in increased T cell effector functions and anti-tumor efficacy (e.g. increased effector cytokine production and cytotoxicity). Furthermore, co-expressing a CAR and a CoCAR was shown to be capable of enhancing CAR-T cell activity in response to tumor cells expressing a very low antigen density, resulting in increased T cell effector functions and anti-tumor efficacy (e.g. increased effector cytokine production and cytotoxicity). Furthermore, it was surprisingly found that immune cells (e.g. T cells) co-expressing a CAR and a CoCAR showed less signs of T cell exhaustion (e.g. reduced antigen-independent effector cytokine production and expression of inhibitory receptors such as TIM-3 and LAG-3) compared to immune cells expressing a conventional CAR. Thus, co-expressing a CAR and a CoCAR may be capable of lowering the T cell activation threshold, improving CAR function in immune cells, and improving immune cell functions by enhancing proximal antigen-induced CAR signalling, while reducing antigen-independent CAR-CD35 phosphorylation. Thus, the inventors assume that immune cells co-expressing a CAR and a CoCAR will generate more potent clinical cell products with reduced probability of relapses due to outgrowth of antigen-low tumor cells and/or termination of CAR-T cell responses due T cell exhaustion.

There is an urgent and unmet clinical need for the development of novel CAR constructs having improved anti-tumor properties to overcome these limitations to enhance the reach of these therapeutics.

It is one aim of the invention to provide a CoCAR capable of binding LCK to improve the properties of CARs known in the art by enhancing activation and increase the potency of immune cells (e.g. T cells) in response to tumor cells, even when the antigen is expressed at a very low density. It is one further aim of the present invention to provide an immune response against diseased cells expressing at least one tumor-associated antigen, and to treat a disease such as a cancer disease involving cells expressing at least one tumor-associated antigen. Preferably the invention involves the administration of antigen receptor-engineered immune effector cells such as T cells targeted against diseased cells expressing at least one tumor-associated antigen. In general, cells expressing an antigen on the surface can be targeted by immune effector cells carrying an antigen receptor targeted to the antigen.

Exemplary tumor-associated antigens to be used in the present invention include, but are not limited to, those described in Table 1.

TABLE 1
Exemplary tumor-associated antigens

| Antigen | Description |
|---|---|
| B7-H3 (CD276) | This member of the B7 family functions as an immune inhibitor and is |
| | expressed in various kinds of tumours including prostate cancer and non-small- |
| | cell lung cancer. (Yang, 2020, Int. J. Biol. Sci.) |
| BCMA (CD269) | Ubiquitously expressed on plasma cells and is therefore an attractive target for |
| | the treatment of multiple myeloma, which is already approved for two CAR T- |
| | cell products (Abecma, Carvykti). (Yu, 2020, Journal of Hematology & |
| | Oncology) |
| CD19 | CD19 is expressed on pro-B, pre-B and mature B cells and is targeted by four |
| | approved CAR T-cell products (Kymriah, Yescarta, Tecartus, Breyanzi) for the |
| | treatment of B cell malignancies. (Nagro, 2005, Immunologic Research) |
| CD20 | During their differentiation B cells express the surface antigen CD20, which |
| | makes it an interesting alternative to CD19 to treat B cell malignancies. |
| | (Shanehbandi, 2017, Curr. Cancer Drug Targets) |
| CD22 | CD22 from the immunoglobulin superfamily represents another B-cell |
| | restricted surface protein, which can be targeted to treat various B-cell |
| | malignancies. (Tu, 2011, J. Exp. Ther. Oncol.) |
| CD44v6 | The CD44 is involved in cell-cell and cell-matrix interaction and comprises a |
| | variety of splice variants. Isoforms containing the CD44 variant domain 6 |
| | (CD44v6) have been associated with tumor progression and metastasis. |
| | (Heider, 2004, Cancer Immunol. Immunother.) |
| CD70 | CD70 is a promising target to treat AML since it is expressed on leukemic |
| | blasts as well as on leukemic stem cells, while normal hematopoietic stem cells |
| | display no detectable expression. (Sauer, 2021, Blood) |
| CD79A | The CD79A/CD79B heterodimer is part of the B cell antigen complex and is |
| | specifically expressed in B lineage lymphocytes from early developmental |
| | stages on until terminal differentiation to plasma cells. It therefore represents a |
| | valuable target for immunotherapeutic agents to treat B cell malignancies. |
| CD79B | The CD79A/CD79B heterodimer is part of the B cell antigen complex and is |
| | specifically expressed in B lineage lymphocytes from early developmental |
| | stages on until terminal differentiation to plasma cells. It therefore represents a |
| | valuable target for immunotherapeutic agents to treat B cell malignancies. |
| CD123 | In contrast to normal hematopoietic stem cells, leukemic stem cells highly |
| | express CD123 and it is used as an important marker for identification and |
| | targeting of refractory or relapsed acute myeloid leukemia. (Shi, 2019, |
| | Cardiovasc. Hematol. Disord. Drug Targets) |
| CEA | Even though the carcinoembryonic antigen (CEA) is normally not expressed in |
| | higher quantities after birth, colorectal cancer and some other carcinomas |
| | display elevated abundancies. Furthermore, high level of CEA expression has |
| | been associated with poor prognosis. (Hall, 2019, Ann. Coloproctol.) |
| Claudin-6 | Claudin-6 (CLDN6) is one of the 27 family members of claudins and majorly |
| | involved in the tight junction and cell-to-cell adhesion of epithelial cell sheets, |
| | playing a significant role in cancer initiation and progression. |
| Claudin-18.2 | Claudin 18.2, a member of the claudin family, is commonly expressed in |
| | multiple cancers, including GC and PC. Claudin 18.2 is not expressed in any |
| | healthy tissues with the exception of gastric mucosa. |
| CLL1 | The expression of the human C-type lectin-like molecule-1 (CLL-1) is |
| | restricted to myeloid cells and is present on most AML blasts as well as |
| | leukemia stem cells. However, it is absent in normal hematopoietic stem cells. |
| | (Wang, 2018, J. Hematol. Oncol.) |
| CXCR5 | Due to its expression on various lymphocytes, the chemokine receptor 5 |
| | (CXCR5) represents an interesting target for treating different lymphomas, but |
| | also other cancer types. (Wang, 2022, Biochim. Biophys. Acta. Rev. |
| | Cancer)(Wang, 2021, Nat. Commun.) |
| EGFR/EGFRvIII | Epidermal growth factor receptor (EGFR) is one of the most frequently altered |
| | genes in human cancer, leading to extensive proliferation and resistance to |
| | apoptosis. EGFR and especially its constantly activated variant EGFRvIII |
| | represents therefore a highly interesting antigen for targeting a variety of |
| | cancers. (Talukdar, 2020, Adv. Cancer Res.)(Wykosky, 2011, Chin. J. Cancer) |
| HER2 | HER2 is largely focused for the treatment of breast cancer, even though it has |
| | been reported to be also overexpressed in other cancer types. Several approved |
| | agents to target HER2-positive breast cancer have already proven the feasibility |
| | to target HER2 in solid tumors. (Oh, 2020, Nat. Rev. Clin. Oncol.) |
| GD2 | The disialoganglioside GD2 is strongly overexpressed by neuroblastomas, |
| | melanomas and a variety of other cancer types, making it a very attractive |
| | tumor associated antigen for targeted therapy approaches. (Nazha, 2020, Front. |
| | Oncol.) |
| Mesothelin | Mesothelin expression in cancer includes malignant mesothelioma, pancreatic, |
| | ovarian and lung adenocarcinomas. On healthy tissue it is only present on |
| | mesothelial cells, which are dispensable. (Hassan, 2016, J. Clin. Oncol.) |
| MUC-1 | The transmembrane glycoprotein mucin 1 (MUC1) is a mucin family member |
| | that has different functions in normal and cancer cells. In cancer cells, MUC1 |
| | often undergoes aberrant glycosylation and overexpression. It is involved in |
| | cancer invasion, metastasis, angiogenesis, and apoptosis by virtue of its |
| | participation in intracellular signaling processes and the regulation of related |
| | biomolecules. |
| MUC-16 | MUC16 (previously known as CA125) has been extensively used as a |
| | biomarker for ovarian cancer, and its expression has been associated with |
| | disease progression. Aberrant overexpression of MUC16 has been observed in |
| | several human malignancies, including ovarian, pancreatic, breast, and lung. |
| | Due to their aberrant overexpression and functional involvement, MUC16 and |
| | its ligands have emerged as potential targets for therapeutic intervention using |
| | monoclonal antibodies and immunotherapy. |
| PSMA | The Prostate-specific membrane antigen (PSMA) is highly expressed in |
| | prostate cancer, making it a feasible target for diagnostic as well as therapeutic |
| | purposes. (Wang, 2022, Prostate Cancer Prostatic Dis.) |
| ROR1 | Several blood and solid malignancies display higher expression of ROR1 |
| | compared to healthy cells, indicating its potential for a targeted therapy. |
| | (Karvonen, 2017, Biochem. Soc. Trans.) |
| SLAMF7 | SLAMF7 is overexpressed in multiple myeloma, which is already utilized by |
| | various antibody based drugs. (Malaer, 2017, Am. J. Cancer Res.) |

The skilled person understands that a coreceptor-CAR (CoCAR) of the present invention, comprising a first extracellular antigen-recognition domain recognizing any tumor-associated antigen can be designed and produced.

The term “antigen” relates to an agent comprising an epitope against which an immune response is to be generated and/or is directed. Preferably, an antigen in the context of the present invention is a molecule which, optionally after processing, induces an immune reaction, which is preferably specific for the antigen or cells expressing the antigen, preferably on the cell surface. The term “antigen” includes in particular proteins and peptides. An antigen is preferably a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present invention, an antigen corresponds to a naturally occurring protein or a part thereof, which is expressed on the cell surface preferably of malignant cells.

An antigen expressed at an ultra-low density refers in the context of the present invention to an antigen that is expressed on a cell surface at a density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell. Antigen density can be measured using methods well-known in the state of the art, such as staining the cells with fluorescent antibodies specific for that antigen and acquiring by flow cytometry the stained cells and quantification beads (e.g. Quantibrite, Becton Dickinson).

The term “cell surface” is used in accordance with its normal meaning in the art, and thus includes the outside of the cell which is accessible to binding by proteins and other molecules. An antigen is expressed on the surface of cells if it is located at the surface of said cells and is accessible to binding by antigen-binding molecules such as antigen receptors or antigen-specific antibodies added to the cells. An antigen expressed on the surface of cells can be an integral membrane protein having an extracellular portion recognized by an antigen receptor. An antigen receptor is expressed on the surface of cells if it is located at the surface of said cells and is accessible to binding by e.g. antigen to which the antigen receptor is specific added to the cells. An antigen receptor expressed on the surface of cells can also be an integral membrane protein having an extracellular portion recognizing antigen.

The term “extracellular portion” or “ectodomain” in the context of the present invention refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

According to the invention, an antigen and in particular a tumor-associated antigen is expressed in a cell if the level of expression is above the detection limit and/or if the level of expression is high enough to allow binding by antigen-specific antibodies or antigen receptors added to the cell. Preferably, an antigen and in particular a tumor-associated antigen expressed in a cell is expressed or exposed, i.e. is present, on the surface of said cell and, thus, available for binding by antigen-specific molecules such as antibodies or antigen receptors added to the cell.

The term “tumor-associated antigen”, as used herein, relates to an antigen expressed in a tumor cell and/or on the surface of a tumor cell. In particular a tumor-associated antigen is specifically expressed in and/or on a tumor cell.

According to the invention, the term “antigen receptor” includes engineered receptors, which confer an arbitrary specificity such as the specificity of a monoclonal antibody onto an immune effector cell such as a T cell. In this way, a large number of antigen-specific T cells can be generated for adoptive cell transfer. Thus, an antigen receptor may be present on T cells, e.g. instead of or in addition to the T cell's own T cell receptor. Such T cells do not necessarily require processing and presentation of an antigen for recognition of the target cell but rather may recognize preferably with specificity any antigen present on a target cell. Preferably, said antigen receptor is expressed on the surface of the cells. For the purpose of the present invention, T cells comprising an antigen receptor are comprised by the term “T cell” as used herein. Specifically, according to the invention, the term “antigen receptor” includes artificial receptors comprising a complex of domains which recognize, i.e. bind to, a target structure (e.g. an antigen) on a target cell such as a cancer cell (e.g. by binding of an antigen binding site or antigen binding domain to an antigen expressed on the surface of the target cell) and may confer specificity onto an immune effector cell such as a T cell expressing said antigen receptor on the cell surface. Preferably, recognition of the target structure by an antigen receptor results in activation of the immune effector cell expressing said antigen receptor. According to the invention the term “antigen receptor” is preferably synonymous with the terms “chimeric antigen receptor (CAR)”, “chimeric T cell receptor” and “artificial T cell receptor”.

The term “coreceptor” is used in the context of the present invention according to its traditional definition (Rolf König, Handbook of Cell Signaling 2^{nd} Ed., Vol. 3, p. 2679-2688, 2010) to refer to a protein that associate with an antigen receptor, such as a CAR, exert complex regulatory effects on T cell activation, and is characterized by a temporal association with the CAR, which is only triggered by binding to its specific antigen expressed on a tumor cell.

The term “coreceptor-CAR”, “coreceptor” CAR or “CoCAR”, as used herein, describes a chimeric antigen receptor comprising (a) a first extracellular antigen-recognition domain, and (b) at least one cytosolic domain capable of binding LCK and/or a domain comprising LCK or a variant or fragment thereof. The domains (a) and (b) can be linked by a first hinge domain, for example a CD4, CD8, CD28 or IgG-Fc hinge domain, and a first transmembrane domain, for example a CD4, CD8, CD28, or CD35 transmembrane domain. The “coreceptor-CAR” as used in the context of the present invention does not comprise a TCR-recruitment domain, as is the case for the TAC disclosed by Helsen et al., (2018). In addition, the “coreceptor-CAR” as used in the context of the present invention does not comprise an intracellular cell activation domain, such as a CD3ζ (CD3zeta) T cell activation domain. Therefore, the “coreceptor-CAR” is not able to induce immune cell activation (Example 9), has a different structure from a chimeric antigen receptor (CAR), and in the context of the present invention is also referred to as “chimeric coreceptor”.

The term “target cell” shall mean a cell which is a target for an immune response such as a cellular immune response. Target cells include any undesirable cell such as a cancer cell. The target cell can be a cell expressing a target antigen, in particular a tumor-associated antigen, which is preferably present on the cell surface.

The term “antibody” or “immunoglobulin” refers in the context of the present invention to an antigen binding polypeptide comprising two heavy chains that are linked to each other by disulfide bonds and each heavy chain is linked to a light chain by a disulfide bond. There are two types of light chain, lambda (λ) and kappa (κ). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody binding site or paratope and the antigenic determinant. Antibody binding sites are made up of residues that are primarily from the hypervariable or complementarity determining regions (CDRs). Occasionally, residues from non-hypervariable or FR influence the overall domain structure and hence the antigen binding site. CDRs refer to amino acid sequences that together define the binding affinity and specificity of the natural Fv region of a native immunoglobulin binding site. Examples of an antibody or immunoglobulin include IgM, IgD, IgG, IgA or IgE. CDRs of antigen binding polypeptides can be grafted into antibodies, bispecific antibodies, or multispecific antibodies. Knowing the amino acid sequence of the CDRs of for example an antibody, a TCR or an antigen binding polypeptide of the invention, one skilled in the art can determine the framework regions, such as the antibody framework regions or TCR framework regions. However, in cases where the CDRs are not indicated, the skilled person in the art can first determine the CDR amino acid sequences based on the IMGT definition for antibodies and then determine the amino acid sequences of the framework regions.

The term “humanized antibody” refers in the context of the present invention to an antibody which is completely or partially of non-human origin and which has been modified to replace certain amino acids, in particular in the framework regions of the heavy and light chains, in order to avoid or minimize an immune response in humans. The constant domains of a humanized antibody are mainly human heavy and light chain domains. Methods for humanization of antibody sequences are known in the art; (Almagro & Fransson (2008) Front Biosci. 13:1619-1633). One commonly used method is CDR grafting, or antibody reshaping, which involves grafting of the CDR sequences of a donor antibody, generally a mouse antibody, into the framework scaffold of a human antibody of different specificity. Since CDR grafting may reduce the binding specificity and affinity, and thus, the biological activity of a CDR grafted non-human antibody, back mutations may be introduced at selected positions of the CDR grafted antibody in order to retain the binding specificity and affinity of the parent antibody. Amino acid residues that are part of a CDR will typically not be altered, although in certain cases it may be desirable to alter individual CDR amino acid residues, for example to remove a glycosylation site, a deamidation site, an isomerization site, or an undesired cysteine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, in particular by way of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ser. When such a deamidation site, in particular Asn-Gly, is present in a CDR sequence, it may therefore be desirable to remove the site, typically by conservative substitution to remove one of the implicated residues. Substitution in a CDR sequence to remove one of the implicated residues is also intended to be encompassed by the present invention.

According to the invention, an antigen and in particular a tumor-associated antigen can be recognized by the antigen receptor of the invention through any antigen-recognition domains or binding domains (herein also referred to simply as “domains”) able to form an antigen binding site such as through antigen-binding portions of an antibody. Specifically, the present invention makes use of antibody light chain and heavy chain variable domains binding a tumor-associated antigen.

According to the present invention, an antigen receptor is capable of binding to (targeting) a predetermined target if it has a significant affinity for said predetermined target and binds to said predetermined target in standard assays. “Affinity” or “binding affinity” can be measured by equilibrium dissociation constant (K_{D}). Preferably, the term “significant affinity” refers to the binding to a predetermined target with a dissociation constant (K_{D}) of 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, or 10⁻¹² M or lower.

An antigen receptor is specific for a predetermined target if it is capable of binding to said predetermined target while it is not (substantially) capable of binding to other targets, i.e. has no significant affinity for other targets and does not significantly bind to other targets in standard assays. Preferably, an antigen receptor is specific for a predetermined target if the affinity for and the binding to such other targets does not significantly exceed the affinity for or binding to proteins which are unrelated to a predetermined target such as bovine serum albumin (BSA), casein or human serum albumin (HSA). Preferably, an antigen receptor is specific for a predetermined target if it binds to said target with a K_{D} that is at least 10-fold, 100-fold, 10³-fold, 10⁴-fold, 10⁵-fold, or 10⁶-fold lower than the K_{D} for binding to a target for which it is not specific. For example, if the K_{D} for binding of an antigen receptor to the target for which it is specific is 10⁻⁷ M, the K_{D} for binding to a target for which it is not specific would be at least 10⁻⁶ M, 10⁻⁵ M, 10⁻⁴ M, 10⁻³ M, 10⁻² M, or 10⁻¹ M.

Binding of an antigen receptor to a target can be determined experimentally using any suitable method as disclosed for example in Berzofsky et al., “Antibody-Antigen Interactions”, Fundamental Immunology, Paul, W. E., Ed., Raven Press New York, N Y (1984), Kuby, Janis Immunology, W. H. Freeman and Company New York, NY (1992), and methods described herein. Affinities may be readily determined using conventional techniques, such as by equilibrium dialysis by surface plasmon resonance e.g. using the BIAcore 2000 instrument, using the general procedures outlined by the manufacturer, by radioimmunoassay using radiolabeled target antigen, or by another method known to the skilled artisan. The affinity data may be analyzed, for example, by the method of Scatchard et al., Ann N.Y. Acad. ScL, 51:660 (1949). The measured affinity of a particular antibody-antigen interaction can vary if measured under different conditions, e.g., salt concentration, pH. Thus, measurements of affinity and other antigen-binding parameters, e.g., K_{D}, IC₅₀, are preferably made with standardized solutions of antibody and antigen, and a standardized buffer.

The term “immune effector cell” or “immunoreactive cell” in the context of the present invention relates to a cell which exerts effector functions during an immune reaction. An “immune effector cell” preferably is capable of binding an antigen such as a tumor-associated antigen expressed on the surface of a cell and mediating an immune response. For example, such cells secrete cytokines and/or chemokines, kill microbes, secrete antibodies, recognize infected or cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells. Preferably, in the context of the present invention, “immune effector cells” are T cells, preferably CD4⁺ and/or CD8⁺ T cells. As used herein, the term “immune effector cell” also includes a cell which can mature into an immune cell (such as T cell, in particular T helper cell, or cytolytic T cell) with suitable stimulation. Immune effector cells comprise CD34⁺ hematopoietic stem cells, immature and mature T cells and immature and mature B cells. The differentiation of T cell precursors into a cytolytic T cell, when exposed to an antigen, is similar to clonal selection of the immune system.

Preferably, an “immune effector cell” recognizes an antigen such as a tumor-associated antigen with some degree of specificity, in particular if present on the surface of antigen presenting cells or diseased cells such as cancer cells. Preferably, said recognition enables the cell that recognizes an antigen to be responsive or reactive. If the cell is a helper T cell (CD4⁺ T cell) such responsiveness or reactivity may involve the release of cytokines and/or the activation of CD8⁺ lymphocytes (CTLs) and/or B-cells. If the cell is a CTL such responsiveness or reactivity may involve the elimination of cells, i.e., cells characterized by expression of an antigen, for example, via apoptosis or perforin-mediated cell lysis. CTL responsiveness may include sustained calcium flux, cell division, production of cytokines such as IFN-γ, IL-2 and TNF-α, up-regulation of activation markers such as CD25, CD44 and CD69, and specific cytolytic killing of antigen expressing target cells. CTL responsiveness may also be determined using an artificial reporter that accurately indicates CTL responsiveness. Such CTL that recognizes an antigen and are responsive or reactive are also termed “antigen-responsive CTL” herein.

The term “specifically binding” or “specificity” refer in the context of this invention to the binding of an antigen binding polypeptide or fragments thereof to a specific binding site of its target when the target comprises specific and non-specific binding sites. However, sometimes binding of a polypeptide to closely related proteins is unavoidable, then the actual binding to the target may be specific but the antigen binding polypeptide is deemed to be non-specific in relation to the intended target binding. An antigen binding polypeptide as comprised in the chimeric antigen receptor of the present invention is considered to specifically bind if it binds stronger or enhanced to its target than to one or more similar antigens.

A “lymphoid cell” is a cell which, optionally after suitable modification, e.g. after transfer of a T cell receptor or antigen receptor, is capable of producing an immune response such as a cellular immune response, or a precursor cell of such cell, and includes lymphocytes, preferably T lymphocytes, lymphoblasts, and plasma cells. A lymphoid cell may be an immunoreactive cell or immune effector cell as described herein. A preferred lymphoid cell is a T cell which can be modified to express a T cell receptor or antigen receptor on the cell surface. According to one example, the lymphoid cell lacks endogenous expression of a T cell receptor.

The terms “T cell” and “T lymphocyte” are used interchangeably herein and include T helper cells (CD4+ T cells) and cytotoxic T cells (CTLs, CD8+ T cells), which comprise cytolytic T cells. T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptors (TCR). The thymus is the principal organ responsible for maturation of T cells. Several different subsets of T cells have been discovered, each with a distinct function. T helper cells assist other white blood cells in immunologic processes, including maturation of B cells into plasma cells and activation of cytotoxic T cells and macrophages, among other functions. These cells are also known as CD4+ T cells because they express the CD4 protein on their surface.

Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response. Cytotoxic T cells destroy virally infected cells and tumor cells, and are also implicated in transplant rejection. These cells are also known as CD8+ T cells since they express the CD8 glycoprotein at their surface. These cells recognize their targets by binding to antigen associated with MHC class I, which is present on the surface of nearly every cell of the body.

A majority of T cells has a T cell receptor (TCR) existing as a complex of several proteins. The actual T cell receptor is composed of two separate peptide chains, which are produced from the independent T cell receptor alpha and beta (TCRα and TCRβ) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells. Each chain of a T cell receptor is composed of two extracellular domains: a variable (V) region and a constant (C) region. The constant region is proximal to the cell membrane, followed by a transmembrane region and a short cytoplasmic tail, while the variable region binds to the peptide/MHC complex. For the purpose of the present invention, the term “constant region of a T cell receptor chain or a portion thereof” also includes cases in which the constant region of a T cell receptor chain is (from N terminus to C terminus) followed by a transmembrane region and a cytoplasmic tail, such as a transmembrane region and a cytoplasmic tail which are naturally linked to the constant region of a T cell receptor chain.

All T cells originate from hematopoietic stem cells in the bone marrow. Hematopoietic progenitors derived from hematopoietic stem cells populate the thymus and expand by cell division to generate a large population of immature thymocytes. The earliest thymocytes express neither CD4 nor CD8, and are therefore classed as double-negative (CD4−CD8−) cells. As they progress through their development they become double-positive thymocytes (CD4+CD8+), and finally mature to single-positive (CD4+CD8− or CD4−CD8+) thymocytes that are then released from the thymus to peripheral tissues.

T cells may generally be prepared in vitro or ex vivo, using standard procedures. For example, T cells may be isolated from bone marrow, peripheral blood or a fraction of bone marrow or peripheral blood of a mammal, such as a patient, using a commercially available cell separation system. Alternatively, T cells may be derived from related or unrelated humans, non-human animals, cell lines or cultures. A sample comprising T cells may, for example, be peripheral blood mononuclear cells (PBMC).

T cells to be used according to the invention may express an endogenous T cell receptor or may lack expression of an endogenous T cell receptor.

Nucleic acids such as RNA or DNA encoding a CoCAR according to the invention may be introduced into cells such as immune effector cells and specifically T cells or other cells with lytic potential, in particular lymphoid cells.

The antigen receptor of the present invention, when e.g. present on an immune effector cell such as a T cell, recognizes the antigen on for example the surface of antigen presenting cells or diseased cells such as cancer cells, such that the immune effector cell is stimulated, primed and/or expanded or exerts effector functions of immune effector cells as described above.

The term “antigen-specific T cell” or similar terms relate to a T cell which, in particular when provided with an antigen receptor, recognizes the antigen to which the antigen receptor is targeted such as on the surface of antigen presenting cells or diseased cells such as cancer cells and preferably exerts effector functions of T cells as described above. T cells and other lymphoid cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured.

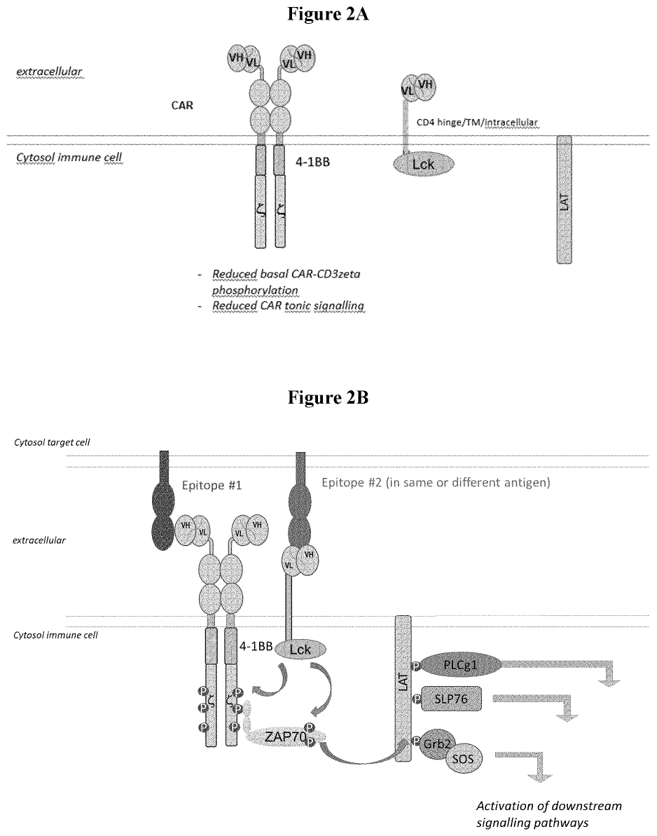

The clinical administration of CAR-T cells has far proven highly efficacious, however, frequently occurring class-specific severe and life-threatening adverse events have become a major challenge for the clinical management of patients being treated with these novel immunotherapeutics. Major class-specific toxicities of these compounds include the development of a so-called cytokine release syndrome (CRS) clinically manifesting with high fever, hypoxia, hypotension, tachycardia, tachypnea, rigor and subsequent dysfunction of major organ systems. Neurologic toxicity termed Immune effector Cell-Associated Neurotoxicity Syndrome (ICANS) may occur after CRS subsiding and manifests with dysphasia, aphasia, somnolence, seizures, tremor and cerebral edema as clinical signs of treatment-induced encephalopathy. Clinical trials employing CD19 CAR T cells in adult ALL have reported fatal CRS/ICANS related outcome between 2% and 16% of cases (reviewed in Sheth and Gauthier, Bone Marrow Transplant 56(3):552-566, 2021). For CD22 CAR-T cell therapeutics utilizing the m971 antigen-recognition domain, 86% of treated patients experienced CRS, and a late onset hemophagocytic lymphohistiocytosis/macrophage activation syndrome (HLH/MAS) manifesting with fever, pan-cytopenia, and potential for subsequent multi-organ failure has been additionally reported at a high frequency (38%) for patients developing CRS after CAR-T cell infusion. High ferritin levels may indicate HLH/MAS and have therefore been suggested as potential biomarker for detection of this condition (La Rosee et al., Blood 133(23):2465-2477, 2019). The development of CRS could be correlated to treatment-induced pro-inflammatory cytokine and chemokine release, and it has been suggested that host myeloid cells play a crucial role in the induction of CRS. Preclinical mouse models of CAR-T cell-induced CRS suggest that the secretion of proinflammatory cytokines such as IL-1 and IL-6 by activated macrophages represents a central mechanism in CRS pathophysiology (Giavridis et al., Nat Med 24(6):731-738, 2018; Norelli et al., Nat Med 24(6):739-748). One critical factor that may determines the incidence and severity of CRS, HLH/MAS and/or ICANS is the design of the CAR, as clinical trials revealed higher rates of toxicities induced by CARs containing CD28 versus 4-1BB costimulatory domains (Cappell & Kochenderfer, Nat Rev Clin Oncol, 2021). The reason for this discrepancy has not been resolved yet, but it seems that the more extensive activation and rapid proliferation of CD28-based CARs correlates with higher toxicities in patients (Salter et al., (2018) Sci Signal. 11(544); Cappell & Kochenderfer, (2021) Nat Rev Clin Oncol. (11) 715-727). The increased signal intensity induced by CD28-based CARs is related to the constitutive association of LCK with the CAR synapse, where it promotes high basal CAR-CD3ζ phosphorylation and tonic CAR signalling (Salter et al., (2018) Sci Signal. 11(544); Sun et al., (2020) Cancer Cell, (2) 216-225). A high degree of tonic CAR signalling has also been described for CARs containing 4-1BB/CD3ζ signalling domains, as for instance the anti-CD22 CAR utilizing the m971 antigen-recognition domain (Singh et al., (2021) Nat Med. (5) 842-850), and notably such CAR-T cells induced inflammatory toxicities in an unexpected high number of treated patients (Shah et al., (2020) J Clin Oncol. (17) 1938-1959). Together, these observations indicate a correlation between the severity of treatment-related toxicities and CARs capable to induce antigen-independent (tonic) CAR signalling. Tonic CAR signalling can be induced by high basal CAR-CD35 phosphorylation, which is the result of constitutive association of LCK with the CAR synapse, and which may induce a distinct functional state of the immune cell (e.g. T cells) that favours the development of toxicities such as CRS, ICANs and/or HLH/MAS. It is therefore a further aim of the present invention to provide a CoCAR to improve the safety of CARs known in the art by reducing the constitutive association of LCK with the CAR synapse, thereby preventing basal CAR-CD3ζ phosphorylation, tonic CAR signalling and antigen-independent immune cell activation, without compromising the activation and the potency of immune cells (e.g. T cells) in response to tumor cells (FIG. 2A).

The present invention provides inter alia coreceptor-CAR (CoCARs) as well as the polypeptide chains of such CoCARs determining the individual domains of said CoCARs, such as the variable light and heavy domains of the antigen binding domain of the antigen-recognition domain comprising the CDRs and framework regions, the hinge domain or region, the transmembrane domain, and one or more intracellular signaling domains.

The CoCAR of the present invention comprises a first extracellular antigen-recognition domain. As used herein, an “extracellular antigen-recognition domain” is capable of specifically recognizing an antigen. The extracellular antigen-recognition domain can comprise a heavy chain variable domain (VH) and light chain variable domain (VL) with complementary determining regions (CDRs) and framework regions (FR) of an antibody or a fragment thereof as defined herein. Preferably, the antigen-recognition domain is as described herein. Preferably, the antigen-recognition domain comprises as antigen binding polypeptides a VH and VL as defined herein.

The extracellular antigen-recognition domain can comprise a light chain variable domain and a heavy chain variable domain, wherein the light chain variable domain and the heavy chain variable domain form an antigen binding polypeptide or antigen-binding site specifically binding a tumor-associated tumor antigen, as described herein.

The CAR, to be combined with the CoCAR of the present invention, comprises a second extracellular antigen-recognition domain. The extracellular antigen-recognition domain of the CAR can comprise a heavy chain variable domain (VH) and light chain variable domain (VL) with complementary determining regions (CDRs) and framework regions (FR) of an antibody or a fragment thereof as defined herein. Preferably, the antigen-recognition domain of the CAR is as described herein. Preferably, the antigen-recognition domain of the CAR comprises as antigen binding polypeptides a VH and VL as defined herein.

The first and the second antigen recognition domain can be independently any antigen recognition domain, as described herein. The first and the second antigen recognition domain can (i) recognize different epitopes located in the same antigen expressed on the surface of a target cell, or (ii) can recognize the same antigen expressed on the surface of a target cell. More preferably, the first and the second antigen recognition domain can (i) recognize different epitopes located in the same antigen expressed on the surface of a target cell, or (ii) can recognize different antigens expressed on the surface of a target cell. Thus, both the first and the second antigen recognition domains recognize tumor-specific or tumor-associated antigens.

The term “antigen binding polypeptide” or “antigen binding domain” refers in the context of this invention to a polypeptide that comprises a paratope (alternatively referred to as “antigen binding site”) that specifically binds to an antigen. Examples of antigen binding polypeptides are inter alia antibodies or fragments thereof or single chain antibodies.

The term “variable domain” refers in the context of this invention to a region of an immunoglobulin, which is defined on the basis of sequence homologies as known to the skilled person. Typically, two variable domains form an antigen binding site. Non-exhausting examples of such domains are the variable light chain domain comprised in the antibody light chain (VL), the variable heavy chain domain comprised in the antibody heavy chain (VH), the alpha variable domain comprised in the alpha chain of a T cell receptor (TCR) molecule (Va) or the beta variable domain comprised in the beta chain of a TCR (VB).

The term “complementary determining region” (CDR) refers in the context of this invention to the non-contiguous antigen combining sites found within the variable domains of immunoglobulins, e.g. in VH, VL, Vα and Vβ. CDRs have been described by Lefranc et al. (2003) Developmental and Comparative Immunology 27:55; Kabat et al., J. Biol. Chem. 252:6609-66I6 (1977); Kabat et al., U.S. Dept. of Health and Human Services, “Sequences of proteins of immunological interest”, 1991; Chothia et al., J. Mol. Biol. I96:90I-917, 1987; and Contact annotation (MacCallum et al., J. Mol. Biol. 262:732-745 (1996) for the Contact annotation); Abhinandan and Martin, Mol. Immunol. (2008), 45(14):3832-9. for AbM annotation; IMGT (Lefranc MP. Unique database numbering system for immunogenetic analysis; Immunol Today (1997) 18:509), where the definitions include overlapping or subsets of amino acid residues when compared against each other. Nevertheless, application of either definition to refer to a CDR of an antibody or grafted antibodies or variants or fragments thereof, is intended to be within the scope of the term as defined and used herein.

The terms “HCDR1” or “VH CDR1”, “HCDR2” or ““VH CDR2”, “HCDR3” or “VH CDR3” refer in the context of the present invention to the first, second, and third CDR in a heavy chain variable domain of an antigen binding polypeptide, for example an antibody or functional fragment thereof. As used herein, the terms “LCDR1” or “VL CDR1”, “LCDR2” or “VL CDR2”, “LCDR3” or “VL CDR3” refer, respectively, to the first, second, and third CDR in a light chain variable domain of an antigen binding polypeptide, for example an antibody or a fragment thereof. As used herein, the terms “CDR1”, “CDR2”, and “CDR3” refer, respectively, to the first, second and third CDRs of either chain's variable region of an antigen binding polypeptide, for example an antibody or functional fragment thereof.

The positions of the CDRs and framework regions as defined herein are assigned according to Kabat or Chothia, in particular according to Kabat numbering. Thus, according to a particularly preferred embodiment of the present invention, the numbering of the light and heavy chain variable regions described herein is in accordance with Kabat.

According to the present invention, the antigen-recognition domain can be an anti-CD22 antigen-recognition domain. The anti-CD22 antigen-recognition domain can comprise a LCDR1 of SEQ ID NO: 2, LCDR2 of SEQ ID NO: 3 and LCDR of SEQ ID NO: 4. The anti-CD22 antigen-recognition domain can also comprise an HCDR1 of SEQ ID NO: 6, HCDR2 of SEQ ID NO: 7 and HCDR3 of SEQ ID NO: 8. The anti-CD22 antigen-recognition domain can comprise an IGHV3-23 leader sequence of SEQ ID NO: 1.

The VL domain anti-CD22 antigen-recognition domain can comprise a LFR1 of SEQ ID NO: 11, LFR2 of SEQ ID NO: 12, LFR3 of SEQ ID NO: 13, and LFR4 of SEQ ID NO: 14.

The VH domain anti-CD22 antigen-recognition domain can comprise a HFR1 of SEQ ID NO: 15, HFR2 of SEQ ID NO: 16, HFR3 of SEQ ID NO: 17, and HFR4 of SEQ ID NO: 18.

The anti-CD22 antigen-recognition domain can comprise a VL of SEQ ID NO: 5.

The anti-CD22 antigen-recognition domain can comprise a VH of SEQ ID NO: 9.

The anti-CD22 antigen-recognition domain can comprise a linker sequence, such as SEQ ID NO: 10, located between the VH and VL sequence, such as between SEQ ID NOs: 5 and 9.

According to the present invention, the antigen-recognition domain can be an anti-CD22 antigen-recognition domain (mouse LL2 scFv). The anti-CD22 antigen-recognition domain can comprise a LCDR1 of SEQ ID NO: 20, LCDR2 of SEQ ID NO: 21 and LCDR3 of SEQ ID NO: 22. The anti-CD22 antigen-recognition domain can also comprise an HCDR1 of SEQ ID NO: 24, HCDR2 of SEQ ID NO: 25 and HCDR3 of SEQ ID NO: 26. The anti-CD22 antigen-recognition domain can comprise a mouse IgH leader sequence of SEQ ID NO: 19.

The VL domain anti-CD22 antigen-recognition domain can comprise a LFR1 of SEQ ID NO: 29, LFR2 of SEQ ID NO: 30, LFR3 of SEQ ID NO: 31, and LFR4 of SEQ ID NO: 32.

The VH domain anti-CD22 antigen-recognition domain can comprise a HFR1 of SEQ ID NO: 33, HFR2 of SEQ ID NO: 34, HFR3 of SEQ ID NO: 35, and HFR4 of SEQ ID NO: 36.

The anti-CD22 antigen-recognition domain can comprise a VL of SEQ ID NO: 23.

The anti-CD22 antigen-recognition domain can comprise a VH of SEQ ID NO: 27.

The anti-CD22 antigen-recognition domain can comprise a linker sequence, such as SEQ ID NO: 28, located between the VH and VL sequence, such as between SEQ ID NOs: 23 and 27.

According to the present invention, the antigen-recognition domain can be an anti-CD22 antigen-recognition domain comprising human m971 scFv. The anti-CD22 antigen-recognition domain can comprise a LCDR1 of SEQ ID NO: 117, LCDR2 of SEQ ID NO: 118 and LCDR3 of SEQ ID NO: 119. The anti-CD22 antigen-recognition domain can also comprise an HCDR1 of SEQ ID NO: 121, HCDR2 of SEQ ID NO: 122 and HCDR3 of SEQ ID NO: 123. The anti-CD22 antigen-recognition domain can comprise a CSFR2a leader sequence of SEQ ID NO: 116.

The VL domain anti-CD22 antigen-recognition domain can comprise a LFR1 of SEQ ID NO: 126, LFR2 of SEQ ID NO: 127, LFR3 of SEQ ID NO: 128, and LFR4 of SEQ ID NO: 129.

The VH domain anti-CD22 antigen-recognition domain can comprise a HFR1 of SEQ ID NO: 130, HFR2 of SEQ ID NO: 131, HFR3 of SEQ ID NO: 132, and HFR4 of SEQ ID NO: 133.

The anti-CD22 antigen-recognition domain can comprise a VL of SEQ ID NO: 120.

The anti-CD22 antigen-recognition domain can comprise a VH of SEQ ID NO: 124.

The anti-CD22 antigen-recognition domain can comprise a linker sequence, such as SEQ ID NO: 125, located between the VH and VL sequence, such as between SEQ ID NOs: 120 and 124.

In one embodiment, the antigen-recognition domain can be an anti-CD22 antigen-recognition domain comprising humanized SGIII scFv.

According to the present invention, the antigen-recognition domain can be an anti-CD19 antigen-recognition domain. The anti-CD19 antigen-recognition domain can comprise a LCDR1 of SEQ ID NO: 38, LCDR2 of SEQ ID NO: 39 and LCDR3 of SEQ ID NO: 40. The anti-CD22 antigen-recognition domain can also comprise an HCDR1 of SEQ ID NO: 42, HCDR2 of SEQ ID NO: 43 and HCDR of SEQ ID NO: 44. The anti-CD19 antigen-recognition domain can comprise an IGHV3-23 leader sequence of SEQ ID NO: 37.

The VL domain anti-CD19 antigen-recognition domain can comprise a LFR1 of SEQ ID NO: 47, LFR2 of SEQ ID NO: 48, LFR3 of SEQ ID NO: 49, and LFR4 of SEQ ID NO: 50.

The VH domain anti-CD19 antigen-recognition domain can comprise a HFR1 of SEQ ID NO: 51, HFR2 of SEQ ID NO: 52, HFR3 of SEQ ID NO: 53, and HFR4 of SEQ ID NO: 54.

The anti-CD19 antigen-recognition domain can comprise a VL of SEQ ID NO: 41.

The anti-CD19 antigen-recognition domain can comprise a VH of SEQ ID NO: 45.

The anti-CD19 antigen-recognition domain can comprise a linker sequence, such as SEQ ID NO: 46, located between the VH and VL sequence, such as between SEQ ID NOs: 41 and 45.

According to the present invention, the antigen-recognition domain can be an anti-CEA antigen-recognition domain. The anti-CEA antigen-recognition domain can comprise a LCDR1 of SEQ ID NO: 56, LCDR2 of SEQ ID NO: 57 and LCDR of SEQ ID NO: 58. The anti-CEA antigen-recognition domain can also comprise an HCDR1 of SEQ ID NO: 60, HCDR2 of SEQ ID NO: 61 and HCDR3 of SEQ ID NO: 62. The anti-CEA antigen-recognition domain can comprise a leader sequence of SEQ ID NO: 55.

The VL domain anti-CEA antigen-recognition domain can comprise a LFR1 of SEQ ID NO: 65, LFR2 of SEQ ID NO: 66, LFR3 of SEQ ID NO: 67, and LFR4 of SEQ ID NO: 68.

The VH domain anti-CEA antigen-recognition domain can comprise a HFR1 of SEQ ID NO: 69, HFR2 of SEQ ID NO: 70, HFR3 of SEQ ID NO: 71, and HFR4 of SEQ ID NO: 72.

The anti-CEA antigen-recognition domain can comprise a VL of SEQ ID NO: 59.

The anti-CEA antigen-recognition domain can comprise a VH of SEQ ID NO: 63.

The anti-CEA antigen-recognition domain can comprise a linker sequence, such as SEQ ID NO: 64, located between the VH and VL sequence, such as between SEQ ID NOs: 59 and 63.

According to the present invention, the antigen-recognition domain can be an anti-EGFR antigen-recognition domain. The anti-EGFR antigen-recognition domain can comprise a scFv derived from anti-EGFR antibody Cetuximab. The anti-EGFR antigen-recognition domain can also comprise a fully human scFv derived from a phage display library selected against recombinant human EGFR protein. It will be appreciated by those skilled in the art that in particular in the sequences of the CDRs, hypervariable and variable regions can be modified without losing the ability to bind to a target. For example, CDR regions may be either identical or highly homologous to the CDRs disclosed herein. By “highly homologous” it is contemplated that from 1 to 3, preferably from 1 to 2, or 1 substitution may be made in the CDRs.

The term “framework region” (FR) refers in the context of the present invention to all amino acid residues outside the CDR regions within the variable domain of an antigen binding polypeptide, for example an antibody or a fragment thereof. A framework region is generally a discontinuous amino acid sequence between about 100-120 amino acids in length but is intended to reference only those amino acids outside of the CDRs. As used herein, the term “framework region” is intended to mean each domain of the framework that is separated by the CDRs. “FR1 to FR4” refer to framework region 1 which is the first N-terminal amino acid sequence of a variable domain, followed by FR2, FR3 and FR4 which are interspersed with CDR1, 2 and 3, respectively.

As used herein the framework regions of the VL are termed LFR1, LFR2, LFR3, and LFR4, respectively. As used herein the framework regions of the VL are termed HFR1, HFR2, HFR3, and HFR4, respectively.

According to the present invention, antigen-recognition domain of the present invention comprises variants or fragments of the framework regions as defined herein.

According to a preferred embodiment of the present invention, the antigen-recognition domain preferably comprises one or more of the light and heavy chain frameworks as defined herein. Preferably, the extracellular antigen-recognition domain comprises at least one of light chain framework region 1 (LFR1), light chain framework region 2 (LFR2), light chain framework region 3 (LFR3) and light chain framework region 4 (LFR4) having a at least 90% amino acid sequence identity to the amino acid sequence of the respective framework regions of the light chain variable region according to SEQ ID NO: 5, 23, 41, or 59. Further or alternatively, the extracellular antigen-recognition domain comprises at least heavy chain framework region 2 (HFR2), heavy chain framework region 3 (HFR3) and heavy chain framework region 4 (HFR4) having a at least 90% amino acid sequence identity to the amino acid sequence of the respective framework regions of the heavy chain variable region according to SEQ ID NO: 9, 27, 45, or 63.

According to a preferred embodiment, LFR1 comprises the sequence as set forth in SEQ ID NO: 11 or a sequence at least 90% identical thereto. Preferably, LFR1 comprises Q at position 3, S at position 7, P at position 8, V at position 15, and/or T at position 22 of SEQ ID NO: 11.

According to a preferred embodiment, LFR2 comprises the sequence as set forth in SEQ ID NO: 12 or a sequence at least 90% identical thereto. Preferably, LFR2 comprises L at position 2, P at position 6, G at position 7, K at position 8, A at position 9, P at position 10, L at position 12 and/or Y at position 15 of SEQ ID NO: 12.

According to a preferred embodiment, LFR3 comprises the sequence as set forth in SEQ ID NO: 13 or a sequence at least 90% identical thereto. Preferably, LFR3 comprises E at position 14, F at position 15, T at position 16, S at position 21, Q at position 23, P at position 24 and/or Y at position 31 of SEQ ID NO: 13.

According to a preferred embodiment, LFR4 comprises the sequence as set forth in SEQ ID NO: 14 or a sequence at least 90% identical thereto. Preferably, LFR4 comprises Q at position 3 of SEQ ID NO: 14.

According to a preferred embodiment, HFR1 comprises the sequence as set forth in SEQ ID NO: 15 or a sequence at least 90% identical thereto. Preferably, HFR1 comprises E at position 6, Q at position 13 and/or R at position 19 of SEQ ID NO: 15.

According to a preferred embodiment, HFR2 comprises the sequence as set forth in SEQ ID NO: 16 or a sequence at least 90% identical thereto. Preferably, HFR2 comprises V at position 5, G at position 7, G at position 9 and/or S at position 14 of SEQ ID NO: 16.

According to a preferred embodiment, HFR3 comprises the sequence as set forth in SEQ ID NO: 17 or a sequence at least 90% identical thereto. Preferably, HFR3 comprises S at position 9, R at position 10, D at position 14, N at position 18, R at position 21, V at position 22 and/or V at position 27 of SEQ ID NO: 17.

According to a preferred embodiment, HFR4 comprises the sequence as set forth in SEQ ID NO: 18 or a sequence at least 90% identical thereto. Preferably, HFR4 comprises V at position 9 and/or S at position 11 of SEQ ID NO: 18.

It is to be understood that the sequences for LFR1 to 4 (SEQ ID NOs: 11 to 14) and the sequences for HFR1 to 4 (SEQ ID NOs: 15 to 18) are comprised in the larger sequences for VL (SEQ ID NO: 5) and VH (SEQ ID NO: 9), respectively. Thus, the specific amino acids identified above with respect to individual LFR and HFR sequences are likewise comprised in the larger sequences for VL and VH, respectively, which the skilled person understands to involve a different numbering when defining the position in the larger sequences. For example, the sequence for LFR2 starts at position W35 and ends with position Y49 of SEQ ID NO: 5. Accordingly, position 2 in LFR2 of SEQ ID NO: 12 corresponds to position 36 of SEQ ID NO: 5, position 3 in LFR2 corresponds to position 37 of SEQ ID NO: 5, etc.

According to a preferred embodiment, LFR1 comprises the sequence as set forth in SEQ ID NO: 29 or a sequence at least 90% identical thereto.

According to a preferred embodiment, LFR2 comprises the sequence as set forth in SEQ ID NO: 30 or a sequence at least 90% identical thereto.

According to a preferred embodiment, LFR3 comprises the sequence as set forth in SEQ ID NO: 31 or a sequence at least 90% identical thereto.

According to a preferred embodiment, LFR4 comprises the sequence as set forth in SEQ ID NO: 32 or a sequence at least 90% identical thereto.

According to a preferred embodiment, HFR1 comprises the sequence as set forth in SEQ ID NO: 33 or a sequence at least 90% identical thereto.

According to a preferred embodiment, HFR2 comprises the sequence as set forth in SEQ ID NO: 34 or a sequence at least 90% identical thereto.

According to a preferred embodiment, HFR3 comprises the sequence as set forth in SEQ ID NO: 35 or a sequence at least 90% identical thereto.

According to a preferred embodiment, HFR4 comprises the sequence as set forth in SEQ ID NO: 36 or a sequence at least 90% identical thereto.

It is to be understood that the sequences for LFR1 to 4 (SEQ ID NOs: 29 to 32) and the sequences for HFR1 to 4 (SEQ ID NOs: 33 to 36) are comprised in the larger sequences for VL (SEQ ID NO: 23) and VH (SEQ ID NO: 29), respectively. Thus, the specific amino acids identified above with respect to individual LFR and HFR sequences are likewise comprised in the larger sequences for VL and VH, respectively, which the skilled person understands to involve a different numbering when defining the position in the larger sequences. For example, the sequence for LFR2 starts at position L41 and ends with position Y57 of SEQ ID NO: 23. Accordingly, position 2 in LFR2 of SEQ ID NO: 23 corresponds to position 42 of SEQ ID NO: 23, position 3 in LFR2 corresponds to position 43 of SEQ ID NO: 23, etc.

According to a preferred embodiment, LFR1 comprises the sequence as set forth in SEQ ID NO: 47 or a sequence at least 90% identical thereto.

According to a preferred embodiment, LFR2 comprises the sequence as set forth in SEQ ID NO: 48 or a sequence at least 90% identical thereto.

According to a preferred embodiment, LFR3 comprises the sequence as set forth in SEQ ID NO: 49 or a sequence at least 90% identical thereto.

According to a preferred embodiment, LFR4 comprises the sequence as set forth in SEQ ID NO: 50 or a sequence at least 90% identical thereto.

According to a preferred embodiment, HFR1 comprises the sequence as set forth in SEQ ID NO: 51 or a sequence at least 90% identical thereto.

According to a preferred embodiment, HFR2 comprises the sequence as set forth in SEQ ID NO: 52 or a sequence at least 90% identical thereto.

According to a preferred embodiment, HFR3 comprises the sequence as set forth in SEQ ID NO: 53 or a sequence at least 90% identical thereto.

According to a preferred embodiment, HFR4 comprises the sequence as set forth in SEQ ID NO: 54 or a sequence at least 90% identical thereto.

It is to be understood that the sequences for LFR1 to 4 (SEQ ID NOs: 47 to 50) and the sequences for HFR1 to 4 (SEQ ID NOs: 51 to 54) are comprised in the larger sequences for VL (SEQ ID NO: 41) and VH (SEQ ID NO: 45), respectively. Thus, the specific amino acids identified above with respect to individual LFR and HFR sequences are likewise comprised in the larger sequences for VL and VH, respectively, which the skilled person understands to involve a different numbering when defining the position in the larger sequences. For example, the sequence for LFR2 starts at position L33 and ends with position Y49 of SEQ ID NO: 41. Accordingly, position 2 in LFR2 of SEQ ID NO: 41 corresponds to position 34 of SEQ ID NO: 41, position 3 in LFR2 corresponds to position 35 of SEQ ID NO: 41, etc.

According to a preferred embodiment, LFR1 comprises the sequence as set forth in SEQ ID NO: 65 or a sequence at least 90% identical thereto.

According to a preferred embodiment, LFR2 comprises the sequence as set forth in SEQ ID NO: 66 or a sequence at least 90% identical thereto.

According to a preferred embodiment, LFR3 comprises the sequence as set forth in SEQ ID NO: 67 or a sequence at least 90% identical thereto.

According to a preferred embodiment, LFR4 comprises the sequence as set forth in SEQ ID NO: 68 or a sequence at least 90% identical thereto.

According to a preferred embodiment, HFR1 comprises the sequence as set forth in SEQ ID NO: 69 or a sequence at least 90% identical thereto.

According to a preferred embodiment, HFR2 comprises the sequence as set forth in SEQ ID NO: 70 or a sequence at least 90% identical thereto.

According to a preferred embodiment, HFR3 comprises the sequence as set forth in SEQ ID NO: 71 or a sequence at least 90% identical thereto.

According to a preferred embodiment, HFR4 comprises the sequence as set forth in SEQ ID NO: 72 or a sequence at least 90% identical thereto.

It is to be understood that the sequences for LFR1 to 4 (SEQ ID NOs: 65 to 68) and the sequences for HFR1 to 4 (SEQ ID NOs: 69 to 72) are comprised in the larger sequences for VL (SEQ ID NO: 59) and VH (SEQ ID NO: 63), respectively. Thus, the specific amino acids identified above with respect to individual LFR and HFR sequences are likewise comprised in the larger sequences for VL and VH, respectively, which the skilled person understands to involve a different numbering when defining the position in the larger sequences. For example, the sequence for LFR2 starts at position V34 and ends with position Y50 of SEQ ID NO: 59. Accordingly, position 2 in LFR2 of SEQ ID NO: 59 corresponds to position 35 of SEQ ID NO: 59, position 3 in LFR2 corresponds to position 36 of SEQ ID NO: 59, etc.

According to a preferred embodiment, LFR1 comprises the sequence as set forth in SEQ ID NO: 126 or a sequence at least 90% identical thereto.

According to a preferred embodiment, LFR2 comprises the sequence as set forth in SEQ ID NO: 127 or a sequence at least 90% identical thereto.

According to a preferred embodiment, LFR3 comprises the sequence as set forth in SEQ ID NO: 128 or a sequence at least 90% identical thereto.

According to a preferred embodiment, LFR4 comprises the sequence as set forth in SEQ ID NO: 129 or a sequence at least 90% identical thereto.

According to a preferred embodiment, HFR1 comprises the sequence as set forth in SEQ ID NO: 130 or a sequence at least 90% identical thereto.

According to a preferred embodiment, HFR2 comprises the sequence as set forth in SEQ ID NO: 131 or a sequence at least 90% identical thereto.

According to a preferred embodiment, HFR3 comprises the sequence as set forth in SEQ ID NO: 132 or a sequence at least 90% identical thereto.

According to a preferred embodiment, HFR4 comprises the sequence as set forth in SEQ ID NO: 133 or a sequence at least 90% identical thereto.

It is to be understood that the sequences for LFR1 to 4 (SEQ ID NOs: 126 to 129) and the sequences for HFR1 to 4 (SEQ ID NOs: 130 to 133) are comprised in the larger sequences for VL (SEQ ID NO: 120) and VH (SEQ ID NO: 124), respectively.

The preferred positions in the framework regions LFR1 to 4 and HFR1 to 4 as set out in the above embodiments are indicated in accordance with the respective SEQ ID Nos, however, the skilled person is aware that these positions as indicated above are different according to Kabat numbering of the respective variable heavy and light chain domains.

Exemplary framework and CDR sequences for the variable regions disclosed herein are shown e.g. in SEQ ID NOs: 5, 9, 23, 27, 41, 45, 59, and 63, 120 and 124. With the exception of HCDR1, the antigen binding loops that were grafted onto the human framework regions were defined according to Kabat et al. (1991) Sequences of Proteins of Immunological Interest. (NIH Publication No. 91-3242, Bethesda). As residues H26 to H32 comprise the structural loop of HCDR1 (Chothia et al, Nature 342:877-883, 1989), residues H26 to H35 were applied as HCDR1 according to the combined Kabat/Chothia definition of HCDR1. The CDRs and framework regions were thus defined according to Kabat, except said HCDR1, for which the combined Chothia/Kabat definition was applied.

Example of positions for LCDR1 to LCDR3 and HCDR1 to HCDR3 (SEQ ID NO: 5 and 9):

| CDR | Kabat no. |
|---|---|
| L1 | L24-L34 |
| L2 | L50-L56 |
| L3 | L89-L97 |
| H1 | H26-H35* |
| H2 | H50-H65 |
| H3 | H95-H102 |

*defined according to the combined Kabat/Chothia definition of HCDR1

As outlined above, the sequences identified herein as SEQ ID NOs: 2 to 4 and 11 to 14 together form the VL sequence as shown in SEQ ID NO: 5, likewise SEQ ID NOs: 6 to 8 and 15 to 18 together form the VH sequence as shown in SEQ ID NO: 9. Accordingly, the positions in said SEQ ID NOs are different according to Kabat numbering of the respective variable heavy and light chain domains.

According to a preferred embodiment, the present invention provides an extracellular antigen-recognition domain comprising the VL region as defined herein, comprising one or more of the following amino acid positions: 3Q, 40P, 46L, 49Y and/or 71Y. In addition or alternatively, the extracellular antigen-recognition domain of the present invention comprises the VH region as defined herein, comprising one or more of the following amino acid positions: 6E, 40T, 79Y and 84S.

According to a further preferred embodiment, the extracellular antigen-recognition domain of the present invention comprises amino acid 6E in VH and 3Q in VL. According to a further preferred embodiment, the extracellular antigen-recognition domain of the present invention comprises amino acid 6E in VH, and 3Q, 40P and 49Y in VL. According to a further preferred embodiment, the extracellular antigen-recognition domain of the present invention comprises amino acid 6E in VH and 3Q, 40P, 46L and 49Y in VL. According to a further preferred embodiment, the extracellular antigen-recognition domain of the present invention comprises amino acid 6E and 79Y in VH and 3Q, 40P, 46L, 49Y and 71Y in VL. According to a further preferred embodiment, the extracellular antigen-recognition domain of the present invention comprises amino acid 6E, 40T, 79Y and 84S in VH and 3Q, 40P, 46L, 49Y and 71Y in VL.

According one preferred embodiment, the extracellular antigen-recognition domain of the present invention comprises at least LFR2 of SEQ ID NO: 12 or a sequence at least 90% identical thereto, and LFR 3 of SEQ ID NO: 13 or a sequence at least 90% identical thereto.

According one preferred embodiment, the extracellular antigen-recognition domain of the present invention comprises at least HFR3 of SEQ ID NO: 17 or a sequence at least 90% identical thereto.

According to one preferred embodiment, the extracellular antigen-recognition domain comprises at least LFR2 and/or LFR3 as defined herein. According to a further preferred embodiment, the extracellular antigen-recognition domain comprises at least HFR2 and/or HFR3 as defined herein. According to a particularly preferred embodiment, the extracellular antigen-recognition domain comprises at least LFR2 and LFR3; LFR2 and HFR2; LFR2 and HFR3; LFR3 and HFR2; LFR3 and HFR3; LFR2, LFR3 and HFR2; LFR2, LFR3 and HFR3; HFR2, HFR3 and LFR2; HFR2, HFR3 and LFR3; or LFR2, LFR3, HFR2 and HFR3 as defined herein.

According to a preferred embodiment, the extracellular antigen-recognition domain of the present invention comprises LFR1 of SEQ ID NO: 11, LFR2 of SEQ ID NO: 12, LFR3 of SEQ ID NO: 13, and/or LFR4 of SEQ ID NO: 14.

According to a further preferred embodiment, the extracellular antigen-recognition domain of the present invention comprises HFR1 of SEQ ID NO: 15, HFR2 of SEQ ID NO: 16, HFR3 of SEQ ID NO: 17, and/or LFR4 of SEQ ID NO: 18.

According to one particularly preferred embodiment, the extracellular antigen-recognition domain of the present invention comprises LFR1 of SEQ ID NO: 11, LFR2 of SEQ ID NO: 12, LFR3 of SEQ ID NO: 13, and LFR4 of SEQ ID NO: 14, as well as HFR1 of SEQ ID NO: 15, HFR2 of SEQ ID NO: 16, HFR3 of SEQ ID NO: 17, and LFR4 of SEQ ID NO: 18.

According to a preferred embodiment, the extracellular antigen-recognition domain of the present invention comprises LFR1 of SEQ ID NO: 29, LFR2 of SEQ ID NO: 30, LFR3 of SEQ ID NO: 31, and/or LFR4 of SEQ ID NO: 32.

According to a further preferred embodiment, the extracellular antigen-recognition domain of the present invention comprises HFR1 of SEQ ID NO: 33, HFR2 of SEQ ID NO: 34, HFR3 of SEQ ID NO: 35, and/or LFR4 of SEQ ID NO: 36.

According to one particularly preferred embodiment, the extracellular antigen-recognition domain of the present invention comprises LFR1 of SEQ ID NO: 29, LFR2 of SEQ ID NO: 30, LFR3 of SEQ ID NO: 31, and LFR4 of SEQ ID NO: 32, as well as HFR1 of SEQ ID NO: 33, HFR2 of SEQ ID NO: 34, HFR3 of SEQ ID NO: 35, and LFR4 of SEQ ID NO: 36.

According to a preferred embodiment, the extracellular antigen-recognition domain of the present invention comprises LFR1 of SEQ ID NO: 47, LFR2 of SEQ ID NO: 48, LFR3 of SEQ ID NO: 49, and/or LFR4 of SEQ ID NO: 50.

According to a further preferred embodiment, the extracellular antigen-recognition domain of the present invention comprises HFR1 of SEQ ID NO: 51, HFR2 of SEQ ID NO: 52, HFR3 of SEQ ID NO: 53, and/or LFR4 of SEQ ID NO: 54.

According to one particularly preferred embodiment, the extracellular antigen-recognition domain of the present invention comprises LFR1 of SEQ ID NO: 47, LFR2 of SEQ ID NO: 48, LFR3 of SEQ ID NO: 49, and LFR4 of SEQ ID NO: 50, as well as HFR1 of SEQ ID NO: 51, HFR2 of SEQ ID NO: 52, HFR3 of SEQ ID NO: 53, and LFR4 of SEQ ID NO: 54.

According to a preferred embodiment, the extracellular antigen-recognition domain of the present invention comprises LFR1 of SEQ ID NO: 65, LFR2 of SEQ ID NO: 66, LFR3 of SEQ ID NO: 67, and/or LFR4 of SEQ ID NO: 68.

According to a further preferred embodiment, the extracellular antigen-recognition domain of the present invention comprises HFR1 of SEQ ID NO: 69, HFR2 of SEQ ID NO: 70, HFR3 of SEQ ID NO: 71, and/or LFR4 of SEQ ID NO: 72.

According to one particularly preferred embodiment, the extracellular antigen-recognition domain of the present invention comprises LFR1 of SEQ ID NO: 65, LFR2 of SEQ ID NO: 66, LFR3 of SEQ ID NO: 67, and LFR4 of SEQ ID NO: 68, as well as HFR1 of SEQ ID NO: 69, HFR2 of SEQ ID NO: 70, HFR3 of SEQ ID NO: 71, and LFR4 of SEQ ID NO: 72.

According to a preferred embodiment, the extracellular antigen-recognition domain of the present invention comprises LFR1 of SEQ ID NO: 126, LFR2 of SEQ ID NO: 127, LFR3 of SEQ ID NO: 128, and/or LFR4 of SEQ ID NO: 129.

According to a further preferred embodiment, the extracellular antigen-recognition domain of the present invention comprises HFR1 of SEQ ID NO: 130, HFR2 of SEQ ID NO: 131, HFR3 of SEQ ID NO: 132, and/or LFR4 of SEQ ID NO: 133.

According to one particularly preferred embodiment, the extracellular antigen-recognition domain of the present invention comprises LFR1 of SEQ ID NO: 126, LFR2 of SEQ ID NO: 127, LFR3 of SEQ ID NO: 128, and LFR4 of SEQ ID NO: 129, as well as HFR1 of SEQ ID NO: 130, HFR2 of SEQ ID NO: 131, HFR3 of SEQ ID NO: 132, and LFR4 of SEQ ID NO: 133.

According to a preferred embodiment of the present invention, the extracellular antigen-recognition domain preferably comprises the amino acid sequences of SEQ ID NO: 5 and SEQ ID NO: 9), or variants or fragment thereof, wherein the variants or fragments maintain the CDRs as defined above. Preferably, said variants or fragments have at least 80% sequence identity with SEQ ID NO: 5 and SEQ ID NO: 9, respectively.

According to a preferred embodiment of the present invention, the extracellular antigen-recognition domain preferably comprises the amino acid sequences of SEQ ID NO: 23 and SEQ ID NO: 27), or variants or fragments thereof, wherein the variants or fragments maintain the CDRs as defined above. Preferably, said variants have at least 80% sequence identity with SEQ ID NO: 23 and SEQ ID NO: 27, respectively.

According to a preferred embodiment of the present invention, the extracellular antigen-recognition domain preferably comprises the amino acid sequences of SEQ ID NO: 41 and SEQ ID NO: 45), or variants or fragments thereof, wherein the variants or fragments maintain the CDRs as defined above. Preferably, said variants or fragments have at least 80% sequence identity with SEQ ID NO: 41 and SEQ ID NO: 45, respectively.

According to a preferred embodiment of the present invention, the extracellular antigen-recognition domain preferably comprises the amino acid sequences of SEQ ID NO: 59 and SEQ ID NO: 63), or variants or fragments thereof, wherein the variants or fragments maintain the CDRs as defined above. Preferably, said variants or fragments have at least 80% sequence identity with SEQ ID NO: 59 and SEQ ID NO: 63, respectively. For the purposes of the present invention, “variant” or “variants” of an amino acid sequence comprise amino acid insertion variants, amino acid addition variants, amino acid deletion variants, amino acid substitution variants, and every combination thereof.

For the purposes of the present invention, “fragment” or “fragments” of an amino acid sequence comprise a partial sequence of said amino acid sequence, preferably exhibiting the functional properties of said amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

Amino acid addition variants comprise amino- and/or carboxy-terminal fusions of one or more amino acids, such as 1, 2, 3, 4, 5, 6, 7, 8, 9, 10, 15, 20, 25, 30 or more amino acids.

Amino acid deletion variants are characterized by the removal of one or more amino acids from the sequence, such as by removal of 1, 2, 3, 4, 5, 6, 7, 8, 9, 10, 15, 20, 25, 30 or more amino acids. The deletions may be in any position of the protein.

Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids.

In accordance with the present invention, the degree of similarity, preferably identity, between a given amino acid sequence and an amino acid sequence which is a variant of said given amino acid sequence will be at least about 80%, 81%, 82%, 83%, 84%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, preferably continuous amino acids. In preferred embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5, or using the NCBI Blast Protein tool “blastp suite”.

The term “sequence similarity” indicates the percentage of amino acids that either are identical or that represent conservative amino acid substitutions. “Sequence identity” between two amino acid sequences indicates the percentage of amino acids that are identical between the sequences.

The term “percentage identity” is intended to denote a percentage of amino acid residues which are identical between the two sequences to be compared, obtained after the best alignment, this percentage being purely statistical and the differences between the two sequences being distributed randomly and over their entire length. The percentage identity is calculated by determining the number of identical positions between the two sequences being compared, dividing this number by the number of positions compared and multiplying the result obtained by 100 so as to obtain the percentage identity between these two sequences.

According to the invention, a variant, fragment, part, portion or derivative of an amino acid sequence, peptide or protein preferably has a functional property of the amino acid sequence, peptide or protein, respectively, from which it has been derived, i.e. it is functionally equivalent. The skilled person will understand that sequence variants of the peptides, domains and regions described herein can be used without adversely impacting the invention, where the variants have the same or similar activity as the domain on which they are modeled. As described above, such variants will have at least about 80%, 81%, 82%, 83%, 84%, 84%, 85%, 86%, 87%, 88%, 89%, 90%, 91%, 92%, 93%, 94%, 95%, 96%, 97%, 98%, or 99% sequence identity to the amino acid sequence of the domain from which they are derived. Preferably, such variants will have at least about 90%, 91%, 92%, 93%, 94%, 95%, 96%, 97%, 98%, or 99% sequence identity to the amino acid sequence of the domain from which they are derived. A variant, fragment, part, portion or derivative of an amino acid sequence, peptide or protein is preferably functionally equivalent such as immunologically equivalent to the amino acid sequence, peptide or protein, respectively, from which it is derived. In one embodiment, the functional property is the property to bind to a tumor-associated antigen or to transduce the binding signal within an immune effector cell.

The term “derived” means according to the invention that a particular entity, in particular a particular sequence, is present in the object from which it is derived, in particular an organism or molecule. In the case of amino acid sequences, especially particular sequence regions, “derived” in particular means that the relevant amino acid sequence is derived from an amino acid sequence in which it is present.

According to one particularly preferred embodiment, the extracellular antigen-recognition domain of the present invention comprises the VL and VH domains as set forth in SEQ ID NO: 5 and SEQ ID NO: 9.

According to one particularly preferred embodiment, the extracellular antigen-recognition domain of the present invention comprises the VL and VH domains as set forth in SEQ ID NO: 23 and SEQ ID NO: 27.

According to one particularly preferred embodiment, the extracellular antigen-recognition domain of the present invention comprises the VL and VH domains as set forth in SEQ ID NO: 41 and SEQ ID NO: 45.

According to one particularly preferred embodiment, the extracellular antigen-recognition domain of the present invention comprises the VL and VH domains as set forth in SEQ ID NO: 59 and SEQ ID NO: 63.

According to one particularly preferred embodiment, the extracellular antigen-recognition domain of the present invention comprises the VL and VH domains as set forth in SEQ ID NO: 120 and SEQ ID NO: 124.

In the antigen-binding domain or the antigen binding polypeptide as described herein, the heavy chain variable region (VH) and the light chain variable region (VL) are preferably connected by a flexible peptide linker having ten or more amino acids.

The term “linker” or “peptide linker” as used in the context of the present invention refers to an amino acid sequence which sterically separates two parts or moieties of a complex, e.g. two peptides, polypeptides or proteins. According to one embodiment, such linker comprises or consists of more than 10 amino acid residues, preferably of 11, 12, 13, 14, 15, 16, 17, 18, 19, 20, 21, 22, 23, 24, 25 or more amino acids, most preferably 15 amino acid residues. Peptide linkers provide flexibility among the two moieties that are linked together. Flexibility is generally increased if the amino acids are small. Accordingly, flexible peptide linkers comprise an increased content of small amino acids, in particular of glycine and/or alanine, and/or hydrophilic amino acids such as serine, threonine, asparagine and glutamines. It is believed that peptide linkers, e.g. one or more amino acid residues, inserted between two domains provide sufficient mobility for the domains, for example in single chain constructs, or between the variable domains of light and heavy chain variable domains, and allow correct folding to form the antigen binding site. Linkers in context of the present invention are abbreviated as L1, L2, L3, L4 etc.

According to a preferred embodiment, linker L1 connecting VH and VL of the CoCAR of the present invention comprises glycine and alanine residues. According to a more preferred embodiment, linker L1 connecting VH and VL of the CoCAR of the present invention comprises glycine (G) and serine(S) residues. More preferably, said linker has a G₄S format, meaning that in the linker four glycine residues are followed by one alanine residue, which format can be repeated several times, preferably between 3 and 6 times. In other words, the linker preferably has the structure of (G₄S)ₓ with x denoting any integer of between 3 and 6. Most preferably, linker L1 has the amino acid sequence of GGGGSGGGGSGGGGS ([G₄S]₃) (SEQ ID NO: 10) or GSTSGSGKPGSGEGSTKG (SEQ ID NO: 46).

According to a preferred embodiment, the CoCAR of the present invention comprises at least one spacer region, as defined herein.

According to a preferred embodiment, the CoCAR of the present invention comprises the following structural elements in the order indicated: (i) an extracellular antigen-recognition domain as described herein, comprising the light chain variable domain and the heavy chain variable domain as described herein above, preferably separated by a linker as defined herein above, (ii) a first hinge region, (iii) a first transmembrane domain, and (iv) at least one cytosolic domain (a) capable of binding LCK, and/or (b) comprising LCK, or a variant or fragment thereof.

The CoCAR of the present invention preferably does not comprise a CD3zeta domain.

In the CoCAR of the present invention, the at least one cytosolic domain preferably does not comprise a CD3zeta domain.

In one embodiment, the coreceptor-CAR (CoCAR) of the present invention comprises:
(i) a first extracellular antigen-recognition domain,
(ii) a first hinge domain,
(iii) a first transmembrane domain, and
(iv) at least one cytosolic domain capable of binding LCK, and

wherein the CoCAR does not comprise a CD3zeta domain.

In one embodiment, the coreceptor-CAR (CoCAR) of the present invention comprises:
(i) a first extracellular antigen-recognition domain,
(ii) a first hinge domain derived from CD8α,
(iii) a first transmembrane domain derived from CD8α, and
(iv) at least one cytosolic domain capable of binding LCK derived from CD28, and

wherein the CoCAR does not comprise a CD3zeta domain.

The coreceptor-CAR with the domains above is also referred to as [first antigen/scFv]-28. In one embodiment, the coreceptor-CAR (CoCAR) of the present invention comprises:
(i) a first extracellular antigen-recognition domain,
(ii) a first hinge domain derived from CD8α,
(iii) a first transmembrane domain derived from CD8α, and
(iv) at least one cytosolic domain capable of binding LCK derived from CD44, and

wherein the CoCAR does not comprise a CD3zeta domain.

The coreceptor-CAR with the domains above is also referred to as [first antigen/scFv]-44.

In one embodiment, the coreceptor-CAR (CoCAR) of the present invention comprises:
(i) a first extracellular antigen-recognition domain,
(ii) a first hinge domain derived from CD8α,
(iii) a first transmembrane domain derived from CD44, and
(iv) at least one cytosolic domain capable of binding LCK derived from CD44, and

wherein the CoCAR does not comprise a CD3zeta domain.

The coreceptor-CAR with the domains above is also referred to as [first antigen/scFv]-44v2.

In one embodiment, the coreceptor-CAR (CoCAR) of the present invention comprises:
(i) a first extracellular antigen-recognition domain,
(ii) a first hinge domain derived from CD8α,
(iii) a first transmembrane domain derived from CD8α, and
(iv) at least one cytosolic domain capable of binding LCK derived from CD146, and

wherein the CoCAR does not comprise a CD3zeta domain.

The coreceptor-CAR with the domains above is also referred to as [first antigen/scFv]-146.

In one embodiment, the coreceptor-CAR (CoCAR) of the present invention comprises:
(i) a first extracellular antigen-recognition domain,
(ii) a first hinge domain derived from CD8α,
(iii) a first transmembrane domain derived from CD146, and
(iv) at least one cytosolic domain capable of binding LCK derived from CD146, and

wherein the CoCAR does not comprise a CD3zeta domain.

The coreceptor-CAR with the domains above is also referred to as CD146v2 CoCAR or [first antigen/scFv]-146v2.

In a further embodiment, the coreceptor-CAR (CoCAR) of the present invention comprises:
(i) a first extracellular antigen-recognition domain,
(ii) a first hinge domain derived from CD28,
(iii) a first transmembrane domain derived from CD28, and
(iv) at least one cytosolic domain capable of binding LCK derived from CD28, and

wherein the CoCAR does not comprise a CD3zeta domain.

The “spacer” or “hinge” region preferably imparts flexibility to the domains forming the antigen binding site and allows strong binding to the desired antigen. As used herein a “hinge”, “hinge region” or “hinge domain”, referred to in the following simply as “hinge”, are used interchangeably.

The first hinge region is preferably based on a CD4, CD8α, CD28 or IgG-Fc domain, preferably based on a CD4 domain.

The term “Fc domain” as used in the context of the present invention encompasses native Fc domains and Fc domain variants and sequences as further defined herein below. In the context of Fc variants and native Fc molecules, the term “Fc domain” includes molecules in monomeric or multimeric form, whether digested from whole antibodies or produced by other means.

The term “native Fc” as used herein refers to a molecule comprising the sequence of a non-antigen-binding fragment resulting from digestion of an antibody or produced by other means, whether in monomeric or multimeric form, and may contain the hinge region. The original immunoglobulin source of the native Fc is, in particular, of human origin and can be any of the immunoglobulins, preferably IgG1, IgG2, or IgG4, most preferably IgG1. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgAQ1, and IgGA2). One example of a native Fc is a disulfide-bonded dimer resulting from papain digestion of an IgG. The term “native Fc” is generic to the monomeric, dimeric, and multimeric forms.

The present invention further provides a CoCAR comprising (i) a first extracellular antigen-recognition domain as described herein, comprising a light chain variable domain and the heavy chain variable domain not limited to the VH and VL domains as described herein, preferably separated by a linker as defined herein above, (ii) a first hinge region, (iii) a first transmembrane domain, and (iv) at least one cytosolic domain (a) capable of binding LCK, and/or (b) comprising LCK, or a variant or fragment thereof.

The CoCAR of the present invention, and/or the CAR, to be combined with the CoCAR of the invention, may comprise a spacer, independently selected from the spacers described herein.

As used herein, the spacer region may include a hinge region, as described herein, and any other sequence suitable to connect the extracellular antigen-recognition domain and the at least one cytosolic domain.

The CoCAR of the present invention may comprise a spacer, as described herein, which may include the first hinge.

The CAR, to be combined with the CoCAR of the invention, may comprise a spacer, as described herein, which may include the second hinge region.

The first and second hinge region may be independently selected from the hinge regions as described herein. In particular, the first and second hinge region may be different.

The spacer region used in the present invention preferably comprises a CH2 domain comprising one or more amino acid mutations reducing or preventing off-target interactions with host myeloid cells. According to a particularly preferred embodiment, the CH2 domain comprises the amino acid sequence of SEQ ID NO: 76 or a dimerizing variant thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 76. According to a particularly preferred embodiment, the CH2 domain comprises a polypeptide having the amino acid sequence of SEQ ID NO: 76. In addition or alternatively, the spacer region used in the present invention preferably comprises a CH2 domain comprising one or more amino acid mutations preventing glycosylation of the spacer region.

The spacer region used in the present invention may further comprise a CH3 domain. The CH3 domain is preferably derived from IgG1. More preferably, the CH3 domain is an IgG1 CH3 domain. According to a particularly preferred embodiment, the CH3 domain comprises the amino acid sequence of SEQ ID NO: 77 or a dimerizing variant thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 77. According to a particularly preferred embodiment, the CH3 domain comprises a polypeptide having the amino acid sequence of SEQ ID NO: 77.

As used herein, the hinge domain may any hinge domain known in the art. The skilled person knows suitable hinge domains. It is preferred that the hinge comprises a sequence selected from SEQ ID NOs: 73, 78, 82 and 85, and variants and fragments thereof.

Preferably, the hinge domain is a CD4 hinge domain. According to a particularly preferred embodiment, the hinge comprises the amino acid sequence of SEQ ID NO: 82 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 82.

Preferably, the hinge domain is a CD8 hinge domain, preferably a CD8α hinge domain. According to a particularly preferred embodiment, the hinge comprises the amino acid sequence of SEQ ID NO: 73 or 85 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 73 or 85.

Preferably, the hinge domain is a CD28 hinge domain. According to a particularly preferred embodiment, the hinge comprises the amino acid sequence of SEQ ID NO: 78 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 78.

Preferably, the hinge is derived from an immunoglobulin. More preferably, the hinge is an IgG1 derived hinge. Even more preferably, the hinge is an IgG1 hinge. According to a particularly preferred embodiment, the hinge comprises the amino acid sequence of SEQ ID NO: 75 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 75. According to a particularly preferred embodiment, the hinge comprises a polypeptide having the amino acid sequence of SEQ ID NO: 75.

The CAR to be combined with the CoCAR of the present invention, may comprise a second hinge, as described herein. It is preferred that the second hinge comprises SEQ ID NO: 75, or a variant or fragment thereof.

The CAR to be combined with the CoCAR of the present invention, may comprise a spacer, as described herein, which may include the second hinge.

It is preferred that the second hinge comprises a hinge derived from CD28, in particular the second hinge comprises SEQ ID NO: 78, or a variant or fragment thereof. It is also preferred that (1) the second hinge comprises a hinge derived from CD28, in particular comprising SEQ ID NO: 78, or a variant or fragment thereof, and (2) the CoCAR of the invention does not comprise a hinge region derived from CD28, in particular a hinge comprising SEQ ID NO: 78, or a variant or fragment thereof. For example, (1) the CAR, to be combined with the CoCAR of the invention, comprises a second hinge domain derived from CD28, as described herein, in particular a second hinge comprising SEQ ID NO: 78, and (2) the CoCAR of the invention comprises a first hinge region derived from CD4, in particular comprising SEQ ID NO: 82.

It is also preferred that the second hinge comprises a hinge derived from CD8, preferably CD8α, in particular the second hinge comprises SEQ ID NO: 73 or 85, or a variant or fragment thereof. It is also preferred that (1) the second hinge comprises a hinge derived from CD8, preferably CD8α, in particular the second hinge comprises SEQ ID NO: 73 or 85, or a variant or fragment thereof, and (2) the CoCAR does not comprise a hinge derived from CD8, preferably CD8α, in particular a hinge comprising SEQ ID NO: 73 or 85, or a variant or fragment thereof. For example, (1) the CAR, to be combined with the CoCAR of the invention, comprises a second hinge comprises a hinge derived from CD8, preferably CD8α, in particular the second hinge comprises SEQ ID NO: 73 or 85, or a variant or fragment thereof, and (2) the CoCAR of the invention comprises a first hinge region derived from CD28, in particular comprising SEQ ID NO: 78.

The CAR, to be combined with the CoCAR of the present invention, may comprise a spacer said spacer comprising a CH2 comprising SEQ ID NO: 76, or a variant or fragment thereof, and/or a CH3 comprising SEQ ID NO: 77, or a variant or fragment thereof.

The CoCAR of the present invention may comprise a first transmembrane domain, as described herein.

The CAR, to be combined with the CoCAR of the present invention, may comprise a second transmembrane domain, as described herein.

The first and second transmembrane domain may be independently selected from the transmembrane domains as described herein. In particular, the first and second transmembrane domain may be different.

As used herein, the transmembrane domain may any transmembrane domain known in the art. The skilled person knows suitable transmembrane domains.

The transmembrane domain serves to anchor the antigen receptor on the membrane of a respective cell. The transmembrane domain can be a hydrophobic alpha helix that spans the membrane. Preferably, the transmembrane region is derived from CD28, more preferably from human CD28. Thus, according to a preferred embodiment of the present invention the transmembrane region preferably comprises a polypeptide having the amino acid sequence of SEQ ID NO: 79 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 79. According to a particularly preferred embodiment, the transmembrane region comprises a polypeptide having the amino acid sequence of SEQ ID NO: 79.

Preferably, the transmembrane region of the present invention is derived from CD4, more preferably from human CD4. Thus, according to a preferred embodiment of the present invention the transmembrane region preferably comprises a polypeptide having the amino acid sequence of SEQ ID NO: 83 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 83. According to a particularly preferred embodiment, the transmembrane region comprises a polypeptide having the amino acid sequence of SEQ ID NO: 83.

Preferably, the transmembrane region of the present invention is derived from CD8 or CD8α, more preferably from human CD8 or CD8α. Thus, according to a preferred embodiment of the CAR of the present invention the transmembrane region preferably comprises a polypeptide having the amino acid sequence of SEQ ID NO: 74 or 86 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 74 or 86. According to a particularly preferred embodiment, the transmembrane region comprises a polypeptide having the amino acid sequence of SEQ ID NO: 74 or 86.

In some embodiments, the transmembrane domain of the present invention is derived from CD44, preferably from human CD44. Thus, according to a preferred embodiment of the present invention the transmembrane domain preferably comprises a polypeptide having the amino acid sequence of SEQ ID NO: 136 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 136. According to a particularly preferred embodiment, the transmembrane domain comprises a polypeptide having the amino acid sequence of SEQ ID NO: 136.

In some embodiments, the transmembrane domain of the present invention is derived from CD146, preferably from human CD146. Thus, according to a preferred embodiment of the present invention the transmembrane domain preferably comprises a polypeptide having the amino acid sequence of SEQ ID NO: 137 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 137. According to a particularly preferred embodiment, the transmembrane domain comprises a polypeptide having the amino acid sequence of SEQ ID NO: 137.

“LCK” or “lymphocyte-specific protein tyrosine kinase” is known in the art. LCK can be endogenously expressed in a T cell. In particular, LCK is non-covalently bound to the intracellular signaling domain capable of binding LCK. As used herein, LCK can be recombinantly expressed in the recombinant cell, for example linked to a transmembrane domain of a CoCAR. For example, LCK can comprise SEQ ID NO: 103, or a variant or fragment thereof.

As used herein a “cytosolic domain capable of binding LCK” or “intracellular signaling domain capable of binding LCK” is a domain comprising one or more motifs capable of binding LCK. For example, a signaling domain capable of binding LCK can be derived from the cytoplasmic tail of a receptor. Examples of amino acid sequences comprising one or more motifs capable of binding LCK are SEQ ID NO: 84, 87, 89, 90, 91, 92, 93, 94, 95, 96, 97, 98, 99, 100, 101, 102. A preferred sequence comprising one or more motifs capable of binding LCK is selected from SEQ ID NO: 89, 90, 91, 93, 94, 96, 97, 98, 100, 102.

According to a preferred embodiment, the at least one intracellular signaling domain capable of binding LCK comprises at least one CD4, CD8α, CD28, CD3ε, CD44, and/or CD146 intracellular signaling domain, or a variant or fragment thereof.

According to a preferred embodiment, the at least one intracellular signaling domain capable of binding LCK comprises at least one motif derived from CD4, CD8α, CD28, CD3ε, CD44, and/or CD146 intracellular signaling domain, which is capable of binding LCK.

According to a preferred embodiment, the at least one intracellular signaling domain capable of binding LCK comprises at least one CD28, CD44, and/or CD146 intracellular signaling domain, or a variant or fragment thereof.

According to a preferred embodiment, the at least one intracellular signaling domain capable of binding LCK comprises at least one motif capable of binding LCK, which is derived from CD28, CD44, and/or CD146 intracellular signaling domain.

According to a particularly preferred embodiment, the at least one intracellular signaling domain capable of binding LCK comprises at least one CD4 intracellular signaling domain, preferably at least one CD4 intracellular signaling domain having the amino acid sequence of SEQ ID NO: 99 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 99.

According to a particularly preferred embodiment, the at least one intracellular signaling domain capable of binding LCK comprises at least one CD8α intracellular signaling domain, preferably at least one CD4 intracellular signaling domain having the amino acid sequence of SEQ ID NO: 87 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 87.

According to a particularly preferred embodiment, the at least one intracellular signaling domain capable of binding LCK comprises at least one CD38 intracellular signaling domain, preferably at least one CD3ε intracellular signaling domain having the amino acid sequence of SEQ ID NO: 92 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 92.

According to a particularly preferred embodiment, the at least one intracellular signaling domain capable of binding LCK comprises at least one CD28 intracellular signaling domain, preferably at least one CD28 intracellular signaling domain having the amino acid sequence of SEQ ID NO: 95 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 95.

According to a particularly preferred embodiment, the at least one intracellular signaling domain capable of binding LCK comprises at least one CD44 intracellular signaling domain, preferably at least one CD44 intracellular signaling domain having the amino acid sequence of SEQ ID NO: 99 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 99.

According to a particularly preferred embodiment, the at least one intracellular signaling domain capable of binding LCK comprises at least one CD146 intracellular signaling domain, preferably at least one CD146 intracellular signaling domain having the amino acid sequence of SEQ ID NO: 101 or a variant or fragment thereof having preferably at least 80%, 81%, 82%, 83%, 84%, 84%, 85%, 86%, 87%, 88%, 89%, 90%, 91%, 92%, 93%, 94%, 95%, 96%, 97%, 98% or 99% amino acid sequence identity with SEQ ID NO: 101.

According to a preferred embodiment, the at least one intracellular signaling domain of the CAR, to be combined with the CoCAR of the present invention, comprises at least one of a 4-1BB intracellular domain and a CD32 intracellular domain, or a combination of both.

The 4-1BB intracellular signaling domain preferably comprises the amino acid sequence of SEQ ID NO: 80, or a variant or fragment thereof having at least 80%, 81%, 82%, 83%, 84%, 84%, 85%, 86%, 87%, 88%, 89%, 90%, 91%, 92%, 93%, 94%, 95%, 96%, 97%, 98% or 99% sequence identity to SEQ ID NO: 80. According to a particularly preferred embodiment, the 4-1BB intracellular signaling domain comprises a polypeptide having the amino acid sequence of SEQ ID NO: 80.

The CD32 intracellular signaling domain preferably comprises the amino acid sequence of SEQ ID NO: 81, or a variant or fragment thereof having at least 80%, 81%, 82%, 83%, 84%, 84%, 85%, 86%, 87%, 88%, 89%, 90%, 91%, 92%, 93%, 94%, 95%, 96%, 97%, 98% or 99% sequence identity to SEQ ID NO: 81. According to a particularly preferred embodiment, the CD32 intracellular signaling domain comprises a polypeptide having the amino acid sequence of SEQ ID NO: 81.

The at least one intracellular signaling domain of the CAR, to be combined with the CoCAR of the present invention, can also comprise a CD28 intracellular domain, wherein the CD28 intracellular domain preferably comprises or consists of the sequence as set forth in SEQ ID NO: 95 or a variant or fragment thereof having at least 80%, 81%, 82%, 83%, 84%, 84%, 85%, 86%, 87%, 88%, 89%, 90%, 91%, 92%, 93%, 94%, 95%, 96%, 97%, 98% or 99% sequence identity to SEQ ID NO: 95.

The antigen-recognition domain preferably is a scFv.

The CoCAR of the present invention is preferably a single chain CoCAR (also termed herein “sc CoCAR”). Such a single chain CoCAR represents essentially a single-chain variable fragment (scFv) which is a fusion protein of the variable regions of the heavy (VH) and light chains (VL) of immunoglobulins, preferably connected by a linker as defined herein. The linker can either connect the N-terminus of the VH with the C-terminus of the VL, or vice versa. Divalent (or bivalent) single-chain variable fragments (di-scFvs, bi-scFvs) can be engineered by linking two scFvs. This can be done by producing a single peptide chain with two VH and two VL regions, yielding tandem scFvs.

The CAR to be combined with the CoCAR of the present invention, is preferably a single chain CAR (also termed herein “sc CAR”). Such a single chain CAR represents essentially a single-chain variable fragment (scFv) which is a fusion protein of the variable regions of the heavy (VH) and light chains (VL) of immunoglobulins, preferably connected by a linker as defined herein. The linker can either connect the N-terminus of the VH with the C-terminus of the VL, or vice versa. Divalent (or bivalent) single-chain variable fragments (di-scFvs, bi-scFvs) can be engineered by linking two scFvs. This can be done by producing a single peptide chain with two VH and two VL regions, yielding tandem scFvs.

Other formats in addition to the single chain format are also possible, e.g. a Fab-like format or dimer format, comprising two peptide chains each comprising the VH and VL as well as the hinge region, the transmembrane domain, and the one or more intracellular signaling domains. In such double chain formats, the signaling domain on one peptide chain will preferably form a dimer with the signaling domain on the second chain, for example, through disulfide bridges.

While not wanting to be limited to a particular mechanism of action, it is believed that the two peptide chains of a dimeric antigen receptor of the invention, when expressed on the surface of an immune cell, form a dimer due to interactions (e.g., disulfide bonding) at least between the individual immunoreceptor signal transmission domains on the two chains.

The CAR to be combined with the CoCAR of the present invention comprises the VL domain as set forth in SEQ ID NO: 5 and the VH domain as set forth in SEQ ID NO: 9, including the VHCDR1 to VHCDR3 and VLCDR1 to VLCDR3 sequences as defined in SEQ ID NO: 2, SEQ ID NO: 3, SEQ ID NO: 4, SEQ ID NO: 6, SEQ ID NO: 7, and SEQ ID NO: 8, a linker as set forth in SEQ ID NO: 10, a CH2 domain as set forth in SEQ ID NO: 76, a CH3 domain as set forth in SEQ ID NO: 77 or a hinge domain as set forth in SEQ ID NO: 73 or 75, a transmembrane domain as set forth in SEQ ID NO: 74, a 4-1BB domain as set forth in SEQ ID NO: 80, and a CD3ζ domain as set forth in SEQ ID NO: 81.

SEQ ID NO: 104 describes an anti-CD22 CAR with humanized RFB4 scFv and CD8a backbone, encoded by SEQ ID NO: 110.

SEQ ID NO: 105 describes an anti-CD22 CAR with humanized RFB4 scFv and IgG-Fc backbone, encoded by SEQ ID NO: 111.

The term “IgG-Fc backbone” is used in the context of the present invention to refer to a IgG1-Fc spacer (hinge-CH2-CH3) und CD28 transmembrane domain.

The term “CD8a backbone” is used in the context of the present invention to refer to a CD8a hinge and a CD8a transmembrane domain. The term “CD28 backbone” is used in the context of the present invention to refer to a CD28 hinge and a CD28 transmembrane domain.

The CAR to be combined with the CoCAR of the present invention preferably comprises an amino acid sequence selected from SEQ ID NOs: 104, 105, 134, 185, 186, and 187 or variants and fragments thereof having at least 80%, 81%, 82%, 83%, 84%, 84%, 85%, 86%, 87%, 88%, 89%, 90%, 91%, 92%, 93%, 94%, 95%, 96%, 97%, 98% or 99% sequence identity to SEQ ID NO: 104, 105, 134, 185, 186, or 187.

Antigen receptors according to the invention or peptide chains of antigen receptors of the invention can further comprise other domains, such as additional domains involved in or enhancing antigen binding, signal sequences for membrane bound expression or for secretion, and/or domains that provide improved dimerization.

It will be understood that because the domains forming the antigen binding sites must be free to bind an antigen, the placement of these domains in the protein will generally be such that display of the region on the exterior of the cell is achieved. In the same manner, because the co-stimulation domains and signaling domains serve to induce activity and proliferation of the immune effector cells, the CoCAR of the invention and/or the CAR, to be combined with the CoCAR of the present invention preferably displays these domains in the interior of the cell.

The CoCAR of the present invention preferably include additional elements, such as a signal or leader peptide to ensure proper export of the fusion protein to the cells surface. A signal peptide or leader peptide is a sequence or peptide that allows for sufficient passage through the secretory pathway and expression on the cell surface such that an antigen receptor, for example, may bind an antigen present in the extracellular environment. Preferably, the leader peptide is cleavable and is removed from the mature peptide chains. The signal or leader peptide is preferably chosen with respect to the cell or organism wherein the peptide chains are produced in. According to a preferred embodiment, the CoCAR of the present invention comprises a signal peptide selected from the amino acid sequences as set forth in SEQ ID NO: 1, 19, 37, and 55 and variants and fragments thereof having at least 80%, 81%, 82%, 83%, 84%, 84%, 85%, 86%, 87%, 88%, 89%, 90%, 91%, 92%, 93%, 94%, 95%, 96%, 97%, 98% or 99% sequence identity to SEQ ID NO: 1, 19, 37, and 55.

The present invention further provides a CoCAR comprising the amino acid sequence selected from SEQ ID NO: 106-109, 138-151, 188-190, and variants and fragments thereof having at least 80%, 81%, 82%, 83%, 84%, 84%, 85%, 86%, 87%, 88%, 89%, 90%, 91%, 92%, 93%, 94%, 95%, 96%, 97%, 98% or 99% sequence identity to SEQ ID NO: 106-109, 138-151, 188-190. Preferably, the CDR sequences of such CoCAR are as defined herein. According to one embodiment of the present invention, the CoCAR consists of the amino acid sequence set forth in SEQ ID NO: SEQ ID NO: 106-109, 138-151, 188-190.

SEQ ID NO: 106 describes an anti-CD19 CD4-CoCAR, encoded by SEQ ID NO: 112.

SEQ ID NO: 107 describes an anti-CD22 CD4-CoCAR encoded by SEQ ID NO: 113.

SEQ ID NO: 108 describes an anti-CEA CD4-CoCAR encoded by SEQ ID NO: 114.

SEQ ID NO: 109 describes a bicistronic anti-CD22-CAR-T2A-anti-CD22-CD4-CoCAR, encoded by SEQ ID NO: 115.

The CoCAR of the present invention in combination with a CAR preferably not only elicits antigen-dependent antitumor responses at least as efficient as a CAR known in the art, but also prevents antigen-independent CAR signaling, myeloid-lineage cell activation and proinflammatory cytokine production, thereby providing outstanding efficacy and safety profiles for the treatment of cancer, such as B cell malignancies.

In the CoCAR of the present invention, the first antigen-recognition domain may be capable of binding a first antigen on the surface of a target cell, wherein binding of the CoCAR to the first antigen does not induce activation of a recombinant cell expressing the CoCAR. In particular, the CoCAR of the present invention may be co-expressed with a CAR comprising a second antigen-recognition domain, wherein the second antigen-recognition domain may be capable of binding a second antigen on the surface of a target cell, wherein binding of the CAR to the second antigen induces activation of the recombinant cell. Binding of the CoCAR to the first antigen may enhance the activation of the recombinant cell induced by binding of the CAR to the second antigen. The CoCAR may attenuate antigen-independent activation and differentiation of the recombinant cell induced by the CAR. The CAR may be any CAR as described herein.

Yet another aspect of the present invention is a CoCAR of the present invention in combination with a chimeric antigen receptor (CAR), in particular a CAR as described herein. The CoCAR of the present invention may be combined with any CAR know by the skilled person.

In some embodiments, the combination of the present invention comprises:
(a) an anti-CD19 CoCAR as described herein, and preferably selected from 19-28, 19-44, 19-44v2, 19-146, 19-146v2.
(b) an anti-CD22 CAR as described herein, preferably selected from 22-BBz, and m971-Fc-BBz,
wherein the combination is expressed on a cell surface.

In some embodiments, the combination of the present invention comprises:
(a) an anti-CD19 CoCAR as described herein, and preferably selected from 19-28, 19-44, 19-44v2, 19-146, 19-146v2.
(b) an anti-CD22 CAR as described herein, wherein the second antigen-recognition domain preferably comprises or consists of a SG-III scFv, LL2 scFv, or m971 scFv,
wherein the combination is expressed on a cell surface.

In some embodiments, the combination of the present invention comprises:
(a) an anti-CEA CoCAR as described herein,
(b) an anti-EGFR CAR as described herein,
wherein the combination is expressed on a cell surface.

In some embodiments, the combination of the present invention comprises:
(a) an anti-EGFR CoCAR as described herein, and preferably selected from EGFR-28, EGFR-44, EGFR-44v2, EGFR-146, EGFR-146v2.
(b) an anti-HER2 CAR as described herein, preferably aHER2-BBz,
wherein the combination is expressed on a cell surface.

According to a further aspect, the present invention provides a nucleic acid, nucleic acids, a nucleic acid construct and/or nucleic acid constructs encoding the CoCAR of the present invention.

The nucleic acid as described herein may also encode the CAR to be combined with the CoCAR of the present invention, as described herein.

The term “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the invention, a nucleic acid is preferably an isolated nucleic acid.

According to one embodiment, the nucleic acid or nucleic acid construct comprises the nucleic acid sequence selected from SEQ ID NO: 112-115, 152-166 or 198-207, or sequences being at least about 80%, 81%, 82%, 83%, 84%, 84%, 85%, 86%, 87%, 88%, 89%, 90%, 91%, 92%, 93%, 94%, 95%, 96%, 97%, 98%, or 99% identical thereto.

The nucleic acid or nucleic acid construct encoding the antigen receptor of the present invention can be administered to a patient in need thereof in naked form or in a carrier. Respective carriers, e.g. lipid carriers include any substance or vehicle with which nucleic acids such as DNA or RNA can be associated, e.g. by forming complexes with the nucleic acid or forming vesicles in which the nucleic acid is enclosed or encapsulated. This may result in increased stability of the nucleic acid compared to naked nucleic acid. In particular, stability of the nucleic acid in blood may be increased. For example, nanoparticulate RNA formulations with defined particle size, such as lipoplexes from RNA and liposomes, e.g. lipoplexes comprising DOTMA and DOPE or DOTMA and Cholesterol, can be used.

The term “combination of a first and second nucleic acid” refers to a nucleic acid bicistronic construct encoding the combination of a CAR and CoCAR, wherein the first nucleic acid encodes the CoCAR (a), and the second nucleic acid encodes the CAR (b).

In the bicistronic constructs or in the nucleic acid constructs, the first and second nucleic acids are functionally linked to at least one expression control sequence, such as EF1α.

In the bicistronic constructs or in the nucleic acid constructs, the first and second nucleic acids are separated by a sequence (such as P2A, E2A, F2A, T2A, and IRES, and preferably T2A) encoding a cleaving peptide, which allows separation of the CoCAR and CAR polypeptides after protein translation.

As used herein, the term “nanoparticle” refers to any particle having a diameter making the particle suitable for systemic, in particular parenteral, administration, of, in particular, nucleic acids, typically a diameter of less than 1000 nanometers (nm). Preferably, a nanoparticle has a diameter of less than 600 nm or a diameter of less than 400 nm.

In the nucleic acid construct of the invention, the nucleic acid, as described herein, may be functionally linked to at least one expression control sequence. The at least one expression control sequence may be selected from promoters, ribosome binding sites, enhancers and control elements which regulate transcription of the nucleic acid(s) or translation of mRNA(s) and enhancer sequences or upstream activator sequences.

Yet another aspect of the present invention relates to a vector, comprising the nucleic acid or the combination of the first and second nucleic acid, as described herein, the nucleic acid construct or the combination of nucleic acid constructs as described herein. The vector and/or the combination of vectors of the invention may be independently selected from vectors suitable to introduce antigen receptor constructs into T cells, for example from lentiviruses, γ-retroviruses and adeno-associated viruses.

The present invention also provides a recombinant cell expressing the nucleic acid or nucleic acid construct of the invention as defined herein.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

The term “cell” or “host cell” preferably relates to an intact cell, i.e. a cell with an intact membrane that has not released its normal intracellular components such as enzymes, organelles, or genetic material. An intact cell preferably is a viable cell, i.e. a living cell capable of carrying out its normal metabolic functions. Preferably, the term “cell” or “host cell” relates to any cell which can be transfected with an exogenous nucleic acid. Preferably, the cell when transfected with an exogenous nucleic acid and transferred to a recipient can express the nucleic acid in the recipient. The term “cell” includes bacterial cells and other useful cells such as yeast cells, fungal cells or mammalian cells. Suitable bacterial cells include cells from gram-negative bacterial strains such as strains of Escherichia coli, Proteus, and Pseudomonas, and gram-positive bacterial strains such as strains of Bacillus, Streptomyces, Staphylococcus, and Lactococcus. Suitable fungal cell includes cells from species of Trichoderma, Neurospora, and Aspergillus. Suitable yeast cells include cells from species of Saccharomyces (Tor example Saccharomyces cerevisiae), Schizosaccharomyces (for example Schizosaccharomyces pombe), Pichia (for example Pichia pastoris and Pichia methanolicd), and Hansenula. Suitable mammalian cells include for example CHO cells, BHK cells, HeLa cells, COS cells, 293 HEK and the like. However, amphibian cells, insect cells, plant cells, and any other cells used in the art for the expression of heterologous proteins can be used as well. Mammalian cells are particularly preferred for adoptive transfer, such as cells from humans, mice, hamsters, pigs, goats, and primates. The cells may be derived from a large number of tissue types and include primary cells and cell lines such as cells of the immune system, in particular antigen-presenting cells such as dendritic cells and T cells, stem cells such as hematopoietic stem cells and mesenchymal stem cells and other cell types.

The present invention also provides a recombinant cell expressing the nucleic acid or nucleic acid construct of the invention as defined herein. The recombinant cell may co-express the CoCAR of the present invention, and a BBz-CAR, such as a BBz-CAR as described herein. The CoCAR of the present invention, and a BBz-CAR may be co-expressed on the cell surface.

According to a preferred embodiment of the present invention, the recombinant cell is an immune effector cell. The immune effector cell is preferably selected from the group consisting of but not limited to T cells (cytotoxic T cells, helper T cells, tumor infiltrating T cells), B cells, natural killer cells, neutrophils, macrophages, and dendritic cells. Preferably, in the context of the present invention, the immune effector cells is a T cell, preferably a CD4⁺ and/or CD8⁺ T cell. Particularly preferred cells for use according to the invention are T cells, in particular cytotoxic lymphocytes, preferably selected from cytotoxic T cells, natural killer (NK) cells, and lymphokine-activated killer (LAK) cells. Upon activation, each of these cytotoxic lymphocytes triggers the destruction of target cells. For example, cytotoxic T cells trigger the destruction of target cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target cells. The cytotoxic lymphocytes will preferably be autologous cells, although heterologous cells or allogenic cells can be used.

In the recombinant cell of the invention, expressing the CoCAR of the invention on the surface, the first antigen-recognition domain may be capable of binding a first antigen on the surface of a target cell, wherein binding of the CoCAR to the first antigen does not induce activation of the recombinant cell. The recombinant cell of the invention may further express a CAR comprising a second antigen-recognition domain, wherein the second antigen-recognition domain may be capable of binding a second antigen on the surface of a target cell, wherein binding of the CAR to the second antigen induces activation of the recombinant cell. In the recombinant cell of the invention, binding of the CoCAR to the first antigen may enhance the activation of the recombinant cell induced by binding of the CAR to the second antigen. In the recombinant cell of the invention, the CoCAR may attenuate antigen-independent activation and differentiation of the recombinant cell induced by the CAR. The CAR may be any CAR as described herein.

The invention may involve introduction, i.e. transfection, of nucleic acids encoding the CoCAR of the present invention or polypeptides thereof into respective (host) cells such as T cells in vitro or in vivo.

For purposes of the present invention, the term “transfection” includes the introduction of a nucleic acid into a cell or the uptake of a nucleic acid by a cell, wherein the cell may be present in a subject, e.g., a patient. Thus, according to the present invention, a cell for transfection of a nucleic acid described herein can be present in vitro or in vivo, e.g. the cell can form part of an organ, a tissue and/or an organism of a patient. According to the invention, transfection can be transient or stable. For some applications of transfection, it is sufficient if the transfected genetic material is only transiently expressed. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. RNA can be transfected into cells for transiently expressing its coded protein.

Any technique useful for introducing, i.e. transferring or transfecting, nucleic acids into cells may be used. Preferably, nucleic acids such as DNA or RNA are transfected into cells by standard techniques. Such techniques include electroporation, lipofection and microinjection. The nucleic acid can be introduced into cells by electroporation. Electroporation or electro-permeabilization relates to a significant increase in the electrical conductivity and permeability of the cell plasma membrane caused by an externally applied electrical field. It is usually used in molecular biology as a way of introducing some substance into a cell. It is preferred that introduction of nucleic acid encoding a protein or peptide into cells results in expression of said protein or peptide.

A variety of methods may be used to introduce antigen receptor constructs into T cells including non-viral-based DNA transfection, transposon-based systems and viral-based systems. Non-viral-based DNA transfection has low risk of insertional mutagenesis. Transposon-based systems can integrate transgenes more efficiently than plasmids that do not contain an integrating element. Viral-based systems include the use of γ-retroviruses and lentiviral vectors. γ-Retroviruses are relatively easy to produce, efficiently and permanently transduce T cells, and have preliminarily proven safe from an integration standpoint in primary human T cells. Lentiviral vectors also efficiently and permanently transduce T cells but are more expensive to manufacture. They are also potentially safer than retrovirus-based systems.

For transfection of cells in vivo a pharmaceutical composition comprising nucleic acids encoding the antigen receptor may be used. A delivery vehicle that targets the nucleic acid to a specific cell such as a T cell may be administered to a patient, resulting in transfection that occurs in vivo.

Preferably, the cell of the present invention expresses the CoCAR of the present invention on the cell surface.

Preferably, the cell of the present invention comprises (i) a chimeric antigen receptor (CAR), and (ii) the nucleic acid and/or nucleic acid construct of the present invention. In particular, the CAR comprises a second extracellular antigen-recognition domain, a second hinge region, a second transmembrane domain, a 4-1BB domain and a CD3zeta domain, as described herein. In particular, the cell expresses (i) the CAR, and (ii) the CoCAR on the cell surface. The CAR and the CoCAR of the present invention can recognize different epitopes located in the same antigen expressed on the surface of a target cell. The CAR and the CoCAR of the present invention can recognize different antigens expressed on the surface of a target cell.

Nucleic acids may be comprised in a vector. The term “vector” as used herein includes any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In the context of the present invention, the term “transcription” relates to a process, wherein the genetic code in a DNA sequence is transcribed into RNA. Subsequently, the RNA may be translated into a protein. According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present invention encompassed by the term “vector”.

The term “translation” in the context of the present invention relates to the process in the ribosomes of a cell by which a strand of messenger RNA directs the assembly of a sequence of amino acids to make a peptide or protein.

Nucleic acids may, in accordance with the present invention, be present alone or in combination with other nucleic acids, which may be homologous or heterologous. A nucleic acid can be functionally linked to expression control sequences which may be homologous or heterologous with respect to said nucleic acid. The term “homologous” means that the nucleic acids are also functionally linked naturally and the term “heterologous” means that the nucleic acids are not functionally linked naturally.

A nucleic acid and an expression control sequence are “functionally” linked to one another, if they are covalently linked to one another in such a way that expression or transcription of said nucleic acid is under the control or under the influence of said expression control sequence. If the nucleic acid is to be translated into a functional protein, then, with an expression control sequence functionally linked to a coding sequence, induction of said expression control sequence results in transcription of said nucleic acid, without causing a frame shift in the coding sequence or said coding sequence not being capable of being translated into the desired protein or peptide.

The term “expression control sequence” or “expression control element” comprises promoters, ribosome binding sites, enhancers and other control elements which regulate transcription of a gene or translation of an mRNA. In particular embodiments of the invention, the expression control sequences can be regulated. The exact structure of expression control sequences may vary as a function of the species or cell type, but generally comprises 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences comprise a promoter region which includes a promoter sequence for transcriptional control of the functionally linked nucleic acid. Expression control sequences may also comprise enhancer sequences or upstream activator sequences.

The term “expression” is used in the context the invention in its most general meaning and comprises the production of RNA and/or peptides or proteins, e.g. by transcription and/or translation. With respect to RNA, the term “expression” or “translation” relates in particular to the production of peptides or proteins. It also comprises partial expression of nucleic acids. Moreover, expression can be transient or stable. According to the invention, the term expression also includes an “aberrant expression” or “abnormal expression”.

“Aberrant expression” or “abnormal expression” means according to the invention that expression is altered, preferably increased, compared to a reference, e.g. a state in a subject not having a disease associated with aberrant or abnormal expression of a certain protein, e.g., a tumor antigen. An increase in expression refers to an increase by at least 10%, in particular at least 20%, at least 50% or at least 100%, or more.

According to a preferred embodiment, immune effector cells comprise T cells (cytotoxic T cells, helper T cells, tumor infiltrating T cells), B cells, natural killer cells, neutrophils, macrophages, and dendritic cells. Preferably, “immune effector cells” are T cells, more preferably CD4⁺ and/or CD8⁺ T cells. Upon activation/stimulation, each of these cytotoxic lymphocytes triggers the destruction of target cells. For example, cytotoxic T cells trigger the destruction of target cells by either or both of the following means. First, upon activation, the T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target tumor cells. The T cells and other cytotoxic lymphocytes will preferably be autologous cells, although heterologous cells or allogenic cells can be used in accordance with the present invention.

Thus, an antigen receptor of the present invention may replace the function of a T cell receptor as described above and, in particular, may confer reactivity such as cytolytic activity to a cell such as a T cell as described above. However, in contrast to the binding of the T cell receptor to an antigen peptide-MHC complex, the antigen receptor binds to an antigen, in particular when expressed on the cell surface.

The present invention also provides a population of respective recombinant cells, preferably of respective immune effector cells described herein, which population can be a clonally expanded population. Recombinant immune effector cells or populations thereof provide for therapeutic or prophylactic immune effector function in an antigen-specific manner.

Preferably, the chimeric antigen receptor of the invention is expressed on the cell surface of such recombinant cell and in particular on the cell surface of such immune effector cell.

According to a further aspect, the present invention provides a pharmaceutical composition comprising the CoCAR of the invention, the combination of the present invention, the nucleic acid, the combination of nucleic acids, the nucleic acid construct, the combination of nucleic constructs, the vector, the combination of vectors of the invention, and/or the recombinant cell of the invention, and a pharmaceutically acceptable carrier.

A pharmaceutical composition according to the present invention (also termed simply as “composition”) is preferably sterile and contains an effective amount of the antigen receptors, peptide chains, nucleic acids, recombinant cells, immune effector cells of the invention, as well as other compounds and agents described herein, and optionally further agents as discussed herein to generate the desired reaction or the desired effect.

Pharmaceutical compositions are usually provided in a uniform dosage form and may be prepared in a manner known per se. A pharmaceutical composition may e.g. be in the form of a solution or suspension.

A pharmaceutical composition may comprise salts, buffer substances, preservatives, carriers, diluents and/or excipients in pharmaceutically acceptable form. The term “pharmaceutically acceptable” refers to the non-toxicity of a material which does not interact with the action of the active component of the pharmaceutical composition.

Salts which are not pharmaceutically acceptable may be used for preparing pharmaceutically acceptable salts and are included in the invention. Pharmaceutically acceptable salts of this kind comprise in a non-limiting way those prepared from the following acids: hydrochloric, hydrobromic, sulfuric, nitric, phosphoric, maleic, acetic, salicylic, citric, formic, malonic, succinic acids, and the like. Pharmaceutically acceptable salts may also be prepared as alkali metal salts or alkaline earth metal salts, such as sodium salts, potassium salts or calcium salts.

Suitable buffer substances for use in a pharmaceutical composition include acetic acid in a salt, citric acid in a salt, boric acid in a salt and phosphoric acid in a salt.

Suitable preservatives for use in a pharmaceutical composition include benzalkonium chloride, chlorobutanol, paraben and thimerosal.

The term “carrier” refers to an organic or inorganic component, of a natural or synthetic nature, in which the active component is combined in order to facilitate, enhance or enable application. According to the invention, the term “carrier” also includes one or more compatible solid or liquid fillers, diluents or encapsulating substances, which are suitable for administration to a patient.

Possible carrier substances for parenteral administration are e.g. sterile water, Ringer, Ringer lactate, sterile sodium chloride solutions, polyalkylene glycols, hydrogenated naphthalenes and, in particular, biocompatible lactide polymers, lactide/glycolide copolymers or polyoxyethylene/polyoxy-propylene copolymers.

The term “excipient” when used herein is intended to indicate all substances which may be present in a pharmaceutical composition and which are not active ingredients such as, e.g., carriers, binders, lubricants, thickeners, surface active agents, preservatives, emulsifiers, buffers, flavoring agents, or colorants.

The compositions described herein may be administered via any conventional route, such as by parenteral administration including by injection or infusion. Administration is preferably parenterally, e.g. intravenously, intraarterially, subcutaneously, intradermally or intramuscularly.

Compositions suitable for parenteral administration usually comprise a sterile aqueous or nonaqueous preparation of the active compound, which is preferably isotonic to the blood of the recipient. Examples of compatible carriers and solvents are Ringer solution and isotonic, slightly hypertonic or slightly hypotonic sodium chloride solutions. In addition, usually sterile, fixed oils can be used as solution or suspension medium.

The pharmaceutical compositions described herein are preferably administered in effective amounts. An “effective amount” refers to the amount which achieves a desired reaction or a desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition.

An effective amount of a composition described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the agents and compositions described herein may depend on various of such parameters. In case a reaction in a patient is insufficient with an initial dose, higher doses (or effectively higher doses achieved by a different, more localized route of administration) may be used.

The compositions described herein can be administered to patients to treat or prevent a variety of disorders such as those described herein. Patients preferably include human patients having disorders that can be corrected or ameliorated by administering the agents and compositions described herein. This includes disorders involving cells characterized by expression of an antigen, in particular by expression of CD22.

For example, in one embodiment, the compositions described herein can be used to treat a patient with a cancer disease, e.g., a cancer disease such as described herein characterized by the presence of cancer cells expressing a tumor-associated antigen.

The pharmaceutical compositions described according to the invention may also be used for immunization or vaccination to prevent a disease described herein.

The pharmaceutical composition of the invention may be administered together with supplementing immunity-enhancing substances such as one or more adjuvants and may comprise one or more immunity-enhancing substances to further increase its effectiveness, preferably to achieve a synergistic effect of immune-stimulation. The term “adjuvant” relates to compounds which prolongs or enhances or accelerates an immune response. Various mechanisms are possible in this respect, depending on the various types of adjuvants. For example, compounds which allow the maturation of the DC, e.g. lipopolysaccharides or CD40 ligand, form a first class of suitable adjuvants. Generally, any agent which influences the immune system of the type of a “danger signal” (LPS, GP96, dsRNA etc.) or cytokines, such as GM-CSF, can be used as an adjuvant which enables an immune response to be intensified and/or influenced in a controlled manner. CpG oligodeoxynucleotides can optionally also be used in this context, although their side effects which occur under certain circumstances, as explained above, are to be considered. Particularly preferred adjuvants are cytokines, such as monokines, lymphokines, interleukins or chemokines, e.g. IL-1, IL-2, IL-3, IL-4, IL-5, IL-6, IL-7, IL-8, IL-9, IL-10, IL-12, IFNα, IFNγ, GM-CSF, LT-α, or growth factors, e.g. hGH. Further known adjuvants are aluminium hydroxide, Freund's adjuvant or oil such as Montanide®, most preferred Montanide® ISA51. Lipopeptides, such as Pam3Cys, are also suitable for use as adjuvants in the pharmaceutical composition of the present invention.

The pharmaceutical composition of the invention can be administered locally or systemically, preferably systemically.

The term “systemic administration” refers to the administration of an agent such that the agent becomes widely distributed in the body of an individual in significant amounts and develops a desired effect. For example, the agent may develop its desired effect in the blood and/or reaches its desired site of action via the vascular system. Typical systemic routes of administration include administration by introducing the agent directly into the vascular system or oral, pulmonary, or intramuscular administration wherein the agent is adsorbed, enters the vascular system, and is carried to one or more desired site(s) of action via the blood.

The systemic administration by parenteral administration is a particularly preferred route. The term “parenteral administration” refers to administration of an agent such that the agent does not pass the intestine. The term “parenteral administration” includes intravenous administration, subcutaneous administration, intradermal administration or intraarterial administration but is not limited thereto.

Administration may also be carried out, for example, orally, intraperitoneally or intramuscularly.

The compositions described herein may be used alone or in combination with conventional therapeutic regimens such as surgery, irradiation, chemotherapy and/or bone marrow transplantation (autologous, syngeneic, allogeneic or unrelated).

According to the present invention, the CoCAR, its peptide chains, the combination, the nucleic acid, the combination of nucleic acids, the nucleic acid construct, the combination of nucleic constructs, the vector, the combination of vectors, the recombinant cell and/or the pharmaceutical composition described herein can be used in medicine. According to a preferred embodiment of the invention, the CoCAR, its peptide chains, the combination, the nucleic acid, the combination of nucleic acids, the nucleic acid construct, the combination of nucleic constructs, the vector, the combination of vectors, the recombinant cell and/or the pharmaceutical composition described herein are for use in treating cancer. More preferably, the CoCAR, its peptide chains, the nucleic acid, the combination of nucleic acids, the nucleic acid construct, the combination of nucleic constructs, the vector, the combination of vectors, the recombinant cell and/or the pharmaceutical composition described herein is for use in treating a malignancy associated with cell-surface expression of at least one tumor-associated antigen.

The tumor-associated antigen can be selected from the group consisting of B7-H3 (CD276), BCMA, CD3, CD5, CD19, CD20, CD22, CD23, CD30, CD33, CD38, CD44, CD44v6, CD52, CD70, CD79A, CD79B, CD123, CD138, CD171, CEA, Claudin-6, Claudin-18.2, CLL1, CXCR5, EGFR, EGFRvIII, EPH-receptor A2, IGLV3-21, GP-2, GP-40, HER2, ErbB3, ErbB4, FBP, AchR, Fr-a, GD2, GD3, HMW-MAA, IL13Ra2, Kappa-LC, IGLV3-21-R110, Lewis Y, Mesothelin, MUC1, MUC16, NKG2D Ligands, NCAM, NY-ESO1, Oncofetal antigen h5T4, PSCA, PSMA, ROR1, TAG-72, VEGFR, GOLPH2, and SLAMF7. A preferred tumor-associated antigen is selected from the group consisting of BCMA, CD22, CD79B, GOLPH2, EGFR/EGFRvIII, HER2, CEA, and GOLPH2. A more preferred tumor-associated antigen is selected from the group consisting of CD22, CD19, HER2, and EGFR.

More preferably, the CoCAR, its peptide chains, the nucleic acid, the combination of nucleic acids, the nucleic acid construct, the combination of nucleic constructs, the vector, the combination of vectors, the recombinant cell and/or the pharmaceutical composition described herein is for use in treating a malignancy associated with cell-surface expression of at least one tumor-associated antigen, and wherein the at least one tumor-associated antigen has a density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell.

More preferably, the CoCAR, its peptide chains, the nucleic acid, the combination of nucleic acids, the nucleic acid construct, the combination of nucleic constructs, the vector, the combination of vectors, the recombinant cell and/or the pharmaceutical composition described herein is for use in treating a malignancy associated with cell-surface expression of at least one tumor-associated antigen, wherein the at least one tumor-associated antigen is selected from the group consisting of CD22, CD19, HER2, and EGFR, and wherein the at least one tumor-associated antigen has a density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell.

According to a preferred embodiment, the malignancy is a B cell malignancy.

It is preferred that the disease is characterized by the presence of diseased cells expressing CD22. According to a particularly preferred embodiment, the malignancy associated with cell-surface CD22 expression is a B cell malignancy.

Accordingly, the CoCAR, its peptide chains, the nucleic acid, the combination of nucleic acids, the nucleic acid construct, the combination of nucleic constructs, the vector, the combination of vectors, the recombinant cell and/or the pharmaceutical composition described herein can be used to treat a subject with a disease, particularly for treating cancer. According to a preferred embodiment, the CoCAR, its peptide chains, the nucleic acid, the combination of nucleic acids, the nucleic acid construct, the combination of nucleic constructs, the vector, the combination of vectors, the recombinant cell and/or the pharmaceutical composition described herein can be used to treat a malignancy associated with cell-surface CD22 expression in a subject in need thereof, for example a disease characterized by the presence of diseased cells expressing CD22. The malignancy associated with cell-surface CD22 expression is preferably a B cell malignancy.

The CoCAR, the nucleic acid, the combination of nucleic acids, the nucleic acid construct, the combination of nucleic constructs, the vector, the combination of vectors, the recombinant cell and/or the pharmaceutical composition described herein may also be used for immunization or vaccination to prevent a disease described herein.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality. According to the invention, the term “disease” includes infectious diseases and cancer diseases, in particular those forms of cancer described herein. Any reference herein to cancer or particular forms of cancer also includes cancer metastasis thereof.

A disease to be treated according to the invention is preferably a disease involving expression of CD22 antigen. “Disease involving an antigen”, “disease associated with expression or elevated expression of an antigen” or similar expressions means according to the invention that the antigen is expressed in cells of a diseased tissue or organ. Expression in cells of a diseased tissue or organ may be increased compared to the state in a healthy tissue or organ. In one embodiment, expression is only found in a diseased tissue, while expression in a healthy tissue is not found, e.g. expression is repressed. According to the invention, diseases involving an antigen include infectious diseases and cancer diseases, wherein the disease-associated antigen is preferably an antigen of the infectious agent and a tumor antigen, respectively. Preferably a disease involving an antigen preferably is a disease involving cells expressing an antigen, preferably on the cell surface.

The term “healthy” or “normal” refer to non-pathological conditions, and preferably means non-infected or non-cancerous.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer, lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the invention also comprises cancer metastases. Preferably, a “cancer disease” is characterized by cells expressing CD22.

In one embodiment, a cancer disease is a malignant disease which is characterized by the properties of anaplasia, invasiveness, and metastasis. A malignant tumor may be contrasted with a non-cancerous benign tumor in that a malignancy is not self-limited in its growth, is capable of invading into adjacent tissues, and may be capable of spreading to distant tissues (metastasizing), while a benign tumor has none of those properties.

According to the invention, the term “tumor” or “tumor disease” refers to a swelling or lesion formed by an abnormal growth of cells (called neoplastic cells or tumor cells). By “tumor cell” is meant an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign, pre-malignant or malignant.

According to the invention, a “carcinoma” is a malignant tumor derived from epithelial cells. This group represents the most common cancers, including the common forms of breast, prostate, lung and colon cancer.

“Adenocarcinoma” is a cancer that originates in glandular tissue. This tissue is also part of a larger tissue category known as epithelial tissue. Epithelial tissue includes skin, glands and a variety of other tissue that lines the cavities and organs of the body. Epithelium is derived embryologically from ectoderm, endoderm and mesoderm. To be classified as adenocarcinoma, the cells do not necessarily need to be part of a gland, as long as they have secretory properties. This form of carcinoma can occur in some higher mammals, including humans. Well differentiated adenocarcinomas tend to resemble the glandular tissue that they are derived from, while poorly differentiated may not. By staining the cells from a biopsy, a pathologist will determine whether the tumor is an adenocarcinoma or some other type of cancer. Adenocarcinomas can arise in many tissues of the body due to the ubiquitous nature of glands within the body. While each gland may not be secreting the same substance, as long as there is an exocrine function to the cell, it is considered glandular and its malignant form is therefore named adenocarcinoma. Malignant adenocarcinomas invade other tissues and often metastasize given enough time to do so. Ovarian adenocarcinoma is the most common type of ovarian carcinoma. It includes the serous and mucinous adenocarcinomas, the clear cell adenocarcinoma and the endometrioid adenocarcinoma.

Lymphoma and leukemia are malignancies derived from hematopoietic (blood-forming) cells.

Blastic tumor or blastoma is a tumor (usually malignant) which resembles an immature or embryonic tissue. Many of these tumors are most common in children.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential. In one embodiment, the term “metastasis” according to the invention relates to “distant metastasis” which relates to a metastasis which is remote from the primary tumor and the regional lymph node system. In one embodiment, the term “metastasis” according to the invention relates to lymph node metastasis.

A relapse or recurrence occurs when a person is affected again by a condition that affected them in the past. For example, if a patient has suffered from a tumor disease, has received a successful treatment of said disease and again develops said disease said newly developed disease may be considered as relapse or recurrence. However, a relapse or recurrence of a tumor disease may but does not necessarily occur at the site of the original tumor disease. Thus, for example, if a patient has suffered from ovarian tumor and has received a successful treatment a relapse or recurrence may be the occurrence of an ovarian tumor or the occurrence of a tumor at a site different to ovary. A relapse or recurrence of a tumor also includes situations wherein a tumor occurs at a site different to the site of the original tumor as well as at the site of the original tumor. The original tumor for which the patient has received a treatment can be a primary tumor and the tumor at a site different to the site of the original tumor is a secondary or metastatic tumor.

The present invention is also directed to the use of the CoCAR, the combination, its peptide chains, the nucleic acid, the combination of nucleic acids, the nucleic acid construct, the combination of nucleic constructs, the vector, the combination of vectors, the recombinant cell and/or the pharmaceutical composition as described herein in therapeutic and prophylactic methods. One such use is in the production of antigen-specific immune cells, which can be administered to a patient for preventing or treating a disease, which disease is characterized by expression of a tumor-associated antigen that can be bound by an antigen receptor of the invention expressed in the immune cells. Preferably, the disease is cancer. Further, an antigen receptor of the invention and related molecules also can be used for the selective eradication of cells expressing a tumor-associated antigen, as well as for immunization or vaccination against a disease wherein the tumor-associated antigen is expressed.

In one embodiment, a method of treating or preventing cancer in a subject or patient in need thereof comprises administering to the subject or patient an effective amount of the CoCAR of the invention. In particular, in the CoCAR, the antigen binding site of the antigen receptor is able to bind to an antigen that is associated with the disease to be treated or prevented. According to a preferred embodiment, the cancer is a malignancy positive for at least one tumor-associated antigen, and preferably a B cell malignancy.

In one embodiment, a method of treating or preventing cancer in a subject or patient in need thereof comprises administering to the subject or patient an effective amount of the nucleic acid, the combination of nucleic acids, the nucleic acid construct, the combination of nucleic constructs, the vector, and/or the combination of vectors according to the invention. According to a preferred embodiment, the cancer is a malignancy positive for at least one tumor-associated antigen, and preferably a B cell malignancy. According to a preferred embodiment, the cancer is a malignancy positive for at least one tumor-associated antigen, and preferably for at least one tumor-associated antigen having a density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell, and preferably for at least one tumor-associated antigen selected from CD22, CD19, HER2 and EGFR.

In one embodiment, a method of treating or preventing cancer in a subject or patient in need thereof comprises administering to the subject or patient an effective amount of the recombinant cell or population of recombinant cells according to the invention. According to a preferred embodiment, the cancer is a malignancy positive for at least one tumor-associated antigen, and preferably a B cell malignancy.

In one embodiment, a method of treating or preventing cancer in a subject or patient in need thereof comprises administering to the subject or patient an effective amount of the pharmaceutical composition according to the invention. According to a preferred embodiment, the cancer is a malignancy positive for at least one tumor-associated antigen, and preferably a B cell malignancy.

The present invention also provides a method of immunizing or vaccinating against a disease associated with expression of at least one tumor-associated antigen, which method comprises administering to a patient an effective amount of the CoCAR, its peptide chains, the nucleic acid, the combination of nucleic acids, the nucleic acid construct, the combination of nucleic constructs, the vector, and/or the combination of vectors, the recombinant cell and/or the pharmaceutical composition described herein, in which the antigen binding site of the antigen receptor is able to bind to an antigen that is associated with the disease (e.g., CD22).

The at least one tumor-associated antigen can be selected from the group consisting of B7-H3 (CD276), BCMA, CD3, CD5, CD19, CD20, CD22, CD23, CD30, CD33, CD38, CD44, CD44v6, CD52, CD70, CD79A, CD79B, CD123, CD138, CD171, CEA, Claudin-6, Claudin-18.2, CLL1, CXCR5, EGFR, EGFRvIII, EPH-receptor A2, IGLV3-21, GP-2, GP-40, HER2, ErbB3, ErbB4, FBP, AchR, Fr-a, GD2, GD3, HMW-MAA, IL13Ra2, Kappa-LC, IGLV3-21-R110, Lewis Y, Mesothelin, MUC1, MUC16, NKG2D Ligands, NCAM, NY-ESO1, Oncofetal antigen h5T4, PSCA, PSMA, ROR1, TAG-72, VEGFR, GOLPH2, and SLAMF7. A preferred tumor-associated antigen is selected from the group consisting of BCMA, CD22, CD79B, GOLPH2, EGFR/EGFRvIII, HER2, CEA, and GOLPH2. A more preferred tumor-associated antigen is selected from the group consisting of CD22, CD19, HER2, and EGFR.

According to a preferred embodiment, the at least one tumor-associated antigen has a density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell.

The term “treatment” or “therapeutic treatment” relates to any treatment which improves the health status and/or prolongs (increases) the lifespan of an individual. Said treatment may eliminate the disease in an individual, arrest, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that are intended to prevent a disease from occurring in an individual. The terms “prophylactic treatment” and “preventive treatment” are used herein interchangeably.

The terms “individual” and “subject” are used herein interchangeably. They refer to human beings, non-human primates or other mammals (e.g. mouse, rat, rabbit, dog, cat, cattle, swine, sheep, horse or primate) that can be afflicted with or are susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In preferred embodiments of the present invention, the “individual” or “subject” is a “patient”. The term “patient” means a subject for treatment, in particular a diseased subject.

By “being at risk” or “in need of” is meant to refer to a subject, i.e. a patient, that is identified as having a higher than normal chance of developing a disease, in particular cancer, compared to the general population. In addition, a subject who has had, or who currently has, a disease, in particular cancer, is a subject who has an increased risk for developing a disease, as such a subject may continue to develop a disease. Subjects who currently have, or who have had, a cancer also have an increased risk for cancer metastases.

In the context of the present invention, terms such as “protect”, “prevent”, “prophylactic”, “preventive”, or “protective” relate to the prevention or treatment or both of the occurrence and/or the propagation of a disease in a subject and, in particular, to minimizing the chance that a subject will develop a disease or to delaying the development of a disease. For example, a person at risk for a tumor, as described above, would be a candidate for therapy to prevent a tumor.

A prophylactic administration of an immunotherapy, for example, a prophylactic administration of an agent or composition of the invention, preferably protects the recipient from the development of a disease. A therapeutic administration of an immunotherapy, for example, a therapeutic administration of an agent or composition of the invention, may lead to the inhibition of the progress/growth of the disease. This comprises the deceleration of the progress/growth of the disease, in particular a disruption of the progression of the disease, which preferably leads to elimination of the disease.

Immunotherapy may be performed using any of a variety of techniques, in which agents provided herein preferably function to remove antigen-expressing cells from a patient. Such removal may take place as a result of enhancing or inducing an immune response in a patient specific for antigen or a cell expressing antigen.

The term “immunization” or “vaccination” describes the process of treating a subject with the purpose of inducing an immune response for therapeutic or prophylactic reasons.

In accordance with the above, the invention preferably relates to the following items:
1. A coreceptor-CAR (CoCAR), comprising
   (i) a first extracellular antigen-recognition domain,
   (ii) a first hinge domain,
   (iii) a first transmembrane domain, and
   (iv) at least one cytosolic domain
      (a) capable of binding LCK, and/or
      (b) comprising LCK, or a variant or fragment thereof.
2. The CoCAR according to Item 1, wherein the first antigen-recognition domain is a scFv.
3. The CoCAR according to Item 1 or 2, wherein the first antigen-recognition domain specifically recognizes at least one tumor-associated antigen selected from B7-H3 (CD276), BCMA, CD3, CD5, CD19, CD20, CD22, CD23, CD30, CD33, CD38, CD44, CD44v6, CD52, CD70, CD79A, CD79B, CD123, CD138, CD171, CEA, Claudin-6, Claudin-18.2, CLL1, CXCR5, EGFR, EGFRvIII, EPH-receptor A2, IGLV3-21, GP-2, GP-40, HER2, ErbB3, ErbB4, FBP, AchR, Fr-a, GD2, GD3, HMW-MAA, IL13Ra2, Kappa-LC, IGLV3-21-R110, Lewis Y, Mesothelin, MUC1, MUC16, NKG2D Ligands, NCAM, NY-ESO1, Oncofetal antigen h5T4, PSCA, PSMA, ROR1, TAG-72, VEGFR, GOLPH2, and SLAMF7.
4. The CoCAR according to Item 3, wherein the at least one tumor-associated antigen is selected from the group consisting of BCMA, CD22, CD79B, GOLPH2, EGFR/EGFRvIII, HER2, CEA, and GOLPH2.
5. The CoCAR according to any one of the Items 1-4, wherein the first antigen-recognition domain comprises
   (a) a light chain variable domain (VL domain) comprising a light chain complementarity determining region (LCDR) 1 of SEQ ID NO: 2, a LCDR2 of SEQ ID NO: 3 and a LCDR of SEQ ID NO: 4; and
   (b) a heavy chain variable domain (VH domain) comprising a heavy chain complementarity determining region (HCDR) 1 of SEQ ID NO: 6, a HCDR2 of SEQ ID NO: 7 and a HCDR of SEQ ID NO: 8, wherein the light chain variable domain and the heavy chain variable domain specifically bind CD22.
6. The CoCAR according to any one of the Items 1-5,
   (a) one or more of the light chain frameworks light chain framework region 1 (LFR1), light chain framework region 2 (LFR2), light chain framework region 3 (LFR3) and light chain framework region 4 (LFR4) having a at least 90% amino acid sequence identity to the amino acid sequence of the respective framework regions of the light chain variable region according to SEQ ID NO: 5 and/or
   (b) one or more of the heavy chain frameworks heavy chain framework region 1 (HFR1), heavy chain framework region 2 (HFR2), heavy chain framework region 3 (HFR3) and heavy chain framework region 4 (HFR4) having a at least 90% amino acid sequence identity to the amino acid sequence of the respective framework regions of the heavy chain variable region according to SEQ ID NO: 9.
7. The CoCAR according to any one of the Items 1-6, wherein
   LFR1 comprises SEQ ID NO: 11,
   LFR2 comprises SEQ ID NO: 12,
   LFR3 comprises SEQ ID NO: 13,
   LFR4 comprises SEQ ID NO: 14,
   HFR1 comprises SEQ ID NO: 15,
   HFR2 comprises SEQ ID NO: 16,
   HFR3 comprises SEQ ID NO: 17, and/or
   HFR4 comprises SEQ ID NO: 18.
8. The CoCAR according to any one of the Items 1-4, wherein the first antigen-recognition domain comprises
   (a) a light chain variable domain comprising a light chain complementarity determining region (LCDR) 1 of SEQ ID NO: 20, a LCDR2 of SEQ ID NO: 21 and a LCDR of SEQ ID NO: 22; and
   (b) a heavy chain variable domain comprising a heavy chain complementarity determining region (HCDR) 1 of SEQ ID NO: 24, a HCDR2 of SEQ ID NO: 25 and a HCDR of SEQ ID NO: 26, wherein the light chain variable domain and the heavy chain variable domain specifically bind CD22.
9. The CoCAR according to any one of the Items 1-4 and 8, comprising
   (a) one or more of the light chain frameworks light chain framework region 1 (LFR1), light chain framework region 2 (LFR2), light chain framework region 3 (LFR3) and light chain framework region 4 (LFR4) having a at least 90% amino acid sequence identity to the amino acid sequence of the respective framework regions of the light chain variable region according to SEQ ID NO: 23 and/or
   (b) one or more of the heavy chain frameworks heavy chain framework region 1 (HFR1), heavy chain framework region 2 (HFR2), heavy chain framework region 3 (HFR3) and heavy chain framework region 4 (HFR4) having a at least 90% amino acid sequence identity to the amino acid sequence of the respective framework regions of the heavy chain variable region according to SEQ ID NO: 27.
10. The CoCAR according to any one of the Items 1-4, 8 and 9, wherein
   LFR1 comprises SEQ ID NO: 29,
   LFR2 comprises SEQ ID NO: 30,
   LFR3 comprises SEQ ID NO: 31,
   LFR4 comprises SEQ ID NO: 32,
   HFR1 comprises SEQ ID NO: 33,
   HFR2 comprises SEQ ID NO: 34,
   HFR3 comprises SEQ ID NO: 35, and/or
   HFR4 comprises SEQ ID NO: 36.
11. The CoCAR according to any one of the Items 1-4, wherein the first antigen-recognition domain comprises
   (a) a light chain variable domain comprising a light chain complementarity determining region (LCDR) 1 of SEQ ID NO: 38, a LCDR2 of SEQ ID NO: 39 and a LCDR of SEQ ID NO: 40; and
   (b) a heavy chain variable domain comprising a heavy chain complementarity determining region (HCDR) 1 of SEQ ID NO: 42, a HCDR2 of SEQ ID NO: 43 and a HCDR of SEQ ID NO: 44, wherein the light chain variable domain and the heavy chain variable domain specifically bind CD19.
12. The CoCAR according to any one of the Items 1-4 and 11, comprising
   (a) one or more of the light chain frameworks light chain framework region 1 (LFR1), light chain framework region 2 (LFR2), light chain framework region 3 (LFR3) and light chain framework region 4 (LFR4) having a at least 90% amino acid sequence identity to the amino acid sequence of the respective framework regions of the light chain variable region according to SEQ ID NO: 41 and/or
   (b) one or more of the heavy chain frameworks heavy chain framework region 1 (HFR1), heavy chain framework region 2 (HFR2), heavy chain framework region 3 (HFR3) and heavy chain framework region 4 (HFR4) having a at least 90% amino acid sequence identity to the amino acid sequence of the respective framework regions of the heavy chain variable region according to SEQ ID NO: 45.
13. The CoCAR according to any one of the Items 1-4, 11 and 12, wherein
   LFR1 comprises SEQ ID NO: 47,
   LFR2 comprises SEQ ID NO: 48,
   LFR3 comprises SEQ ID NO: 49,
   LFR4 comprises SEQ ID NO: 50,
   HFR1 comprises SEQ ID NO: 51,
   HFR2 comprises SEQ ID NO: 52,
   HFR3 comprises SEQ ID NO: 53, and/or
   HFR4 comprises SEQ ID NO: 54.
14. The CoCAR according to any one of the Items 1-4, wherein the first antigen-recognition domain comprises
   (a) a light chain variable domain comprising a light chain complementarity determining region (LCDR) 1 of SEQ ID NO: 56, a LCDR2 of SEQ ID NO: 57 and a LCDR of SEQ ID NO: 58; and
   (b) a heavy chain variable domain comprising a heavy chain complementarity determining region (HCDR) 1 of SEQ ID NO: 60, a HCDR2 of SEQ ID NO: 61 and a HCDR of SEQ ID NO: 62, wherein the light chain variable domain and the heavy chain variable domain specifically bind CEA.
15. The CoCAR according to any one of the Items 1-4 and 14, comprising
   (a) one or more of the light chain frameworks light chain framework region 1 (LFR1), light chain framework region 2 (LFR2), light chain framework region 3 (LFR3) and light chain framework region 4 (LFR4) having a at least 90% amino acid sequence identity to the amino acid sequence of the respective framework regions of the light chain variable region according to SEQ ID NO: 59 and/or
   (b) one or more of the heavy chain frameworks heavy chain framework region 1 (HFR1), heavy chain framework region 2 (HFR2), heavy chain framework region 3 (HFR3) and heavy chain framework region 4 (HFR4) having a at least 90% amino acid sequence identity to the amino acid sequence of the respective framework regions of the heavy chain variable region according to SEQ ID NO: 63.
16. The CoCAR according to any one of the Items 1-4, 14 and 15, wherein
   LFR1 comprises SEQ ID NO: 65,
   LFR2 comprises SEQ ID NO: 66,
   LFR3 comprises SEQ ID NO: 67,
   LFR4 comprises SEQ ID NO: 68,
   HFR1 comprises SEQ ID NO: 69,
   HFR2 comprises SEQ ID NO: 70,
   HFR3 comprises SEQ ID NO: 71, and/or
   HFR4 comprises SEQ ID NO: 72.
17. The CoCAR according to any one of the Items 5-16, wherein in the first antigen-recognition domain, the VH domain and the VL domain are connected by a linker.
18. The CoCAR according to Item 17, the linker comprises glycine and/or alanine residues.
19. The CoCAR according to Item 17, the linker is selected from SEQ ID NO: 10, SEQ ID NO: 46, and fragments and variants thereof.
20. The CoCAR according to any one of the preceding Items, capable of specifically binding to an antigen on a target cell.
21. The CoCAR according to any one of the Items 1-20, wherein the first antigen-recognition domain is capable of binding a first antigen on the surface of a target cell, wherein binding of the CoCAR to the first antigen does not induce activation of a recombinant cell expressing the CoCAR.
22. The CoCAR according to Item 21, wherein the recombinant cell further expresses a CAR comprising a second antigen-recognition domain, wherein the second antigen-recognition domain is capable of binding a second antigen on the surface of a target cell, wherein binding of the CAR to the second antigen induces activation of the recombinant cell.
23. The CoCAR according to Item 22, wherein binding of the CoCAR to the first antigen enhances the activation of the recombinant cell induced by binding of the CAR to the second antigen.
24. The CoCAR according to Item 22 or 23, wherein the CoCAR attenuates antigen-independent activation and differentiation of the recombinant cell induced by the CAR.
25. The CoCAR according to any one of the preceding Items, wherein the first hinge domain is derived from a CD4, CD8, CD28, IgG-Fc domain.
26. The CoCAR according to any one of the preceding Items, wherein the first hinge domain comprises a sequence selected from SEQ ID NOs: 73, 78, 82 and 85 and variants and fragments thereof.
27. The CoCAR according to any one of the preceding Items, comprising at least one spacer region.
28. The CoCAR according to Item 27, wherein the spacer comprises the first hinge domain.
29. The CoCAR according to any one of the preceding Items, wherein the first transmembrane domain is derived from a CD4, CD8, CD28, or CD3zeta transmembrane domain.
30. The CoCAR according to any one of the preceding Items, wherein the first transmembrane domain comprises a sequence selected from SEQ ID NOs: 74, 79, 83 and 86 and variants and fragments thereof, preferably SEQ ID NO: 83 and 86 and variants and fragments thereof.
31. The CoCAR according to any one of the preceding Items, wherein the at least one cytosolic domain (a) capable of binding LCK
   (1) is an intracellular signaling domain derived from a CD4, CD8α, CD28, CD3ε, CD44, or CD146 intracellular signaling domain, or a variant or fragment thereof, or
   (2) comprises one or more motifs capable of binding LCK.
32. The CoCAR according to Item 31, wherein the intracellular signaling domain (1) is derived from a CD4, CD8α, CD3ε, CD44, or CD146 intracellular signaling domain, or a variant or fragment thereof.
33. The CoCAR according to Item 31, wherein the intracellular signaling domain (1) is derived from a CD4, CD8α, CD28, CD44, or CD146 intracellular signaling domain, or a variant or fragment thereof.
34. The CoCAR according to any one of Items 31-33, wherein the intracellular domain (1) is derived from a CD4, CD8α, CD44, or CD146 intracellular signaling domain, or a variant or fragment thereof.
35. The CoCAR according to any one of Items 31-34, wherein the intracellular domain (1) is derived from a CD4 or CD8α intracellular signaling domain, or a variant or fragment thereof.
36. The CoCAR according to any one of the Items 31-35, wherein the motif capable of binding LCK is derived from a CD4, CD8α, CD28, CD3ε, CD44, or CD146 intracellular signaling domain.
37. The CoCAR according to any one of the Items 31-36, wherein the motif capable of binding LCK is derived from a CD4, CD8α, CD3ε, CD44, or CD146 intracellular signaling domain, or a variant or fragment thereof.
38. The CoCAR according to any one of the Items 31-36, wherein the motif capable of binding LCK is derived from a CD4, CD8α, CD28, CD44, or CD146 intracellular signaling domain, or a variant or fragment thereof.
39. The CoCAR according to any one of Items 31-38, wherein the motif capable of binding LCK is derived from a CD4, CD8α, CD44, or CD146 intracellular signaling domain, or a variant or fragment thereof.
40. The CoCAR according to any one of Items 31-39, wherein the motif capable of binding LCK is derived from a CD4 or CD8α intracellular signaling domain, or a variant or fragment thereof,
41. The CoCAR according to any one of the preceding Items 31-40, wherein the motif capable of binding LCK comprises a sequence selected from SEQ ID NO: 84, 87, and 89-102, and fragments and variants thereof.
42. The CoCAR according to any one of the preceding Items, wherein the at least one cytosolic domain comprises LCK.
43. The CoCAR according to Item 42, wherein LCK comprises SEQ ID NO: 103, or a fragment or variant thereof.
44. The CoCAR according to any one on the preceding Items, wherein the at least one cytosolic domain does not comprise a CD3zeta domain.
45. A combination comprising
   (a) the CoCAR of any one of the Items 1-44, and
   (b) a chimeric antigen receptor (CAR).
46. The combination of Item 45, wherein the chimeric antigen receptor (b) comprises
   (i) a second extracellular antigen-recognition domain,
   (ii) a second hinge region,
   (iii) a second transmembrane domain,
   (iv) a 4-1BB domain and/or a CD28 domain, and
   (v) a CD3zeta domain,
47. The combination of Item 45 or 46, wherein in the chimeric antigen receptor (b), the 4-1BB domain comprises SEQ ID NO: 80, or a fragment or variant thereof.
48. The combination of any one of the Items 45-47, wherein in the chimeric antigen receptor (b), the CD3zeta domain comprises SEQ ID NO: 81, or a fragment or variant thereof.
49. The combination of any one of the Items 45-48, wherein in the CAR (b), the second hinge comprises a hinge derived from CD28, in particular the second hinge comprises SEQ ID NO: 78, or a variant or fragment thereof.
50. The combination of Item 49, wherein in the CoCAR (a) the first hinge does not comprise a sequence derived from CD28, in particular the first hinge does not comprise SEQ ID NO: 78.
51. The combination of any one of the Items 49-50, wherein in the CAR (b), the second hinge comprises a hinge derived from CD28, in particular the second hinge comprises SEQ ID NO: 78, or a variant or fragment thereof, and the CoCAR (a) comprises a first hinge region derived from CD4, in particular comprising SEQ ID NO: 82, or a variant or fragment thereof.
52. The combination of any one of the Items 49-50, wherein in the CAR (b), the second hinge comprises a hinge derived from CD28, in particular the second hinge comprises SEQ ID NO: 78, or a variant or fragment thereof, and the CoCAR (a) comprises a first hinge region derived from CD8, preferably CD8α, in particular the second hinge comprises SEQ ID NO: 73 or 85, or a variant or fragment thereof.
53. The combination of any one of the Items 45-52, wherein in the CAR (b), the second hinge comprises a hinge derived from CD8, preferably CD8α, in particular the second hinge comprises SEQ ID NO: 73 or 85, or a variant or fragment thereof.
54. The combination of Item 53, wherein in the CoCAR (a) the first hinge does not comprise SEQ ID NO: 73 or 85.
55. The combination of any one of the Items 53-54, wherein in the CAR (b), the second hinge comprises a hinge derived from CD8, preferably CD8α, in particular the second hinge comprises SEQ ID NO: 73 or 85, or a variant or fragment thereof, and the CoCAR (a) comprises a first hinge region derived from CD4, in particular comprising SEQ ID NO: 82.
56. The combination of any one of the Items 53-54, wherein in the CAR (b), the second hinge comprises a hinge derived from CD8, preferably CD8α, in particular the second hinge comprises SEQ ID NO: 73 or 85, or a variant or fragment thereof, and the CoCAR (a) comprises a first hinge region derived from CD28, in particular comprising SEQ ID NO: 78.
57. A nucleic acid encoding the CoCAR according to any one of Items 1 to 44, or the combination of any one if the Items 45-56.
58. A combination of a first and second nucleic acid, encoding the combination of any one of the Item 45-56, wherein the first nucleic acid encodes the CoCAR (a), and the second nucleic acid encodes the CAR (b).
59. A nucleic acid construct, comprising the nucleic acid of Item 57 and/or the combination of a first and second nucleic acid of Item 58.
60. A combination of a first and second nucleic acid construct, comprising the combination of Item 58, wherein the first nucleic acid construct comprises the first nucleic acid, and the second nucleic acid construct comprises the second nucleic acid.
61. The nucleic acid construct of Item 59 or the combination of nucleic acid constructs of Item 60, wherein the nucleic acid and/or the first and second nucleic acids independently are functionally linked to at least one expression control sequence.
62. The nucleic acid construct or the combination of nucleic acid constructs of Item 61, wherein the at least one expression control sequence is selected from promoters, ribosome binding sites, enhancers and control elements which regulate transcription of the nucleic acid(s) or translation of mRNA(s) and enhancer sequences or upstream activator sequences.
63. A vector, comprising the nucleic acid of Item 57, the combination of the first and second nucleic acid of Item 58, the nucleic acid construct of Item 59 and/or the combination of nucleic acid constructs of any one of the Items 60-62.
64. A combination of a first and second vector, comprising the combination of nucleic acids of Item 58 or the combination of nucleic acid constructs of any one of the Item 60-62, wherein the first vector comprises the first nucleic acid and/or the first nucleic acid construct, and the second vector comprises the second nucleic acid and/or the second nucleic acid construct.
65. The vector of Item 63 or the combination of the first and second vector of Item 64, wherein (a) the vector is selected from lentiviruses, γ-retroviruses and adeno-associated viruses, and/or (b) in the combination, the vectors are independently selected from lentiviruses, γ-retroviruses and adeno-associated viruses.
66. A recombinant cell, comprising the nucleic acid, the combination of nucleic acids, the nucleic acid construct, the combination of nucleic constructs, the vector, and/or the combination of vectors according to any one of the Item 57-65.
67. The recombinant cell of Item 66, which is an immune effector cell.
68. The recombinant cell of any one of the Items 66-67, expressing the CoCAR of any one of the Items 1-44 and/or the combination of any one of the Items 45-56 on the cell surface.
69. The recombinant cell of Item 68, wherein the first antigen-recognition domain is capable of binding a first antigen on the surface of a target cell, wherein binding of the CoCAR to the first antigen does not induce activation of the recombinant cell.
70. The recombinant cell of any one of the Items 66-69, further expressing a CAR comprising a second antigen-recognition domain, wherein the second antigen-recognition domain is capable of binding a second antigen on the surface of a target cell, wherein binding of the CAR to the second antigen induces activation of the recombinant cell.
71. The recombinant cell of Item 70, wherein binding of the CoCAR to the first antigen enhances the activation of the recombinant cell induced by binding of the CAR to the second antigen.
72. The recombinant cell of Item 70 or 71, wherein the CoCAR attenuates antigen-independent activation and differentiation of the recombinant cell induced by the CAR.
73. The recombinant cell of any one of the Items 66-72 wherein (i) the CAR and (ii) the CoCAR recognize different epitopes located in the same antigen expressed on the surface of a target cell.
74. The recombinant cell of any one of the Items 66-73, wherein (i) the CAR and (ii) the CoCAR recognize different antigens expressed on the surface of a target cell.
75. A pharmaceutical composition comprising
   (i) the CoCAR according to any one of Items 1-44,
   (ii) the combination of any of the Items 45-56,
   (iii) the nucleic acid, the combination of nucleic acids, the nucleic acid construct, the combination of nucleic constructs, the vector, and/or the combination of vectors of any one of the Items 57-65, and/or
   (iv) the recombinant cell according to any one of the Items 66-74,
   and a pharmaceutically acceptable carrier.
76. The CoCAR according to any one of Items 1-44, the combination of any one of the Items 45-56, the nucleic acid, the combination of nucleic acids, the nucleic acid construct, the combination of nucleic constructs, the vector, the combination of vectors of any one of the Items 57-65, the recombinant cell according to any one of the Items 66-74, and/or the pharmaceutical composition according to Item 75, for use in medicine.
77. The CoCAR according to any one of Items 1-44, the combination of any one of the Items 45-56, the nucleic acid, the combination of nucleic acids, the nucleic acid construct, the combination of nucleic constructs, the vector, the combination of vectors of any one of the Items 57-65, the recombinant cell according to any one of the Items 66-74, and/or the pharmaceutical composition according to Item 75, for use in treating cancer.
78. The CoCAR, the combination, the nucleic acid construct, the recombinant cell, or the pharmaceutical composition for use according to Item 76 or 77, in treating a malignancy with cell-surface expression of at least one tumor-associated antigen preferably selected from the group consisting of B7-H3 (CD276), BCMA, CD3, CD5, CD19, CD20, CD22, CD23, CD30, CD33, CD38, CD44, CD44v6, CD52, CD70, CD79A, CD79B, CD123, CD138, CD171, CEA, Claudin-6, Claudin-18.2, CLL1, CXCR5, EGFR, EGFRvIII, EPH-receptor A2, IGLV3-21, GP-2, GP-40, HER2, ErbB3, ErbB4, FBP, AchR, Fr-a, GD2, GD3, HMW-MAA, IL13Ra2, Kappa-LC, IGLV3-21-R110, Lewis Y, Mesothelin, MUC1, MUC16, NKG2D Ligands, NCAM, NY-ESO1, Oncofetal antigen h5T4, PSCA, PSMA, ROR1, TAG-72, VEGFR, GOLPH2, and SLAMF7.
79. The CoCAR, the combination, the nucleic acid construct, the recombinant cell, or the pharmaceutical composition for use according to any one of the Items 76-78, wherein the at least one tumor-associated antigen is selected from the group consisting of BCMA, CD22, CD79B, GOLPH2, EGFR/EGFRvIII, HER2, CEA, and GOLPH2
80. The CoCAR, the combination, the nucleic acid construct, the recombinant cell, and/or the pharmaceutical composition for use according to any one of the Items 76-79, for use in treating a B cell malignancy.
81. A method of treating cancer in a subject in need thereof, comprising the step of administering to the subject an effective amount of
   (i) the CoCAR according to any one of Items 1-44,
   (ii) the combination of any of the Items 45-56,
   (iii) the nucleic acid, the combination of nucleic acids, the nucleic acid construct, the combination of nucleic constructs, the vector, and/or the combination of vectors of any one of the Items 57-65, and/or
   (iv) the recombinant cell according to any one of the Items 64-74.
82. The method according to Item 81, wherein the cancer is a malignancy positive for at least one tumor-associated antigen selected from the group consisting of B7-H3 (CD276), BCMA, CD3, CD5, CD19, CD20, CD22, CD23, CD30, CD33, CD38, CD44, CD44v6, CD52, CD70, CD79A, CD79B, CD123, CD138, CD171, CEA, Claudin-6, Claudin-18.2, CLL1, CXCR5, EGFR, EGFRvIII, EPH-receptor A2, IGLV3-21, GP-2, GP-40, HER2, ErbB3, ErbB4, FBP, AchR, Fr-a, GD2, GD3, HMW-MAA, IL13Ra2, Kappa-LC, IGLV3-21-R110, Lewis Y, Mesothelin, MUC1, MUC16, NKG2D Ligands, NCAM, NY-ESO1, Oncofetal antigen h5T4, PSCA, PSMA, ROR1, TAG-72, VEGFR, GOLPH2, and SLAMF7.
83. The method according to Item 81 or 82, wherein the at least one tumor-associated antigen is selected from the group consisting of BCMA, CD22, CD79B, GOLPH2, EGFR/EGFRvIII, HER2, CEA, and GOLPH2.
84. The method according to any one of the Item 81-83, wherein the cancer is a B cell malignancy.
85. The CoCAR according to any one of the Items 1-44, comprising
   (i) a first extracellular antigen-recognition domain,
   (ii) a first hinge domain,
   (iii) a first transmembrane domain, and
   (iv) at least one cytosolic domain capable of binding LCK,
   wherein the chimeric coreceptor does not comprise a TCR-recruitment domain.
86. The recombinant cell of Item 71, wherein the second antigen has an antigen density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell.
87. The CoCAR, the combination, the nucleic acid construct, the recombinant cell, or the pharmaceutical composition for use according to any one of the Items 76-79, wherein the at least one tumor-associated antigen is selected from the group consisting of CD22, CD19, HER2, and EGFR, and/or wherein the at least one tumor-associated antigen has a density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell.
88. The method according to any one of the Items 81 to 84, wherein the at least one tumor-associated antigen is selected from the group consisting of CD22, CD19, HER2, and EGFR, and/or wherein the at least one tumor-associated antigen has a density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell.
89. The CoCAR according to Item 3, wherein the at least one tumor-associated antigen is selected from the group consisting of CD19, CD22, HER2, and EGFR, and/or wherein the at least one tumor-associated antigen has a density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell.
90. The CoCAR according to any one of the Items 1-4 or 89, wherein the first antigen-recognition domain comprises
   (a) a light chain variable domain (VL domain) comprising a light chain complementarity determining region LCDR1 of SEQ ID NO: 117, a LCDR2 of SEQ ID NO: 118 and a LCDR3 of SEQ ID NO: 119; and
   (b) a heavy chain variable domain (VH domain) comprising a heavy chain complementarity determining region HCDR1 of SEQ ID NO: 121, a HCDR2 of SEQ ID NO: 122 and a HCDR3 of SEQ ID NO: 123, wherein the light chain variable domain and the heavy chain variable domain specifically bind CD22.
91. The CoCAR according to any one of the Items 1-4 or 90,
   (a) one or more of the LFR1, LFR2, LFR3 and LFR4 have at least 90% amino acid sequence identity to the amino acid sequence of the respective framework regions of the light chain variable region according to SEQ ID NO: 120 and/or
   (b) one or more of the HFR1, HFR2, HFR3 and HFR4 have at least 90% amino acid sequence identity to the amino acid sequence of the respective framework regions of the heavy chain variable region according to SEQ ID NO: 124.
92. The CoCAR according to any one of the Items 1-4 or 90-91, wherein
   LFR1 comprises SEQ ID NO: 126,
   LFR2 comprises SEQ ID NO: 127,
   LFR3 comprises SEQ ID NO: 128,
   LFR4 comprises SEQ ID NO: 129,
   HFR1 comprises SEQ ID NO: 130,
   HFR2 comprises SEQ ID NO: 131,
   HFR3 comprises SEQ ID NO: 132, and/or
   HFR4 comprises SEQ ID NO: 133.
93. The CoCAR according to any one of the Items 90-92, wherein in the first antigen-recognition domain, the VH domain and the VL domain are connected by a linker, wherein the linker preferably comprises glycine and/or serine residues.
94. The CoCAR according to Item 93, the linker is selected from SEQ ID NO: 10, 46, 125, or 176, or from fragments and variants thereof.
95. The CoCAR according to any one of the Items 90-94, capable of specifically binding to a first antigen on a target cell, and wherein the first antigen-recognition domain is capable of binding to the first antigen on the surface of a target cell, wherein binding of the CoCAR to the first antigen does not induce activation of a recombinant cell expressing the CoCAR.
96. The CoCAR according to any one of the preceding Items, wherein the recombinant cell further expresses a CAR comprising a second antigen-recognition domain, wherein the second antigen-recognition domain is capable of binding to a second antigen on the surface of a target cell, wherein binding of the CAR to the second antigen induces activation of the recombinant cell.
97. The CoCAR according to any one of the preceding Items, wherein binding of the CoCAR to the first antigen enhances the activation of the recombinant cell induced by binding of the CAR to the second antigen, and/or wherein the CoCAR attenuates antigen-independent activation and differentiation of the recombinant cell induced by the CAR.
98. The CoCAR according to any one of the preceding Items, wherein the first hinge domain is derived from a CD8 or CD28 hinge domain, and wherein the first hinge domain preferably comprise a sequence selected from SEQ ID NOs: 73, 78, and variants and fragments thereof.
99. The CoCAR according to any one of the preceding Items, wherein the first transmembrane domain is derived from a CD8, CD28, CD44 or 146 transmembrane domain, and wherein the first transmembrane domain preferably comprises a sequence selected from SEQ ID NOs: 74, 79, 86, 136, 137 and variants and fragments thereof, more preferably SEQ ID NO: 79, 136, 137 and variants and fragments thereof.
100. The CoCAR according to any one of the preceding Items, wherein the at least one cytosolic domain capable of binding LCK
   (1) is an intracellular signaling domain derived from a CD28, CD44, or CD146 intracellular signaling domain, or a variant or fragment thereof, or
   (2) comprises one or more motifs capable of binding LCK.
101. The CoCAR according to Item 100, wherein the intracellular domain (1) is derived from a CD28 intracellular signaling domain, or a variant or fragment thereof.
102. The CoCAR according to Item 100, wherein the intracellular domain (1) is derived from a CD44 intracellular signaling domain, or a variant or fragment thereof.
103. The CoCAR according to Item 100, wherein the intracellular domain (1) is derived from a CD146 intracellular signaling domain, or a variant or fragment thereof.
104. The CoCAR according to any one of the preceding Items, wherein the at least one cytosolic domain does not comprise a CD3zeta domain.
105. A coreceptor-CAR (CoCAR), comprising
   (i) a first extracellular antigen-recognition domain, and
   A) (ii) the first hinge domain is derived from a CD8α hinge domain,
      (iii) the first transmembrane domain is derived from a CD8α transmembrane domain, and
      (iv) the at least one cytosolic domain derives from a CD28 intracellular signaling domain; or
   B) (ii) the first hinge domain is derived from a CD8α hinge domain,
      (iii) the first transmembrane domain is derived from a CD8α transmembrane domain, and
      (iv) the at least one cytosolic domain derives from a CD44 intracellular signaling domain; or
   C) (ii) the first hinge domain is derived from a CD8α hinge domain,
      (iii) the first transmembrane domain is derived from a CD44 transmembrane domain, and
      (iv) the at least one cytosolic domain derives from a CD44 intracellular signaling domain; or
   D) (ii) the first hinge domain is derived from a CD8α hinge domain,
      (iii) the first transmembrane domain is derived from a CD8α transmembrane domain, and
      (iv) the at least one cytosolic domain derives from a CD146 intracellular signaling domain; or
   E) (ii) the first hinge domain is derived from a CD8α hinge domain,
      (iii) the first transmembrane domain is derived from a CD146 transmembrane domain, and
      (iv) the at least one cytosolic domain derives from a CD146 intracellular signaling domain; or
   F) (ii) the first hinge domain is derived from a CD28 hinge domain,
      (iii) the first transmembrane domain is derived from a CD28 transmembrane domain, and
      (iv) the at least one cytosolic domain derives from a CD28 intracellular signaling domain; or
   and wherein the CoCAR does not comprise a CD3zeta domain.
106. The CoCAR according to anyone of the Items 1-44 or 90-105, wherein the CoCAR has an amino acid sequence comprising any of SEQ ID NO: 106-108, 138-144, 188-190, or a variant thereof.
107. A combination comprising
   (a) the CoCAR of any one of the Items 85, or 90-106, and
   (b) a chimeric antigen receptor (CAR),
   wherein the combination is expressed on a cell surface.
108. The combination of Item 107, wherein the chimeric antigen receptor (b) comprises
   (i) a second extracellular antigen-recognition domain,
   (ii) a second hinge region,
   (iii) a second transmembrane domain,
   (iv) a 4-1BB domain and/or a CD28 domain, and
   (v) a CD3zeta domain.
109. A nucleic acid encoding the CoCAR according to any one of the preceding Items, or the combination of any one of the Items 107 or 108, wherein the nucleic acid preferably comprises a nucleic acid sequences selected from SEQ ID NOs: 153-166, 201-207.
110. A combination of a first and second nucleic acid, encoding the combination of any one of the Items 107 or 108, wherein the first nucleic acid encodes the CoCAR (a), and the second nucleic acid encodes the CAR (b).
111. A nucleic acid construct, comprising the nucleic acid of Item 109 and/or the combination of a first and second nucleic acid of Item 110.
112. A combination of a first and second nucleic acid construct, comprising the combination of Item 110, wherein the first nucleic acid construct comprises the first nucleic acid, and the second nucleic acid construct comprises the second nucleic acid.
113. The nucleic acid construct of Item 111 or the combination of nucleic acid constructs of Item 112, wherein the nucleic acid and/or the first and second nucleic acids independently are functionally linked to at least one expression control sequence.
114. The nucleic acid construct or the combination of nucleic acid constructs of Item 113, wherein the at least one expression control sequence is selected from promoters, ribosome binding sites, enhancers and control elements which regulate transcription of the nucleic acid(s) or translation of mRNA(s) and enhancer sequences or upstream activator sequences.
115. A vector, comprising the nucleic acid of Item 109, the combination of the first and second nucleic acid of Item 110, the nucleic acid construct and/or the combination of nucleic acid constructs of any one of the Items 111-114.
116. A combination of a first and second vector, comprising the combination of nucleic acids of Item 110 or the combination of nucleic acid constructs of any one of the Item 112-114, wherein the first vector comprises the first nucleic acid and/or the first nucleic acid construct, and the second vector comprises the second nucleic acid and/or the second nucleic acid construct.
117. The vector of Item 115 or the combination of the first and second vector of Item 116, wherein
   (a) the vector is selected from lentiviruses, γ-retroviruses and adeno-associated viruses, and/or
   (b) in the combination, the vectors are independently selected from lentiviruses, γ-retroviruses and adeno-associated viruses.
118. A recombinant cell comprising the nucleic acid, the combination of nucleic acids, the nucleic acid construct, the combination of nucleic constructs, the vector, and/or the combination of vectors according to any one of the Item 109-117, wherein the recombinant cell is preferably an immune effector cell.
119. The recombinant cell of Item 118, expressing the CoCAR of and/or the combination on the cell surface.
120. The recombinant cell of Item 119, wherein the first antigen-recognition domain is capable of binding a first antigen on the surface of a target cell, wherein binding of the CoCAR to the first antigen does not induce activation of the recombinant cell.
121. The recombinant cell of any one of the Items 118-120, further expressing a CAR comprising a second antigen-recognition domain, wherein the second antigen-recognition domain is capable of binding a second antigen on the surface of a target cell, wherein binding of the CAR to the second antigen induces activation of the recombinant cell.
122. The recombinant cell of Item 121, wherein binding of the CoCAR to the first antigen enhances the activation of the recombinant cell induced by binding of the CAR to the second antigen, and/or wherein the CoCAR attenuates antigen-independent activation and differentiation of the recombinant cell induced by the CAR.
123. The recombinant cell of any one of the Items 118-122, wherein (i) the CAR and (ii) the CoCAR recognize different epitopes located in the same antigen expressed on the surface of a target cell, or wherein (i) the CAR and (ii) the CoCAR recognize different antigens expressed on the surface of a target cell.
124. The recombinant cell of any one of the Items 118-123, wherein wherein the second antigen has an antigen density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell.
125. A pharmaceutical composition comprising
   (i) the CoCAR according to any one of Items 85, or 90-106,
   (ii) the combination of any of the Items 107-108,
   (iii) the nucleic acid, the combination of nucleic acids, the nucleic acid construct, the combination of nucleic constructs, the vector, and/or the combination of vectors of any one of the Items 109-117, and/or
   (iv) the recombinant cell according to any one of the Items 118-124,
   and a pharmaceutically acceptable carrier.
126. The CoCAR according to any one of Items 85, or 90-106, the combination of any one of the Items 107-108, the nucleic acid, the combination of nucleic acids, the nucleic acid construct, the combination of nucleic constructs, the vector, the combination of vectors of any one of the Items 109-117, the recombinant cell according to any one of the Items 118-124, and/or the pharmaceutical composition according to Item 125, for use in medicine.
127. The CoCAR according to any one of Items 85, or 90-106, the combination of any one of the Items 110-111, the nucleic acid, the combination of nucleic acids, the nucleic acid construct, the combination of nucleic constructs, the vector, the combination of vectors of any one of the Items 109-117, the recombinant cell according to any one of the Items 118-124, and/or the pharmaceutical composition according to Item 125, for use in treating cancer.
128. The CoCAR, the combination, the nucleic acid construct, the recombinant cell, or the pharmaceutical composition for use according to Item 126 or 127, in treating a malignancy with cell-surface expression of at least one tumor-associated antigen preferably selected from the group consisting of B7-H3 (CD276), BCMA, CD3, CD5, CD19, CD20, CD22, CD23, CD30, CD33, CD38, CD44, CD44v6, CD52, CD70, CD79A, CD79B, CD123, CD138, CD171, CEA, Claudin-6, Claudin-18.2, CLL1, CXCR5, EGFR, EGFRvIII, EPH-receptor A2, IGLV3-21, GP-2, GP-40, HER2, ErbB3, ErbB4, FBP, AchR, Fr-a, GD2, GD3, HMW-MAA, IL13Ra2, Kappa-LC, IGLV3-21-R110, Lewis Y, Mesothelin, MUC1, MUC16, NKG2D Ligands, NCAM, NY-ESO1, Oncofetal antigen h5T4, PSCA, PSMA, ROR1, TAG-72, VEGFR, GOLPH2, and SLAMF7, and preferably from the group consisting of CD19, CD22, HER2, and EGFR.
129. The CoCAR, the combination, the nucleic acid construct, the recombinant cell, or the pharmaceutical composition for use according to Item 126 or 127, for use in treating a B cell malignancy.
130. The CoCAR, the combination, the nucleic acid construct, the recombinant cell, or the pharmaceutical composition for use according to any one of the Items 126 to 129, wherein the at least one tumor-associated antigen has a density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell.
131. A method of treating cancer in a subject in need thereof, comprising the step of administering to the subject an effective amount of
   (i) the CoCAR according to any one of Items 85, or 90-106,
   (ii) the combination of any of the Items 107-108,
   (iii) the nucleic acid, the combination of nucleic acids, the nucleic acid construct, the combination of nucleic constructs, the vector, and/or the combination of vectors of any one of the Items 109-117, and/or
   (iv) the recombinant cell according to any one of the Items 118-124.
132. The method according to Item 131, wherein the cancer is a malignancy positive for at least one tumor-associated antigen selected from the group consisting of B7-H3 (CD276), BCMA, CD3, CD5, CD19, CD20, CD22, CD23, CD30, CD33, CD38, CD44, CD44v6, CD52, CD70, CD79A, CD79B, CD123, CD138, CD171, CEA, Claudin-6, Claudin-18.2, CLL1, CXCR5, EGFR, EGFRvIII, EPH-receptor A2, IGLV3-21, GP-2, GP-40, HER2, ErbB3, ErbB4, FBP, AchR, Fr-a, GD2, GD3, HMW-MAA, IL13Ra2, Kappa-LC, IGLV3-21-R110, Lewis Y, Mesothelin, MUC1, MUC16, NKG2D Ligands, NCAM, NY-ESO1, Oncofetal antigen h5T4, PSCA, PSMA, ROR1, TAG-72, VEGFR, GOLPH2, and SLAMF7, and preferably from the group consisting of CD19, CD22, HER2, and EGFR.
133. The method according to any one of the Item 131-132, wherein the cancer is a B cell malignancy.
134. The method according to any one of the Items 131-133, wherein the at least one tumor-associated antigen has a density lower than about 1000 molecules/cell, about 400 molecules/cell, about 300 molecules/cell, about 200 molecules/cell, about 100 molecules/cell, or about 40 molecules/cell, and preferably lower than about 400 molecules/cell, and more preferably lower than about 40 molecules/cell.

TABLE 2
Sequence List for the sequences of SEQ ID NOs 116-207

| SEQ ID | Description | | Type |
|---|---|---|---|
| NO 116 | anti-CD22 antigen- | CSFR2a leader sequence | Amino Acid |
| NO 117 | recognition domain | VL CDR1 | Amino Acid |
| NO 118 | (human m971 scFv) | VL CDR2 | Amino Acid |
| NO 119 | | VL CDR3 | Amino Acid |
| NO 120 | | VL | Amino Acid |
| NO 121 | | VH CDR1 | Amino Acid |
| NO 122 | | VH CDR2 | Amino Acid |
| NO 123 | | VH CDR3 | Amino Acid |
| NO 124 | | VH | Amino Acid |
| NO 125 | | linker L1 | Amino Acid |
| NO 126 | | LFR1 | Amino Acid |
| NO 127 | | LFR2 | Amino Acid |
| NO 128 | | LFR3 | Amino Acid |
| NO 129 | | LFR4 | Amino Acid |
| NO 130 | | HFR1 | Amino Acid |
| NO 131 | | HFR2 | Amino Acid |
| NO 132 | | HFR3 | Amino Acid |
| NO 133 | | HFR4 | Amino Acid |
| NO 134 | CAR construct | anti-CD22 CAR with m971 scFv and CD28 | Amino Acid |
| | | backbone [22-BBz] | |
| NO 135 | CoCAR construct | CD2 intracellular domain | Amino Acid |
| NO 136 | | CD44 transmembrane domain | Amino Acid |
| NO 137 | | CD146 transmembrane domain | Amino Acid |
| NO 138 | | 19-2 CoCAR | Amino Acid |
| NO 139 | | 19-3e CoCAR | Amino Acid |
| NO 140 | | 19-28 CoCAR | Amino Acid |
| NO 141 | | 19-44 CoCAR | Amino Acid |
| NO 142 | | 19-146 CoCAR | Amino Acid |
| NO 143 | | 19-44v2 CoCAR | Amino Acid |
| NO 144 | | 19-146v2 CoCAR | Amino Acid |
| NO 145 | Combination | 22-BBz/19-2 | Amino Acid |
| NO 146 | CAR/CoCAR | 22-BBz/19-3e | Amino Acid |
| NO 147 | | 22-BBz/19-28 | Amino Acid |
| NO 148 | | 22-BBz/19-44 | Amino Acid |
| NO 149 | | 22-BBz/19-146 | Amino Acid |
| NO 150 | | 22-BBz/19-44v2 | Amino Acid |
| NO 151 | | 22-BBz/19-146v2 | Amino Acid |
| NO 152 | CAR constructs | anti-CD22 CAR with m971 scFv and CD28 | Nucleic acid |
| | | backbone [22-BBz] | |
| NO 153 | CoCAR construct | 19-2 CoCAR | Nucleic acid |
| NO 154 | | 19-3e CoCAR | Nucleic acid |
| NO 155 | | 19-28 CoCAR | Nucleic acid |
| NO 156 | | 19-44 CoCAR | Nucleic acid |
| NO 157 | | 19-146 CoCAR | Nucleic acid |
| NO 158 | | 19-44v2 CoCAR | Nucleic acid |
| NO 159 | | 19-146v2 CoCAR | Nucleic acid |
| NO 160 | Combination | 22-BBz/19-2 | Nucleic acid |
| NO 161 | CAR/CoCAR | 22-BBz/19-3e | Nucleic acid |
| NO 162 | | 22-BBz/19-28 | Nucleic acid |
| NO 163 | | 22-BBz/19-44 | Nucleic acid |
| NO 164 | | 22-BBz/19-146 | Nucleic acid |
| NO 165 | | 22-BBz/19-44v2 | Nucleic acid |
| NO 166 | | 22-BBz/19-146v2 | Nucleic acid |
| NO 167 | Sequence anti-HSV | IGHV3-23 leader sequence | Amino Acid |
| NO 168 | antigen-recognition | VL CDR1 | Amino Acid |
| NO 169 | domain | VL CDR2 | Amino Acid |
| NO 170 | | VL CDR3 | Amino Acid |
| NO 171 | | VL | Amino Acid |
| NO 172 | | VH CDR1 | Amino Acid |
| NO 173 | | VH CDR2 | Amino Acid |
| NO 174 | | VH CDR3 | Amino Acid |
| NO 175 | | VH | Amino Acid |
| NO 176 | | linker | Amino Acid |
| NO 177 | | LFR1 | Amino Acid |
| NO 178 | | LFR2 | Amino Acid |
| NO 179 | | LFR3 | Amino Acid |
| NO 180 | | LFR4 | Amino Acid |
| NO 181 | | HFR1 | Amino Acid |
| NO 182 | | HFR2 | Amino Acid |
| NO 183 | | HFR3 | Amino Acid |
| NO 184 | | HFR4 | Amino Acid |
| NO 185 | CAR construct | anti-CD22 CAR with m971 scFv and IgG1-Fc | Amino Acid |
| | | spacer [m971-Fc-BBz] | |
| NO 186 | | anti-HSV gB CAR with CD28 | Amino Acid |
| | | hinge/transmembrane domains [aHSV-BBz] | |
| NO 187 | | anti-HER CAR with CD28 | Amino Acid |
| | | hinge/transmembrane domains [aHER2-BBz]): | |
| NO 188 | CoCAR construct | SGIII-28 CoCAR | Amino Acid |
| NO 189 | | SGIII-44v2 CoCAR | Amino Acid |
| NO 190 | | SGIII-146v2 CoCAR | Amino Acid |
| NO 191 | Combination | 22-BBz(xxx)/19-28 | Amino Acid |
| NO 192 | CAR/CoCAR | aHSV-BBz(xxx)/19-28 | Amino Acid |
| NO 193 | | HSV-BBz/19-44v2 | Amino Acid |
| NO 194 | | HSV-BBz/19-146v2 | Amino Acid |
| NO 195 | | m971-Fc-BBz/SGIII-28 | Amino Acid |
| NO 196 | | m971-Fc-BBz/SGIII-44v2 | Amino Acid |
| NO 197 | | m971-Fc-BBz/SGIII-146v2 | Amino Acid |
| NO 198 | CAR construct | anti-HSV gB CAR [aHSV-BBz] | Nucleic acid |
| NO 199 | | anti-HER2 CAR [aHER2-BBz] | Nucleic acid |
| NO 200 | | anti-CD22 CAR with m971 scFv and IgG1-Fc | Nucleic acid |
| | | spacer [m971-Fc-BBz] | |
| NO 201 | Combination | 22-BBz(xxx)/19-28 | Nucleic acid |
| NO 202 | CAR/CoCAR | aHSV-BBz(xxx)/19-28 | Nucleic acid |
| NO 203 | | aHSV-BBz/19-44v2 | Nucleic acid |
| NO 204 | | aHSV-BBz/19-146v2 | Nucleic acid |
| NO 205 | | m971-Fc-BBz/SGIII-28 | Nucleic acid |
| NO 206 | | m971-Fc-BBz/SGIII-44v2 | Nucleic acid |
| NO 207 | | m971-Fc-BBz/SGIII-146v2 | Nucleic acid |

The concept of the present invention is described in more detail by the examples described below, which are used only for illustration purposes and are not meant to limit the scope of this disclosure or the claims. Owing to the description and the examples, further embodiments which are likewise included in the invention are accessible to the skilled worker.

### EXAMPLES

The techniques and methods used herein are described herein or carried out in a manner known per se and as described, for example, in Sambrook et al., Molecular Cloning: A Laboratory Manual, 2^{nd} Edition (1989) Cold Spring Harbor Laboratory Press, Cold Spring Harbor, N.Y. All methods including the use of kits and reagents are carried out according to the manufacturers' information unless specifically indicated.

### Example 1: Overexpression of LCK Leads to Terminal Differentiation and Limited Expansion of BBz-CAR-T Cells (Comparative Example)

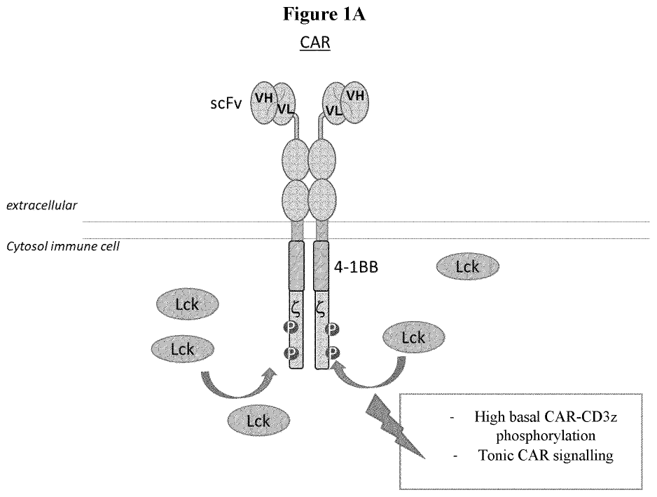

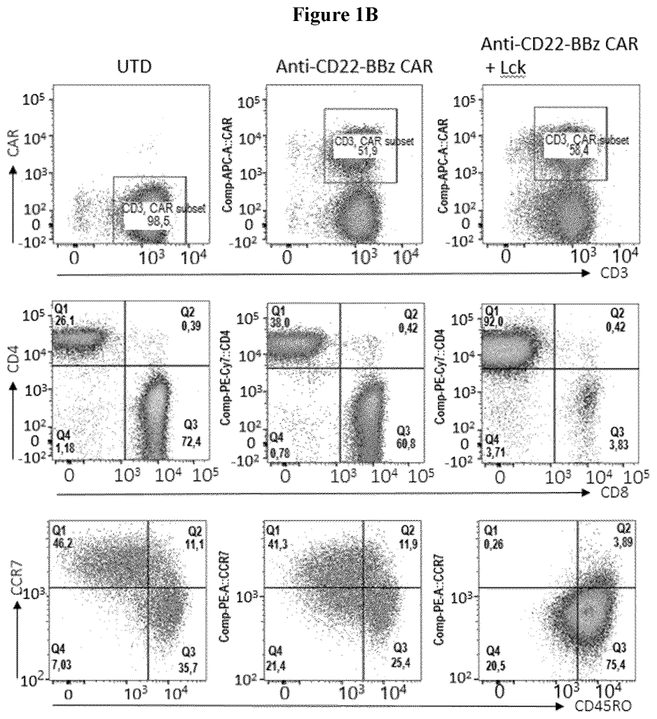

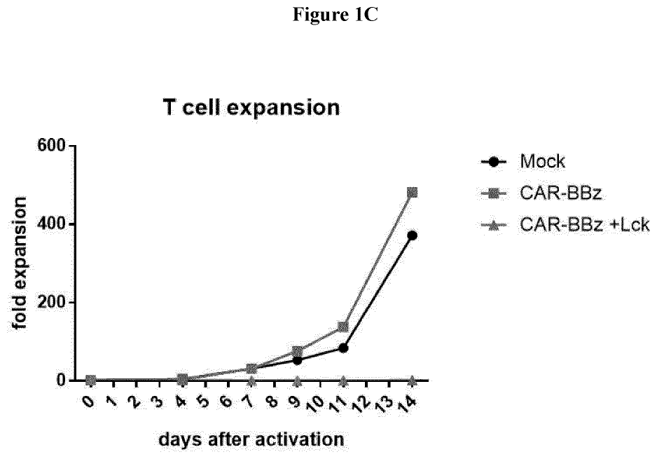

In the course of the development of the present invention, it was tested whether co-expression of LCK together with an anti-CD22 CAR in primary human T cells could improve the function of the resulting CAR-T cells by increasing basal CAR-CD3ζ phosphorylation (FIG. 1A). To this end, primary human T cells were transduced with lentiviral vectors encoding an anti-CD22 CAR (SEQ ID NO: 105) containing a humanized antigen-recognition domain (derived from the RFB4 antibody) linked to IgG1 Fc spacer, CD28 transmembrane, and 4-1BB-CD3zeta (BBz) signalling domains, with or without co-transduction with a vector encoding full-length human LCK. It was surprisingly found that co-expression of LCK together with the anti-CD22 CAR led to depletion of CD8 T cells and preferential differentiation of CD4-positive T cells toward an effector T cell-like phenotype after 14 days in culture. In contrast, T cells expressing the same anti-CD22 CAR contained a mixture of CD8-positive and CD4-positive T cells that showed a memory T cell-like phenotype (FIG. 1B). Moreover, BBz-CAR-T cells overexpressing LCK showed poorer expansion capacities ex vivo compared to their counterparts expressing the BBz-CAR alone, or to CAR-negative T cells (FIG. 1C). Therefore, in contrast to a previous study (Sun et al., (2020) Cancer Cell, (2) 216-225; WO2021087183), these surprising results suggest that the co-expression of a BBz-CAR with LCK may cause uncontrolled and too intense basal CAR-CD3ζ phosphorylation, which triggers enhanced antigen-independent tonic signalling manifesting in advanced T cell differentiation toward an effector phenotype and reduced expansion capacities eventually. These properties are not desired in the context of adoptive therapies because such T cells are prone to exhaust and generally show limited long-term persistence in vivo, and therefore will likely induce insufficient therapeutic responses in patients.

### Example 2: The Concept of Co-Expressing a BBz-CAR and a CoCAR to Regulate LCK Recruitment to CAR Synapse by Antigen Binding

Enhanced CAR-T cell function may be achieved by specifically regulating the recruitment of the kinase LCK to the CAR synapse only in response to CAR ligation by antigen, and not in unstimulated T cells. It is hypothesized that this mechanism may not only prevents tonic CAR signalling, but additionally should improve the efficacy of BBz-CAR-T cells toward antigen-low tumor cells because LCK may be more efficiently recruited to the CAR synapse. To achieve this aim, the inventors developed the CoCAR concept, which is based on the co-expression of two distinct chimeric receptor molecules in the same immune cell (e.g. a T cell): the first receptor molecule consists of a conventional CAR known in the art, which functions as a T cell activating receptor, whereas the second receptor molecule consists of a fusion between an antigen-recognition domain (e.g. a scFv) and coreceptor-derived domains (in this example hinge, transmembrane and cytosolic domains from CD4; here referred to as CoCAR), which functions as a coreceptor to enhance T cell responses activated by the first receptor (the CAR). The antigen-recognition domains of these two receptor molecules recognize different epitopes located either in the same antigen or in a different antigen expressed on the surface of a given target cell. Since the CoCAR contains hinge, transmembrane and intracellular domains capable of binding LCK (in this example, the CD4 intracellular domain contains a “zinc clasp” motif capable to bind LCK) of the coreceptor, it is assumed that the CoCAR associates stably with endogenous LCK molecules in order to perform its function. In unstimulated T cells, the CAR and the CoCAR are spatially separated, thus LCK coupled to the CoCAR is not available to mediate basal CAR-CD3ζ phosphorylation, tonic signalling, and antigen-independent T cell differentiation and exhaustion (FIG. 2A). After binding to cognate antigens, CAR and coreceptor-CAR relocalize to get in proximity, and CoCAR-associated LCK is now available to induce CAR-CD3ζ ITAM phosphorylation and recruitment of ZAP-70. The ITAM-bound ZAP-70 molecules then become efficiently phosphorylated by CoCAR-associated LCK, and activated ZAP-70 in turn phosphorylates its substrates including LAT and SLP-76, which may lead to more efficient assembly of the signalosome and activation of various downstream signalling pathways (FIG. 2B).

### Example 3: Co-Expressing a CAR Recognizing CD22 and a CoCAR Recognizing CD19 Enhances CAR-T Cell Activity Against B Cell Tumor Cells

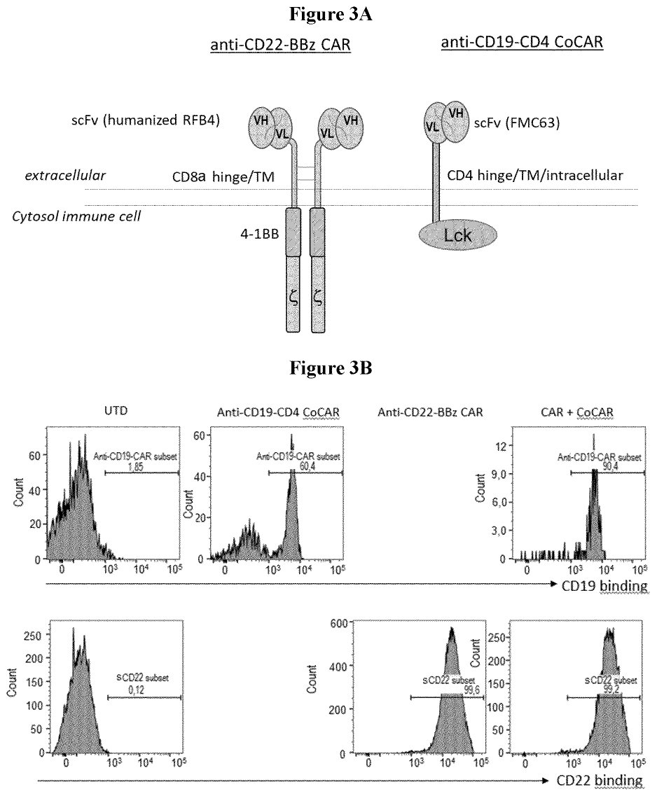

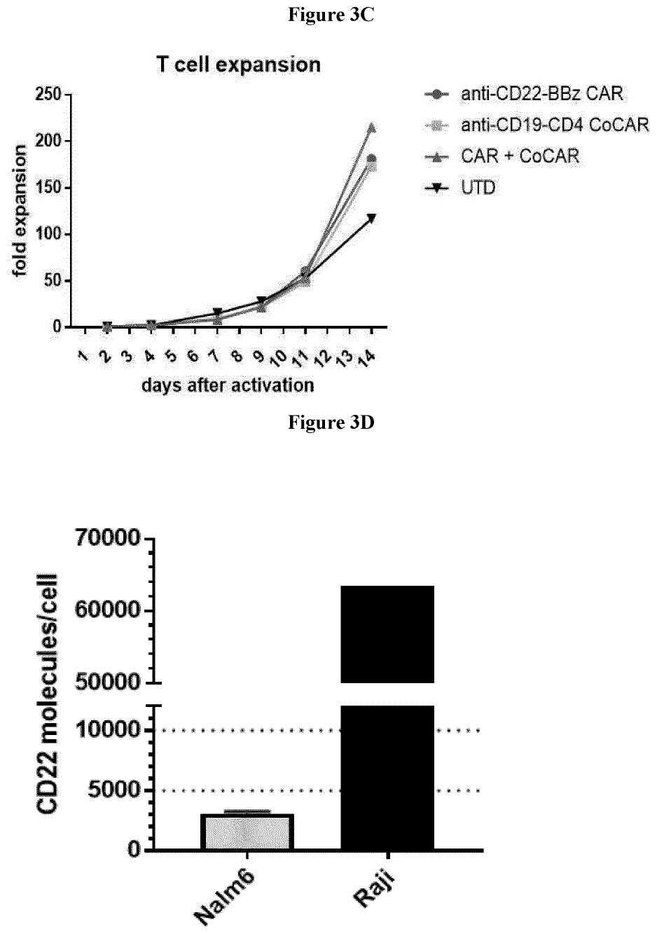

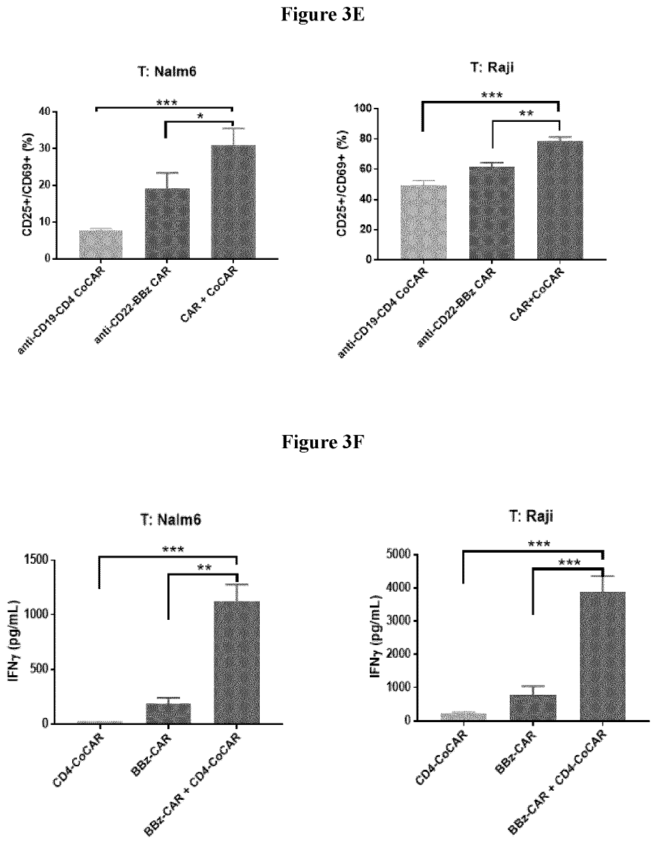

Exemplary CAR and CoCAR molecules recognizing different extracellular antigens exposed on a tumor cell were engineered and tested for their functions to activate primary human T cells expressing these CAR and CoCAR molecules. FIG. 3A shows an example of a T cell co-expressing an anti-CD22 CAR (SEQ ID NO: 104) containing a humanized antigen-recognition domain (derived from the RFB4 monoclonal antibody), a CD8α hinge- and transmembrane region, and 4-1BB/CD3ζ signalling domains, with an anti-CD19 CoCAR (SEQ ID NO: 106) containing the clinically established FMC63 antigen-recognition domain linked to CD4 hinge, transmembrane- and intracellular domains. Primary human T cells were transduced with lentiviral vectors to express the anti-CD22 CAR or the anti-CD19 CoCAR, or were co-transduced with both lentiviral vectors to co-express both molecules. Seven days after transduction, the transduced T cells were sort-purified and CAR and CoCAR expression was determined by flow cytometry. As shown in FIG. 3B, transduced T cells efficiently expressed the anti-CD22 CAR and anti-CD19 CoCAR molecules on the cell-surface, as determined by their capacities to bind their specific fluorescently labelled soluble antigens. Importantly, co-expression of the anti-CD22 CAR with the anti-CD19 CoCAR did not dampen ex vivo T cell expansion (FIG. 3C), as it was observed for anti-CD22 CAR-T cells co-expressing LCK (FIG. 1C). To test for CAR-induced T cell functions, the T cells were incubated with Nalm6 cells (derived from a patient with B cell acute lymphoblastic leukemia) or with Raji cells (derived from a patient with Burkitt lymphoma) representing tumor cells expressing low (~3.000 antigens per cells) and high (~60.000 antigens per cell) CD22 antigen densities (FIG. 3D). Both tumor cell lines also express the CD19 antigen at high densities (>50.000 molecules/cell; Haso et al, (2013) Blood, (7) 1165-1174). When T cells expressing the anti-CD22 CAR were incubated with Nalm6 or Raji tumor cells, the CAR molecules activated the corresponding T cells to upregulate expression of activation markers on their cell-surface, such as CD69, CD25, or both, and to secrete specific effector cytokines including Interferon-gamma (IFNγ) or IL-2. As shown in FIG. 3E, such T cell activation (represented by cell-surface co-expression of CD69 and CD25 molecules) by the anti-CD22 CAR was significantly enhanced when the T cells co-expressed the anti-CD19 CoCAR, whereas the expression of the anti-CD19 CoCAR alone had no effect on T cell activation. Likewise, incubation of T cells expressing the anti-CD22 CAR with Nalm6 or Raji tumor cells led to modest production of IFNγ, but co-expressing the anti-CD22 CAR with the anti-CD19 CoCAR dramatically enhanced effector cytokine production (FIG. 3F). These results indicate that T cells co-expressing a CAR recognizing antigen one (in this example CD22) with a CD4-based CoCAR recognizing antigen two (in this example CD19) on the surface of a target cell show enhanced activity against the target cells expressing high or low antigen densities, compared to T cells expressing the CAR alone.

### Example 4: Co-Expressing a CAR and a CoCAR Recognizing Different Epitopes in the CD22 Antigen Enhances CAR-T Cell Activity Against B Cell Tumor Cells

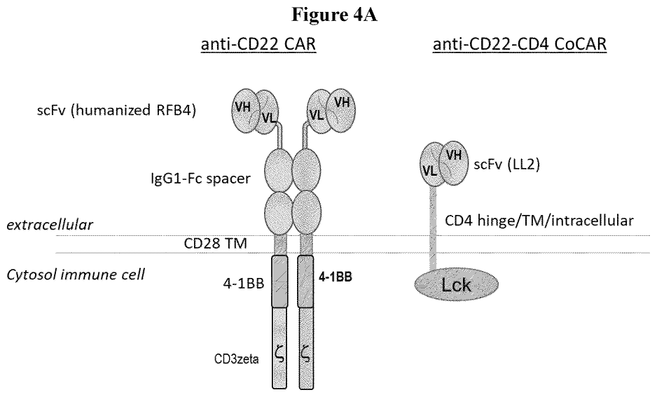

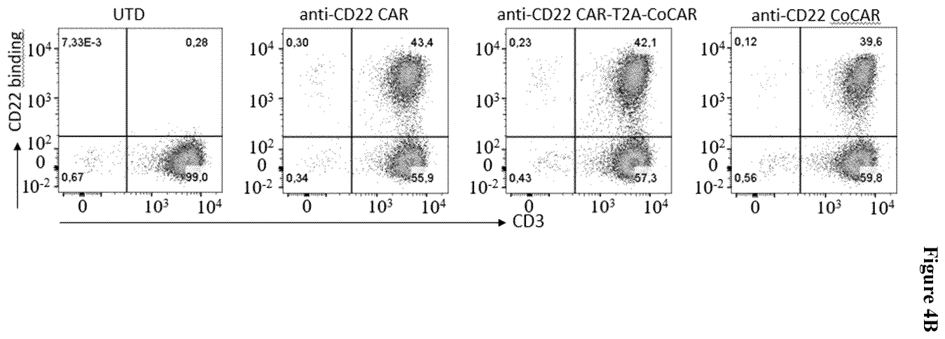

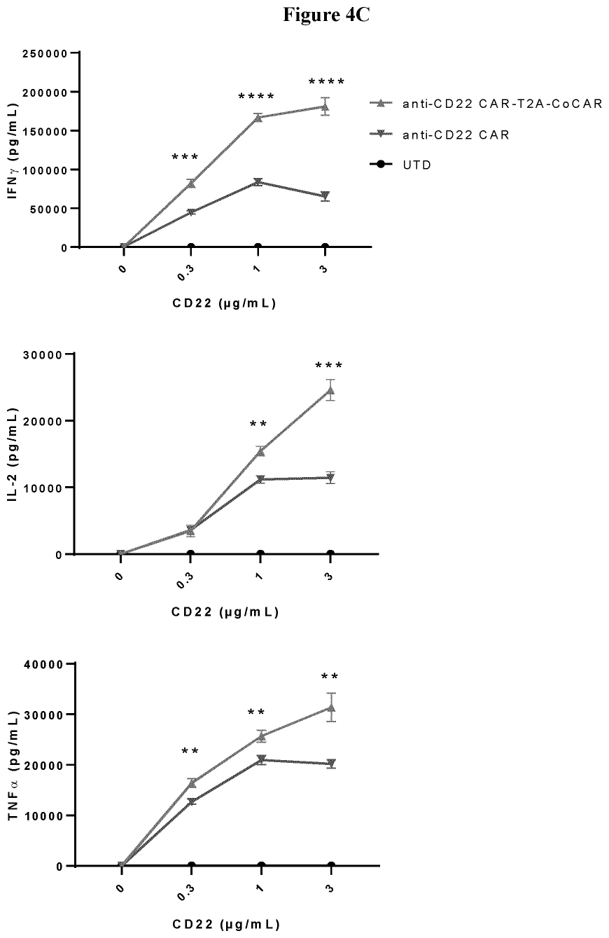

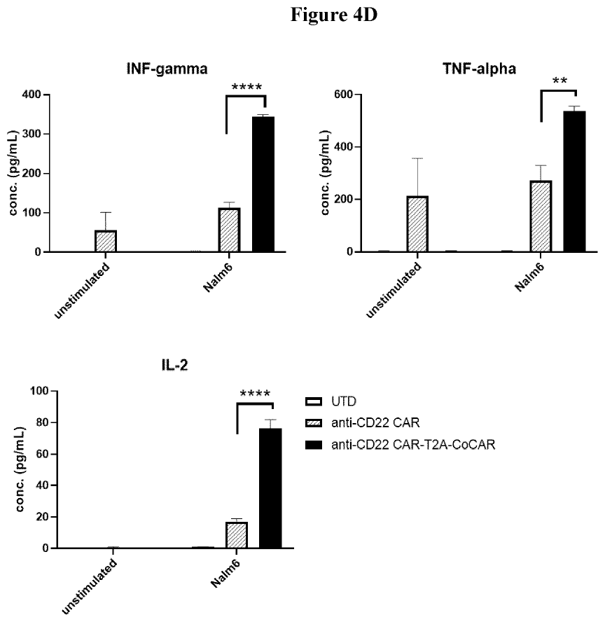

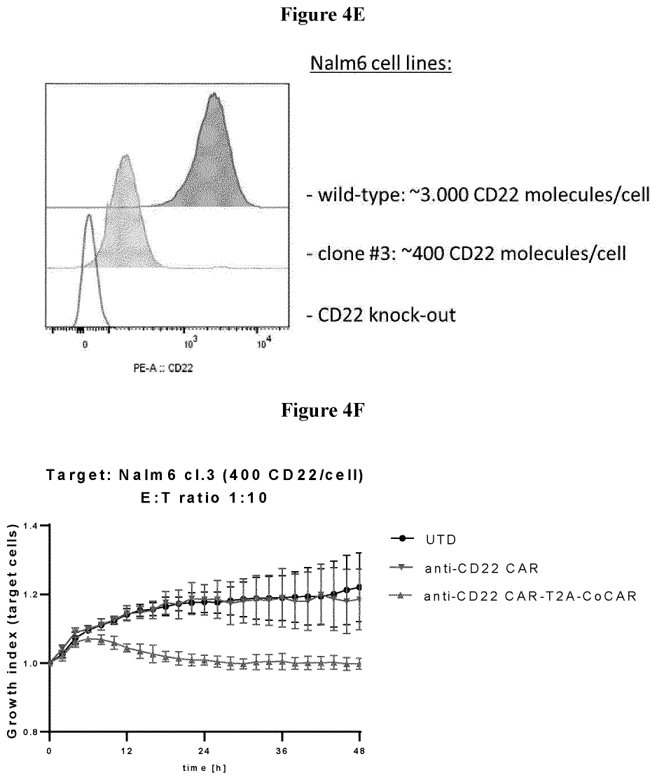

Cancer patients with CD19-positive B cell tumors experience good response rates after treatment with CD19-specific immunotherapeutics (e.g. anti-CD19 CAR-T cells), however many patients relapse due to the outgrowth of CD19-low or CD19-negative tumor cells. Such CD19-low or -negative tumor cells maintain CD22 expression, for which reason it was tested if T cells co-expressing a CAR and a CoCAR, both recognizing different epitopes in the CD22 antigen, can induce superior anti-tumor responses toward tumor cells with low CD22 antigen density compared to T cells expressing the anti-CD22 CAR alone. In this exemplary construct, the CAR contained a humanized anti-CD22 scFv (derived from RFB4 antibody) linked to an IgG1-Fc spacer, a CD28 transmembrane and 4-1BB/CD3ζ signalling domains, and the CoCAR contained a murine anti-CD22 scFv (derived from LL2 antibody) linked to CD4-derived hinge, transmembrane and cytosolic domains. The CAR (SEQ ID NO: 105) and the CoCAR (SEQ ID NO: 107) were encoded in a single bicistronic lentiviral vector, which induces equimolar translation of both molecules in transduced cells. Primary human T cells were separately transduced with a lentiviral vector encoding the CAR, the CoCAR, or the BBz-CAR-T2A-CoCAR, and resulting T cells were incubated with fluorescently labelled CD22 protein to detected cell-surface expression of the CAR and CoCAR molecules. Flow cytometry analysis showed binding of the CD22 antigen to transduced T cells, and not to untransduced T cells, indicating efficient cell-surface expression of both anti-CD22 CAR and CoCAR molecules (FIG. 4B). To compare the responsiveness between conventional anti-CD22 CAR-T cells with T cells co-expressing the anti-CD22 CAR and CoCAR, the T cells were incubated in cell culture plates coated with different concentrations of recombinant human CD22 antigen, and after 24 hours, the concentration of secreted effector cytokines was measured by flow cytometry using a LEGENDPlex human Th1 panel multiplex assay (BioLegend). The results show that co-expressing an anti-CD22 CoCAR significantly enhanced the activity of anti-CD22 CAR-T cells and, importantly, a lower amount of the coated antigen was sufficient to activate CAR-T cells when the CoCAR was co-expressed (FIG. 4C). Furthermore, stimulation with CD22-positive Nalm6 cells induced secretion of significantly higher amounts of effector cytokines by T cells expressing the anti-CD22 BBz-CAR-T2A-CoCAR construct (SEQ ID NO: 109) compared to conventional anti-CD22 CAR-T cells (FIG. 4D). Notably, conventional anti-CD22 CAR-T cells also secreted detectable amounts of IFN-gamma and TNF-alpha in an antigen-independent manner, most likely due to tonic signalling of the CAR, whereas co-expressing the CoCAR completely abolished antigen-independent cytokine secretion (FIG. 4D).

Nalm6 cells express only ~3.000 CD22 molecules per cell (FIG. 3D), which represents the median amount of the CD22 antigen found on primary B-ALL tumor cells (Shah et al., (2015) Pediatr Blood Cancer, (6) 964-969). Clinical studies showed that reduction of CD22 site densities to <1.800 molecules per cell is major cause of relapse after treatment with anti-CD22 CAR-T cells (Fry et al., (2017) Nat Med, (1) 20-28). To test if co-expressing an anti-CD22 CoCAR enhanced the sensitivity of anti-CD22 CAR-T cells to tumor cells with ultra-low CD22 antigen expression, Nalm6 cells were engineered to express reduced CD22 antigen levels. To generate such cell line, the CD22 gene was first disrupted using the CRISPR/Cas9 technology, followed by transduction of resulting CD22-deficient Nalm6 cells with lentiviral vectors encoding human CD22. Subsequent single-cell cloning and analyses of CD22 expression densities using Quantibrite Phycoerythrin (PE) Quantification Beads (Becton Dickinson) identified several Nalm6 cell clones that displayed reduced CD22 antigen densities, including clone #3 that expressed only ~400 CD22 molecules per cell, which is far below the sensitivity limit of 4-1BB-based CARs known in the art (FIG. 4E). The above-described CAR-T2A-CoCAR co-expression system was then tested under this ultra-low antigen density experimental setting. Specifically, T cells expressing the anti-CD22 CAR, with or without co-expression of the anti-CD22 CoCAR, were incubated with Nalm6 cells clone 3, and the growth of GFP-positive tumor cells was monitored in an Incucyte system over a period of 48 hours. As shown in FIG. 4F, co-expression of the anti-CD22 CoCAR significantly improved the sensitivity of the anti-CD22 CAR, as T cells co-expressing both molecules were able to inhibit the outgrowth of tumor cells expressing ultra-low antigen densities, which, in contrast, could not be inhibited by conventional anti-CD22 CAR-T cells. In summary, these examples demonstrate that the co-expression of a CoCAR dramatically enhances the activity and sensitivity of T cells expressing an anti-CD22 CAR against tumor cells expressing high, low or ultra-low antigen levels.

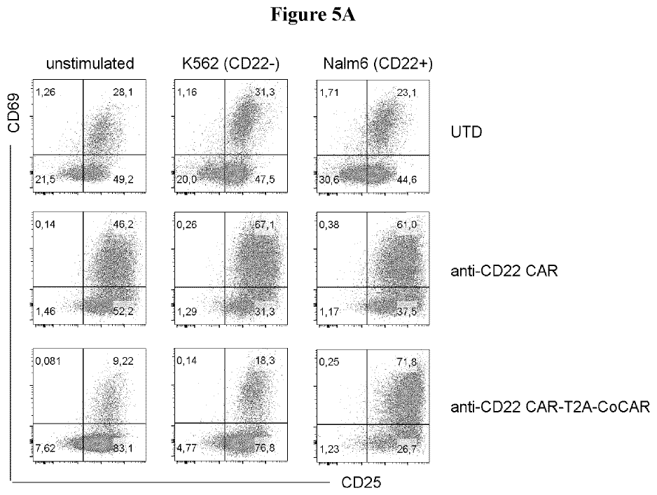

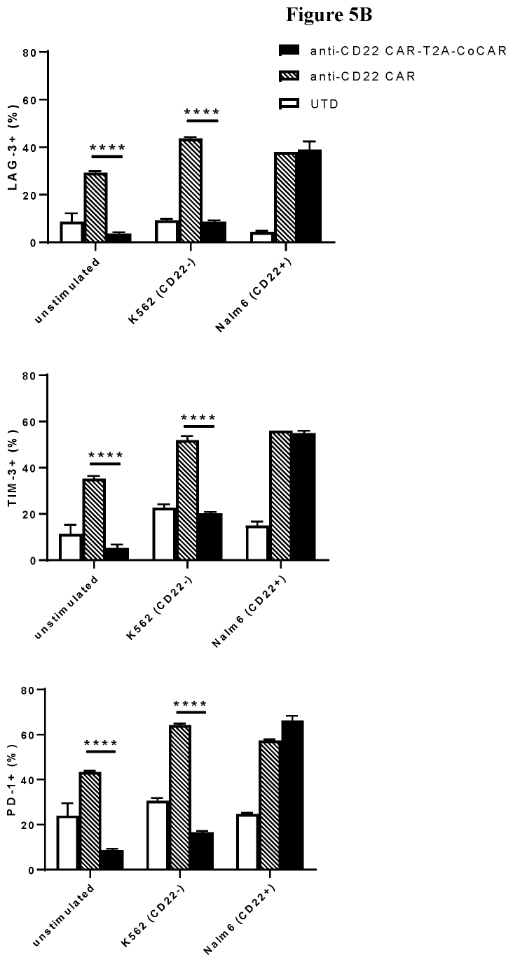

Example 5: Co-expressing a CoCAR reduces the antigen-independent activation of T cells induced by a conventional CAR. CARs may induce antigen-independent (tonic) signaling because of the aggregation tendency of some scFvs, or because of high CAR expression levels, and this may drive T cell differentiation and exhaustion, and thereby limits the anti-tumor activity of the therapeutic cells (Long et al., (2015) Nat Med, (6) 581-590). The anti-CD22 CAR used in the present invention may also induces tonic signaling, as indicated by increased concentrations of effector cytokines in the supernatant of unstimulated CAR-T cells (FIG. 4D). Correspondingly, phenotypic analyses by flow cytometry showed that unstimulated anti-CD22 CAR-T cells displayed an activated phenotype, as indicated by increased expression of CD69 and CD25 activation markers, and co-culture of anti-CD22 CAR-T cells with the CD22-negative myeloid cell line K562 for 24 hours further increased antigen-independent CAR-T cell activation (FIG. 5A). Strikingly, co-expressing the anti-CD22 CoCAR dramatically reduced the antigen-independent activation of T cells induced by the anti-CD22 CAR, and T cells co-expressing the CAR and CoCAR showed an even less activated phenotype than untransduced (CAR-negative) T cells. In contrast, when stimulated with CD22-positive Nalm6 cells, co-expressing the CoCAR markedly enhanced the activation of anti-CD22 CAR-T cells, as indicated by increased expression of CD69 and CD25 (FIG. 5A), which is in line with the effector cytokine secretion pattern depicted in FIG. 4D. In addition to activation markers, T cells expressing the conventional anti-CD22 CAR displayed increased amounts of inhibitory receptors PD-1, LAG-3, and TIM-3 on their surface, irrespective of antigenic stimulation, suggesting T cell exhaustion induced by tonic CAR signaling (FIG. 5B). Importantly, co-expressing the anti-CD22 CoCAR completely abolished upregulation of inhibitory receptors on the surface of anti-CD22 CAR-T cells, indicating that co-expressing a CoCAR may prevents T cell exhaustion induced by tonic CAR signaling (FIG. 5B). Notably, when stimulated with CD22-positive tumor cells, T cells co-expressing a CAR and a CoCAR did not show enhanced expression of PD-1, LAG-3 and TIM-3 compared to conventional anti-CD22 CAR-T cells, although such T cells showed enhanced antigen-dependent activation and immune responses. To test if co-expressing a CoCAR with a CAR also prevents T cell differentiation, T cells were analyzed for expression of differentiation markers including CD4, CD8, CD45RA, CD62L, CD95 by flow cytometry analyses. Indeed, T cells co-expressing the anti-CD22 CAR and CoCAR contained a significant higher fraction of CD4-positive cells with an undifferentiated stem-cell memory-like phenotype (indicated as CD45RA and CD62L double-positive cells) compared to T cells expressing the anti-CD22 CAR alone, or to untransduced T cells. A similar trend was seen for CD8-positive T cells, suggesting that co-expressing a CoCAR inhibits CAR-induced T cell differentiation. Taken together, these results clearly demonstrate that co-expressing a CoCAR with an anti-CD22 CAR improves the properties of the therapeutic cells by multiple mechanisms, including the prevention of antigen-independent T cell exhaustion and differentiation, while enhancing immune responses to antigen-expressing cells.

### Example 6: Co-Expressing an Anti-CEA CoCAR Enhances the Anti-Tumor Activity of T Cells Expressing an Anti-EGFR CAR Against Antigen-Low Breast Cancer Cells

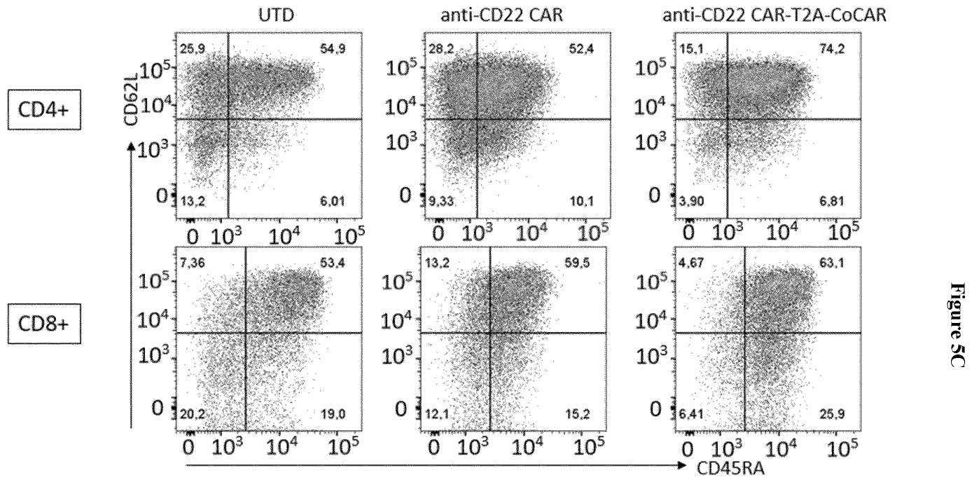

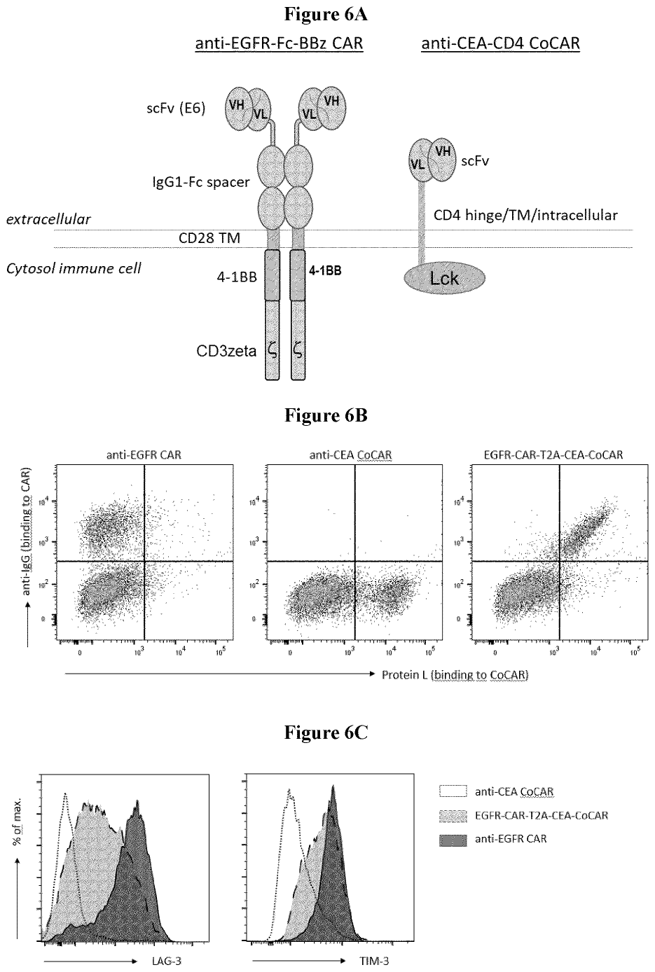

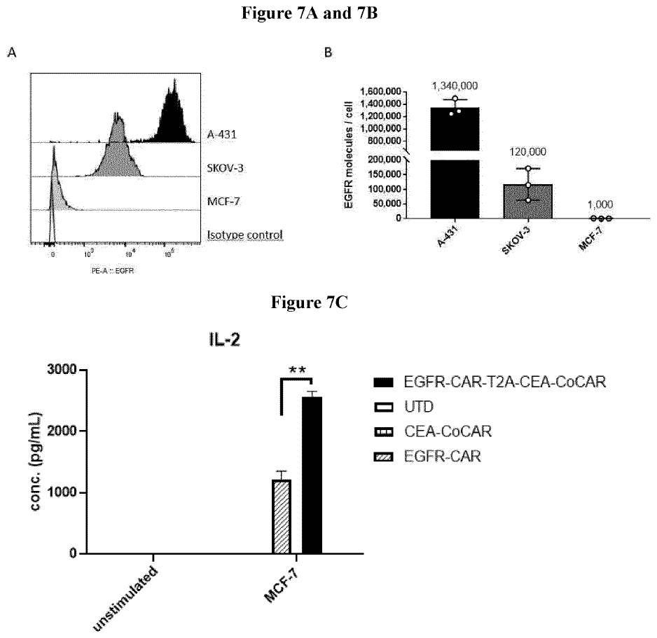

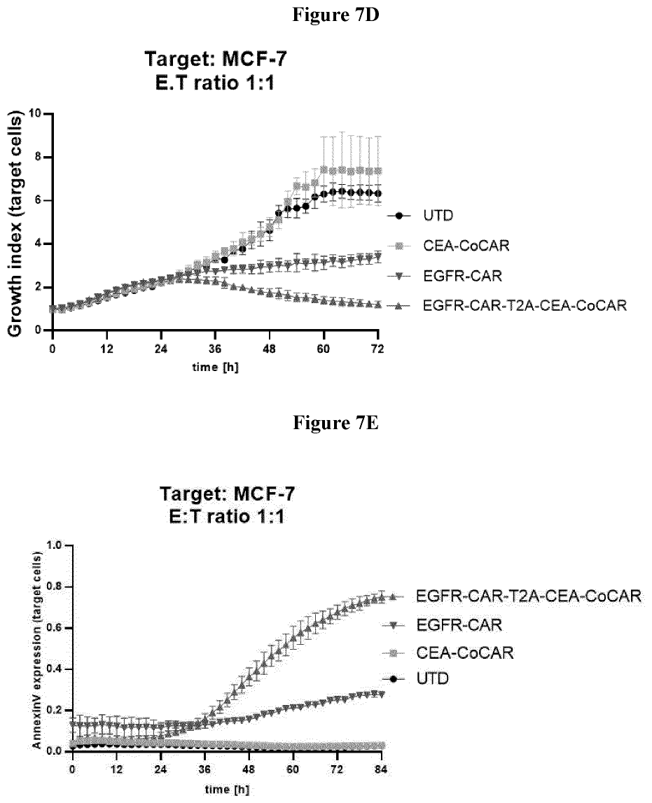

This example describes experiments performed to test the effects of co-expressing a CoCAR with a CAR to enhance T cell activation and function toward antigen-low target cells of a solid tumor. In an exemplary experiment, T cells were transduced to express an Epidermal Growth Factor Receptor (EGFR)-targeting CAR construct containing a human scFv linked to IgG1-Fc spacer, CD28 transmembrane and 4-1BB/CD3ζ signalling domains, with or without a CoCAR containing a carcinoembryonic antigen (CEA)-specific antigen-recognition domain fused to CD4-derived hinge, transmembrane and intracellular domains (FIG. 6A). Both the anti-EGFR CAR and the anti-CEA CoCAR (SEQ ID NO: 108) were expressed on the surface of transduced T cells as determined by flow cytometry (FIG. 6B). Notably, three weeks after transduction, unstimulated anti-EGFR CAR-T cells were highly positive for LAG-3 and TIM-3 expression, indicating tonic signalling of the anti-EGFR CAR. However, T cells co-expressing the anti-EGFR CAR with the anti-CEA CoCAR showed marked less expression of these inhibitory receptors, indicating that CoCAR expression reduces tonic CAR signalling (FIG. 6C), which is in line with the results observed for the anti-CD22 CAR and CoCAR constructs (FIG. 5B). EGFR expression levels were quantified in several target cell lines derived from solid tumors using Quantibrite Phycoerythrin (PE) Quantification Beads (Becton Dickinson). FIG. 7A depicts flow cytometry blots showing EGFR expression in exemplary human cell lines A-431 (squamous carcinoma), SKOV-3 (ovary adenocarcinoma) and MCF-7 (mammary gland adenocarcinoma) expressing very high (>1,300,000), high (>120,000), and low (<1,000) amounts of EGFR molecules on their cell-surface, respectively (FIG. 7B). To test the activity of T cells expressing the anti-EGFR CAR and anti-CEA CoCAR constructs against antigen-low solid tumor cells, T cells were co-cultured with MCF-7 cells, which co-express the EGFR and CEA antigens at low levels on their cell-surface (Abdul Wahid et al, (2014) Molecular Oncology, (2) 337-350). IL-2 production levels were measured by flow cytometry using LEGENDplex Kits (BioLegend) for these T cells 24 hours after incubation, showing that co-expressing the anti-CEA CoCAR significantly enhanced T cell activity induced by the anti-EGFR CAR (FIG. 7C). The capacity of T cells to inhibit the growth of GFP-positive target cells was analyzed in an Incucyte instrument (Sartorius) over a period of 84 hours (FIGS. 7D, E). As shown in FIG. 7D, T cells expressing the anti-EGFR CAR were capable to modestly inhibit the growth of MCF-7 tumor cells compared to T cells expressing the CEA-CoCAR or to untransduced T cells. Strikingly, co-expressing the anti-CEA CoCAR with the anti-EGFR CAR dramatically enhanced T cell activity to counteract tumor cell growth (FIG. 7D), which is based on their enhanced ability to kill the tumor cells (FIG. 7E). Together, these results show that the capacity of enhancing CAR-T cell activity of co-expressing a CoCAR is not limited by specific CAR constructs.

### Example 7: Comparison of CoCAR Constructs with CD8α Hinge and Transmembrane Domains but Different Intracellular Domains Capable of Binding LCK

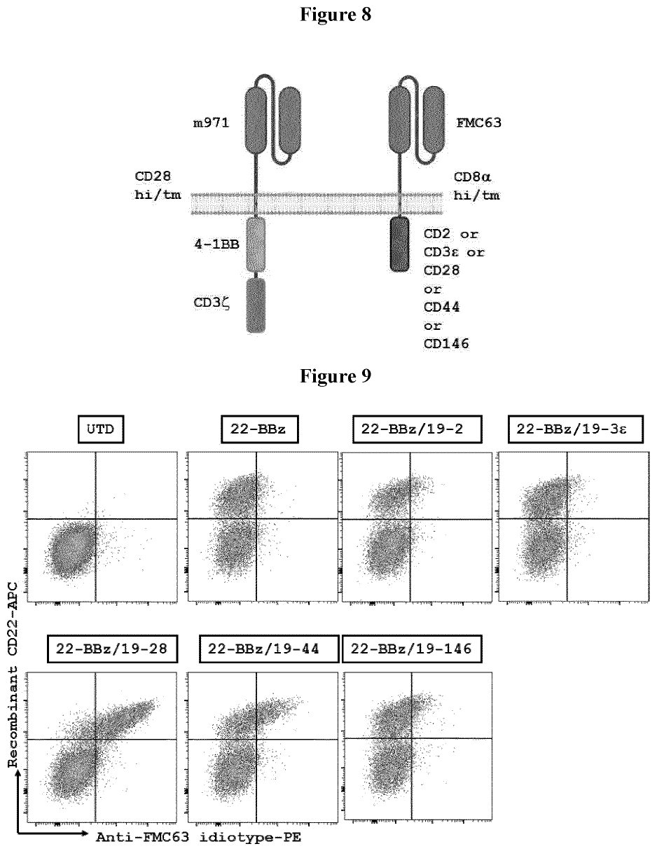

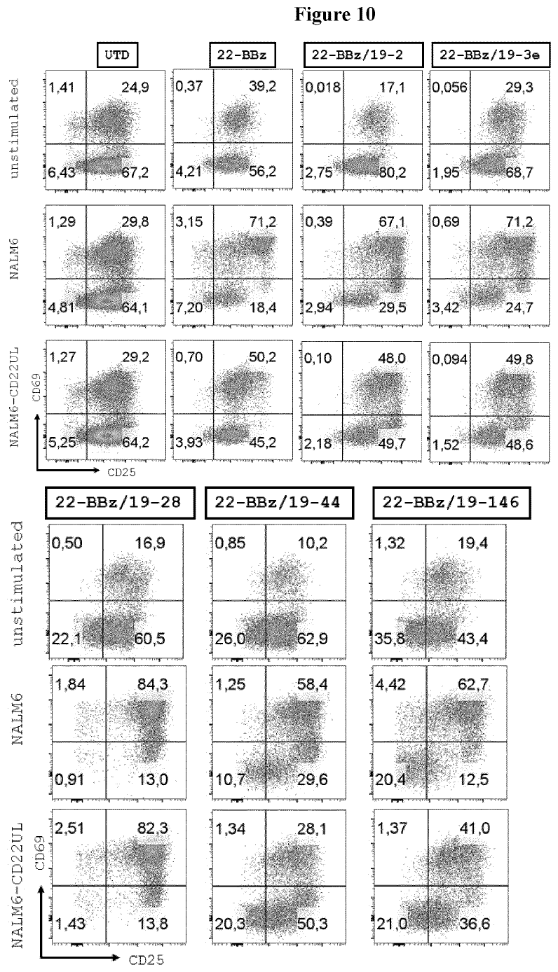

This experiment was performed to determine which intracellular domains capable of binding LCK are useful in CoCARs to enhance CAR-T cell activation and function toward antigen-low tumor cells. The CoCAR constructs contained an anti-CD19 scFv (clone FMC63, SEQ ID NOs: 37-54), CD8α-derived hinge and transmembrane domain, and an intracellular domain derived from CD2, CD3ε, CD28, CD44 or CD146 (SEQ ID NOs: 138-142). Bicistronic constructs were made for expression of an anti-CD22 CAR containing the m971 scFv, CD28-derived hinge and transmembrane domains, and 4-1BB and CD3-zeta signalling domains (designated 22-BBz), and one of the CoCAR polypeptides, where the coding sequences were separated by a T2A sequence. The bicistronic constructs were as follows: 22-BBz (CAR only, SEQ ID NO: 152), 22-BBz/19-2 (anti-CD22 CAR co-expressed with an anti-CD19 CoCAR containing a CD2 intracellular domain, SEQ ID NO: 153), 22-BBz/19-38 (SEQ ID NO: 154), 22-BBz/19-28 (SEQ ID NO: 155), 22-BBz/19-44 (SEQ ID NO: 156) and 22-BBz/19-146 (SEQ ID NO: 157). FIG. 8 illustrates schematically the design of the CAR and CoCAR constructs. None of the CoCAR constructs contains a CD3ζ activating domain, and thus act only in trans to boost the CAR. T cells coexpressing a CAR and CoCAR were generated as described, and resulting T cells were stained with fluorescently labelled recombinant CD22 and anti-FMC63 idiotypic antibody to determine CAR and CoCAR expression by flow cytometry. As shown in FIG. 9, the CoCAR containing a CD28 intracellular domain was efficiently coexpressed with the CAR on the surface of primary human T cells in a 1:1 ratio, as expected. In contrast, the cell-surface expression of the other CoCARs was substantially lower than the 19-28 CoCAR, indicating that the intracellular domain plays an important role in cell-surface deposition of the CoCAR. Nonetheless, none of the CoCAR constructs influenced CAR expression. To test if any of the CoCARs could enhance CAR-T cell activation in response to tumor cells expressing low CD22 antigen densities, each of the CAR-T cell group (100,000 cells) was incubated with wild-type NALM6 cells, or with NALM6 cells expressing ultra-low CD22 densities (NALM6-CD22UL), in an effector:target ratio of 1:1 for 24 hours. After the incubation period, the T cells were harvested and stained for CAR expression and expression of CD69 and CD25 activation markers. As shown in FIG. 10, all CAR-T cell lines were efficiently activated by NALM6 cells expressing 4,000 CD22 molecules/cell and only little, if any, contribution of the CoCAR was detected. In contrast, activation of T cells expressing the conventional CD22 CAR was substantially lower in response to NALM6-CD22UL tumor cells expressing 400 CD22 molecules/cell, and only the CD28-based CoCAR was able to enhance CAR-T cell activation in this antigen-low tumor setting.

To further characterize the effect of coexpressing a CAR and a CoCAR on immune responses toward antigen-low tumor cells, the CAR-T cells (100,000) were co-cultured with an equal number of wild-type or CD22UL NALM6 tumor cells, or with myeloid-lineage K562 tumor cells lacking CD19 and CD22 expression for 24 hours, and the cell-free culture supernatant was analyzed by flow cytometry using the LEGENDPlex Kit (BioLegend) for effector cytokine secretion. As shown in FIG. 11, CAR-T cells having a CoCAR with CD28 intracellular domain outperformed CAR-T cells coexpressing other CoCAR constructs, and CAR-T cells without a CoCAR, in cytokine release against wild-type and CD22UL NALM6 cells, while being unresponsive towards antigen-negative K562 cells. The CD2-CoCAR had little effect on boosting cytokine release by CAR-T cells stimulated by NALM6-CD22UL cells, while the CD44-CoCAR had no effect. CAR-T cells having a CoCAR with CD38 or CD146 intracellular domains also showed enhanced effector cytokine secretion in response to wild-type or CD22UL NALM6 cells, but this was antigen-independent since CD22/CD19-negative K562 cells could also stimulate cytokine secretion by these two CAR-T cell lines.

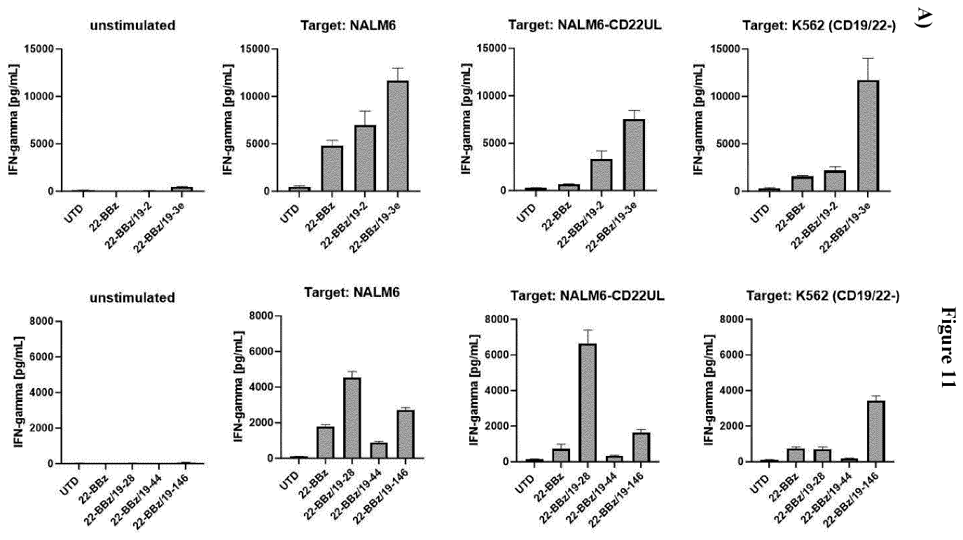

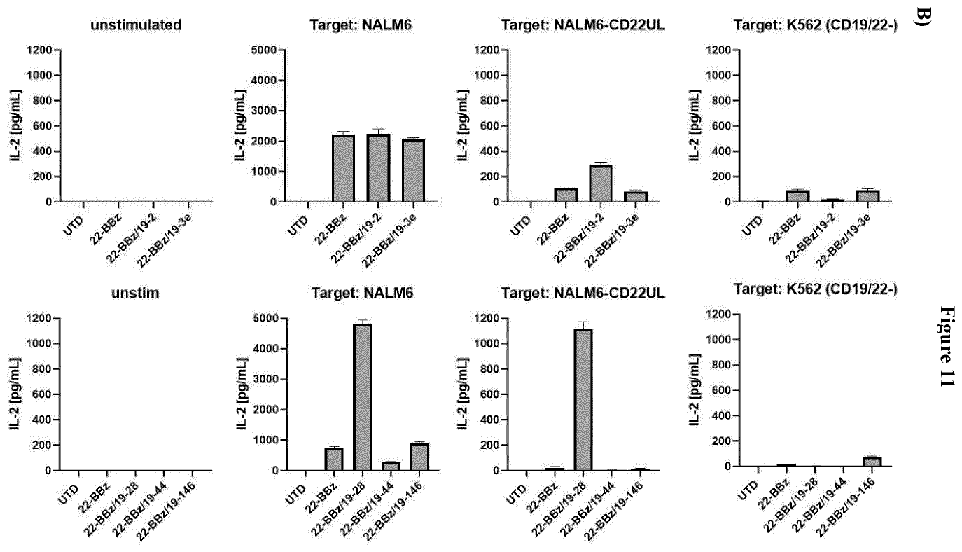

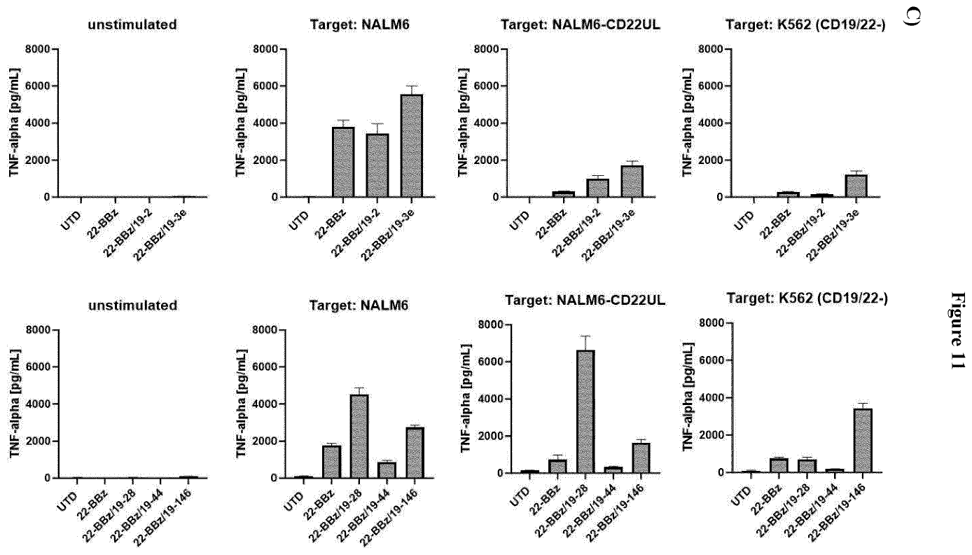

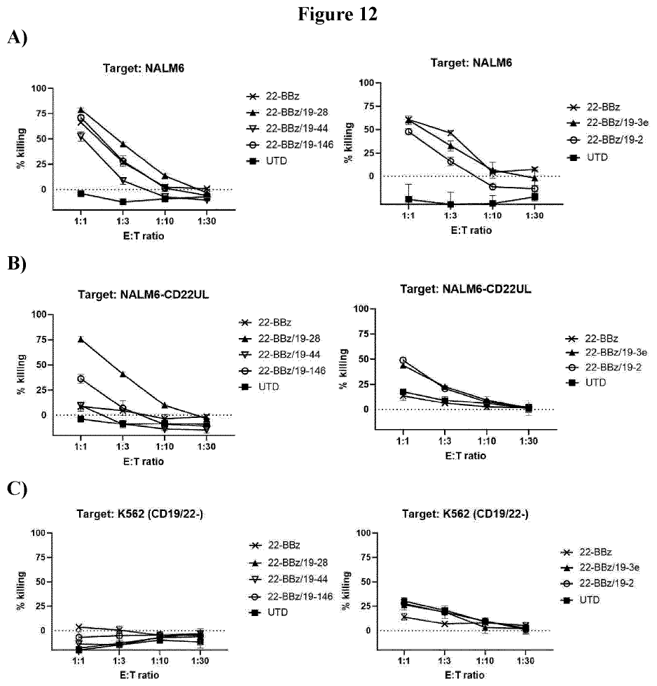

To measure the effect of a coexpressing CoCAR on the cytotoxicity of CD22 CAR-T cells, each of the CAR-T cell lines (50,000 cells) was incubated with firefly-luciferase-expressing wild-type or CD22UL NALM6 cells, or with K562 cells as negative control, at different effector:target ratios in 96-well plates. After 18 hours of co-culture, luciferase substrate (Bright-Glo, Promega) was added to each well, and emitted light was detected in a luminescence plate reader (Tecan). As shown in FIG. 12, all CAR-T cell lines killed wild-type NALM6 cells similarly (FIG. 12A), while only 22-BBz/19-28 CAR-T cells efficiently eliminated NALM6-CD22UL tumor cells (FIG. 12B). CAR-T cells coexpressing a CoCAR with CD2, CD38 or CD146 intracellular domains showed slightly enhanced killing of NALM6-CD22UL tumor cells compared to conventional CD22 CAR-T cells without CoCAR expression, while coexpressing the CD44-CoCAR had no effect on the cytotoxicity of CD22 CAR-T cells. No significant killing of K562 cells above the background seen for untransduced (UTD) T cells was detected for any CAR-T cell line (FIG. 12C), despite induction of unspecific cytokine release by 22-BBz/19-38 and 22-BBz/19-146 CAR-T cells (FIG. 11A-C).

### Example 8: Modification of the Transmembrane Domains Rescued Cell-Surface Expression and Antigen-Induced Functions of CoCARs Containing CD44 or CD146 Intracellular Domains

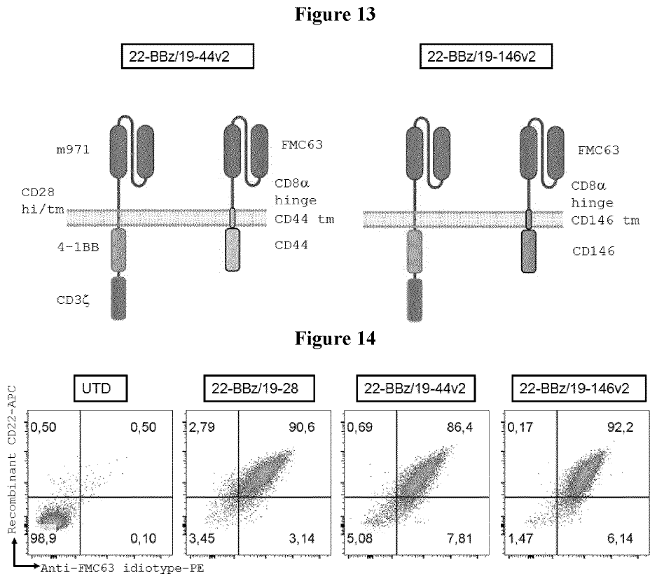

The CoCARs containing CD44 or CD146 intracellular domains showed only weak cell-surface expression on T cells compared to the CoCAR with a CD28 signaling domain (FIG. 9), which may explain their absent or low ability to enhance responsiveness of CAR-T cell to antigen-low tumor cells (FIGS. 10-12). This experiment was performed to test whether the inclusion of the CD44 and CD146 transmembrane domains (e.g. SEQ ID NO: 136, 137) instead of the CD8α transmembrane domain was able to rescue the cell-surface expression of CoCARs with CD44 and CD146 intracellular domains, respectively. FIG. 13 illustrates the domain organization of the combinations 22-BBz/19-44v2 and 22-BBz/19-146v2 encoded by the bicistronic constructs of SEQ ID NO: 165 and 166, respectively.

CAR-T cells were prepared as previously described, and resulting T cells were stained with fluorescently labelled recombinant CD22 and anti-FMC63 idiotypic antibody to detect CAR and CoCAR expression by flow cytometry. As shown in FIG. 14, the modifications of the transmembrane domains restored cell-surface expression of CoCARs containing a CD44 or a CD146 intracellular domain in primary human T cells in a 1:1 ratio with the CAR, as it was observed for 22-BBz/19-28 CAR-T cells. The CAR-T cells were phenotypically characterized by flow cytometry to test for the effect of coexpressing a CAR and a CoCAR on the differentiation of the T cells. As shown in FIG. 15, coexpression of a CD28, CD44 or CD146 CoCAR in addition to a CAR maintained T cell stemness compared to conventional CD22 CAR-T cells, as determined by increased proportions of CD4+CD62L+/CD45RA+ T cells in der CAR/CoCAR-positive population. A similar trend was observed for CD8+ T cells. Without being bound by any theory, this is believed to be due to sequestration of free or membrane-bound LCK molecules in unstimulated CAR-T cells, thereby preventing unspecific CAR-CD3ζ phosphorylation, tonic CAR signalling and T cell differentiation.

To test if 19-44v2 and 19-164v2 CoCARs could enhance 22-BBz CAR function in response to tumor cells expressing ultra-low antigen densities, CAR-T cells were co-cultured with NALM6 tumor cells expressing varying amounts of CD22 on their cell-surface. Wild-type NALM6 cells express approximately 4,000 CD22 molecules per cell and the previously used NALM6-CD22UL clone 3 expressed approximately 400 CD22 molecules per cell. NALM6-CD22UL clone 1 was included in the subsequent functional assays, which expressed only 40 CD22 molecules per cell (FIG. 16A), which is far below the sensitivity limit of conventional 4-1BBz- or 28z-CAR-T cells. Notably, CD19 expression was similar in the different NALM6 clones and was unaffected by CD22 expression (FIG. 16B). Each of the CAR-T cell groups (100,000 cells) was incubated with wild-type NALM6 cells, or with NALM6 cells expressing ultra-low CD22 densities (NALM6-CD22UL cl.3 and cl.1), in an effector:target ratio of 1:1 for 24 hours. After the incubation period, the T cells were harvested and stained for CAR expression and expression of CD69 and CD25 activation markers. As shown in FIG. 17, all CAR-T cell lines were activated by wild-type NALM6 cells expressing 4,000 CD22 molecules/cell, while coexpressing any of the 19-28, 19-44v2 and 19-146v2 CoCARs substantially enhanced T cell activation. Interestingly, CoCAR expression reduced antigen-independent T cell activation mediated by tonic CAR-signaling, which is in line with the increased proportion of undifferentiated CD62L+/CD45RA+ stem cell-like memory T cell subset (Tscm) observed in CoCAR-expressing CAR-T cells (FIG. 15). Stimulation with NALM6-CD22UL3 tumor cells led to weak activation of conventional CAR-T cells, while coexpression of any of the CoCARs enhanced T cell activation. The CD28-based CoCAR was the most efficient polypeptide in enhancing CAR function, followed by the CD44- and CD146-based CoCARs. A similar trend was observed when stimulated with NALM6-CD22UL1 tumor cells expressing only 40 CD22 molecules per cell: conventional 22-BBz CAR-T cells were unresponsive to tumor cells expressing such low antigen densities, as expected, while the CD28-based CoCAR efficiently boosted CAR-T cell activation. The 19-44v2 CoCAR also enhanced CAR-T cell activation in response to NALM6-22ULI cells, albeit to a lesser extend than the 19-28 CoCAR, while the CD146-based CoCAR could not boost CAR-T cell activation in response to 40 CD22 antigens per cell.

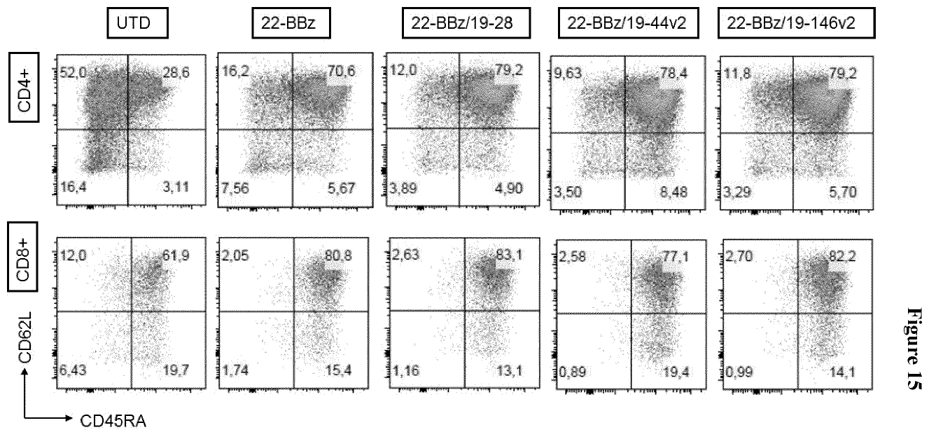

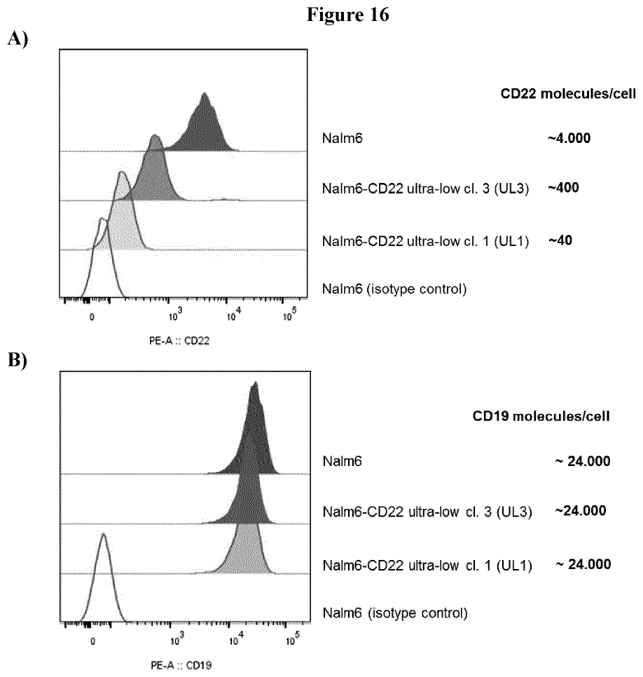

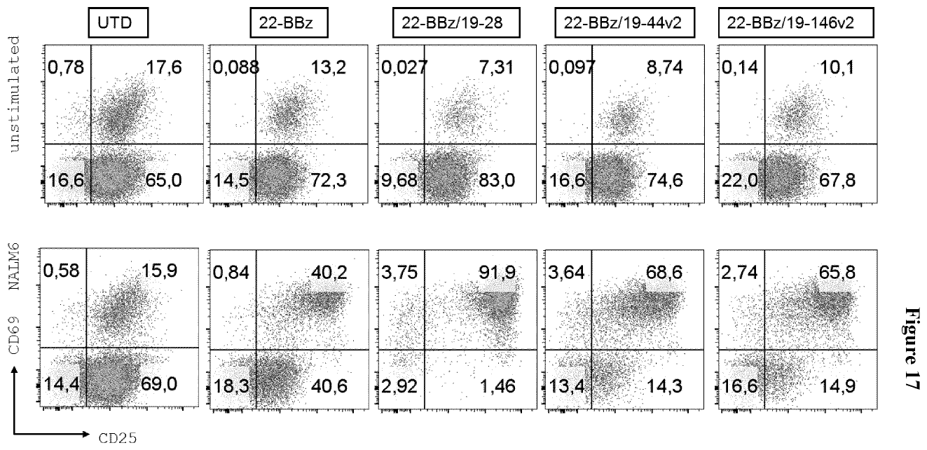

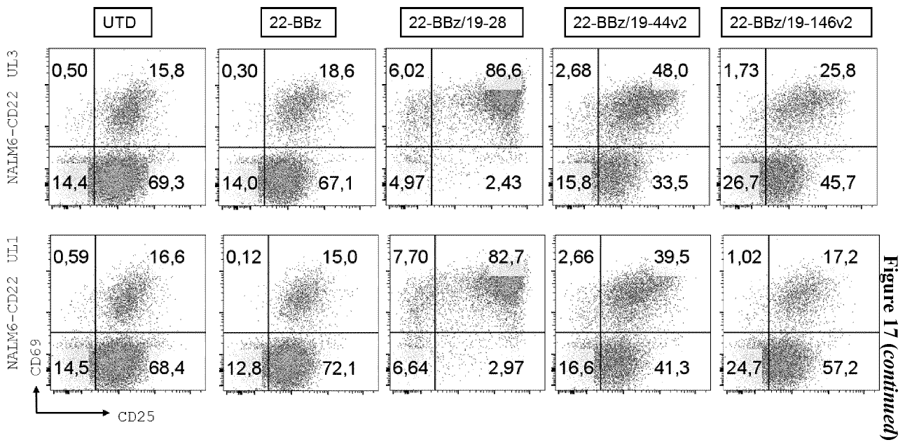

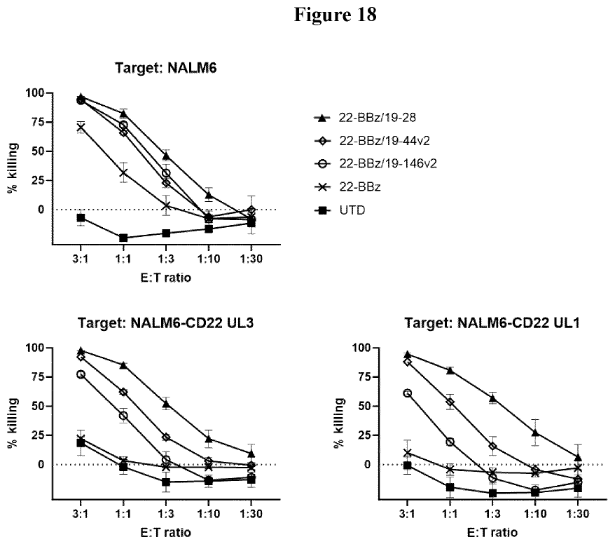

To test if coexpressing the CoCARs enhanced the cytotoxicity of CAR-T cells, each of the CAR-T cell lines (50,000 cells) was incubated with firefly-luciferase-expressing wild-type or CD22 ultra-low NALM6 cell lines at different effector:target ratios in 96-well plates. After 18 hours of co-culture, luciferase substrate (Bright-Glo, Promega) was added to each well, and emitted light was detected in in a luminescence plate reader (Tecan). As shown in FIG. 18, all CAR-T cell lines killed wild-type NALM6 cells efficiently, while only 22-BBz CAR-T cells coexpressing a CoCAR were able to efficiently eliminate NALM6 tumor cells expressing only 400 or 40 CD22 molecules per cell. The 19-28 polypeptide was most efficient in boosting cytotoxicity of 22-BBz CAR-T cells toward antigen ultra-low tumor cells, followed by the 19-44v2 and 19-CD146v2 polypeptides. Notably, conventional 22-BBz CAR-T cells were not able to kill NALM6 tumor cells expressing ultra-low CD22 densities, as expected.

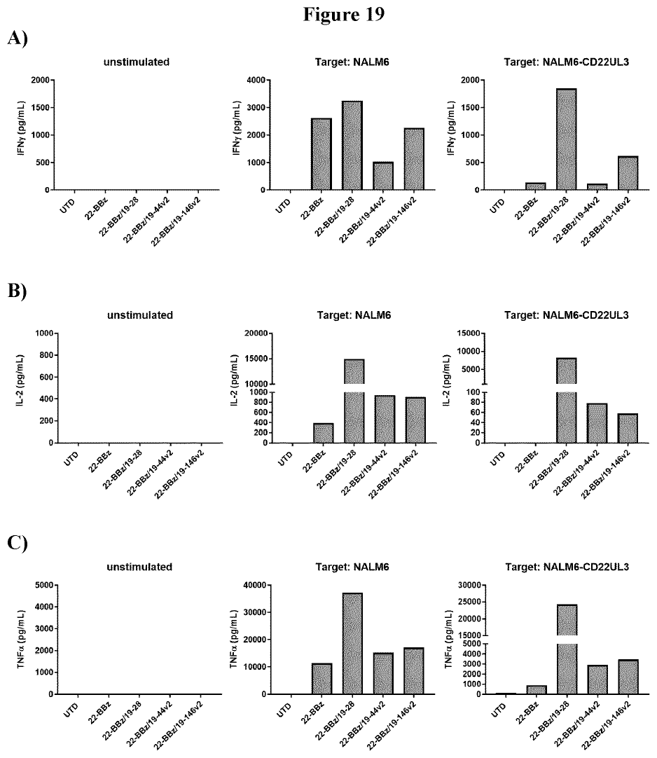

To monitor the release of effector cytokines after stimulation, CAR-T cells (100,000) were co-cultured with an equal number of wild-type or CD22UL3 NALM6 tumor cells, or were left unstimulated, for 24 hours, and the cell-free culture supernatant was examined by a Bio-Plex multiplex assay (Bio-Rad) for cytokine secretion. As shown in FIG. 19, the 19-28 CoCAR significantly enhanced IFNγ (FIG. 19A), TNFα (FIG. 19B) and IL-2 (FIG. 19C) secretion by CD22 CAR-T cells in response to CD22 ultra-low tumor cells. Both 19-44v2 and 19-146v2 CoCARs enhanced TNFα (FIG. 19B) and IL-2 (FIG. 19C) secretion by 22-BBz CAR-T cells in response to CD22 ultra-low tumor cells compared to conventional 22-BBz CAR-T cells, but to a lesser extent than the 19-28 CoCAR. Interestingly, the 19-146v2 CoCAR enhanced IFNγ, TNFα and IL-2 secretion by 22-BBz CAR-T cells after stimulation with NALM6-CD22UL3 cells, while the 19-44v2 CoCAR only enhanced TNFα and IL-2, but not IFNγ secretion. When stimulated with wild-type NALM6 cells, the 19-44v2 CoCAR slightly enhanced TNFα and IL-2 secretion, but substantially reduced IFNγ secretion compared to conventional CAR-T cells, which may be beneficial for the treatment of hematological malignancies, where excessive IFNγ secretion was associated with the onset of toxicities such as cytokine release syndrome.

### Example 9: CoCARs Depend on CAR Signaling to Amplify T Cell Responses

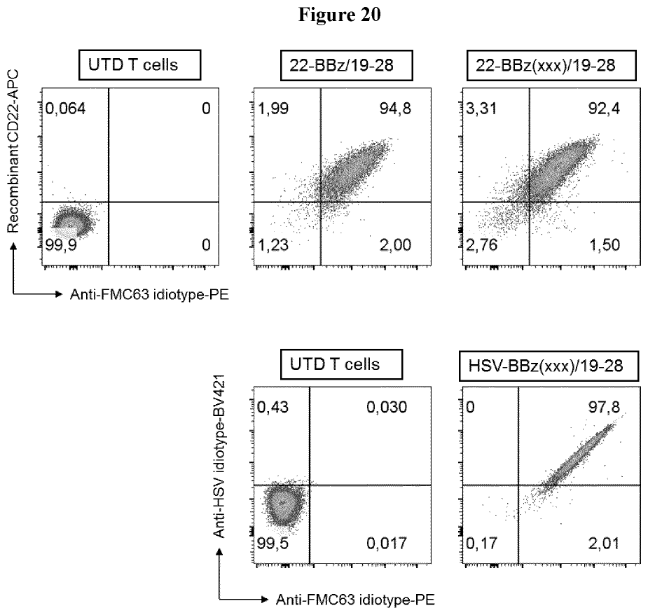

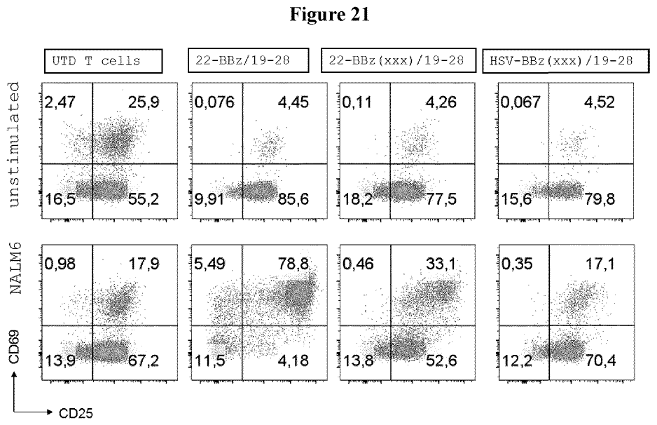

This experiment was performed to study the effect of CoCAR engagement in the absence of CAR signaling on T cell responses. To impede CAR signaling after antigen binding, all six tyrosine residues in the three ITAM motifs within the CAR-CD3ζ activation domain were mutated to phenylalanine, thereby blocking CAR-CD3ζ phosphorylation by SRC-family kinases such as LCK. Such a signaling incompetent CAR, termed 22-BBz(xxx), was coexpressed with the 19-28 CoCAR, the most potent chimeric coreceptor polypeptide, in primary human T cells. As an additional control, the 19-28 CoCAR was coexpressed in T cells with the HSV-BBz(xxx) CAR, which carries the same ITAM mutations and an irrelevant antigen-binding domain specific for the gB protein of Herpes-Simplex-Virus (HSV) 1 and 2. The mutant CARs were similarly expressed as the non-mutated CAR in lentivirally transduced human T cells, as determined by flow cytometry (FIG. 20). The CAR-T cells (100,000) were co-cultured with an equal number of NALM6 tumor cells for 24 hours. After the incubation period, the T cells were harvested and stained for CAR expression and expression of CD69 and CD25 activation markers. As shown in FIG. 21, 22-BBz/19-28 CAR-T cells were efficiently activated by tumor cells, and T cell activation was markedly diminished in CAR-CD3ζ ITAM mutated 22-BBz(xxx)/19-28 CAR-T cells. Weak but notable CAR-T cell activation was detected in NALM6-stimulated 22-BBz(xxx)/19-28 CAR-T cells compared to HSV-BBz(xxx)/19-28 CAR-T cells, with the latter experienced only CoCAR engagement, indicating residual signaling induced by the 4-1BB endodomain or the ITAM-mutated CD3z domain of the 22-BBz(xxx) CAR, or T cell activation mediated by the increased avidity resulting from the expression of two antigen-binding domains (anti-CD22 and anti-CD19).

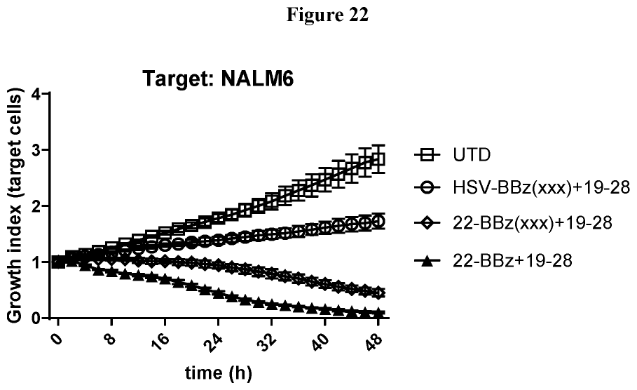

To test if 19-28 CoCAR engagement promotes CAR-T cell cytotoxicity in the absence of CAR signaling, each of the CAR-T cell lines (50,000 cells) was incubated with GFP-expressing NALM6 cells at an effector:target ratio of 1:1 in 96-well plates, and the growth of GFP-positive tumor cells was monitored in an IncuCyte instrument (Sartorius) for 48 hours. As shown in FIG. 22, 22-BBz/19-28 CAR-T cells eliminated NALM6 cells after 48 hours, while cytotoxicity was markedly reduced when the ITAMs of the CAR were mutated. However, 22-BBz(xxx)/19-28 CAR-T cells retained some cytotoxicity toward NALM6 cells compared to HSV-BBz(xxx)/19-28 CAR-T cells, which may be due to the increased avidity of 22-BBz(xxx)/19-28 CAR-T cells for NALM6 cells.

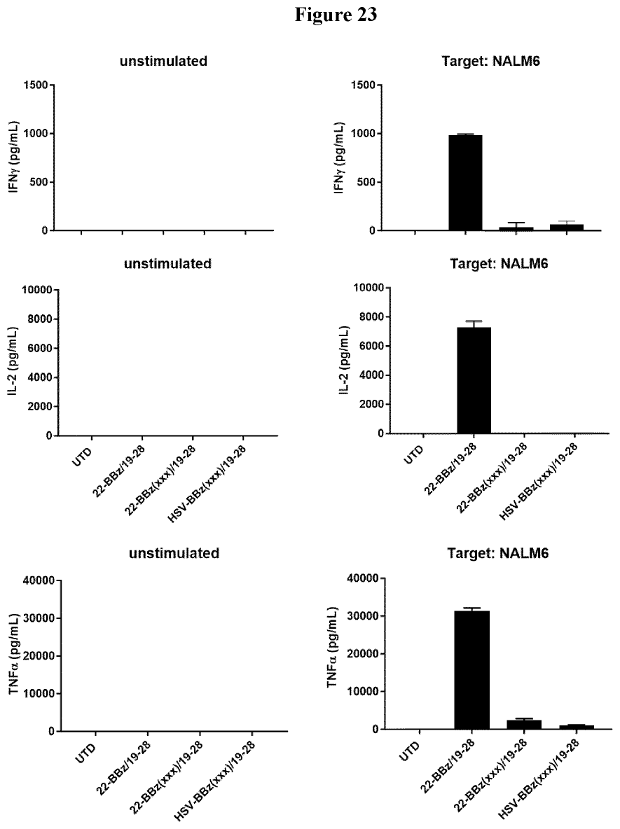

To test if 19-28 CoCAR engagement promotes cytokine release by CAR-T cells in the absence of CAR signaling, CAR-T cells (100,000) were co-cultured with an equal number of NALM6 tumor cells, or were left unstimulated, for 24 hours, and the cell-free culture supernatant was assayed for cytokine secretion by flow cytometry using a LegendPlex assay (BioLegend). As shown in FIG. 23, 22-BB/19-28 CAR-T cells secreted high amounts of effector cytokines IFNγ, TNFα and IL-2, while cytokine secretion was abrogated in 22-BBz(xxx)/19-28 and HSV-BBz(xxx)/19-28 CAR-T cells. In summary, these results suggest that 19-28 CoCAR engagement alone, without CAR engagement, does not lead to T cell activation or T cell immune responses.

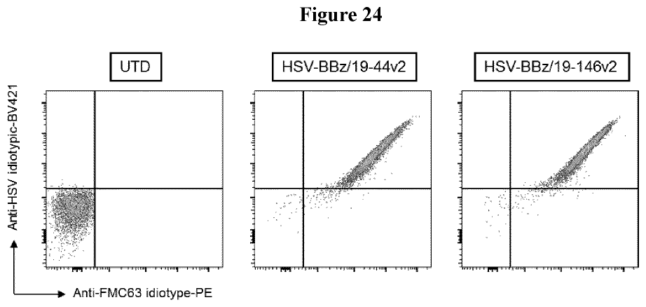

To test whether engagement of CD44- and CD146-based CoCARs promotes T cell activation and immune responses in the absence of CAR engagement, the CoCARs were coexpressed with the anti-gB HSV CAR HSV-BBz in primary human T cells. FIG. 24 shows coexpression of the HSV-BBz CAR and the 19-44v2 and 19-146v2 CoCARs on the surface of transduced T cells. The CAR-T cells (100,000) were co-cultured with an equal number of NALM6 tumor cells for 24 hours. After the incubation period, the T cells were harvested and stained for CAR expression and expression of CD69 and CD25 activation markers. As shown in FIG. 25, engagement of 19-44v2 or 19-146v2 CoCARs did not lead to T cell activation in the absence of CAR engagement.

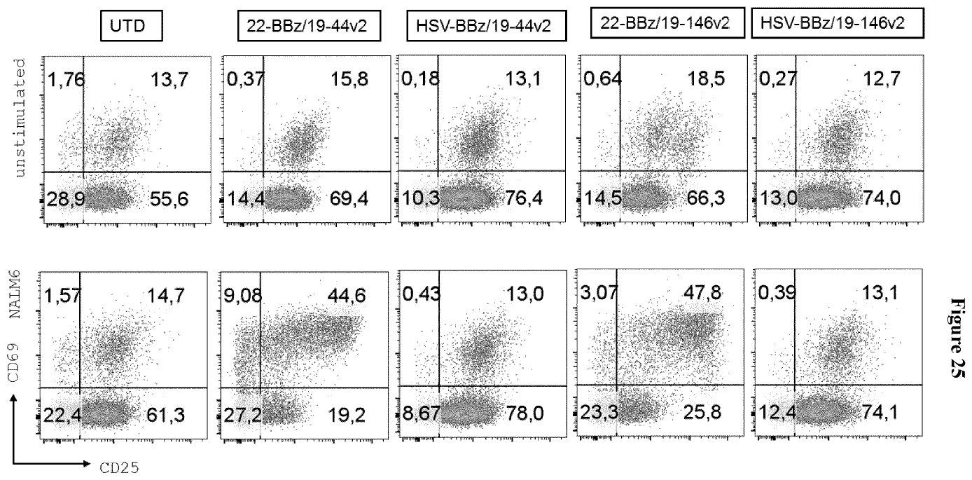

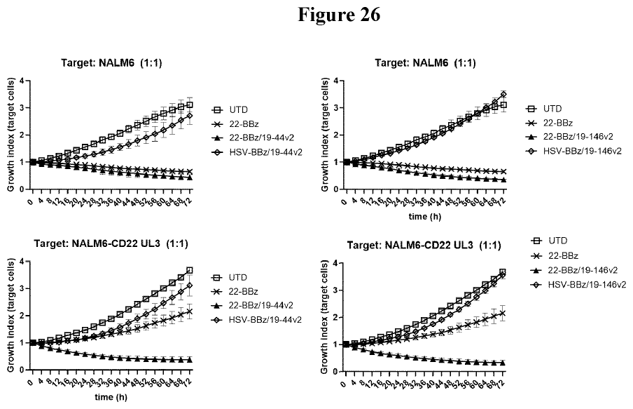

To further test if 19-44v2 or 19-146v2 CoCAR engagement promotes CAR-T cell cytotoxicity in the absence of CAR engagement, each of the CAR-T cell lines (50,000 cells) was incubated with GFP-expressing wild-type or CD22UL cl.3 NALM6 cells at an effector:target ratio of 1:1 in 96-well plates, and the growth of GFP-positive tumor cells was monitored in an IncuCyte instrument (Sartorius) for 72 hours. As shown in FIG. 26, 22-BBz, 22-BBz/19-44v2 and 22-BBz/19-44v2 CAR-T cells eliminated NALM6 cells, while only 22-BBz CAR-T cells that coexpressed a 19-44v2 or 19-146v2 CoCAR were able to eliminate NALM6-CD22UL tumor cells. In contrast, CoCAR engagement in HSV-BBz/19-44v2 and HSV-BBz/19-146v2 CAR-T cells did not induce cytotoxicity toward wild-type or CD22UL cl.3 NALM6 cells.

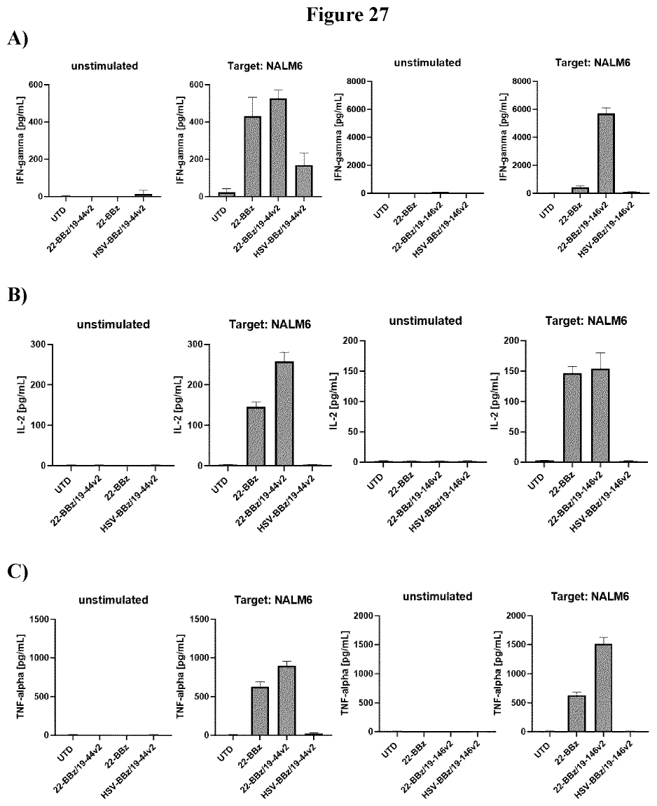

To test if 19-44v2 or 19-146v2 CoCAR engagement promotes cytokine release by CAR-T cells in the absence of CAR engagement, CAR-T cells (100,000) were co-cultured with an equal number of NALM6 tumor cells, or were left unstimulated, for 24 hours, and the cell-free culture supernatant was assayed for cytokine secretion by flow cytometry using a LegendPlex assay (BioLegend). As shown in FIG. 27, 19-44v2 CoCAR expression did not enhance secretion of IFNγ by 22-BBz CAR-T cells, while IFNγ secretion was substantially reduced in the absence of concomitant CAR engagement (FIG. 27A). In contrast, 19-44v2 CoCAR expression enhanced secretion of TNFα and IL-2 by 22-BBz CAR-T cells, while cytokine secretion was abrogated in the absence of concomitant CAR engagement (FIG. 27B, C). 19-146v2 CoCAR expression significantly enhanced IFNγ and TNFα secretion by 22-BBz CAR-T cells when stimulated with NALM6 cells (FIG. 27A, C), while IL-2 secretion was unaffected (FIG. 27B). Importantly, engagement of the 19-146v2 CoCAR did not induce detectable IFNγ, TNFα and IL-2 secretion in the absence of CAR stimulation.

In summary, these results suggest that engagement of 19-28, 19-44v2 and 19-146v2 CoCARs does not lead to T cell activation or T cell immune responses in the absence of concomitant CAR engagement.

### Example 10: CAR-CoCAR-T Cells Targeting Different Epitopes in the Same Antigen

This experiment was performed to study the effect of a CoCAR targeting a different epitope of the same antigen targeted by a CAR. The m971 and SGIII (a humanized version of the RFB4 antibody) scFv target a membrane proximal and a distal epitope of CD22, respectively. In this example, CoCAR constructs comprising the SGIII antigen-binding domain (containing a (G₄S)₃ scFv linker (i.e. Linker L1, SEQ ID NO: 10)) were used in combination with CAR constructs comprising m971 (containing a Whitlow scFv linker, SEQ ID NO: 185). To better match the distances of the CAR and CoCAR polypeptides to the different epitopes, a long IgG1-Fc (hinge-CH2-CH3, SEQ ID NOs: 75-77) spacer was used in the CAR construct, while the CoCARs contained a shorter CD8α hinge domain, i.e. SEQ ID NO: 85.

CAR-T cells were prepared as described, and CAR and CoCAR expression was assessed by flow cytometry using fluorescently labelled anti-Whitlow-Linker and anti-G4S-linker antibodies (Cell Signaling), respectively. As shown in FIG. 28, CAR and CoCAR molecules were efficiently expressed on transduced primary human T cells.

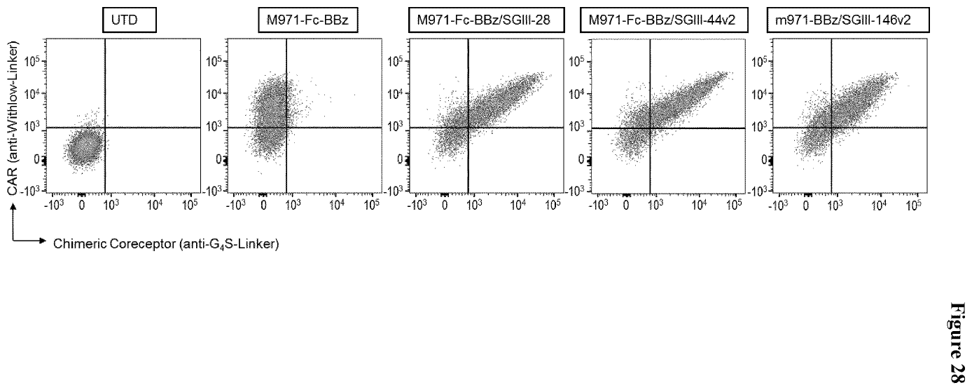

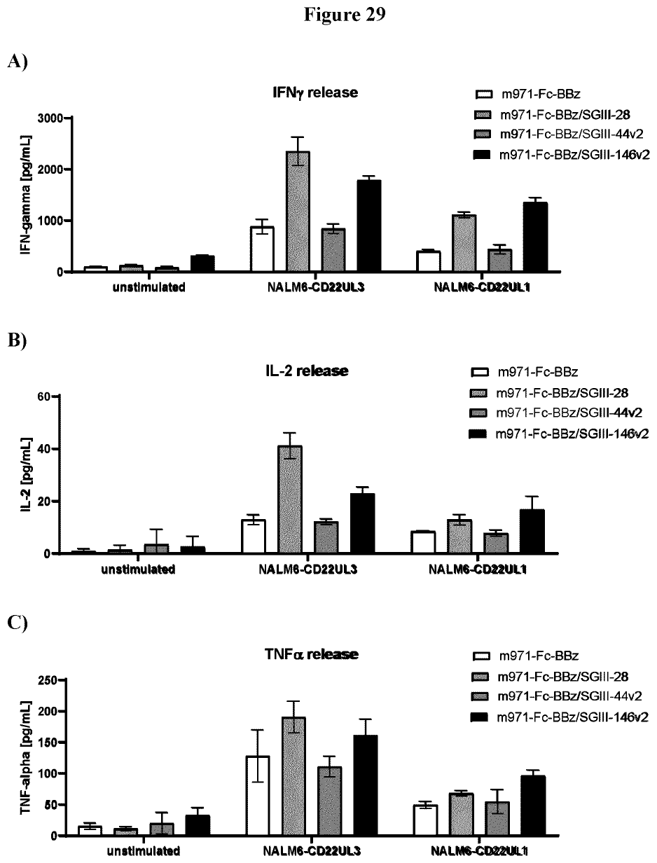

Each group of the CAR-T cells (100,000) was co-cultured with an equal number of NALM6-CD22UL3 or NALM6-CD22UL1 tumor cells expressing 400 and 40 CD22 molecules per cell, respectively, for 24 hours, and the cell-free culture supernatant was assayed for effector cytokine secretion by FACS using a LEGENDPlex multiplex assay kit (BioLegend). As shown in FIG. 29, co-expression of the SGIII-28 (SEQ ID NO: 188) or SGIII-146 CoCAR (SEQ ID NO: 190) enhanced effector cytokine secretion by m971-BBz CAR-T cells in response to the CD22 ultra-low tumor cells compared to conventional anti-CD22 CAR-T cells, while the SGIII-44v2 CoCAR (SEQ ID NO: 189) was not able to enhance cytokine secretion.

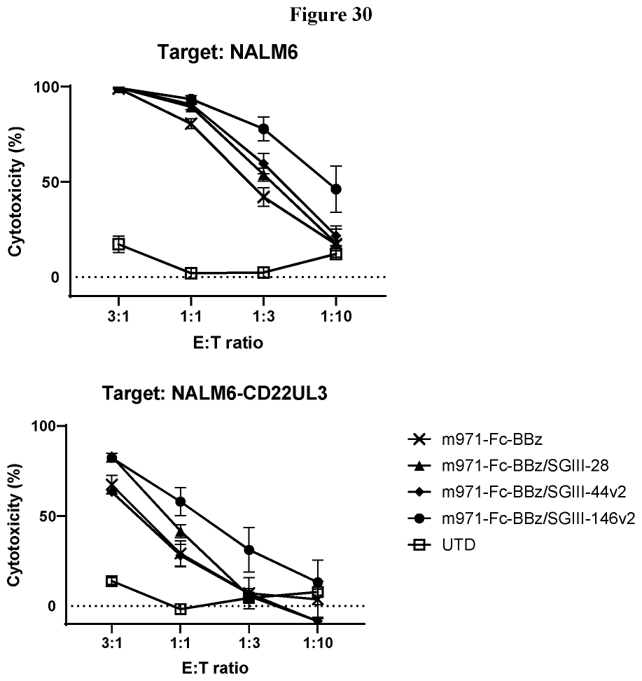

To measure the effect of co-expression of an anti-CD22-CoCAR on the cytotoxicity of anti-CD22-CAR-T cells, each of the CAR-T cell lines was incubated with firefly luciferase-expressing wild-type or CD22UL3-NALM6 cells at different effector:target ratios in 96-well plates. After 18 hours of co-culture, luciferase substrate (Bright-Glo, Promega) was added to each well, and the emitted light was detected in a luminescence plate reader (Tecan). As shown in FIG. 30, all CAR-T cell groups were able to efficiently kill wild-type NALM6 cells, while co-expression of one of the CoCARs slightly enhanced tumor cell killing. In contrast, conventional anti-CD22-CAR-T cells showed reduced killing of CD22-ultralow tumor cells, and co-expression of SGIII-CD28 and SGIII-CD146v2 CoCARs enhanced killing, while the SGIII-44v2 CoCAR had no effect. In conclusion, these results show that CoCAR constructs targeting the same antigen as the CAR construct can enhance the antitumor response of CAR-T cells.

### Example 11: Antitumor Activity of Anti-HER2 CAR T Cells Coexpressing an Anti-EGFR CoCAR Against Solid Tumor Cells

This experiment was performed to demonstrate the effect of co-expression of a CoCAR on the efficacy of CAR-T cells against tumor cells from solid tumors.

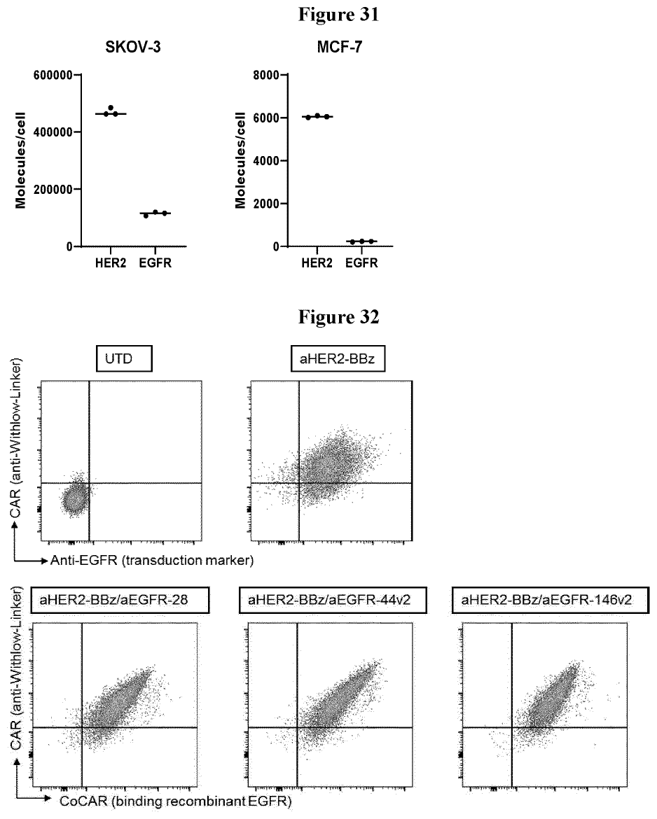

For the experiments, the tumor cell lines SKOV-3 (ovarian adenocarcinoma) and MCF-7 (breast adenocarcinoma) were used, which represent solid tumor models expressing high (about 46,000 HER2 and about 120,000 EGFR molecules per cell) and low (about 6,000 HER2 and about 250 EGFR molecules per cell) antigen densities of the tumor-associated antigens HER2 and EGFR, respectively (FIG. 31). Bicistronic constructs were made for expression of an anti-HER2 CAR (SEQ ID NO: 187) having a scFv targeted to the HER2 antigen (scFv sequence as in WO2017079694A2; leader sequence=SEQ ID NO: 167), CD28-derived hinge/transmembrane and 4-1BB/CD3ζ intracellular signaling domains, and an anti-EGFR CoCAR having the structure CD28, CD44v2, or CD146v2. CAR-T cells expressing these constructs were prepared as described and, as shown in FIG. 32, CAR and CoCAR constructs were efficiently expressed on primary human T cells.

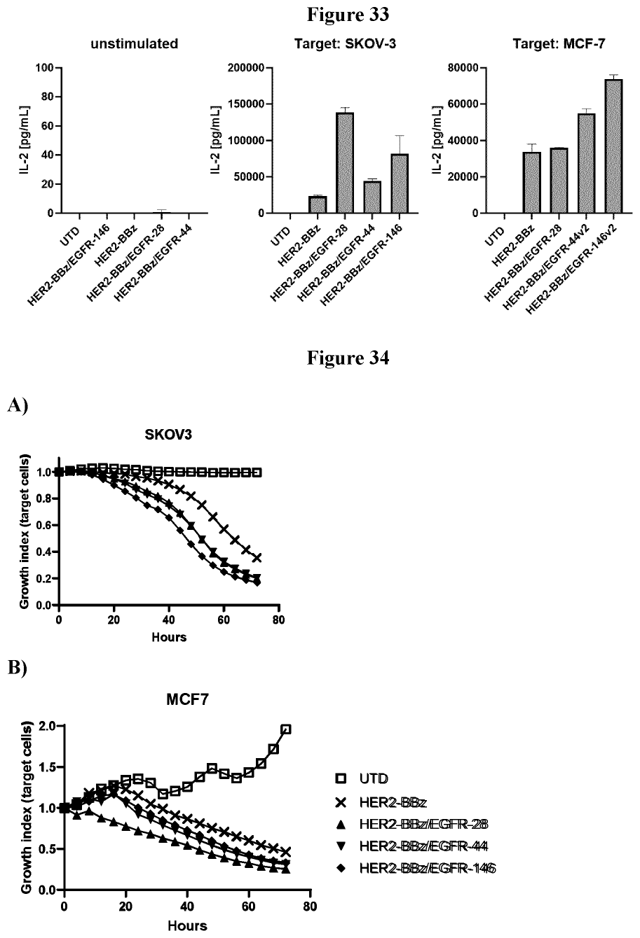

CAR-T cells (100,000) were co-cultured with an equal number of SKOV-3 or MCF-7 tumor cells, or were left unstimulated, for 24 hours, and the cell-free culture supernatant was assayed for IL-2 secretion by ELISA (ACROBiosystems). As shown in FIG. 33, anti-HER2 CAR-T cells secreted IL-2 in response to both tumor cell lines, and co-expression of a CoCAR enhanced IL-2 secretion.

To investigate the cytotoxicity of anti-HER2 CAR-T cells with or without co-expression of a CoCAR, each of the CAR-T cell lines (50,000 cells) was incubated with GFP-expressing SKOV-3 or MCF-7 tumor cells at an effector:target ratio of 1:1 in 96-well plates, and the growth of GFP-positive tumor cells was monitored for 72 hours in an IncuCyte instrument (Sartorius). As shown in FIG. 34, anti-HER2 CAR T cells showed cytotoxicity against both cell lines, and cytotoxicity was enhanced when a CoCAR was co-expressed. This example shows that the CoCARs CD28, CD44v2, CD146v2, comprising, CD28, CD44 and CD146 intracellular domains, respectively, are capable to boost the function of CAR-T cells against solid tumors with high and low antigen expression.

### REFERENCES

Abdul Wahid et al., (2014) Molecular Oncology, (2) 337-350
Borroto et al., (2014) J Immunol, (5) 2042-2053
Brudno et al., (2018) J Clin Oncol, (22) 2267-2280
Cappell & Kochenderfer, (2021) Nat Rev Clin Oncol. (11) 715-727
Cohen et al., (2019) J Clin Invest, (6) 2210-2221
Eshhar et al., (1993) PNAS, (90) 720-724
Fry et al., (2017) Nat Med, (1) 20-28
Hamieh et al., (2019) Nature, (568) 112-116
Hartl et al., (2020) Nat Immunol, (8) 902-913
Hartl et al., (2021) Cells, (4) 834
Haso et al, (2013) Blood, (7) 1165-1174
Heitzeneder et al, (2022) Cancer Cell, (1) 53-69
Helsen et al., (2018) Nat Commun, (1) 3049
Kagoya et al., (2018) Nat Med. (3) 352-359
Kawalekar et al, (2016) Immunity, (2) 380-390
Kim et al, (2003) Science, (5640) 1725-1728
Li et al., (2018) JCI Insight, (18) 121322
Long et al., (2015) Nat Med, (6) 581-590
Majzner et al., (2020) Cancer Discov. (5) 702-723
Melenhorst et al., (2022) Nature, (602) 503-509
Neelapu et al., (2017) N Engl J Med, (377) 2531-2544
Salter et al., (2018) Sci Signal. 11(544)
Salter et al., (2021) Sci Signal. (697) 2606
Shah et al., (2015) Pediatr Blood Cancer, (6) 964-969
Shah et al., (2020) J Clin Oncol. (17) 1938-1959
Singh et al., (2021) Nat Med. (5) 842-850
Sun et al., (2020) Cancer Cell, (2) 216-225
Ying et al., (2019) Mol Ther Oncolytics, (15) 60-68
Zhao et al, (2020) Mol Ther Oncolytics, (18) 272-281

1. A coreceptor-CAR (CoCAR), comprising
(i) a first extracellular antigen-recognition domain,
(ii) a first hinge domain,
(iii) a first transmembrane domain, and
(iv) at least one cytosolic domain
capable of binding LCK;
wherein the CoCAR does not comprise a CD3zeta domain.

2. The CoCAR according to claim 1, wherein the first antigen-recognition domain is a scFv.

3. The CoCAR according to claim 1, capable of specifically binding to an antigen on a target cell.

4. The CoCAR according to claim 1, wherein the first hinge domain is derived from a CD4, CD8, CD28, IgG-Fc domain.

5. The CoCAR according to claim 1, wherein the first transmembrane domain is derived from a CD4, CD8, CD28, CD44, CD146 or CD3zeta transmembrane domain.

6. (canceled)

7. The CoCAR according to claim 5, wherein the motif capable of binding LCK is derived from a CD28, CD44, CD146, CD4, CD8α, or CD3ε intracellular signaling domain.

8. A combination of
(a) the CoCAR of claim 1, and
(b) a chimeric antigen receptor (CAR).

9. A nucleic acid or a combination of a first and second nucleic acid, wherein
(a) the nucleic acid encodes the CoCAR according to claim 1 or the combination of claim 8, or
(b) the combination of a first and second nucleic acid encodes the combination of claim 8, wherein the first nucleic acid encodes the CoCAR (a), and the second nucleic acid encodes the CAR (b).

10. A nucleic acid construct or a combination of a first and second nucleic acid construct, wherein
(a) the nucleic acid construct comprises the nucleic acid or the combination of a first and second nucleic acid of claim 9, wherein the nucleic acid and/or the first and second nucleic acids are functionally linked to at least one expression control sequence, and
(b) in the combination of the first and second nucleic acid construct, the first nucleic construct comprises the first nucleic acid of claim 9, and the second nucleic construct comprises the second nucleic acid of claim 9, wherein the first and second nucleic acids independently are functionally linked to at least one expression control sequence.

11. A vector or a combination of a first and second vector, wherein
(a) the vector comprises the nucleic acid or a combination of a first and second nucleic acid of claim 9 and/or the nucleic acid construct or combination of nucleic acid constructs of claim 10, and/or
(b) in the combination, the first vector comprises the first nucleic acid and/or the first nucleic acid construct, and the second vector comprises the second nucleic acid and/or the second nucleic acid construct of claims 9 and 10.

12. A recombinant cell, comprising the nucleic acid construct according to claim 10.

13. The recombinant cell of claim 12, which is an immune effector cell.

14. The recombinant cell of claim 12, expressing the CoCAR and/or the combination of claim 8 on the cell surface.

15. The recombinant cell of claim 12, wherein (i) the CAR and (ii) the CoCAR recognize different epitopes located in the same antigen expressed on the surface of a target cell.

16. The recombinant cell of claim 12, wherein (i) the CAR and (ii) the CoCAR recognize different antigens expressed on the surface of a target cell.

17. A pharmaceutical composition comprising
(i) the CoCAR of claim 1,
(ii) the combination of claim 8,
(iii) the nucleic acid or the combination of nucleic acids of claim 9, the nucleic acid construct or the combination of nucleic constructs of claim 10, the vector or the combination of vectors according to claim 11, and/or
(iv) the recombinant cell according to claim 12,
and a pharmaceutically acceptable carrier.

18-21. (canceled)

22. A method of treating cancer in a subject in need thereof, comprising the step of administering to the subject an effective amount of
(i) the CoCAR according to claim 1,
(ii) the combination of claim 8,
(iii) the nucleic acid or the combination of nucleic acids according to claim 9, the nucleic acid construct or the combination of nucleic constructs according to claim 10, the vector or the combination of vectors according to claim 11, and/or
(iv) the recombinant cell according to claim 12.

23. The method according to claim 22, wherein the cancer is a malignancy positive for at least one tumor-associated antigen selected from the group consisting of B7-H3 (CD276), BCMA, CD3, CD5, CD19, CD20, CD22, CD23, CD30, CD33, CD38, CD44, CD44v6, CD52, CD70, CD79A, CD79B, CD123, CD138, CD171, CEA, Claudin-6, Claudin-18.2, CLL1, CXCR5, EGFR, EGFRvIII, EPH-receptor A2, IGLV3-21, GP-2, GP-40, HER2, ErbB3, ErbB4, FBP, AchR, Fr-a, GD2, GD3, HMW-MAA, IL13Ra2, Kappa-LC, IGLV3-21-R110, Lewis Y, Mesothelin, MUC1, MUC16, NKG2D Ligands, NCAM, NY-ESO1, Oncofetal antigen h5T4, PSCA, PSMA, ROR1, TAG-72, VEGFR, GOLPH2, and SLAMF7.

24. The method according to claim 22, wherein the cancer is a B cell malignancy.

25. The CoCAR according to claim 1, wherein the at least one cytosolic domain (a) capable of binding LCK
(1) is an intracellular signaling domain derived from a CD28, CD44, CD146, CD4, CD8α, or CD3ε intracellular signaling domain, or a variant or fragment thereof, or
(2) comprises one or more motifs capable of binding LCK.
